(19) United States
(12) Patent Application Publication
(10) Pub. No.: US 2009/0215872 A1
(43) Pub. Date: Aug. 27, 2009

(54) Methods for Controlling Stem Cell Differentiation

(76) Inventor: Jeannie T. Lee, Weston, MA (US)

Correspondence Address: CLARK & ELBING LLP, 101 FEDERAL STREET, BOSTON, MA, 02110, US

(21) Appl. No.: 11/988,321

(22) PCT Filed: Jun. 30, 2006
(86) PCT No.: PCT/US2006/025800

§ 371 Date: Mar. 23, 2009

Related U.S. Application Data
(60) Provisional application No. 60/697,301, filed on Jul. 7, 2005.

(51) Int. Cl. C12N 15/09; C12N 5/10; C07H 21/02; A61K 31/7088; C12N 15/63; A61P 43/00

(52) U.S. Cl. 514/44 R; 435/377; 435/366; 536/23.1; 435/320.1

(57) ABSTRACT

Disclosed herein are methods for controlling stem cell differentiation through the introduction of transgenes having Xic, Tsix, or Xite sequences to block differentiation and the removal of the transgenes to allow differentiation. Also disclosed are small RNA molecules and methods for using the small RNA molecules to control stem cell differentiation. Also disclosed are stem cells genetically modified by the introduction of Xic, Tsix, or Xite sequences.

### STATEMENT AS TO FEDERALLY SPONSORED RESEARCH

This invention was funded in part by grant number RO1 GM58839 from the National Institutes of Health. The government may have certain rights in the invention.

### BACKGROUND OF THE INVENTION

The present invention features improvements for the development and maintenance of mammalian stem cells and their derivatives.

Stem cells are unique cell populations that have the ability to divide (self-renew) for indefinite periods of time, and, under the right conditions or signals, to differentiate into the many different cell types that make up an organism. Stem cells derived from the inner cell mass of the blastocyst are known as embryonic stem (ES) cells. Stem cells derived from the primordial germ cells, and which normally develop into mature gametes (eggs and sperm) are known as embryonic germ (EG) cells. Both of these types of stem cells are known as pluripotent cells because of their unique ability to differentiate into derivatives of all three embryonic germ layers (endoderm, mesoderm, and ectoderm).

The pluripotent stem cells can further specialize into another type of multipotent stem cell often derived from adult tissues. Multipotent stem cells are also able to undergo self-renewal and differentiation, but unlike embryonic stem cells, are committed to give rise to cells that have a particular function. Examples of adult stem cells include hematopoietic stem cells (HSC), which can proliferate and differentiate to produce lymphoid and myeloid cell types, bone marrow-derived stem cells (BMSC), which can differentiate into adipocytes, chondrocytes, osteocytes, hepatocytes, cardiomyocytes and neurons, and neural stem cells (NSC), which can differentiate into astrocytes, neurons, and oligodendrocytes. Multipotent stem cells have also been derived from epithelial and adipose tissues and umbilical cord blood (UCB).

A considerable amount of interest has been generated in the fields of regenerative medicine and gene therapy by recent work relating to the isolation and propagation of stem cells. The ability of stem cells to be propagated indefinitely in culture combined with their ability to generate a variety of tissue types makes the therapeutic potential from these cells almost limitless.

One of the major limitations in the development of stem cells for therapeutic purposes concerns the regulation of the transition from self-renewal to differentiation for a sufficient time to allow the clinician or researcher to manipulate the cells for therapeutic or research purposes. Current methods used for maintaining stem cells in the undifferentiated state include growing the cells on a feeder layer of mouse embryonic fibroblast cells, culturing in bovine serum, culturing in a plate-coating matrix of cells extracted from mouse tumors, and adding reagents such as leukemia inhibitory factor, fibroblast growth factor (FGF), the Map kinase kinase inhibitor PD 98059, and Oct-4 (also known as Oct-3/4). All of these methods are limited in their potential because of their inefficiency in blocking differentiation and because of the potential contamination with animal products, pathogens, feeder cells, or, in the case of human stem cells, contamination with non-human cells.

Improved methods for the growth and manipulation of undifferentiated stem cells are needed to help realize the full therapeutic potential of these cells.

### SUMMARY OF THE INVENTION

The present invention is based on the discovery that X-chromosome inactivation (XCI) enables differentiation in stem cells and that inhibiting or blocking XCI can result in a block to differentiation, thereby providing a mechanism for controlling differentiation of stem cells. Such methods include targeting and inactivating any of the endogenous genes within the X-inactivation center locus or introducing transgenes that can prevent the cells from undergoing X chromosome inactivation. The use of these methods to control stem cell differentiation facilitates and enhances the therapeutic and clinical potential of stem cells.

XCI is the process in which one X-chromosome is shut off in the female cell (XX) to compensate for having an extra X-chromosome as compared to the male (XY) cell. This means that every embryo must be equipped with a mechanism to count X-chromosomes (XX vs. XY), and then randomly choose between two X-chromosomes in the female to start the inactivation process while maintaining the same X-chromosome inactive in all later divisions. The steps are respectively known as “counting,” “choice,” and “silencing.” In addition, interchromosomal pairing is also involved in the XCI process.

These steps are controlled by a master regulatory region called the X-inactivation center (Xic), which contains a number of unusual noncoding genes that work together to ensure that XCI takes place only in the XX female, only on one chromosome, and in a developmentally specific manner. At the Xic, three noncoding genes, Xist, Tsix, and Xite, are involved in this process and each makes RNA instead of protein. Xist is made only from the future inactive X and makes a 20 kb RNA that “coats” the inactive X, thereby initiating the process of gene silencing. Tsix is the antisense regulator of Xist and acts by preventing the spread of Xist RNA along the X-chromosome. Thus, Tsix designates the future active X. Xite works together with Tsix to ensure the active state of the X. Xite makes a series of intergenic RNAs and assumes special chromatin conformation. Its action enhances the expression of antisense Tsix, thereby synergizing with Tsix to designate the future active X. Together Tsix and Xite control the “choice” step, while Xist controls the “silencing” step. Tsix and Xite also regulate counting and mutually exclusive choice through X-X pairing.

The present invention is based on the discovery that disruptions in the XCI process, either by an excess or a depletion of Xic, Tsix, and Xite, can block differentiation. In the present methods, disruptions in the XCI process are achieved through the use of transgenes or small RNAs derived from Xic, Tsix or Xite sequences, or fragments thereof, that are introduced into stem cells and prevent the stem cells from undergoing X chromosome inactivation and from differentiating in culture. Removal of the transgene reverses the block to differentiation and the stem cells can be induced to differentiate as desired. These methods allow the clinician or investigator sufficient time to manipulate the stem cells as needed to enhance their therapeutic potential in the absence of contamination with cells or animal products. The use of small RNA molecules circumvents the need for removal of the transgene because the small RNA molecules have a limited half-life and will naturally degrade. The methods of the invention also reduce or eliminate the need to use feeder cells which also results in cells that are more suitable for therapeutic purposes due to the reduced likelihood of contamination by feeder cells. Thus, these methods and the cells produced from these methods overcome two of the major limitations to stem cell research.

Accordingly, in a first aspect the invention features a method for delaying differentiation of a stem cell that includes introducing into the stem cell at least one transgene selected from the group consisting of an Xic transgene, a Tsix transgene, an Xite transgene, a Tsix/Xite transgene, and any fragments thereof.

In another aspect, the invention features a method of controlling differentiation of a stem cell that includes the steps of (a) introducing into the stem cell at least one transgene selected from the group consisting of an Xic transgene, a Tsix transgene, an Xite transgene, a Tsix/Xite transgene, and fragments thereof, thereby delaying differentiation of the stem cell and (b) when desired, inactivating the transgene thereby allowing differentiation of the stem cell. In this method the transgene can further include a selectable marker. The transgene can also be flanked by recombinase recognition sequences including but not limited to LoxP or FRT sequences. In step (b) of the method, inactivating the transgene can include removing the transgene from the stem cell, for example by expression of a recombinase (e.g., Cre recombinase or flippase (FLP) recombinase) in the stem cell to remove the transgene from the genomic DNA or to remove an episome containing the transgene (e.g., by deleting the origin of replication). In preferred embodiments, the recombinase expression is transient. The method can also include the introduction of a second transgene into the stem cell prior to the inactivation step. If desired, more than one additional transgene can be introduced into the stem cell prior to the inactivation step.

In another aspect, the invention features a method for delaying differentiation of a stem cell that includes introducing into the stem cell a small RNA substantially identical to or complementary to at least 15 nucleotides of a transgene selected from the group consisting of an Xic transgene, a Tsix transgene, an Xite transgene, a Tsix/Xite transgene, an Xist transgene, and any fragments thereof. The small RNA molecule can be a double stranded RNA or an siRNA molecule. The small RNA is at least 15 nucleotides, preferably, 17, 18, 19, 20, 21, 22, 23, 24, 25, 26, 27, 28, 29, 30, 31, 32, 33, 34, or 35, nucleotides in length and even up to 50 or 100 nucleotides in length (inclusive of all integers in between). Desirably, the small RNA molecule is 15 to 32 nucleotides in length.

For any of the above aspects, preferred Xic transgenes include any nucleic acid sequence substantially identical to SEQ ID NOs: 1, 2, 3, 39, or any fragments thereof. Preferred Tsix transgenes include any nucleic acid sequence substantially identical to SEQ ID NOs: 5, 6, 9, 10, 12, 13, 14, 21, 22, 23, 28, 29, 30, 31, 32, 36, 40, or any fragments thereof. Particularly preferred Tsix transgenes include the nucleic acid sequences set forth in SEQ ID NOs: 9, 10, 12, 21, 22 and 28-32. Additional preferred Tsix transgenes include at least one copy, at least two copies, at least three copies, at least four copies, and at least five copies of any of SEQ ID NOs: 13, 14, 28-32, or 40. Preferred Xite transgenes include any nucleic acid sequence substantially identical to SEQ ID NOs: 15, 16, 17, 24, 25, 26, 27, 38, or any fragments thereof. Preferred Tsix/Xite transgenes include any nucleic acid sequence substantially identical to SEQ ID NOs: 4, 11, 19, 37, or any fragments thereof. Preferred transgenes for any of the above regions can inhibit endogenous X-X pairing, for example, by inducing de novo pairing between the X and the transgene, as assayed using the methods described herein.

Any of the transgenes can be used in combination with any additional transgene. In one example, SEQ ID NO: 23 can be used in combination with any of the additional transgenes to enhance the block to differentiation. In addition, the transgenes can be used as a single copy or as a multimer (e.g., multiple copies or a tandem array of the sequence). For example, SEQ ID NOs: 13, 14, 28-32, and 40 are particularly useful as multimers.

In preferred embodiments of the above aspects, the stem cell is an embryonic stem cell, desirably a female embryonic stem cell. Mammalian embryonic stem cells or embryonic stem cells from any agricultural animal are particularly useful in the methods of the invention. In preferred embodiments the stem cell is a human or mouse embryonic stem cell. The stem cell can be an embryonic stem cell at any stage, preferably a blastocyst stage stem cell, an embryonic germ cell, or a cloned stem cell from a somatic nuclei.

In another aspect, the invention features a stem cell that includes an Xic transgene substantially identical to a nucleic acid sequence set forth in SEQ ID NOs: 1, 2, 3, 39, or any fragments thereof.

In yet another aspect, the invention features a stem cell that includes a Tsix transgene substantially identical to a nucleic acid sequence set forth in SEQ ID NOs: 5, 6, 9, 10, 12, 13, 14, 21, 22, 23, 28-32, 36, 40, or any fragments thereof.

In yet another aspect, the invention features a stem cell that includes an Xite transgene substantially identical to a nucleic acid sequence set forth in SEQ ID NOs: 15, 16, 17, 24, 25, 26, 27, 38, or any fragments thereof.

In yet another aspect, the invention features a stem cell that includes a Tsix/Xite transgene substantially identical to a nucleic acid sequence set forth in SEQ ID NOs: 4, 11, 19, 37, or any fragments thereof.

In preferred embodiments of the above aspects, the transgene is expressed in the stem cell. Desirably, the stem cell is an embryonic stem cell, which can be male or female, preferably a female embryonic stem cell. Mammalian embryonic stem cells or embryonic stem cells from any agricultural animal are particularly useful in the methods of the invention. In preferred embodiments the stem cell is a human or mouse embryonic stem cell. The stem cell can be an embryonic stem cell at any stage, preferably a blastocyst stage stem cell, an embryonic germ cell, or a cloned stem cell from a somatic nuclei.

For any of the stem cells of the invention, the cell transgene can further include a selectable marker or be flanked by LoxP or FRT sequences. The stem cells of the invention can also include a recombinase (e.g., Cre or FLP recombinase), preferably one that is expressed transiently. Any of the stem cells of the invention can also further include a second transgene, or if desired additional transgenes.

In another aspect, the invention features an isolated small RNA molecule comprising a nucleic acid sequence substantially identical to or complementary to at least 15 nucleotides of a transgene selected from the group consisting of an Xic transgene, a Tsix transgene, an Xite transgene, a Tsix/Xite transgene, an Xist transgene, or any fragments thereof. The small RNA molecule can be a double stranded RNA or an siRNA molecule, and is at least 15 nucleotides, preferably, 17, 18, 19, 20, 21, 22, 23, 24, 25, 26, 27, 28, 29, 30, 31, 32, 33, 34, or 35 nucleotides in length and even up to 50 or 100 nucleotides in length (inclusive of all integers in between). In one embodiment, the small RNA molecule is an siRNA 15 to 32 nucleotides in length.

In a related aspect, the invention features a composition that includes the small RNA molecule described above formulated to facilitate entry of the small RNA into a cell. In another aspect, the isolated small RNA molecule described above is in a pharmaceutical composition. The pharmaceutical composition can further include a pharmaceutically acceptable carrier. The invention also features a vector that includes the small RNA molecule, wherein the small RNA molecule is operably linked to one or more transcriptional regulatory sequences.

For either of the above aspects relating to small RNAs, the RNA molecule is substantially identical to or complementary to preferred Xic transgenes, which include any nucleic acid sequence substantially identical to SEQ ID NOs: 1, 2, 3, 39, or any fragments thereof. Preferred Tsix transgenes include any nucleic acid sequence substantially identical to SEQ ID NOs: 5, 6, 9, 10, 12, 13, 14, 21, 22, 23, 28, 29, 30, 31, 32, 36, 40, or any fragments thereof. Particularly preferred Tsix transgenes include the nucleic acid sequences set forth in SEQ ID NOs: 9, 10, 12, 21, 22 and 28-32. Preferred Xite transgenes include any nucleic acid sequence substantially identical to SEQ ID NOs: 15, 16, 17, 24, 25, 26, 27, 38, or any fragments thereof. Preferred Tsix/Xite transgenes include any nucleic acid sequence substantially identical to SEQ ID NOs: 4, 11, 19, 37, or any fragments thereof. Preferred Xist transgenes include any nucleic acid sequence substantially identical to or complementary to SEQ ID NOs: 7, 8, 20, and 35.

By “stem cell” is meant any cell with the potential to self-renew and, under appropriate conditions, differentiate into a dedicated progenitor cell or a specified cell or tissue. Stem cells can be pluripotent or multipotent. Stem cells include, but are not limited to embryonic stem cells, embryonic germ cells, a cloned stem cell from a somatic nuclei, adult stem cells, and umbilical cord blood cells.

By “adult stem cell” or “somatic stem cell” is meant an undifferentiated cell found in a differentiated tissue that can renew itself and (with certain limitations) differentiate to yield all the specialized cell types of the tissue from which it originated. Adult stem cells are multipotent. Non-limiting examples of adult stem cells include hematopoietic stem cells, bone marrow-derived stem cells, and neural stem cells (NSC), as well as multipotent stem cells derived from epithelial and adipose tissues and umbilical cord blood (UCB).

By “embryonic stem cell” is meant a cell, derived from an embryo at the blastocyst stage, or before substantial differentiation of the cell into the three germ layers, that can self-renew and that displays morphological characteristics of undifferentiated cells, distinguishing them from differentiated cells of embryonic or adult origin. Exemplary morphological characteristics include high nuclear/cytoplasmic ratios and prominent nucleoli under a microscope. Under appropriate conditions known to the skilled artisan, embryonic stem cells can differentiate into cells or tissues that are derivatives of each of the three germ layers: endoderm, mesoderm, and ectoderm. Assays for identification of an embryonic stem cell include the ability to form a teratoma in a suitable host or to be stained for markers of an undifferentiated cell such as Oct-4.

By “differentiation” is meant the process whereby an unspecialized early embryonic cell acquires the features of a specialized cell such as a heart, liver, bone, nerve, or muscle cell. Differentiation can also refer to the restriction of the potential of a cell to self-renew and is generally associated with a change in the functional capacity of the cell. The terms “undifferentiated,” or “delaying” or “blocking” differentiation, are used broadly in the context of this invention and include not only the prevention of differentiation but also the altering or slowing of the differentiation process of a cell. It will be understood by the skilled artisan that colonies of undifferentiated cells can often be surrounded by neighboring cells that are differentiated; nevertheless, the undifferentiated colonies will persist when the population is cultured or passaged under appropriate conditions, and individual undifferentiated cells will constitute a substantial portion (e.g., at least 5%, 10%, 20%, 40%, 60%, 80%, 90% or more) of the cell population. Differentiation of a stem cell can be determined by methods well known in the art and these include analysis for cell markers or morphological features associated with cells of a defined differentiated state. Examples of such markers and features include measurement of glycoprotein, alkaline phosphatase, and carcinoembryonic antigen expression, where an increase in any one of these proteins is an indicator of differentiation Additional examples are described herein. In preferred embodiments, if less than 10%, 5%, 4%, 3%, 2%, or 1% of the cells in a population express a marker or morphological feature of differentiation after an established number of days in culture (e.g., 2, 3, 4, 5, 6, or 7 days or more), then the cells are undifferentiated. Differentiation can also be determined by assays for X chromosome inactivation. Examples of such assays are described herein and include measurement of Xist expression by fluorescent in situ hybridization (FISH) or RT-PCR or measurement of interchromosomal distances by FISH (X-X pairing). In one example, if after an established number of days in culture (e.g., 2, 3, 4, 5, 6, or 7 days or more), fewer than 20%, 15%, 10%, 5%, 4%, 3%, 2%, or 1% of the cells in a population show an increase in Xist expression as measured by FISH or RT-PCR or show X-X pairing as measured by FISH, then the cells are undifferentiated.

By “fragment” is meant a portion of a nucleic acid molecule that contain at least 1%, 5%, 10%, 20%, 30%, 40%, 50%, 60%, 70%, 80%, or 90% of the entire length of the reference nucleic acid molecule. In the present invention, a fragment includes any fragment of the X inactivation center (Xic) that includes at least 10, 17, 18, 19, 20, 21, 22, 23, 24, 25, 26, 27, 28, 29, 30, 31, 32, 33, 34, 35, 40, 50, 60, 68, 70, 80, 90, 100, 200, 300, 400, 500, 600, 700, 800, 900, 1000, 1500, 2000, 3000, 3700, 4000, 5000, 10,000, 15,000, 19,500, 20,000, or more nucleotides up to the entire length of the Xic (approximately 100 kB). Preferred fragments are described herein and are shown in Tables 1 and 2 and FIGS. 1, 2, 3A, 3B, and 30B. One preferred fragment is a small RNA nucleic acid sequence, often called siRNA, which can serve as a specificity determinant in the RNA interference (RNAi) pathway.

“RNAi,” also referred to in the art as “gene silencing” and/or “target silencing”, e.g., “target mRNA silencing”), refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes. The unifying features of RNA silencing phenomena are the production of small RNAs, at least 15 nt in length, preferably 15-32 nt, most preferably 17 to 26 nt in length, that act as specificity determinants for down-regulating gene expression and the requirement for one or more members of the Argonaute family of proteins (or PPD proteins, named for their characteristic PAZ and Piwi domains). Recently it has been noted that larger siRNA molecules, for example, 25 nt, 30 nt, 50 nt, or even 100 nt or more, can also be used to initiate RNAi. (See for example, Girard et al., Nature Jun. 4, 2006, e-publication ahead of print, Aravin et al., Nature Jun. 4, 2006, e-publication ahead of print, Grivna et al., Genes Dev. Jun. 9, 2006, e-publication ahead of print, and Lau et al., Science Jun. 15, 2006, e-publication ahead of print.)

The term “small RNA” is used throughout the application and refers to any RNA molecule, either single-stranded or double-stranded” that is at least 15 nucleotides, preferably, 17, 18, 19, 20, 21, 22, 23, 24, 25, 26, 27, 28, 29, 30, 31, 32, 33, 34, or 35, nucleotides in length and even up to 50 or 100 nucleotides in length (inclusive of all integers in between). Preferably, the small RNA is capable of mediating RNAi. As used herein the phrase “mediates RNAi” refers to (indicates) the ability to distinguish which RNAs are to be degraded by the RNAi machinery or process. Included within the term small RNA are “small interfering RNAs” and “microRNA.” In general, microRNAs (miRNAs) are small (e.g., 17-26 nucleotides), single-stranded noncoding RNAs that are processed from approximately 70 nucleotide hairpin precursor RNAs by Dicer. Small interfering RNAs (siRNAs) are of a similar size and are also non-coding, however, siRNAs are processed from long dsRNAs and are usually double stranded (e.g., endogenous siRNAs). siRNAs can also include short hairpin RNAs in which both strands of an siRNA duplex are included within a single RNA molecule. Small RNAs can be used to describe both types of RNA. These terms include double-stranded RNA, single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the small RNA or internally (at one or more nucleotides of the RNA). Nucleotides in the RNA molecules of the present invention can also comprise non-standard nucleotides, including non-naturally occurring nucleotides or deoxyribonucleotides. Small RNAs of the present invention need only be sufficiently similar to natural RNA that it has the ability to mediate RNAi.

By the process of “genetic modification” or “genetic alteration” is meant the introduction of an exogenous gene or foreign gene into mammalian cells. The term includes but is not limited to transduction (viral mediated transfer of host DNA from a host or donor to a recipient, either in vivo or in vitro), transfection, liposome mediated transfer, electroporation, calcium phosphate transfection or coprecipitation. Methods of transduction include direct co-culture of cells with producer cells or culturing cells with viral supernatant alone with or without appropriate growth factors and polycations.

The term “identity” is used herein to describe the relationship of the sequence of a particular nucleic acid molecule to the sequence of a reference nucleic acid molecule. For example, if a nucleic acid molecule has the same nucleotide residue at a given position, compared to a reference molecule to which it is aligned, there is said to be “identity” at that position. The level of sequence identity of a nucleic acid molecule to a reference nucleic acid molecule is typically measured using sequence analysis software with the default parameters specified therein, such as the introduction of gaps to achieve an optimal alignment (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). These software programs match identical or similar sequences by assigning degrees of identity to various substitutions, deletions, or other modifications.

A nucleic acid molecule is said to be “substantially identical” to a reference molecule if it exhibits, over its entire length, at least 51%, preferably at least 55%, 60%, or 65%, and most preferably 75%, 85%, 90%, 95%, 96%, 97%, 98%, 99% or even 100% identity to the sequence of the reference molecule. For nucleic acid molecules, the length of comparison sequences is at least 10, 17, 18, 19, 20, 21, 22, 23, 24, 25, 26, 27, 28, 29, 30, 31, 32, 33, 34, 35, 40, 50, 60, 68, 70, 80, 90, 100, 200, 300, 400, 500, 600, 700, 800, 900, 1000, 1500, 2000, 3000, 3700, 4000, 5000, 10,000, 15,000, 19,500, 20,000, or more nucleotides up to and including the entire length of the Xic (approximately 100 kB for the mouse Xic).

It should be noted that while protein-coding genes that are homologous generally share a significant level of homology (generally greater than 70%), the overall level of homology for noncoding genes and cis regulatory elements, such as the regions included in the present invention, is generally less than 60%. For example, the same Xic from different strains of mice have sequence variation on the order of one nucleotide change per 100 nucleotides. In another example, for the DxPas 34 repeats, the repeat length varies from strain to strain from 15-40 nucleotides. In yet another example, within Xite in particular, the sequence variation between strains can include basepair insertions, deletion, and single nucleotide polymorphisms. Furthermore, homology for noncoding genes and cis regulatory elements is often limited to smaller domains (e.g., 30 to 100 nt in length). As a result, more sensitive methods such as PipMaker analysis and Bayesian block analysis can be used to measure the homology or identity of a particular noncoding gene region or cis regulatory element (Schwartz et al., Genome Research 10: 577-586 (2000)).

By “inactivating the transgene” is meant reducing or eliminating the ability of the transgene to block differentiation or XCI. In one example, inactivation of the transgene can be achieved through removal of the transgene (e.g., using a site specific recombinase and DNA recognition sequences flanking the transgene). In another example, if a viral vector is used for introduction of the transgene into the cell, removal of the origin of replication (e.g., using a site specific recombinase and DNA recognition sequences flanking the origin of replication) can result in a loss of the viral sequences, including the transgene, after propagation. Inactivation of the transgene can be measured using the assays for differentiation, XCI, or nucleation of interchromosomal pairing as described herein.

By “isolated” is meant substantially free of other cellular material, or culture medium when produced by recombinant techniques, or substantially free of chemical precursors or other chemicals when chemically synthesized.

By “nucleic acid molecule” is meant any chain of nucleotides or nucleic acid mimetics. Included in this definition are natural and non-natural oligonucleotides, both modified and unmodified.

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20^{th} edition), ed. A. Gennaro, 2000, Lippincott, Williams & Wilkins, Philadelphia, Pa.

By “proliferation” is meant the expansion of a population of cells by the continuous division of single cells into two identical daughter cells.

By “purified” is meant separated from other components that naturally accompany it. Typically, a compound (e.g., nucleic acid) is substantially pure when it is at least 50%, by weight, free from proteins, antibodies, and naturally-occurring organic molecules with which it is naturally associated. Preferably, the compound is at least 75%, more preferably, at least 90%, and most preferably, at least 99%, by weight, pure. A substantially pure compound may be obtained by chemical synthesis, separation of the factor from natural sources, or production of the compound in a recombinant host cell that does not naturally produce the compound. Nucleic acid molecules may be purified by one skilled in the art using standard techniques such as those described by Ausubel et al. (Current Protocols in Molecular Biology, John Wiley & Sons, New York, 2000). The nucleic acid molecule is preferably at least 2, 5, or 10 times as pure as the starting material, as measured using polyacrylamide gel electrophoresis, column chromatography, optical density, HPLC analysis, or western analysis.

By “recombinase” is meant any member of a group of enzymes that can facilitate site specific recombination between defined sites, where the sites are physically separated on a single DNA molecule or where the sites reside on separate DNA molecules. The DNA sequences of the defined recombination sites are not necessarily identical. There are several subfamilies including “integrase” (including, for example, Cre and λ integrase) and “resolvase/invertase” (including, for example, ΨC31 integrase, R4 integrase, and TP-901 integrase). Two preferred recombinases and their DNA recognition sequences are Cre (recombinase)-lox (recognition sequence) or flippase (FLP) (recombinase)-Frt (recognition sequence). (See Fukushige et al., Proc. Natl. Acad. Sci. USA 89:7905-7909 (1992); O'Gorman, et al., Science 251:1351-1335 (1991); Sauer et al., Proc. Natl. Acad. Sci. USA 85:5166-70 (1988); Sauer et al., Nuc. Acids Res. 17:147-161 (1989); Sauer et al., New Biol. 2:441-49 (1990); and Sauer, Curr. Opin. Biotechnol. 5:521-7 (1994)). Desirably, recombinase expression in the cell is “transient.” By “transient expression” is meant expression that diminishes over a relatively brief time span. Transient expression can be achieved by introduction of the recombinase as a purified polypeptide, for example, using liposomes, coated particles, or microinjection. Transient expression can also be achieved by introduction of a nucleic acid sequence encoding the recombinase enzyme operably linked to a promoter in an expression vector that is then introduced into the cell. Expression of the recombinase can also be regulated in other ways, for example, by placing the expression of the recombinase under the control of a regulatable promoter (i.e., a promoter whose expression can be selectively induced or repressed). It is generally preferred that the recombinase be present for only such time as is necessary for removal of the transgene sequences from the cell.

A “recombinase recognition sequence” refers to any DNA sequence recognized by a specific recombinase protein. Examples include the loxP site, which consists of two 13-bp inverted repeats flanking an 8-bp nonpalindromic core region and is recognized by Cre recombinase and the 34-bp FRT site recognized by FLP recombinase. Variants of the wild type recognition sequences are included herein. Variants can be identified by their ability to be recognized by the appropriate recombinase, as described below.

By “syntenic” is meant a corresponding gene or chromosome region occurring in the same order on a chromosome of a different species. Syntenic genes or chromosome regions are not necessarily highly homologous particularly if the conserved elements are noncoding. For example, the syntenic portion of the mouse X-inactivation center is found at human Xq13.

By “teratoma” is meant a tumor composed of tissues from the three embryonic germ layers, usually found in ovary and testis. A teratoma is generally produced experimentally in animals by injecting pluripotent stem cells and is used to determine the ability of the stem cell to differentiate into various types of tissues.

By “Tsix transgene” is meant a nucleic acid fragment substantially identical to a mammalian Tsix sequence, or any fragment thereof, that is introduced into a cell by artificial means. The transgene may or may not be integrated into the cell chromosome and may or may not be expressed. The transgene may or may not be episomal. Non-limiting examples of preferred Tsix transgene sequences include nucleic acid sequences at least substantially identical to the full-length mouse Tsix gene (FIG. 5, SEQ ID NO: 6), or fragments thereof, and nucleic acids at least substantially identical to fragments of the mouse Tsix gene such as pCC3 (SEQ ID NO: 9), p3.7 (SEQ ID NO: 10), DxPas34 (SEQ ID NO: 12), the 34 bp repeat of DxPas34 (SEQ ID NO: 13), the 68 bp repeat of DxPas34 (SEQ ID NO: 14), ns25 (SEQ ID NO: 21), ns41 (SEQ ID NO: 22), ns82 (SEQ ID NO: 23), mouse repeat A1 (SEQ ID NO: 28), mouse repeat A2 (SEQ ID NO: 29), mouse repeat B (SEQ ID NO: 30), rat repeat A (SEQ ID NO: 31), and rat repeat B (SEQ ID NO: 32). Another preferred Tsix transgene sequence includes at least 2 copies of the 34 bp or 68 bp DxPas34 repeat (SEQ ID NOs: 13 or 14, respectively), as well as at least 3 copies, at least 4 copies, and at least 5 copies or more. These preferred fragments are diagrammed in FIGS. 1, 2, and 3A and the sequences are provided in FIGS. 3B, 4, 5, and 30B. Additional non-limiting examples of preferred Tsix transgene sequences include nucleic acid sequences substantially identical to the full-length human Tsix gene (SEQ ID NO: 35), the human repeat A (SEQ ID NO: 40), or any fragments thereof, and nucleic acid sequences substantially identical to any mammalian (e.g., human, primate, bovine, ovine, feline, and canine) homologues, orthologues, paralogues, species variants, or syntenic variants of the mouse Tsix sequence (SEQ ID NO: 6), or fragments thereof. Species variations include polymorphisms in Xite and Tsix that occur between strains of mice including, but not limited to, C57BL/6, 129. and CAST/Ei mice. As indicated above for SEQ ID NOs: 13 and 14, it should be noted that for any of the fragments, particularly the smaller fragments such as SEQ ID NOs: 28, 29, 30, 31, 32, and 40, the transgene can include multiple copies of the sequences, for example, in tandem array (e.g., at least 2 copies, at least 3 copies, at least 4 copies, and at least 5 copies or more).

By “Xite transgene” is meant a nucleic acid fragment substantially identical to a mammalian Xite sequence, or any fragment thereof, that is introduced into a cell by artificial means. The transgene may or may not be integrated into the cell chromosome and may or may not be expressed. The transgene may or may not be episomal. Non-limiting examples of preferred Xite transgene sequences include nucleic acid sequences at least substantially identical to the full-length mouse Xite gene (FIG. 7, SEQ ID NO: 15), or fragments thereof, and nucleic acids at least substantially identical to fragments of the mouse Xite gene such as pXite (SEQ ID NO: 16), Xite Enhancer (SEQ ID NO: 17), ns130 (SEQ ID NO: 24), ns135 (SEQ ID NO: 25), ns155 (SEQ ID NO: 26), ns132 (SEQ ID NO: 27). These preferred fragments are diagrammed in FIGS. 1, 2, and 3A and the sequences are provided in FIGS. 3B, 4, and 7. Additional non-limiting examples of preferred Xite transgene sequences include nucleic acid sequences substantially identical to the human Xite gene (SEQ ID NO: 38), or fragments thereof, and nucleic acid sequences substantially identical to any mammalian (e.g., human, primate, bovine, ovine, feline, and canine) homologues, orthologues, paralogues, species variants, or syntenic variants of the mouse Xite sequence (SEQ ID NO: 15), or fragments thereof. Species variations include polymorphisms in Xite and Tsix that occur between strains of mice including, but not limited to, C57BL/6, 129, and CAST/Ei mice.

By “Tsix/Xite transgene” is meant a nucleic acid substantially identical to a mammalian Tsix, Xite, or combined or intervening Tsix/Xite sequence, or any fragment thereof, that is introduced into a cell by artificial means. The transgene may or may not be integrated into the cell chromosome and may or may not be expressed. The transgene may or may not be episomal. Sequences that include a region that spans all or a portion of both genes or the intervening region between the two genes are known as Tsix/Xite transgene and can also be used in the methods of the invention. Non-limiting examples of preferred Tsix/Xite transgenes include nucleic acid sequences substantially identical to the critical region spanning both genes in the mouse, such as pSxn (SEQ ID NO: 4), pCC4 (SEQ ID NO: 11), and the bipartite enhancer (SEQ ID NO: 19). These preferred fragments are diagrammed in FIGS. 1 and 3A and the sequences are provided in FIGS. 3B and 4. Additional non-limiting examples of preferred Tsix/Xite transgene sequences include nucleic acid sequences substantially identical to the critical region spanning both genes in the human chromosome, such as pSxn human (SEQ ID NO: 37), or fragments thereof, and nucleic acid sequences substantially identical to any mammalian (e.g., human, primate, bovine, ovine, feline, and canine) homologues, orthologues, paralogues, species variants, or syntenic variants of the critical region spanning both Tsix and Xite genes in the mouse, or fragments thereof. Species variations include polymorphisms in Xite and Tsix that occur between strains of mice including, but not limited to, C57BL/6, 129, and CAST/Ei mice.

By “Xic transgene” is meant a nucleic acid molecule substantially identical to a mammalian Xic region that is introduced into a cell by artificial means. The transgene may or may not be integrated into the cell chromosome and may or may not be expressed. The transgene may or may not be episomal. Preferred Xic transgenes include the full-length mouse Xic (SEQ ID NO: 1), nucleotides 80,000 to 180,000 of GenBank Accession No. AJ421479 (SEQ ID NO: 33). Each of the mouse transgenes described herein is found within this 100 kB fragment of AJ421749. For example, mouse Xist is found from nt 106,296 to nt 129,140, the mouse Tsix/Xite sequences are found within nt 157,186 to nt 104,000, and mouse Tsx sequence is found from nt 174,041 to nt 163,932. Another fragment within the mouse Xic is Jpx/Enox, found from nt 95,894 to nt 86,564 of AJ421479. Preferred Xic fragments include πJL2 (SEQ ID NO: 2) and πJL3 (SEQ ID NO: 3). Additional non-limiting examples of preferred Xic transgene sequences include nucleic acid sequences substantially identical to the human Xic (SEQ ID NO: 39), or fragments thereof, and nucleic acid sequences substantially identical to any mammalian (e.g., human, primate, bovine, ovine, feline, and canine) homologues, orthologues, paralogues, species variants, or syntenic variants of the mouse Xic (SEQ ID NO: 1), or fragments thereof.

By “Xist transgene” is meant a nucleic acid substantially identical to a mammalian mammalian Xist sequence, or any fragment thereof, that is introduced into a cell by artificial means. The transgene may or may not be integrated into the cell chromosome and may or may not be expressed. The transgene may or may not be episomal. Non-limiting examples of preferred Xist transgene sequences include nucleic acid sequences at least substantially identical to the full-length mouse Xist gene (FIG. 6, SEQ ID NO: 20), or fragments thereof, and nucleic acids at least substantially identical to fragments of the mouse Xist gene such as pXist 3′ (SEQ ID NO: 7) and pXist 5′ (SEQ ID NO: 8). These preferred fragments are diagrammed in FIG. 1 and the sequences are provided in FIG. 6. Additional non-limiting examples of preferred Xist transgene sequences include nucleic acid sequences substantially identical to the human Xist gene (SEQ ID NO: 35), or fragments thereof, and nucleic acid sequences substantially identical to any mammalian (e.g., human, primate, bovine, ovine, feline, and canine) homologues, orthologues, paralogues, species variants, or syntenic variants of the mouse Xist sequence (SEQ ID NO: 20), or fragments thereof. Species variations include polymorphisms in Xist that occur between strains of mice including, but not limited to, C57BL/6, 129, and CAST/Ei mice.

Stem cell differentiation is an irreversible process and commitment to the differentiation pathway prevents or greatly reduces the clinician's or investigator's ability to modify the stem cell in a way that is therapeutically useful. The enormous therapeutic potential of stem cells relies on the ability to control stem cell differentiation. Thus, there is a need for efficient methods for blocking or delaying differentiation in a stem cell in a manner that is reversible. The present invention provides such novel methods for controlling stem cell differentiation and allows for both the inhibition and induction of stem cell differentiation in a controlled manner. The present invention is based on the discovery that disruptions in the XCI process, either by an excess or a depletion of Xic, Tsix, and Xite, can block differentiation. In the present methods, disruptions in the XCI process are achieved through the use of transgenes or small RNAs derived from Xic, Tsix or Xite sequences, or fragments thereof, that are introduced into stem cells and prevent the stem cells from undergoing X chromosome inactivation and from differentiating in culture. These novel methods for manipulating stem cell differentiation allow the clinician or researcher to maintain the stem cells in the undifferentiated state for a sufficient time to modify the cells as desired (e.g., by introducing therapeutic genes) for therapeutic or research purposes, without having the limitations of cell or cell product contamination or inefficient inhibition of differentiation. The methods also allow the clinician to readily remove the block to differentiation, again in an efficient manner and free from contamination issues, so that the cells can be administered to a subject. The invention also features cells produced by the methods of controlling or delaying differentiation that can self-renew indefinitely in culture and are useful for therapeutic purposes such as regenerative medicine and gene therapy.

Other features and advantages of the invention will be apparent from the following Detailed Description, the drawings, and the claims.

### BRIEF DESCRIPTION OF THE DRAWINGS

FIG. 1 is a diagram of the Xic region showing a set of preferred transgenes for blocking stem cell differentiation.

FIG. 2 is a diagram of a subset of the Xic region showing the Tsix/Xite junction in greater detail. Additional preferred transgenes are indicated.

FIGS. 3A-3B are a diagram and corresponding nucleic acid sequence of the pSxN transgene. FIG. 3A is a diagram of the pSxN6 (also referred to as pSxN) transgene showing a set of preferred transgenes for blocking stem cell differentiation. This region includes the 5′ end of Tsix and Xite and contains elements critical for counting (numerator), cell differentiation, imprinting, choice, and mutual exclusion of X's. FIG. 3B is an annotated sequence map of the pSxN transgene (SEQ ID NO: 4). The sequence map is annotated to show restriction sites and the specific location of each of the transgenes identified in FIG. 3A.

FIG. 4 is an annotated nucleic acid sequence showing the 34 and 68 base pair repeats (SEQ ID NO: 13 and 14, respectively) of the DxPas34 transgene (SEQ ID NO: 12). Each line of sequence represents a 34 base pair repeat. These repeats are located between nt 5074-6630 of SEQ ID NO: 4 (FIG. 3B). Note that the 34 and 68 bp repeats are not exact repeats but vary slightly from one to the next.

FIG. 5 is a nucleic acid sequence showing the mouse Tsix RNA sequence (unspliced form; SEQ ID NO: 6).

FIG. 6 is a nucleic acid sequence showing the full-length mouse Xist RNA (unspliced form; SEQ ID NO: 20).

FIG. 7 is a nucleic acid sequence showing the mouse Xite region (SEQ ID NO: 15). This sequence is oriented in the same direction as the annotated sequence of pSxn (FIG. 3). Xite initiates in multiple locations within two clusters of start sites. The first cluster is around nt 6995-5773 (where there is the 1.2 kb enhancer). The second cluster is around nt 13000-12500. Note that all transcripts proceed in the antisense orientation (e.g., from nt 6995 to nt 1). Also note that Xite does not “end.” It just diminishes when it reaches Tsix. Also note that the second of the two start clusters is outside of the pSxn critical region but is still part of Xite.

FIGS. 8A-8E show the manifestations of a counting defect, candidate counting regions, and isolation of X^{Δ}X^{Δ}, X^{Δ}O, and X^{Δ}Y ES cells. FIG. 8A is a diagram showing the patterns of normal and aberrant counting. Solid black circles, _{Xi}. Clear circles, Xₐ. FIG. 8B is a diagram of the Xic showing existing deletions that are thought to either affect or spare counting. Horizontal dotted lines delineate the extent of each knockout. Hypothetical region for counting elements shown in orange color. FIG. 8C is a Southern blot analysis of select newly isolated X^{Δ}X^{Δ}, X^{Δ}O, and X^{Δ}Y ES lines. FIG. 8D is a photomicrograph showing the results of DNA FISH on Δf5 cells, using an X-linked probe from the Xite locus (pDNT1) demonstrating two Xs in mutant lines. FIG. 8E is a photomicrograph of Y-chromosome painting identifying Δf4 as an X^{Δ}Y clone. Chromosome painting was carried out as recommended by the manufacturer (Cambio, UK).

FIGS. 9A-9F show aberrant differentiation and XCI in X^{Δ}X^{Δ} but not X^{Δ}O or X^{Δ}Y clones. FIG. 9A is a series of phase contrast images of mutant EB taken at the same magnification. X^{Δ}X^{Δ} EB (Δf5 shown) showed poor differentiation between d2 and d6 (d, day), but some EB eventually showed sparse outgrowth by d9. X^{Δ}O (Δf32 shown), X^{Δ}X (BA9, not shown) and X^{Δ}Y (Δf4, not shown) showed normal differentiation throughout. FIG. 9B is a graph showing quantitation of cell death. Day 0 showed <<1% death in all cell lines. Data shown represent averages of three experiments. As calculated by the student t-test, statistical significance (P) is indicated in the table below. Each cell line was tested against the WT control. FIG. 9C is a series of images showing RNA/DNA FISH using probes for Xist RNA (FITC-labeled, green) and Xite DNA (Cy3-labelled, red) to mark the X-chromosome. FIG. 9D is a diagram showing XCI patterns on d3 of differentiation. >200 nuclei were counted for each sample from two experiments. n.a., not applicable. FIG. 9E is an image showing RNA/DNA FISH of a poorly growing Δf25 EB (X^{Δ}X^{Δ}) on day 6 showing numerous nuclei with two Xᵢ. Xist RNA, green. Xite DNA, red. FIG. 9F is an image showing RNA/DNA FISH on later differentiation days, with the majority of surviving X^{Δ}X^{Δ} cells (Δf41 shown) displaying only a single Xᵢ. Xist RNA, green. Xite DNA, red.

FIGS. 10A-10E show the creation of female transgenic ES lines carrying the Xic. FIG. 10A is a map of the Xic and P1 transgenes covering various regions of the Xic. The transgene sequences are: πJL2, an 80 kb P1 plasmid containing Xist and 30 kb upstream and downstream sequence (Lee et al., Proc. Natl. Acad. Sci. U.S.A. (1999), supra); πJL3, an 80 kb P1 plasmid containing Xist and 60 kb of sequence downstream (Lee et al., Proc. Natl. Acad. Sci. U.S.A. (1999), supra); and pSx7, the BssHII-NotI fragment of πJL1. Transgenes were introduced by electroporation, as previously described (Lee et al., Proc. Natl. Acad. Sci. U.S.A. (1999) supra), together with a Neo selectable marker (pGKRN) at 0.1 molar ratio. All transgenic lines used here have autosomal insertions. FIG. 10B is a Southern blot analysis of transgenic cells lines. Copy number analysis was carried out as described previously (Lee et al., Proc. Natl. Acad. Sci. U.S.A. (1999) supra). Xist copy numbers were normalized to Dnmt1. FIG. 10C is a series of phase contrast images of WT and transgenic EB differentiated for 5 days. All images are at the same magnification. FIG. 10D is a series of images showing RNA/DNA FISH for detecting Xist RNA (green) and the X-chromosome (Xite DNA, red). Cells from day 4. X, X-chromosome; T, transgenic autosome. Red arrows point to sparse Xist RNA aggregates seen in high-copy clones. FIG. 10E is a table showing that the frequency of Xist expression (as determined by RNA/DNA FISH) inversely correlated with transgene copy number.

FIGS. 11A-11E show the finer transgene mapping revealing that Tsix and Xite harbor counting elements. FIG. 11A is a map of the Xic and finer transgenes. The sequences carried by each transgene are: pSxn, a 19.5 kb RsrII-NotI fragment of πJL1 (SEQ ID NO: 4); p3.7, the 3.7 kb MluI-SacI sequence deleted from TSix^{ΔCpG} (SEQ ID NO: 10; Lee et al., Cell (1999) supra); pCC3, a 4.3 kb BamHI fragment downstream of the Tsix promoter (SEQ ID NO: 9); pCC4, a 5.9 kb BamHI fragment upstream of and including the Tsix promoter (SEQ ID NO: 11); pXite, a 5.6 kb fragment spanning DHS1-4 of Xite (Ogawa et al., supra); pXist5′, a 4.8 kb XbaI-XhoI fragment from the Xist promoter (SEQ ID NO: 8); pXist3′, a 4.9 kb PstI fragment from Xist exon 7 (SEQ ID NO: 7); and pTsx, bp 41,347-52,236 of Genbank X99946 from the Tsx locus (SEQ ID NO: 18). Except pXist3′, a Neo selectable marker is engineered into each plasmid for selection in ES cells. All lines characterized here have autosomal insertions. Solid purple bars, elements with strongest counting properties; dashed purple bars, elements which also have counting properties, albeit weaker than the former. FIG. 11B is a graph showing cell death analysis of select transgenic cell lines by the trypan blue assay. The data represent averages of three experiments. As calculated by the student t-test, statistical significance (P) is indicated in the adjacent table. Each test cell line was tested against the control, WTneo. FIG. 11C is a series of phase contrast images of transgenic EB and controls taken at the same magnification. For clones 3.7-11 and Xite-11, note large size of EB on d5 and massive degeneration by d8. FIG. 11D is a diagram showing XCI patterns in transgenic and control cell lines on d3. At least 200 nuclei were counted for each sample from two experiments. In Tsix and Xite transgenics, >50,000 cells on the slide were examined to conclude that 0% showed Xist expression. n.a., not applicable. FIG. 11E is a series of images showing repression of XCI in Tsix and Xite female transgenic lines but not in Xist or Tsx lines. RNA/DNA FISH to detect Xist RNA (green) and transgenic chromosome using p3.7, pXite, or pTsx plasmids as probe (red). X, X-chromosome; T, transgenic autosome.

FIGS. 12A-12D show the duality model for counting. FIG. 12A is a diagram showing the singularity model. Counting represents the titration of X-factors (green circles) and A-factors (violet circles). A-factors can originate from multiple distinct autosomes. The coupling of A- to X-factors forms the ‘blocking factor’ (BF). Any untitrated X-factor is degraded. Choice results from the binding of BF to one Xic, which then represses initiation of XCI. The Xi forms by default. X, X-chromosome. A, autosome. FIG. 12B is a diagram showing the duality model. Just as in the singularity model, the complexing of A- and X-factors forms BF. However, untitrated X-factor(s) results in the formation of a second complex dubbed the ‘competence factor’ (CF). The choice step represents the binding of BF and CF to the Xic. BF binding blocks XCI on the future Xₐ, while CF binding to the remaining X induces XCI on the future Xᵢ. The duality model implies that BF and CF bind in a mutually exclusive fashion. BF and CF most likely bind to the 5′ regions of Tsix and Xite. FIG. 12C is a diagram showing aberrant counting and chaotic choice in the X^{Δ}X^{Δ} mutant. Depicted are four equally likely outcomes during cell differentiation. The two top outcomes achieve dosage compensation and result in viable cells. These cells are presumed to be the surviving day 9 EB cells in FIG. 9A and the dosage compensated cells in FIG. 9F. They give rise to the occasional X^{Δ}X^{Δ} mice born (Lee, Nature Genet. (2002), supra). The two bottom outcomes lead to either two Xᵢ's or two Xₐ's (due to loss of mutual exclusion and ensuing ‘chaos’)—states which are not viable. Tsix deletion represented by gap in X. FIG. 12D is a diagram showing the occurrence of multiple Tsix or Xite sequences on autosomes squelches the blocking and competence factors from the endogenous Xic, leading to constitutively active X's in transgenic cells. Black boxes are Xic sequences, in whole or in part. ATg, transgenic autosome.

FIG. 13 is a series of phase contrast images showing aberrant cell differentiation in X^{Δ}X^{Δ} clones. Phase contrast images of mutant EB at the same magnification on days (d) of differentiation are indicated. To generate EZB, ES colonies were trypsinized into detached cellular clusters on d), grown in suspension culture for 4 days in DME/15% FBS without LIF and adhered to gelatinized plates thereafter to obtain outgrowths. Note: While many of the X^{Δ}X^{Δ} EB degenerated by d*, a significant fraction of EB did outgrow (examples shown), but the extent of outgrowth was generally less robust that WT or X^{Δ}O.

FIG. 14 is a series of phase contrast images showing aberrant cell differentiation in Tsix/Xite-containing transgenic lines. Phase contrast images of mutant and wild type EB at the same magnification on the indicated differentiation day are shown. All Tsix/Xite-containing XX transgenes result in poor EB outgrowth on day 5 and died massively by day 8. >99% of unattached cells were dead, as determined by trypan blue uptake. XY lines containing the same transgenes and XX lines containing Xist, Tsx, and vector transgenes did not display this phenotype. Among the Tsix/Xite transgenics, p3.7 and pXite transgenics showed unusually high radial growth (two clones of each shown). As compared to larger transgenes, pCC3, pCC4, and pSxN transgenics also did so but to a lesser and more variable extent. πJL2, πJL3, and pSx7 had elevated cell death rates as well but their EB colonies were not unusually large.

FIGS. 15A-D show evidence for X-X homologous associations. FIG. 15A is a series of images and graphs showing DNA FISH and X-X distribution profiles of wild-type female ES nuclei from day 0 to day 6 of differentiation and of MEFs. Two-probe combination: Xic DNA-green (pSxn-FITC)+Tsx DNA-red (pTsx-Cy3). DAPI (4′,6′-diamidino-2-phenylindole), blue. Each image is a two-dimensional (2D) representation of 3D image stacks of 0.2μ z-sections. The distributions display the normalized distances, ND=X-X distance/d, where d=2×(nuclear area/π)^{0.5}. ND ranges from 0 to 1. Mean distance, open triangle. FIG. 15B is a series of graphs showing the cumulative frequency curves for X-X pairs at 0.0 to 0.2 ND. P (KS test) was calculated in pairwise comparison against day O, Sample sizes for each experiment (n)=174 to 231. FIG. 15C is a graph showing the X-X distances <0.05 ND. Distances were graphed with standard deviations (SD) from three independent experiments. FIG. 15D is a diagram of the Xic and a graph showing proximity pairing is specific to the Xic. X-X distribution profiles for X-linked loci shown in the map. The KS test (P) compared Xic versus flanking loci. n=166 to 188.

FIGS. 16A-D show the homologous association that occurs during the initiation phase of XCI. FIG. 16A is a series of images and graphs showing RNA-DNA FISH for day 2 wild-type XX cells. Xic DNA, green (pSxn-FITC); Xist RNA, red (strand-specific riboprobes-Cy3). FIGS. 16B to 16D are a series of graphs showing the cumulative distributions for day 2 wild-type XX cells, comparing Xist⁺ (n=74) versus Xist⁻ (n=180) cells (FIG. 16B); Ezh2⁺ (n=33) versus Ezh2⁻ cells (n=178) (FIG. 16C); and H3-3meK27⁺ (n=48) versus H3-3meK27⁻ (n=188) cells (FIG. 16D).

FIGS. 17A-F show Tsix and Xite are necessary and sufficient for X-X pairing. FIG. 17A is a map of the Xic, Tsix^{ΔCpG} and Xite^{ΔL}, and various transgenes. FIG. 17B is a series of graphs showing the X-X distributions for Tsix and Xite mutants from day 0 to day 6. n=181 to 223. KS test compares each curve to the day 0 curve. FIG. 17C is a diagram showing the Tsix alleles and primers (red) used for 3C analysis. BamHI sites, blue arrow. FIG. 17D shows the 3C analysis of pairwise interactions in X^{ΔTsix(neo+)}X cells and p3.7 females. Primers pairs are indicated to the right of gels. C, positive control ligations. All minus-crosslinking (N) and minus-ligation controls were negative. FIG. 17E is a graph showing the relative pairing frequencies (X) on day n (dn) was normalized to β-globin (βg) and to day 0 values, using the equation shown. S, signal intensity quantitated by densitometry. Average and SD from three independent experiments. FIG. 17F DNA FISH and X-A distribution curves for transgenic ES cells. The transgene was labeled red by a Neo probe and the X labeled green by a pSx7 probe (for p3.7, pXite, pXist5′, and pTsx cells) or a pTsx probe (for pSx7 cells). The pSx7 partially overlaps the p3.7 and pXite transgenes, but the small overlap makes the signal dim and discernible from the X. For πJL1.4.1, the transgene was labeled green (pSx9 Xist fragment) and the X labeled red (pTsx probe). The KS test compared data sets from day 0 versus day 4. n=170 to 234.

FIGS. 18 A-D show de novo X-A pairing inhibits X-X pairing. FIG. 18A is a series of graphs showing the disruption of X-X pairing in female transgenic cells. n=177 to 221. FIG. 18B shows the KS test comparing data sets from day 0 versus day 2 and from day 0 versus day 4. FIG. 18C is a graph showing the average frequency of X-X pairing with standard deviations from three experiments. FIG. 18D is a model showing X-X pairing is required for counting/choice. Allelic crosstalking results in asymmetric chromosome marking (yellow circles, blocked Xic; red circle, induced Xic) and mutually exclusive designation of Xₐ and Xᵢ. Blue lines, Xist RNA. Ectopic Tsix/Xite transgenes (Tg-Xic) inhibit XCI by titrating away X-X interactions. Loss of pairing in Tsix X^{Δ}X^{Δ} causes aberrant counting/choice.

FIG. 19 is a series of graphs and images showing X-X proximity pairs represent X-chromosome doublets in XX cells rather than sister chromatids of a single X in XO cells. Xic labelling (Tsix probe, red) and X-painting (FITC, green) of WT female ES cells demonstrates that X-X proximity pairs represent two distinct Xs in XX cells rather than sister chromatids of a single X in XO cells. The paired Xs show X-paint signals that occupy twice the nuclear area as single Xs. The X-X distribution profile is shown on the right with KS testing (P) comparing d4 against d0.

FIG. 20 is a series of graphs showing proximity-pairing is specific to the X-chromosome. Distribution profiles of Chr. 1 centromere (1C) and Chr.2 Abca2 gene during ES cell differentiation.

FIGS. 21A-C show proximity-pairing is specific to the Xic. FIG. 21A is a series of images showing DNA FISH of XC and Tsix with Tsix signals being apart (left) or paired (right). FIG. 21B is a series of graphs showing the distribution profile of flanking X-linked probes on d4. FIG. 21C is a series of graphs showing the cumulative frequency curves of X-linked probe between d0 and d6. KS test, P=significance of the difference when tested against d0.

FIGS. 22A-B are a series of images and graphs showing the temporal delineation of proximity-pairing using Ezh2 (FIG. 22A) and H3-3meK27 (FIG. 22B) as markers. ImmunoFISH used an Xic probe (green) in combination with either an Ezh2 or H3-3meK27 antibody (red). X-X distribution profiles are shown on the right.

FIG. 23 is a series of graphs showing X-X distribution profiles of mutant Tsix and Xite ES cells on d0 to d6. These graphs show Tsix and Xite mediate pairing.

FIG. 24 is a diagram of the β-globin locus with 3C primers shown by arrowheads and BamHI sites shown by arrows.

FIG. 25 is a series of graphs showing the X-X distribution profiles of indicated transgenic female ES cells from d0 to d4.

FIG. 26 is a series of images showing female transgenic ES cells maintain the undifferentiated morphology even on day 5 under differentiation conditions. The ns11(vector) and ns82 (Tsix promoter) were used as negative controls.

FIG. 27 is a series of images showing male transgenic ES cells do not show the same undifferentiated morphology under differentiation conditions seen for female ES cells.

FIG. 28 is a series of graphs showing pairing between all subfragments of Tsix and Xite, except for ns82 (Tsix promoter only) in ES cells. The ectopic X-A pairing inhibits endogenous X-X pairing.

FIGS. 29A-I show a heterozygous deletion of the Tsix promoter exerts no obvious effect on choice. FIG. 29A is a schematic of the targeting scheme for deletion of Tsix promoter. RV: EcoRV, B: BamHI. Position of probes 1 and 2 are indicated by numbered grey rectangles. Filled and open triangles represent FRT and LoxP sites, respectively. FIG. 29B shows a Southern blot analysis of genomic DNAs from female clones digested with EcoRV and probed with Probe 1. FIG. 29C shows a Southern blot analysis of genomic DNAs from M. musculus (129) or M. castaneus (cast) liver, and DNA from wild-type or targeted female ES cells, digested with BamHI and probed with Probe 2. FIG. 29D shows a Southern blot analysis of genomic DNAs from male clones digested with EcoRV and probed with Probe 1. FIG. 29E shows allele-specific analysis of Tsix expression at two positions based on ScrFI and MnII SNPs. FIG. 29F is a series of graphs showing real-time RT-PCR quantitation of Tsix expression in male cells. All samples were normalized to the internal control, Rpo2. Error bars indicate one standard deviation. FIG. 29G shows allele-specific RT-PCR for Xist (top) or Mecp2 (bottom) in female cell lines. Days of differentiation are as indicated. FIG. 29H shows fraction of Tsix RNA from the 129 allele in the experiment shown in FIG. 29G. FIG. 29I is a series of images showing RNA/DNA FISH on day 8 female ΔPneo cells. Xist RNA, green; Neo DNA, red. The percentage of each XCI pattern is indicated to the right of the panels (number of nuclei sampled in parentheses). n=138.

FIGS. 30 A-E show DXPas34 is conserved and bears resemblance to transposable elements (TEs). FIG. 30A is a dot-plot of mouse x-axis, 138,745-141,000 of AJ421479) vs. rat (y-axis, 51,001-53,300 of N_W048043) sequences at DXPas34. Positions of different repeat clusters are as shown. FIG. 30B shows the consensus repeat sequences as determined for each species. Human repeat A, SEQ ID NO: 40; mouse repeat A1, SEQ ID NO: 28; mouse repeat A2, SEQ ID NO: 29; mouse repeat B, SEQ ID NO: 30; rat repeat A, SEQ ID NO: 31; rat repeat B, SEQ ID NO: 32. FIG. 30C shows a dot-plot analysis of mouse (x-axis, bp134,001-141,000 of AJ421479) vs. human (y-axis, bp 11,328,000-11,352,000 of NT_{—}011669). Regions 2 and 3 are as marked (Lee et al., Cell 99:47-57 (1999)). 14 kb insertion in human sequence, along with region containing A repeats (grey box), is marked on y-axis. FIG. 30D shows a schematic of human A-repeat region showing positions of ERV/LTRs and SINEs (light and dark grey boxes) and A-repeat units (black triangles). Sequence of a representative ERV/LTR (bp 11345000-11348700 of NT_{—}011669; SEQ ID NO: 43) is shown, with A-repeats boxed. FIG. 30E shows the human repeat A (SEQ ID NO: 40) perfectly matches the corresponding region of the human HERVL repeat (SEQ ID NO: 44). Mouse DXPas34 (A1 motif) (SEQ ID NO: 28) also shows excellent alignment with human HERVL (4 mismatches out of 27 bp) and mouse MERVL/RatERVL (5 mismatches) (SEQ ID NO: 45).

FIGS. 31A-E show DXPas34 displays bidirectional promoter activity. FIG. 31A is a schematic of Tsix 5′ region showing DXPas34, positions of primer pairs used for strand-specific RT-PCR (asterisk numbers), and relevant restriction sites (A: Age I, S: SalI, M: Mlul). Fragments of DXPas34 used in luciferase assays as shown. Luciferase activity was normalized to β-galactosidase and then to a Tsix promoter luciferase construct. Error bars indicate one standard error. FIG. 31B shows the results of strand-specific RT-PCR of Rrm2 or Tsix 5′ region as shown in FIG. 31A. Sense (s) and antisense (as) strands are indicated above each column. FIG. 31C shows the results of 5′ RACE to detect Dxpas-r. M: marker 5′: 5′ RACE amplification products. Sequence of a representative DXPas34 block with major and minor start sites indicated by heavy and light arrows (SEQ ID NO: 46). Repeat A1 units containing start sites are underlined. FIG. 31D shows the results of RT-PCR of ES cells treated with tagetin (T) for 8 hours or α-amanitin for 4 or 8 hours (α4 and α8, respectively). Tsix and Dxpas-r were detected at position 2. 18S RNA (a Pol I transcript) was amplified in parallel as a loading control. FIG. 31E shows the results of RT-PCR of RNAs from indicated samples were amplified at position 2.

FIGS. 32 A-F show targeted deletion of DXPas34 diminishes Tsix transcription. FIG. 32A is a diagram showing the targeting scheme for deletion of DXPas34. Three previous alleles of this locus are shown above in grey (Debrand et al., Mol. Cell. Biol. 19:8513-8525 (1999); Lee et al., Cell 99:47-57 (1999); Sado et al., Development 128 1275-1286 (2001)). Relevant restriction sites are S: StuI, B: BamHI, RV: EcoRV. Probes are indicated by grey boxes. FIG. 32B shows genomic DNAs from female and male clones digested with StuI and detected with Probe 1. FIG. 32C shows genomic DNAs digested with EcoRV and detected with Probe 2. FIG. 32D shows female genomic DNAs digested with BamHI and probed with Probe 2 to determine which allele was targeted. FIG. 32F is an autoradiogram showing quantitative real-time RT-PCR analysis in undifferentiated male cells at positions A and B (as shown in FIG. 29E). FIG. 32 is a series of graphs showing the results of strand-specific RT-PCR analysis on male cells of the indicated genotype as described in FIGS. 31B-E. Position 2 is within the ΔDXPas34 deleted region and is therefore omitted from analysis.

FIGS. 33A-C show deletion of DXPas34 leads to nonrandom XCI patterns. FIG. 33A is a series of images showing RNA/DNA FISH on day 12 female ΔDXPas34 cells. Xist RNA, green; DXPas34 DNA, red. The percentage of each XCI pattern is indicated to the right of the panels (number of nuclei in parentheses). n=48. FIGS. 33B-33C show allele-specific RT-PCR analysis for Xist (FIG. 33B) or Mecp2 (FIG. 33C) as described in FIG. 29G. Note: The ΔDXPas34 experiments were carried out in parallel with cell lines presented in FIG. 29G; therefore, the controls autoradiographs are identical. Charts indicate percent of transcripts from 129 chromosome at day 12 for multiple differentiation experiments. Open circles represent individual experiments; filled circles represent the mean with one standard deviation indicated by the error bars. Pairwise comparisons of samples were performed by student t-tests as shown (bottom).

FIGS. 34A and B show deletion of DXPas34 de-represses Tsix late in differentiation. FIG. 34A shows allele-specific Tsix RT-PCR at ScrFI polymorphism of wildtype and ΔDXPas34 females during differentiation. FIG. 34B is a series of graphs showing the allelic fraction of Tsix RNA from the 129 (left panel) or castaneus (right panel) during cell differentiation. Error bars indicate one standard deviation.

FIG. 35A is a schematic showing a 3-step model for how DXPas34 regulates Tsix expression during cell differentiation, where two enhancers and two functions of DXPas34 act in sequence to control distinct aspects of Tsix dynamics. This model proposes that the bipartite enhancer acts in pre-XCI cells to achieve biallelic Tsix expression. The Xite enhancer may act during this time as well, but it is not absolutely required until the onset of XCI, when its action enables the persistence of Tsix expression on the future Xₐ. Following the establishment of XCI, stable repression of Tsix requires the late-stage negative function of DXPas34. In this model, the antiparallel transcription of Dxpas-r leads to repression of Tsix, possibly through a similar antisense mechanism. FIG. 35B is a schematic showing a model for how Tsix co-opted retrotransposable elements for its regulation. A retrotransposon (rTE) fortuitously inserted into the Xic near the 5′ end of the primordial Tsix gene some 80-200 million years ago. With each TE being a self-sufficient gene expression module containing promoters, enhancers, and insulators, the insertion introduced a repertoire of regulatory elements that were co-opted to regulate Tsix (e.g., CTCF sites, Tsix enhancer, and alternative promoters (Chao et al., Science 295:345-347 (2002); Stavropoulos et al., Mol. Cell. Biol. 25:2757-2769 (2005)). Over time, the rTE might lost nearly all of its original sequences, except for those retained for the regulation of Tsix. The retained elements were repeatedly re-duplicated during this process to yield present-day DXPas34.

FIG. 36 shows a northern blot analyses for the presence of small RNAs at the within Xite, the left one probed with a let7 probe and the right one probed with a Xite probe as indicated in the schematic. Small RNAs within Xite are indicated with arrows. The let7b blot is a positive control that shows that the known mRNA (let7b) can be detected. Note for the Xite panel, the small RNAs can be detected using both sense and antisense probes indicating that the small RNAs are double stranded. Cell lines shown are those from Lee, Science (2005) supra, and Xu et al. Science (2006) supra, and Ogawa and Lee Mol. Cell. (2003) supra. Briefly, J1, wildtype male ES; 16.7, wildtype female ES; J1-ΔCpG is Tsix-deleted male ES; 16.7 Δ/Δ is Tsix−/− female ES; ΔL(Xite) is a 12.5 kb deletion of Xite; female-Tsix3.7 is transgenic female ES with 3.7 kb Tsix sequence deleted in the Tsix-allele; Female-Xite is transgenic female ES with 5.6 Xite transgene. Lanes 0, 4, 10 refer to days of cell differentiation for each cell line.

### DETAILED DESCRIPTION

Stem cells have enormous clinical potential because of their ability to self-renew indefinitely and to differentiate into a large number of cells and tissue types. Their potential use in regenerative therapy and gene therapy is almost limitless but is dependent on the ability to control the otherwise irreversible process of differentiation.

The present invention features a method for controlling such differentiation by introducing Xic, Tsix, Xite, or Tsix/Xite transgenes or fragments thereof, or small RNA derived from Xic, Tsix, Xite, or Tsix/Xite to inhibit differentiation. This allows sufficient time to manipulate the stem cells as desired for therapeutic or research purposes. Subsequent removal of the transgene allows for the induction of differentiation of the stem cells into the desired cell or tissue type, and administration to a patient.

### Transgenes

The present invention is based on the discovery that the introduction of a transgene having Xic, Tsix, Xite, or Tsix/Xite sequences, or fragments thereof, into the stem cell inhibits differentiation. Transgenes useful in the invention can include any Xic, Tsix, or Xite nucleic acid sequences or Tsix/Xite nucleic acid sequences having a part or all of both Tsix and Xite sequences.

Tsix and Xite are non-coding cis-acting genes found in the master regulatory region called the X-inactivation center (Xic). This region contains a number of unusual noncoding genes, including Xist, Tsix, and Xite, that work together to ensure that XCI takes place only in the XX female, only on one chromosome, and in a developmentally specific manner. Each of these genes makes RNA instead of protein. Xist is made only from the future inactive X and makes a 20 kb RNA that “coats” the inactive X, thereby initiating the process of gene silencing. Tsix is the antisense regulator of Xist and acts by preventing the spread of Xist RNA along the X-chromosome. Thus, Tsix designates the future active X. Xite works together with Tsix to ensure the active state of the X. Xite makes a series of intergenic RNAs and assumes special chromatin conformation. Its action enhances the expression of antisense Tsix, thereby synergizing with Tsix to designate the future active X. In addition, Tsix and Xite function together to regulate the counting and choice aspects of XCI through X-X pairing as described herein.

Transgenes having Xic, Tsix, Xite, or Tsix/Xite sequences, or fragments or combinations thereof, are useful in the methods of the invention to delay or control differentiation. It should be noted that although preferred fragments within the Xic, Tsix, Xite, or Tsix/Xite sequences are specified, any nucleic acid sequence within this region is useful in the methods of the invention. The data presented herein identifying the functional redundancy of this region with respect to blocking X-X pairing, counting and cell differentiation supports the use of any fragment from this region. For example, any sequence from this region that can inhibit X-X pairing (e.g., by inducing de novo X-transgene pairing) can be used to block differentiation.

Non-limiting examples of preferred Xic transgene sequences include the mouse Xic (SEQ ID NO: 1) or the human syntenic equivalent (SEQ ID NO: 39), πJL2 (SEQ ID NO: 2), and πJL3 (SEQ ID NO: 3).

Non-limiting examples of preferred Tsix transgene sequences include nucleic acid sequences at least substantially identical to the full-length mouse Tsix gene (SEQ ID NO: 6), or fragments thereof, and nucleic acids at least substantially identical to fragments of the mouse Tsix gene such as the highly conserved region (SEQ ID NO: 5), pCC3 (SEQ ID NO: 9), p3.7 (SEQ ID NO: 10), DxPas34 (SEQ ID NO: 12), the 34 bp repeat of DxPas34 (SEQ ID NO: 13), the 68 bp repeat of DxPas34 (SEQ ID NO: 14), ns25 (SEQ ID NO: 21), ns41 (SEQ ID NO: 22), ns82 (SEQ ID NO: 23), mouse repeat A1 (SEQ ID NO: 28), mouse repeat A2 (SEQ ID NO: 29), mouse repeat B (SEQ ID NO: 30), rat repeat A (SEQ ID NO: 31), and rat repeat B (SEQ ID NO: 32). Another preferred Tsix transgene sequence includes at least 2 copies of the 34 bp or 68 bp DxPas34 repeat (SEQ ID NOs: 13 or 14, respectively), as well as at least 3 copies, at least 4 copies, and at least 5 copies or more. Additional preferred Tsix transgene sequences include nucleic acid sequences at least substantially identical to the human syntenic equivalents: the full-length human Tsix gene (SEQ ID NO: 36), the human repeat A (SEQ ID NO: 40), or any fragments thereof, and nucleic acid sequences substantially identical to any mammalian (e.g., human, primate, bovine, ovine, feline, and canine) homologues, orthologues, paralogues, species variants, or syntenic variants of the mouse Tsix sequence (SEQ ID NO: 6), or fragments thereof.

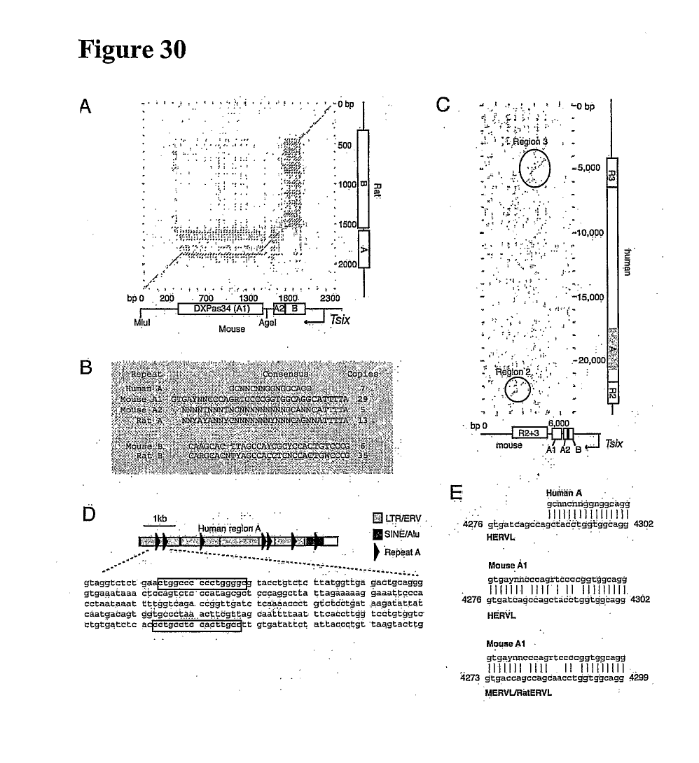

As indicated above for SEQ ID NOs: 13 and 14, it should be noted that for any of the fragments, particularly the smaller fragments such as SEQ ID NOs: 28, 29, 30, 31, 32, and 40, the transgene can include multiple copies of the sequences, for example, in tandem array (e.g., at least 2 copies, at least 3 copies, at least 4 copies, and at least 5 copies or more). The mouse repeat A1 (SEQ ID NO: 28), mouse repeat A2 (SEQ ID NO: 29), mouse repeat B (SEQ ID NO: 30), rat repeat A (SEQ ID NO: 31), rat repeat B (SEQ ID NO: 32), and human repeat A (SEQ ID NO: 40) are all part of the DXPas34 region and include the canonical sequences required for binding the transcription factor, CTCF. These small repeat units of DxPas and any ERV derived multimer of the canonical sequences provided in FIG. 30B are therefore included as preferred Tsix transgene sequences that are useful in the methods of the invention.

Non-limiting examples of preferred Xite transgene sequences include nucleic acid sequences at least substantially identical to the full-length mouse Xite gene (SEQ ID NO: 15), or fragments thereof, and nucleic acids at least substantially identical to fragments of the mouse Xite gene such as pXite (SEQ ID NO: 16), Xite Enhancer (SEQ ID NO: 17), ns130 (SEQ ID NO: 24), ns135 (SEQ ID NO: 25), ns155 (SEQ ID NO: 26), ns132 (SEQ ID NO: 27). Additional non-limiting examples of preferred Xite transgene sequences include nucleic acid sequences substantially identical to the human Xite gene (SEQ ID NO: 38), or fragments thereof, and nucleic acid sequences substantially identical to any mammalian (e.g., human, primate, bovine, ovine, feline, and canine) homologues, orthologues, paralogues, species variants, or syntenic variants of the mouse Xite sequence (SEQ ID NO: 15), or fragments thereof.

Sequences that include a region that spans all or a portion of both genes or the intervening region between the two genes are known as Tsix/Xite transgene and can also be used as transgenes in the methods of the invention. Non-limiting examples include a nucleic acid having the entire critical region spanning both genes of the mouse chromosome, pSxn (SEQ ID NO: 4), pCC4 (SEQ ID NO: 11), and the bipartite enhancer (SEQ ID NO: 19). Additional preferred Tsix/Xite transgene sequences include nucleic acid sequence substantially identical to the intervening region between the human syntenic equivalents of Tsix (SEQ ID NO: 36) and Xite (SEQ ID NO: 38). One example of a human Tsix/Xite transgene sequence is pSxN human (SEQ ID NO: 37).

The preferred fragments are shown in Tables 1 and 2, below. Note that because the fragments are non-coding regions, the exact start and end of the sequence is of little importance. Therefore, for all fragments, the size and nucleotide sequences are approximate values and can be altered by 1, 2, 5, 10, 20, 30, 40, 50, 100, 150, 200, 250, 500, 750, 1000 or more nucleotides. Also note that all sequences are presented in a 5′ to 3′ orientation.

TABLE 1

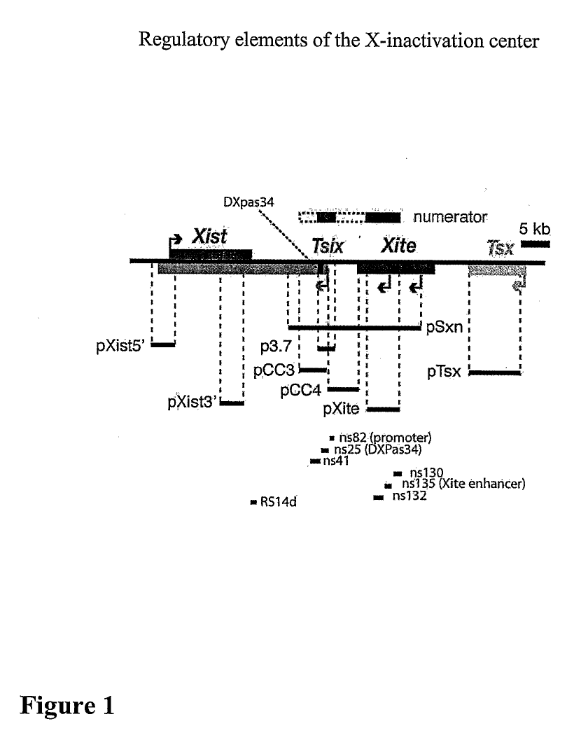

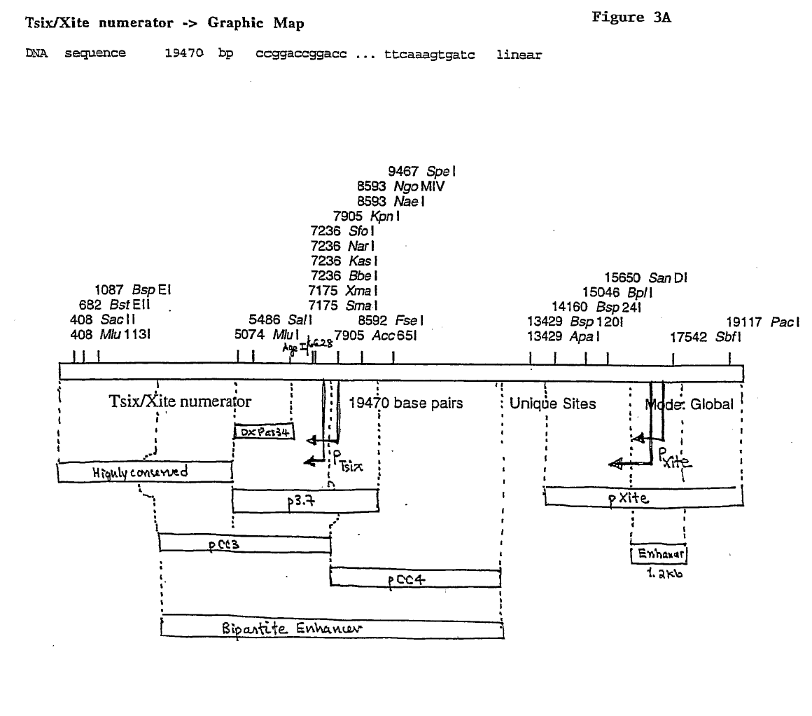

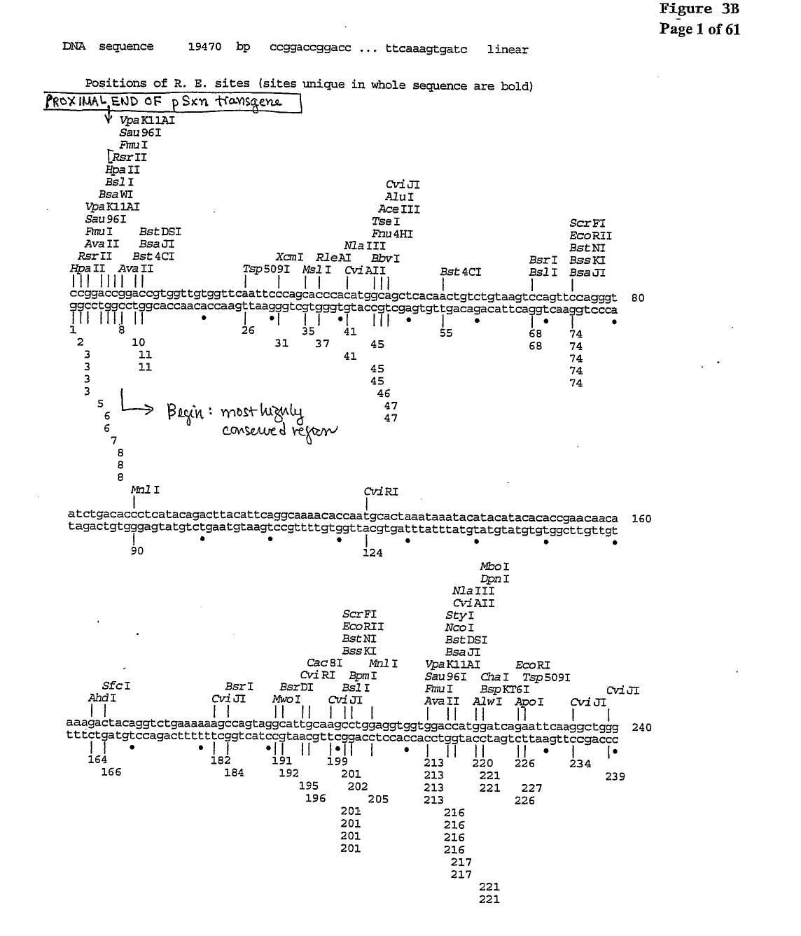

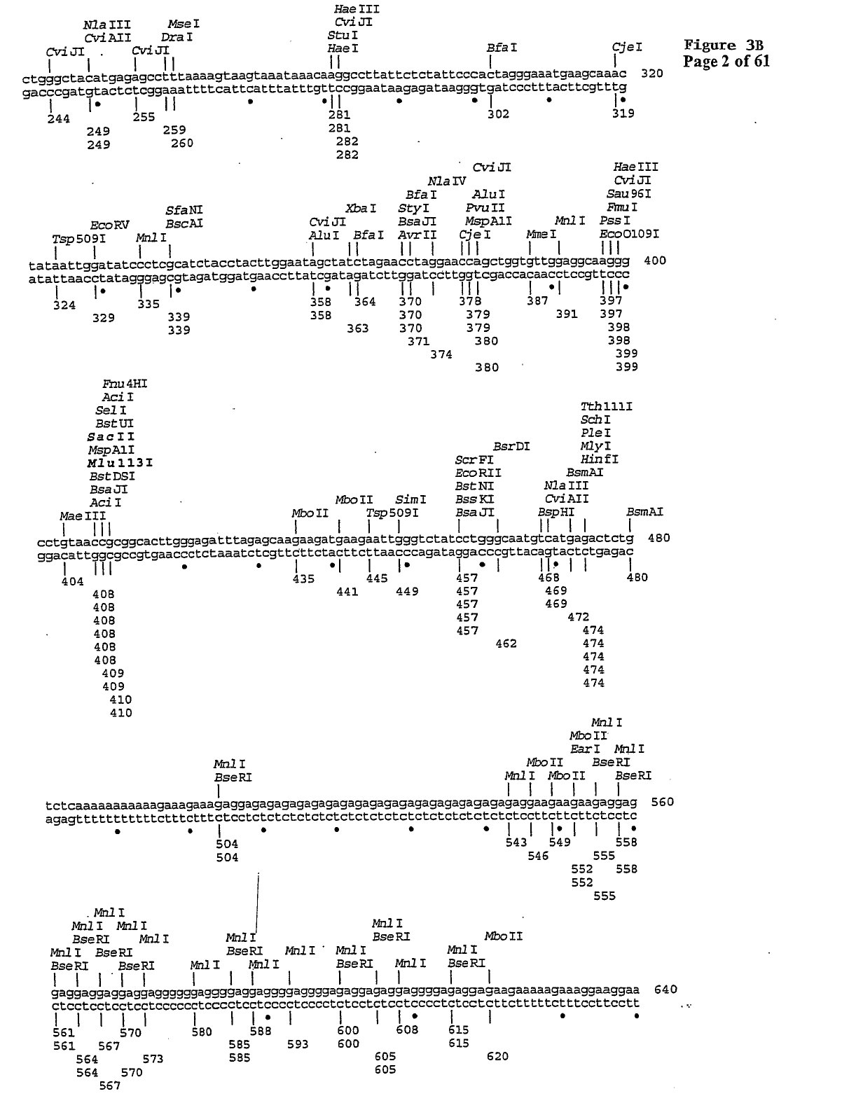

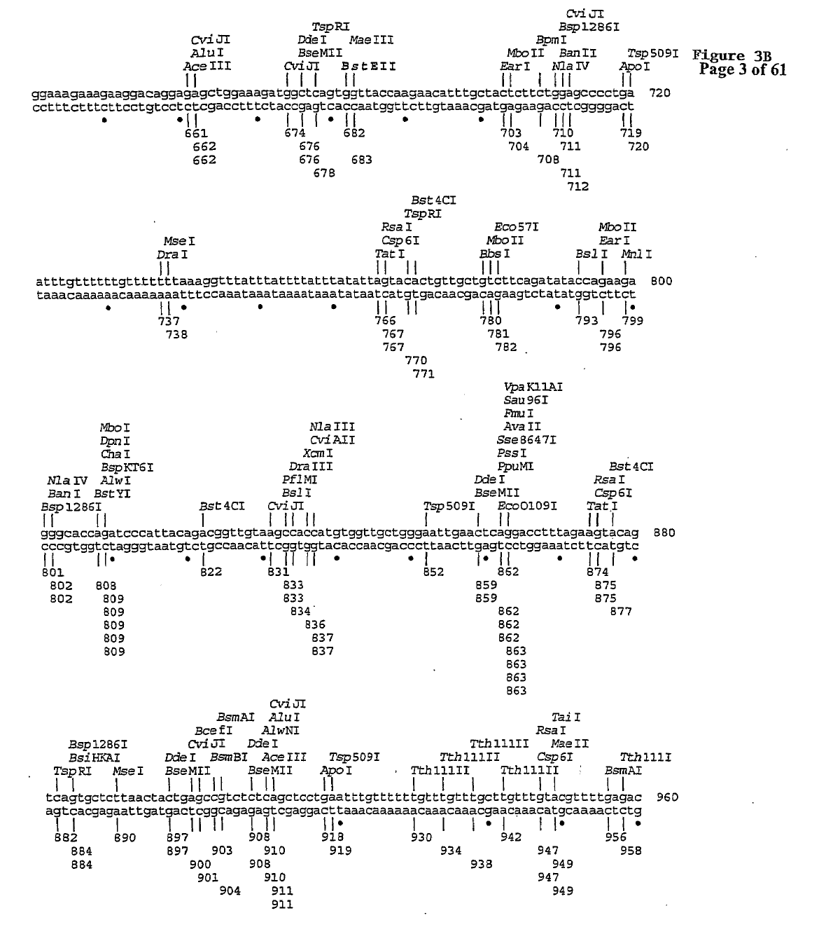

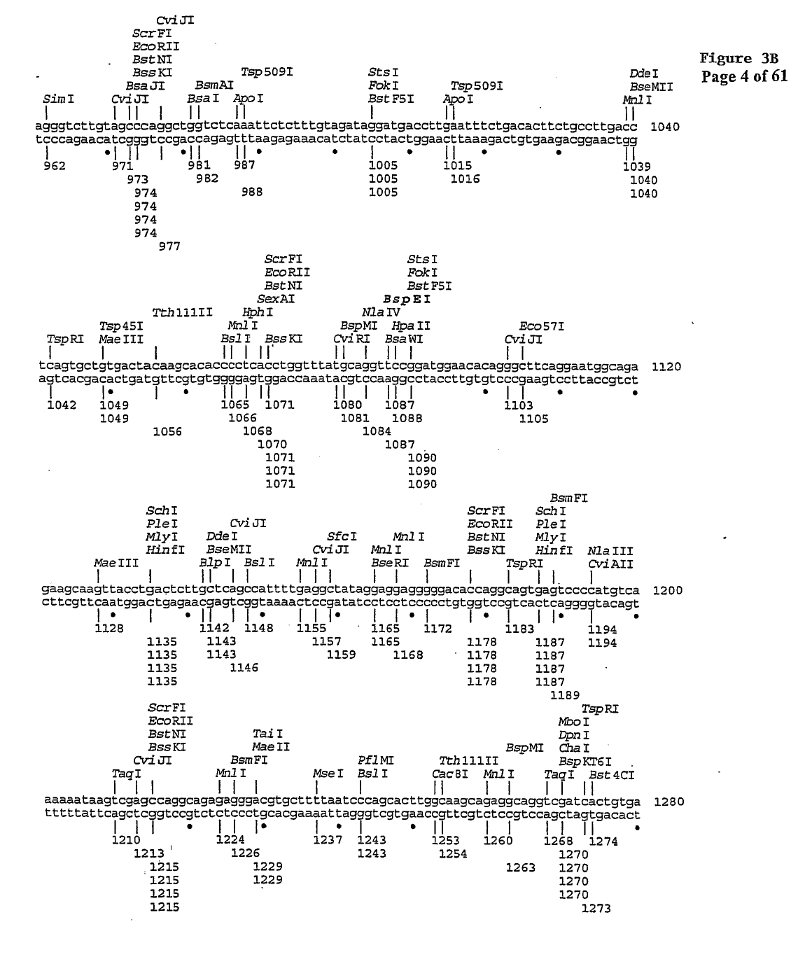

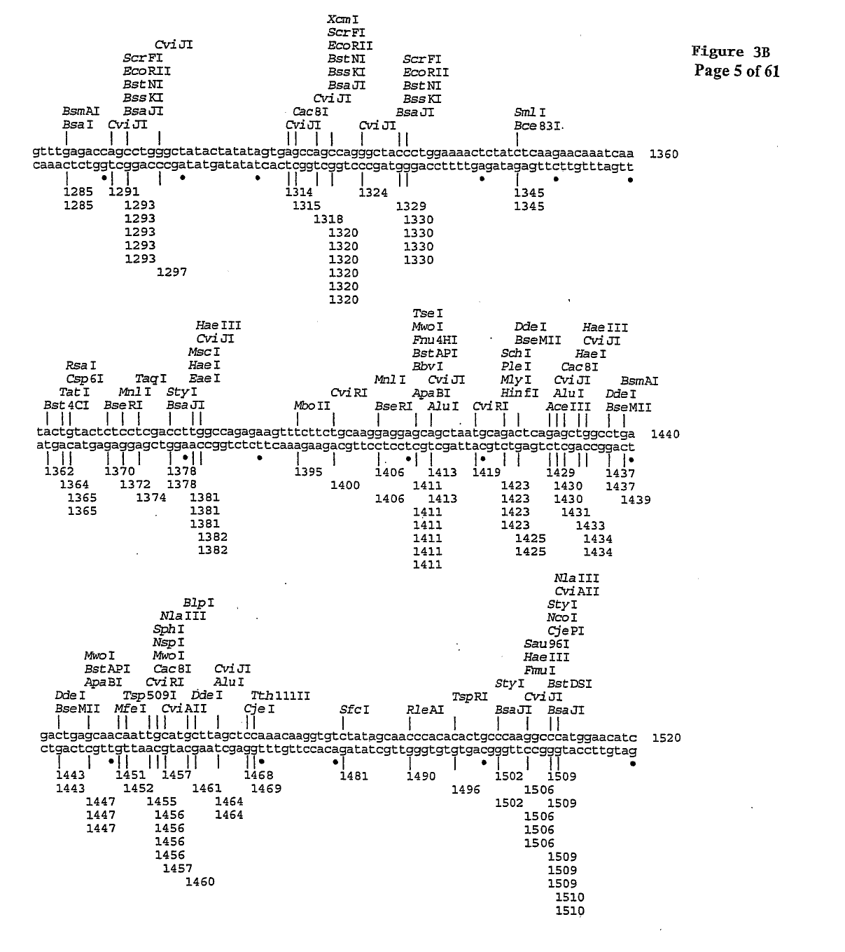

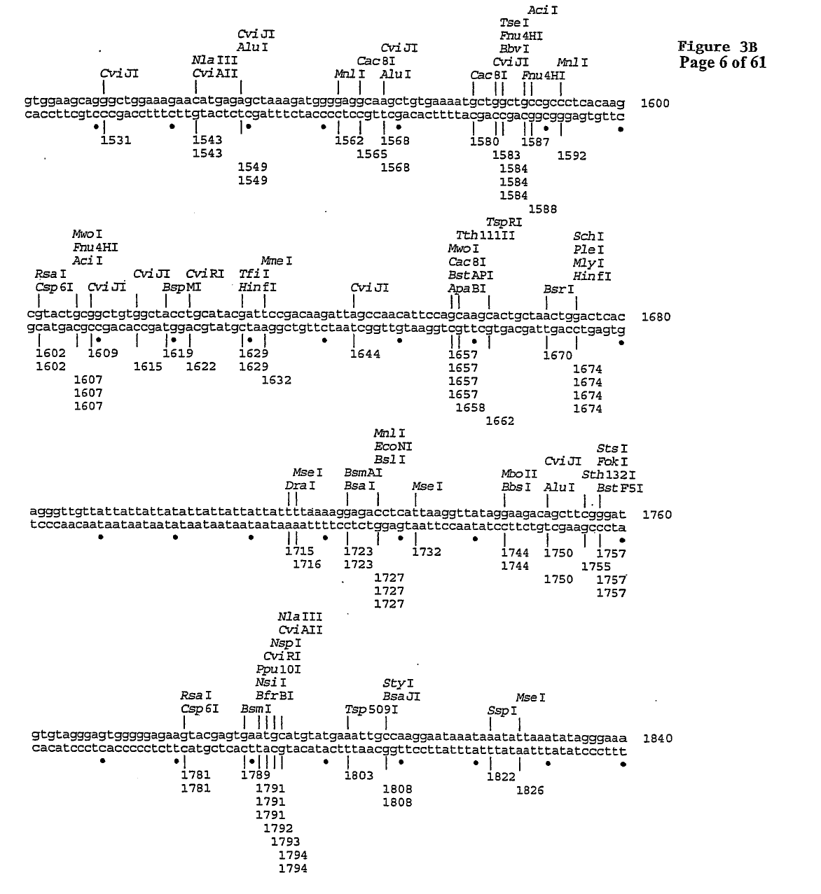

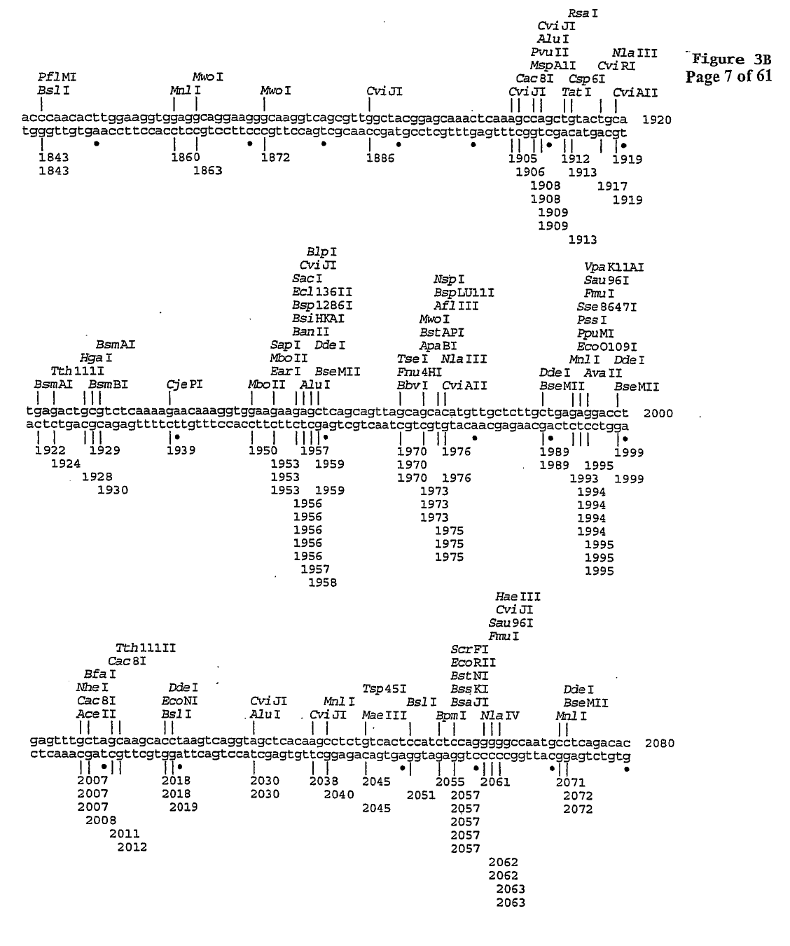

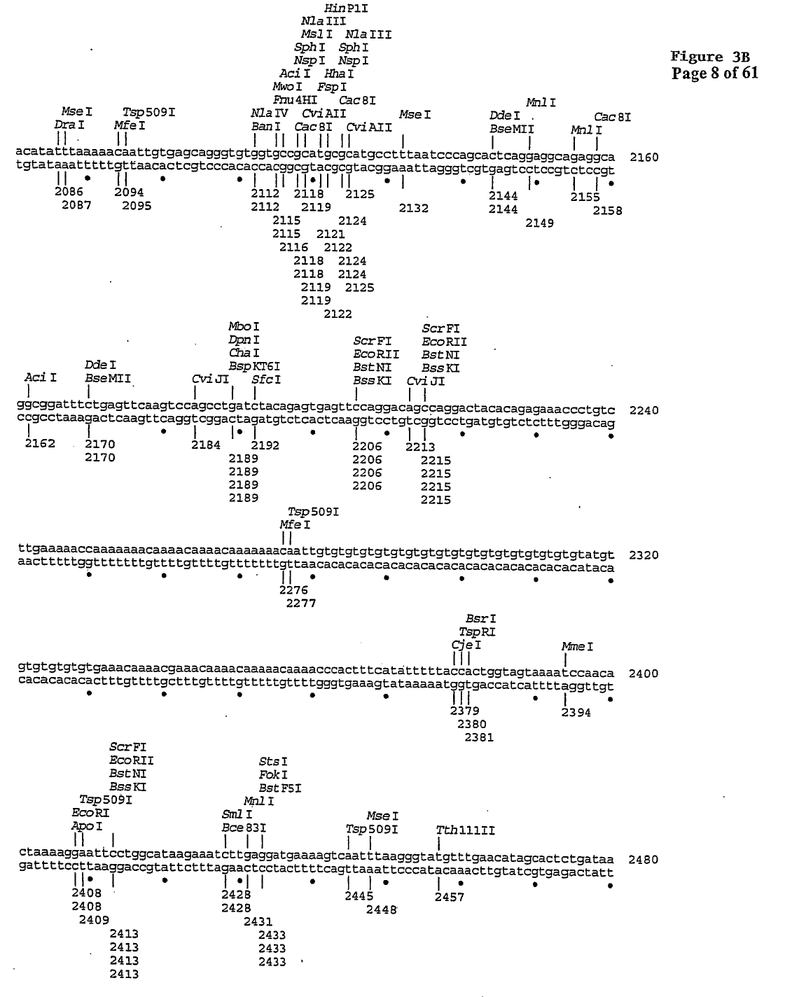

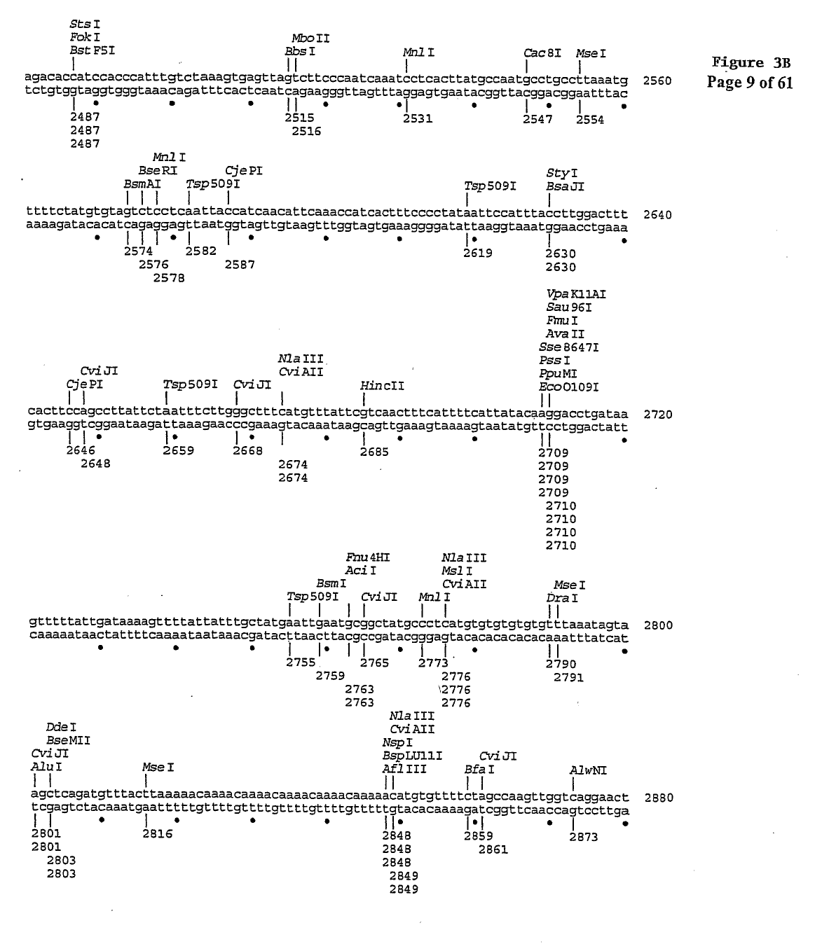

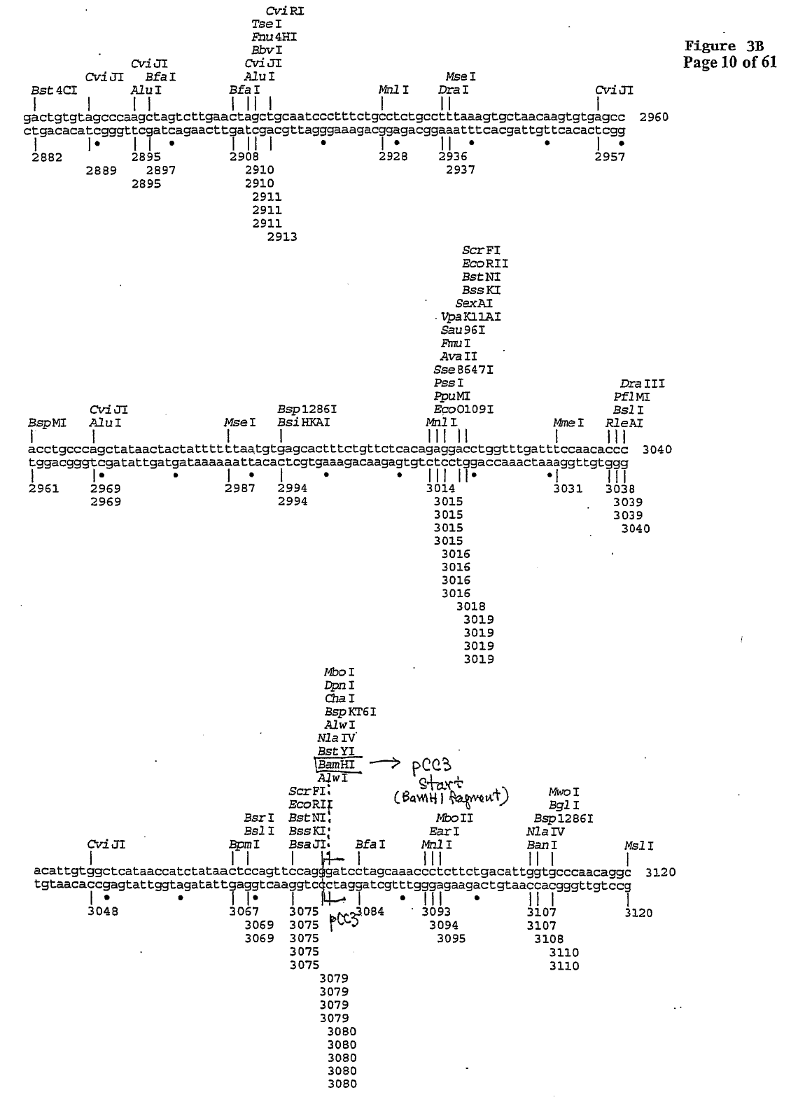

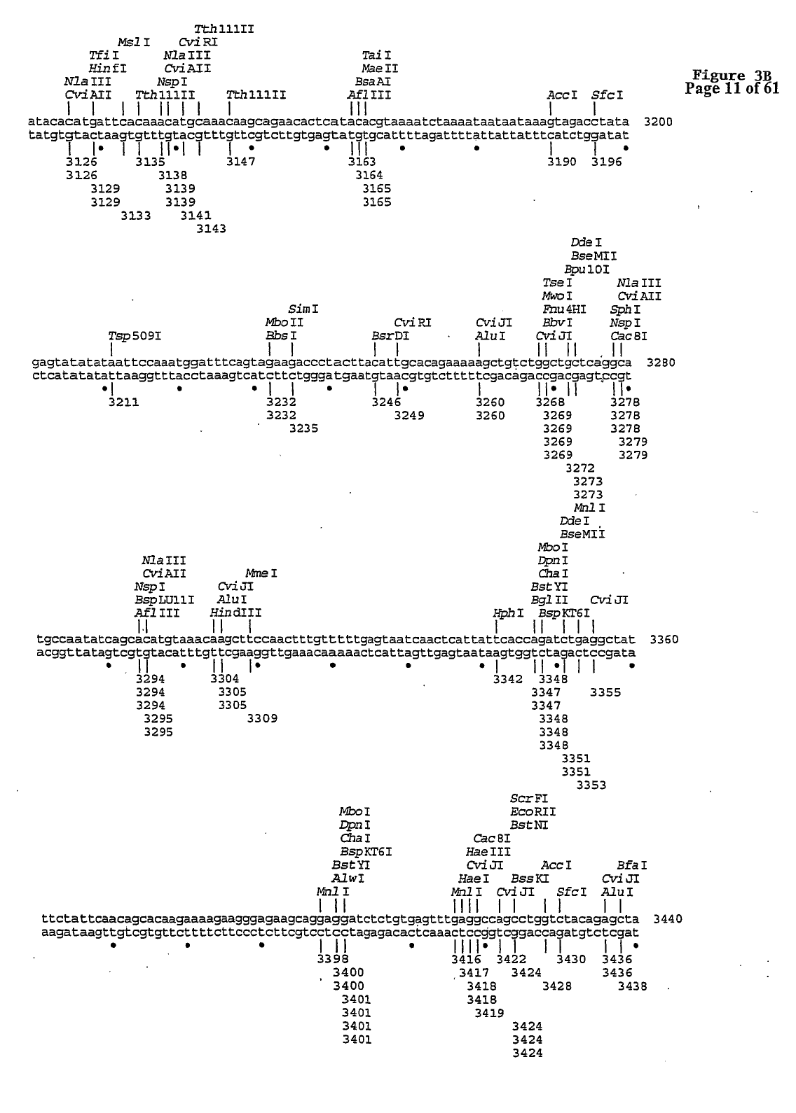

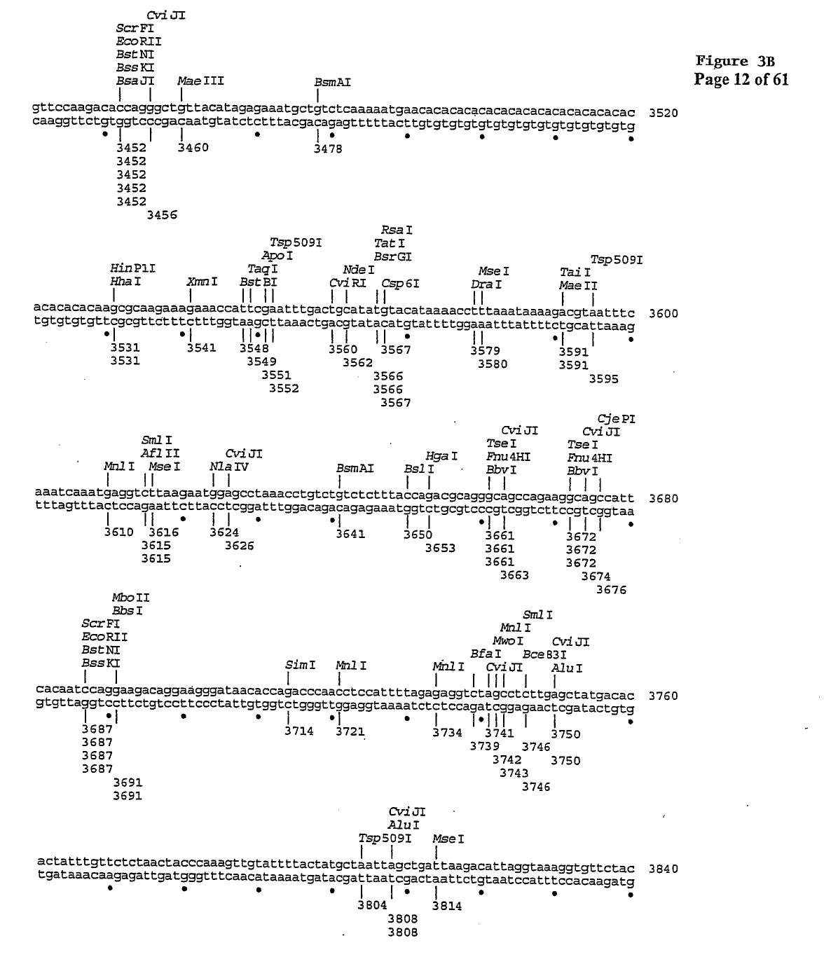

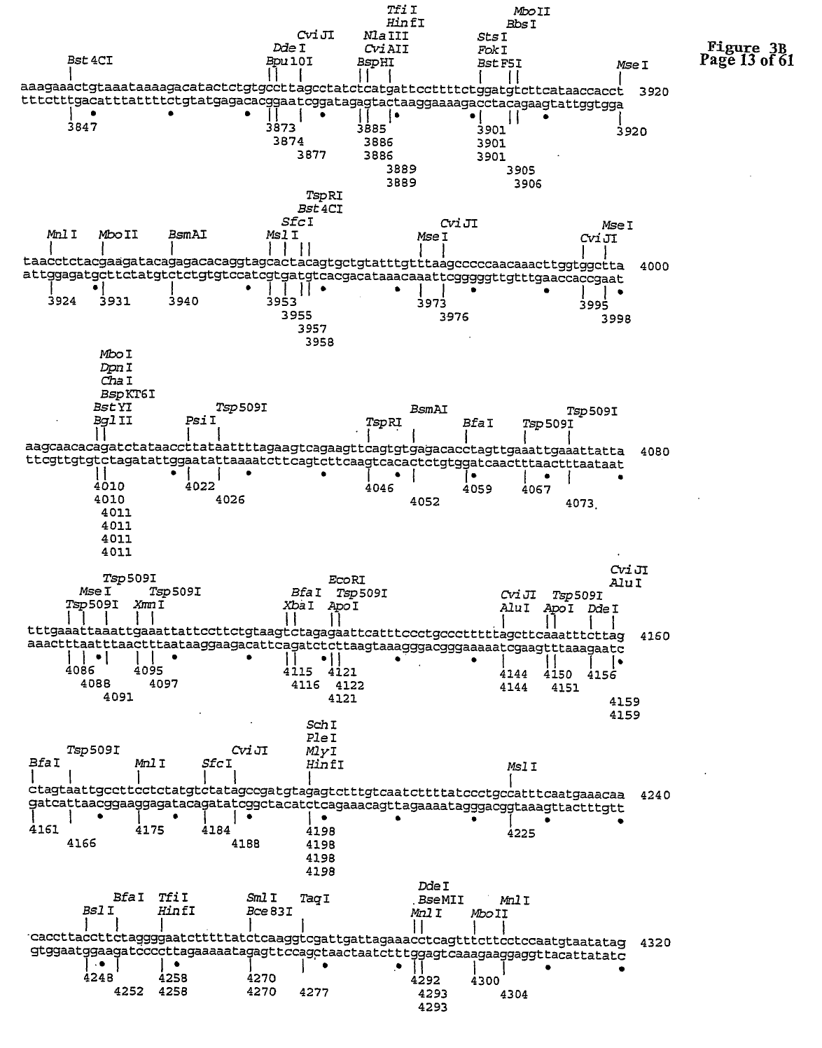

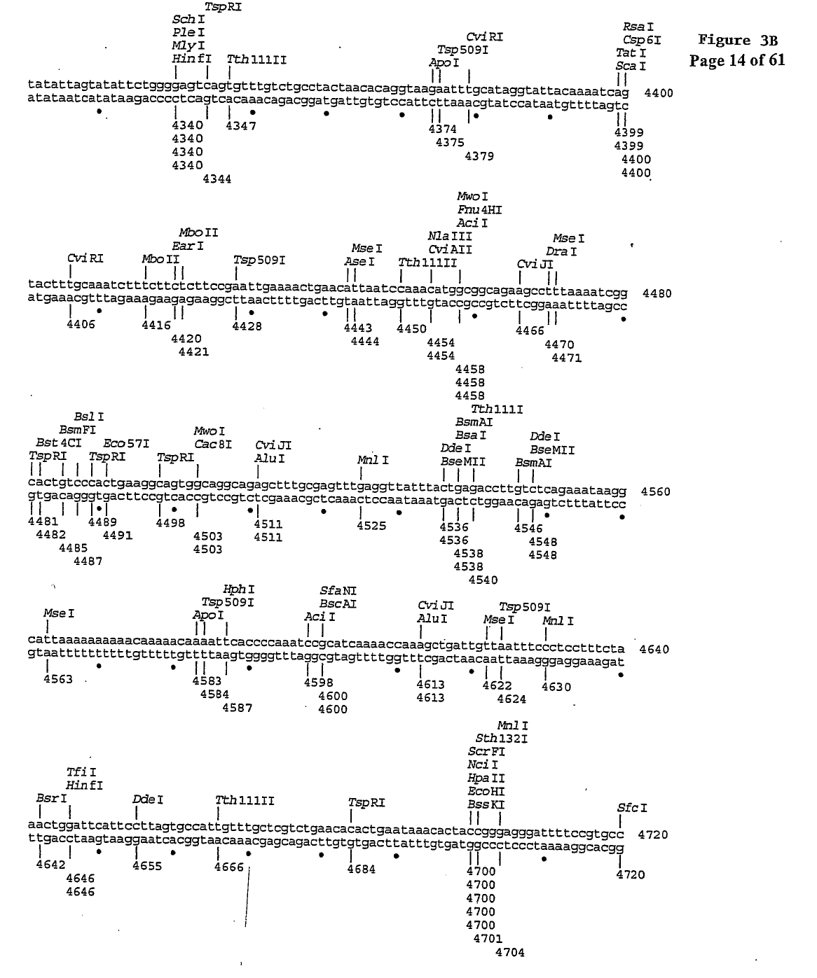

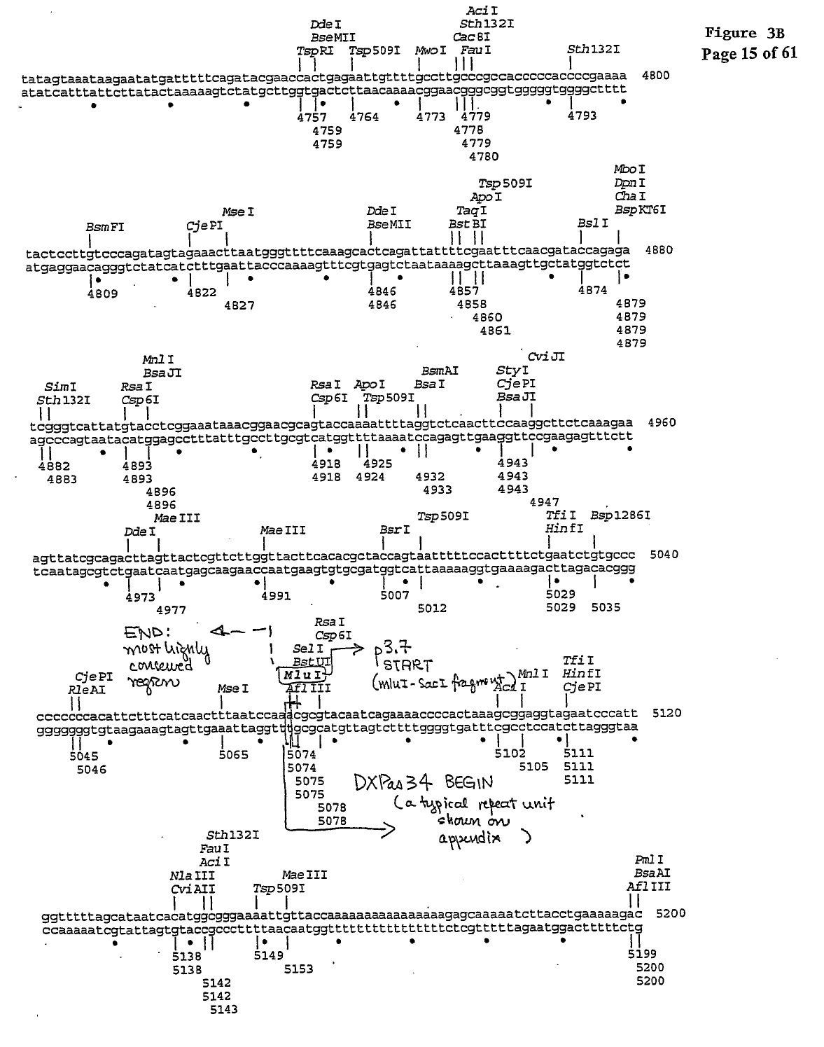

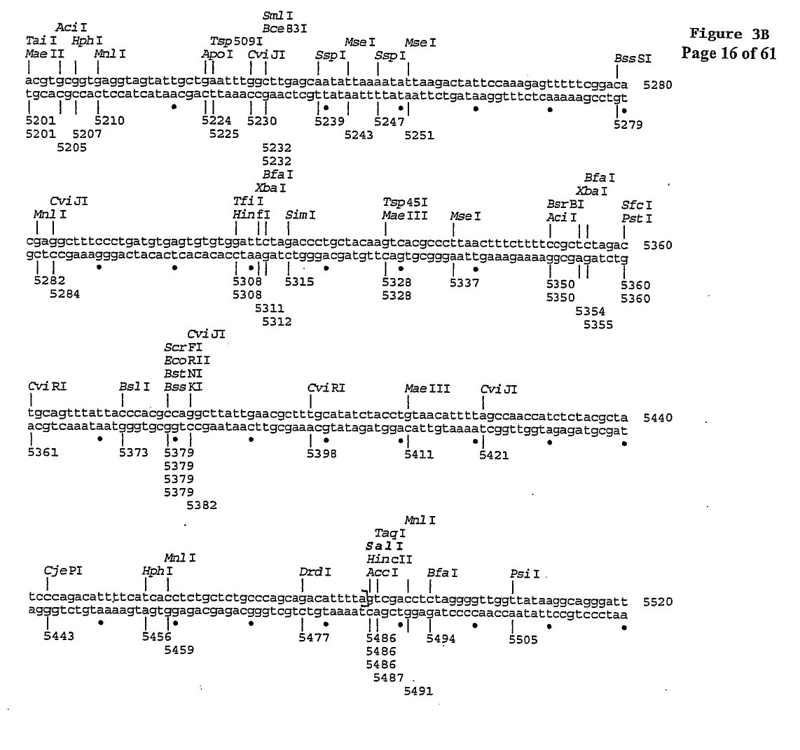

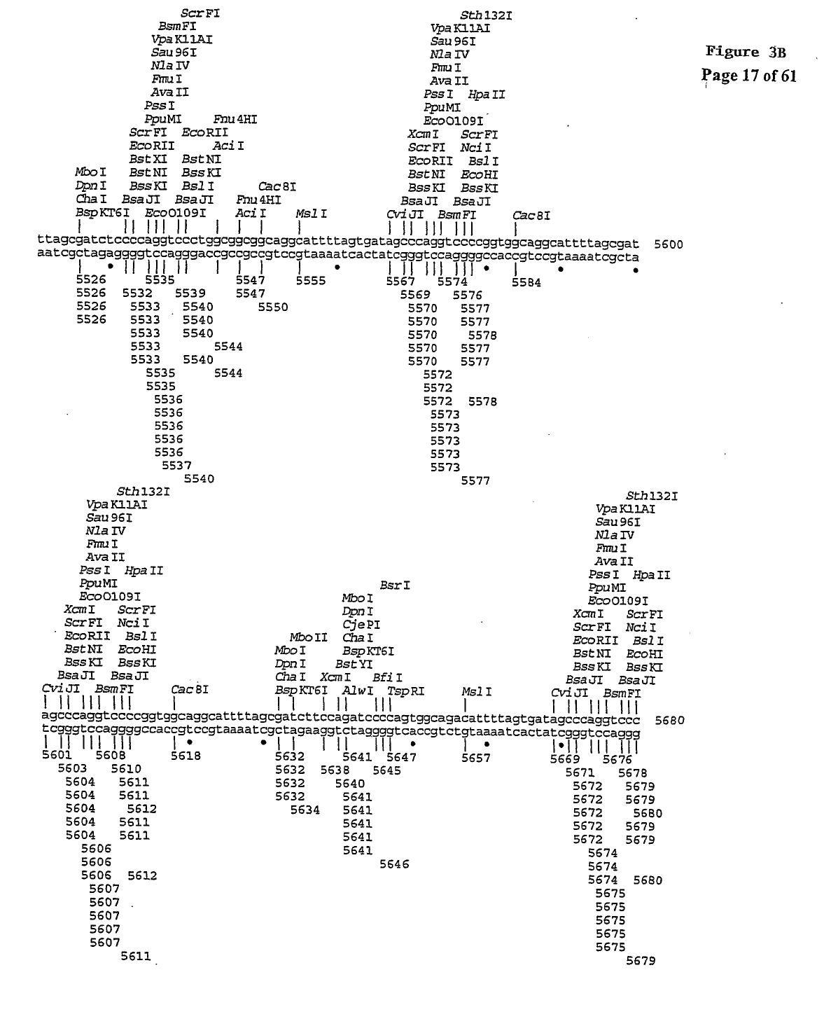

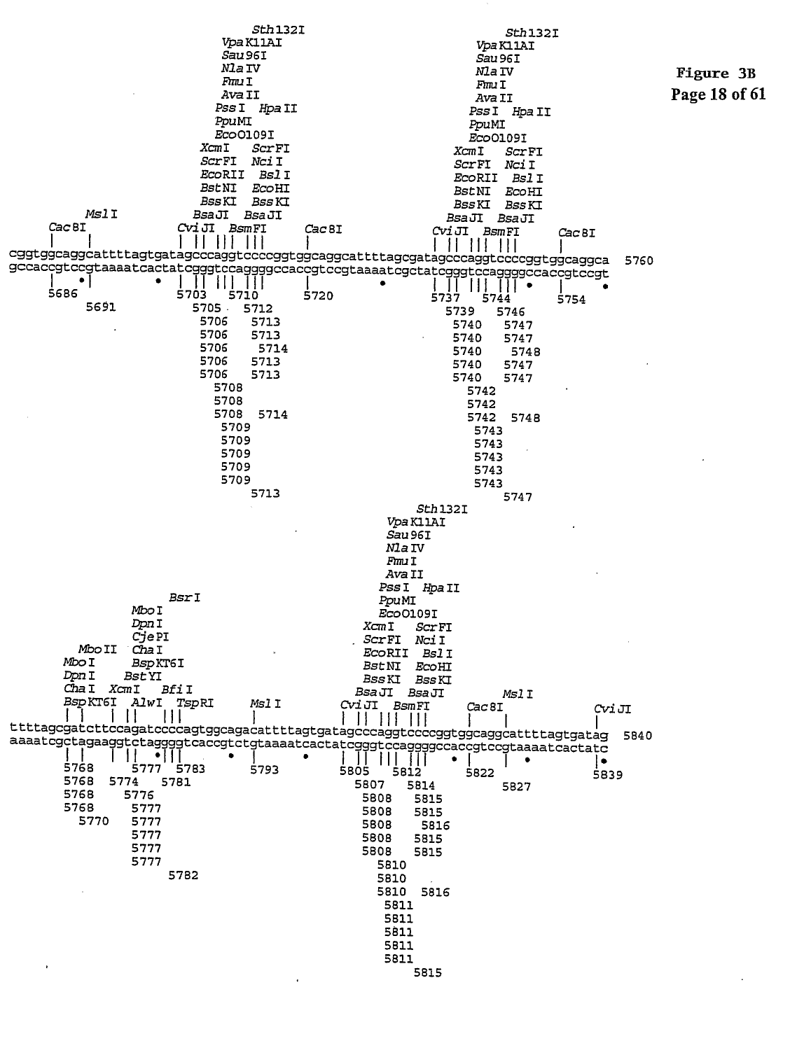

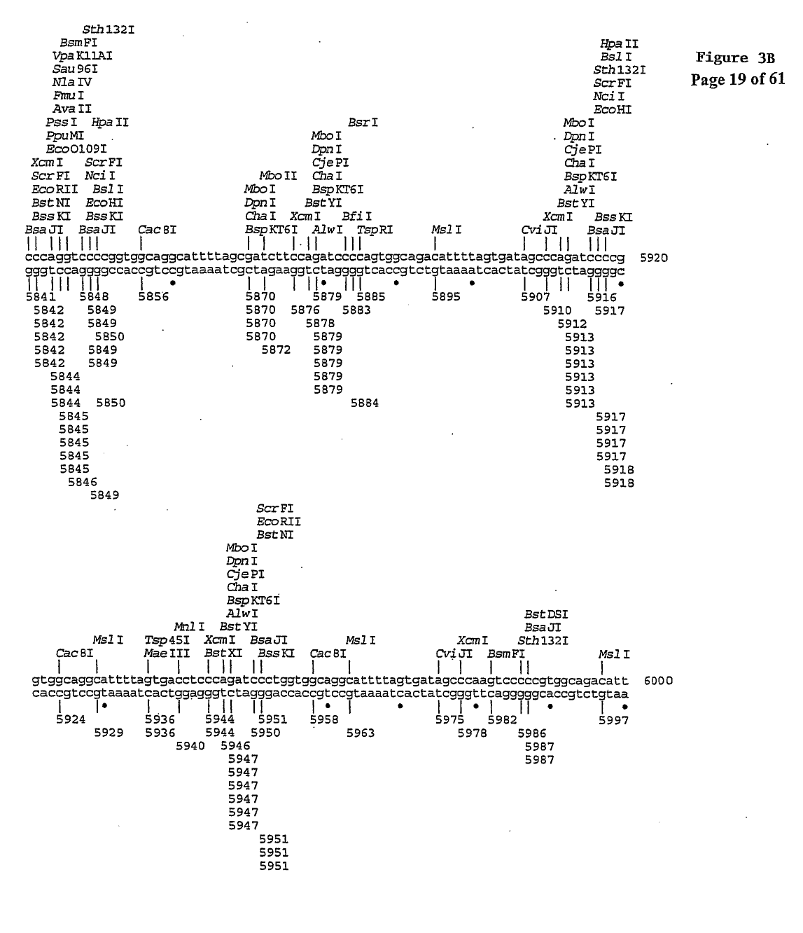

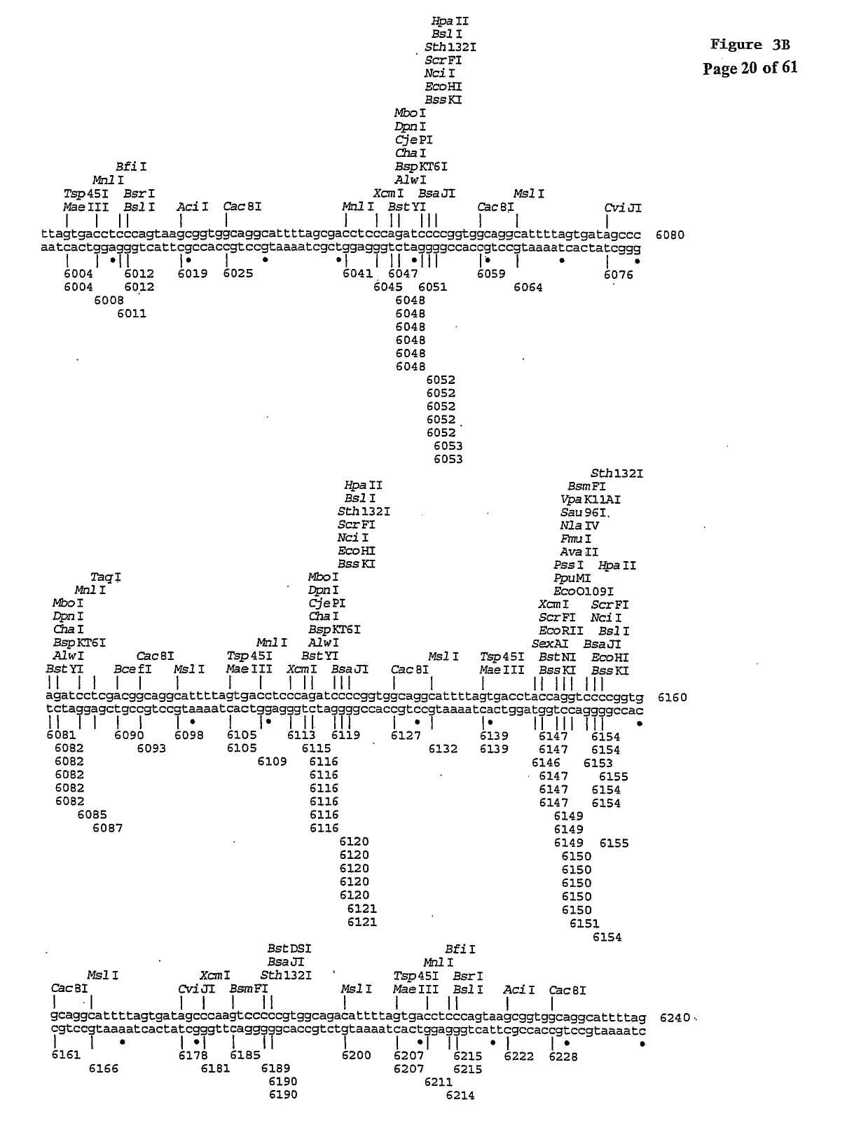

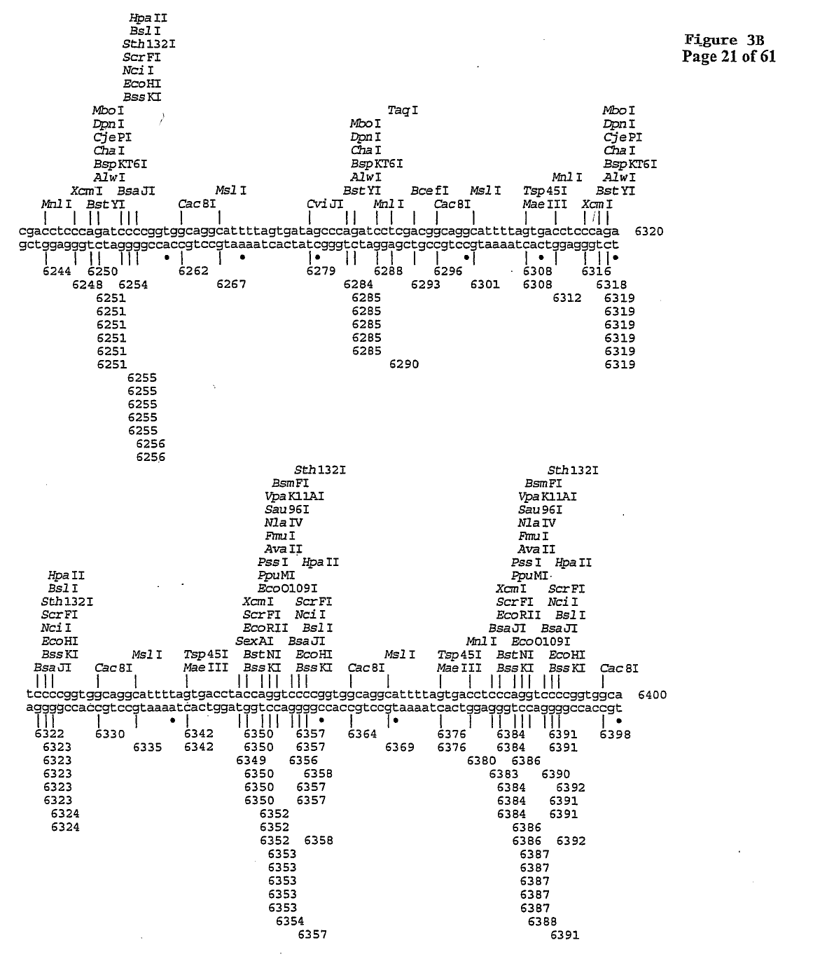

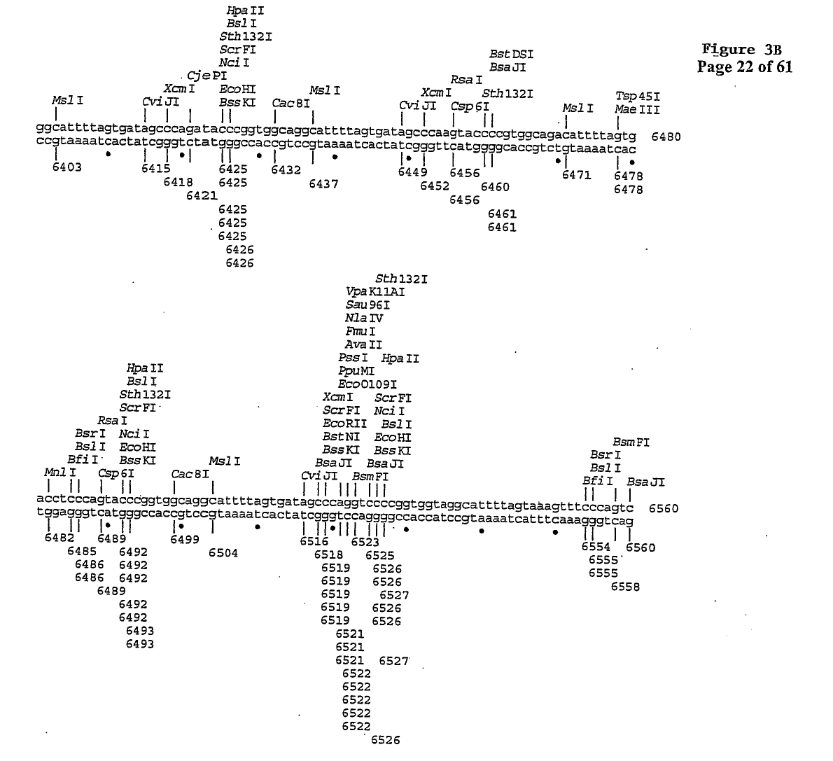

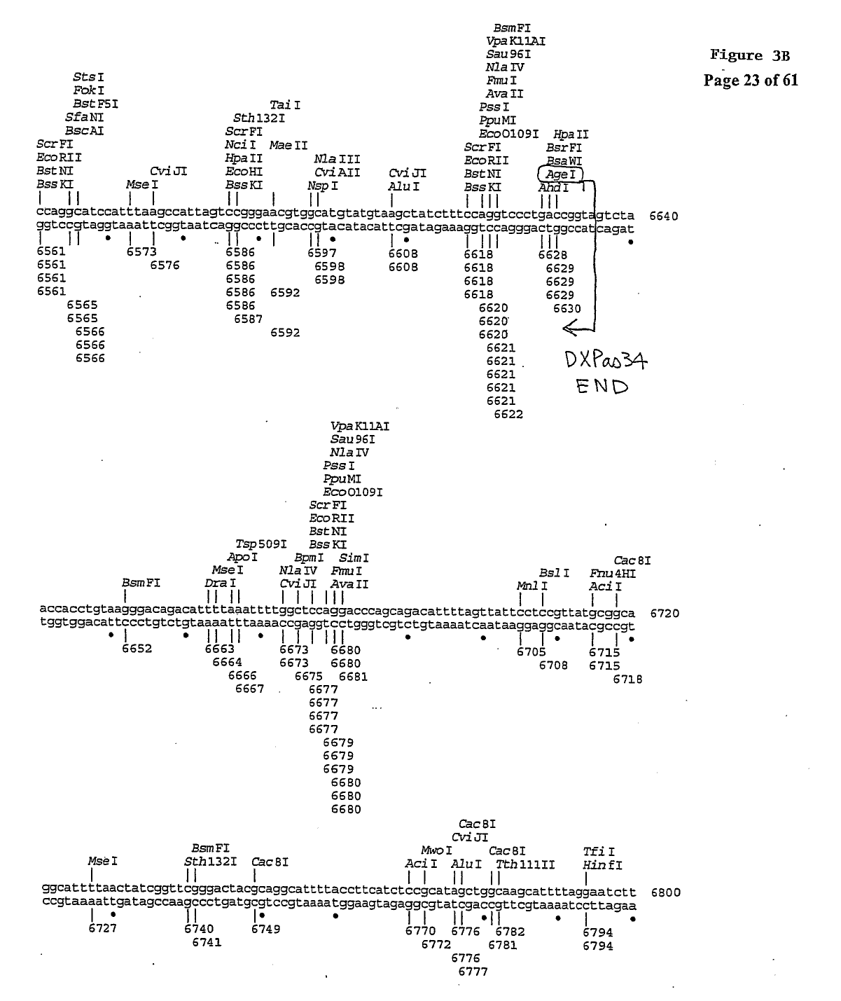

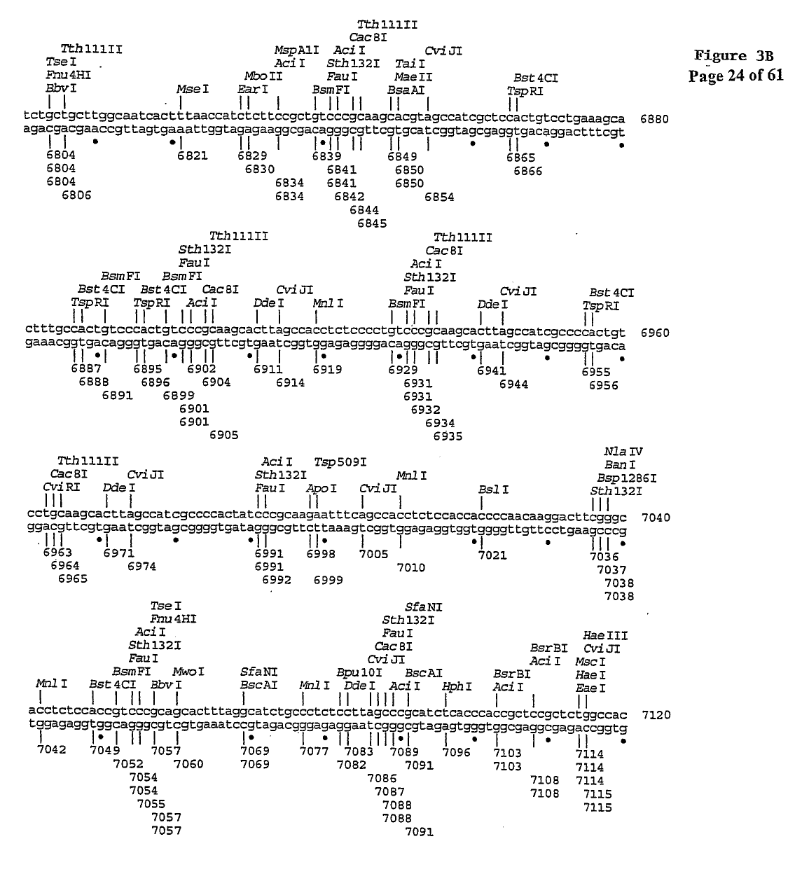

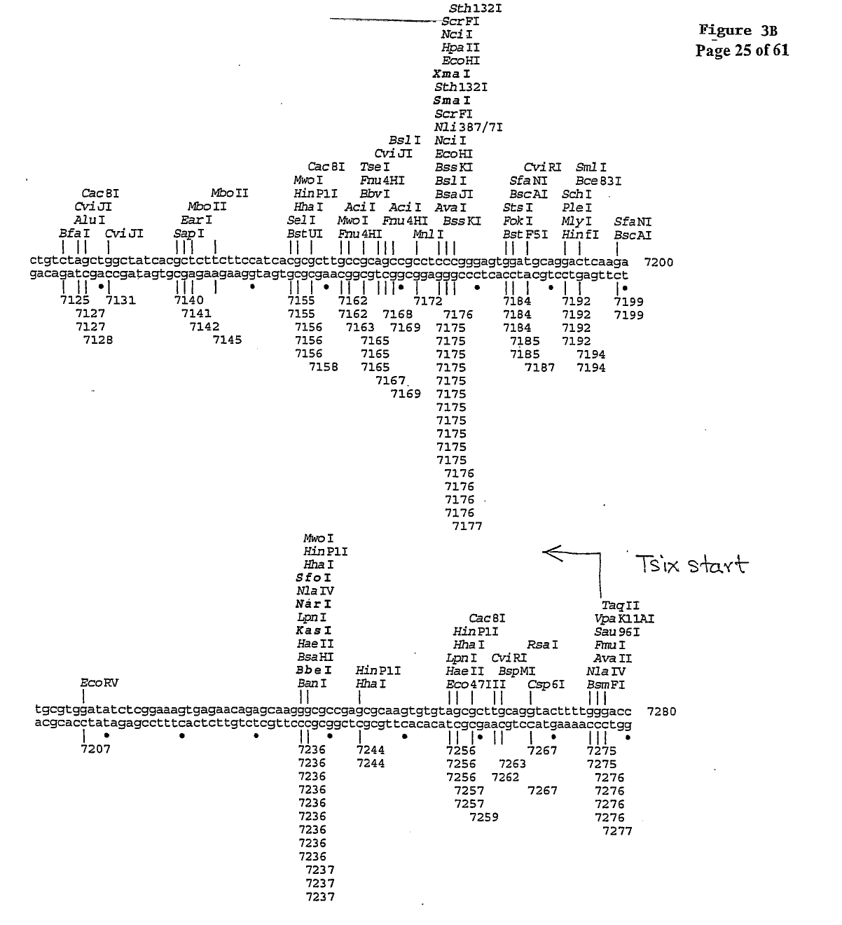

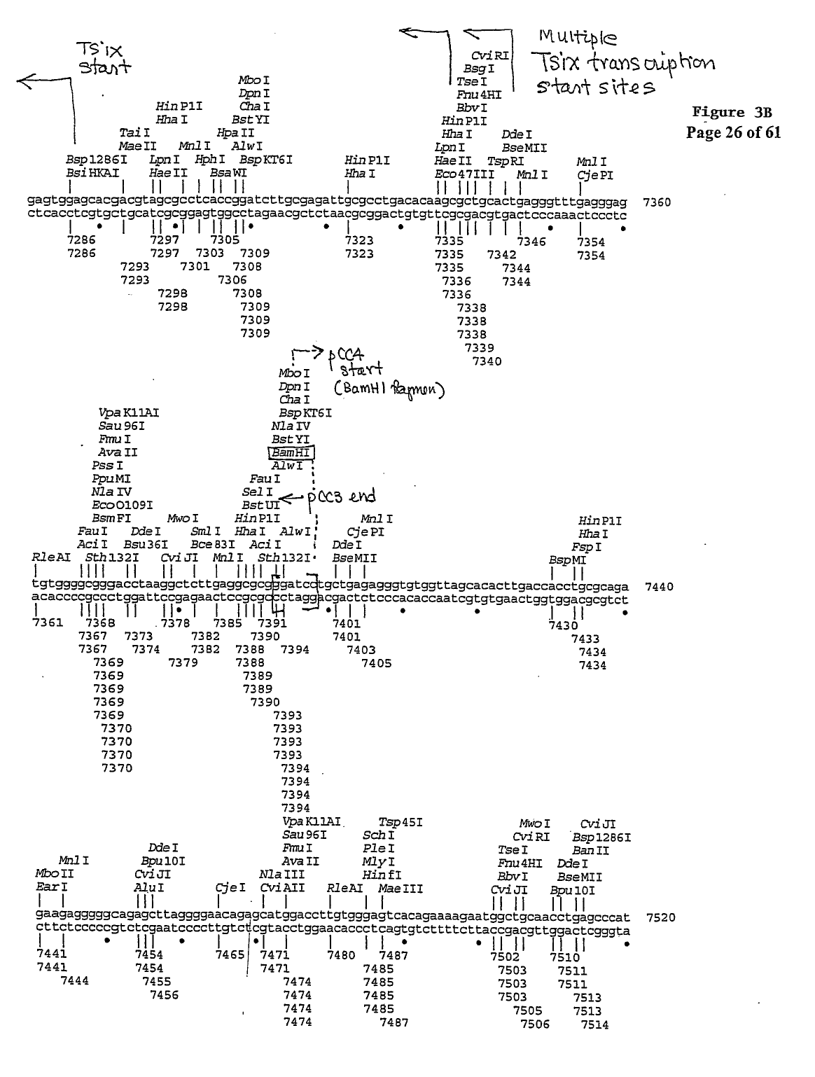

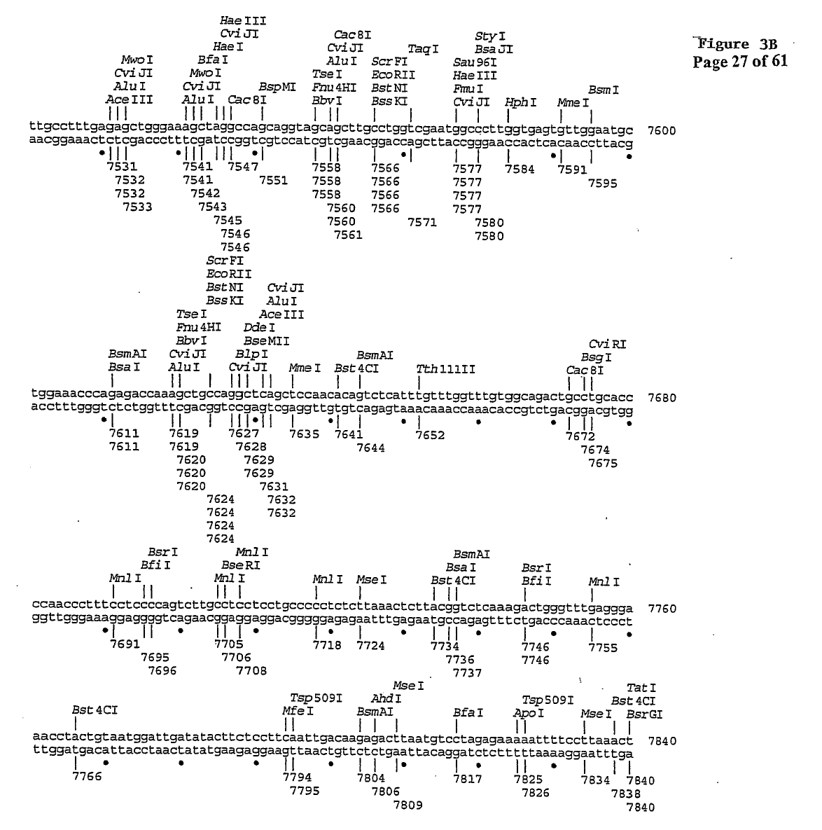

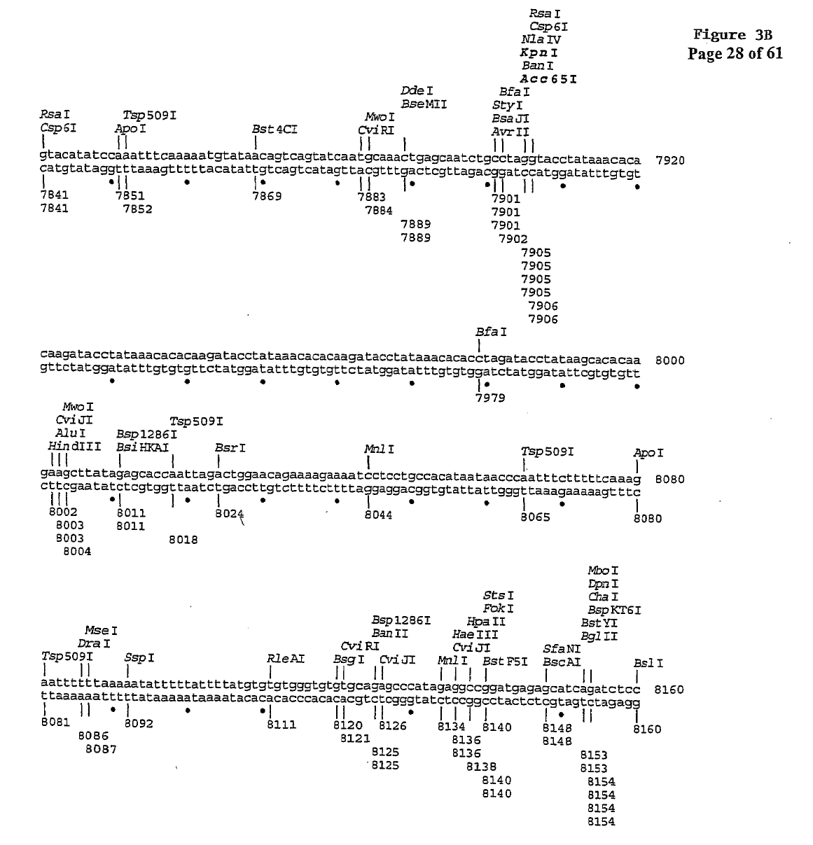

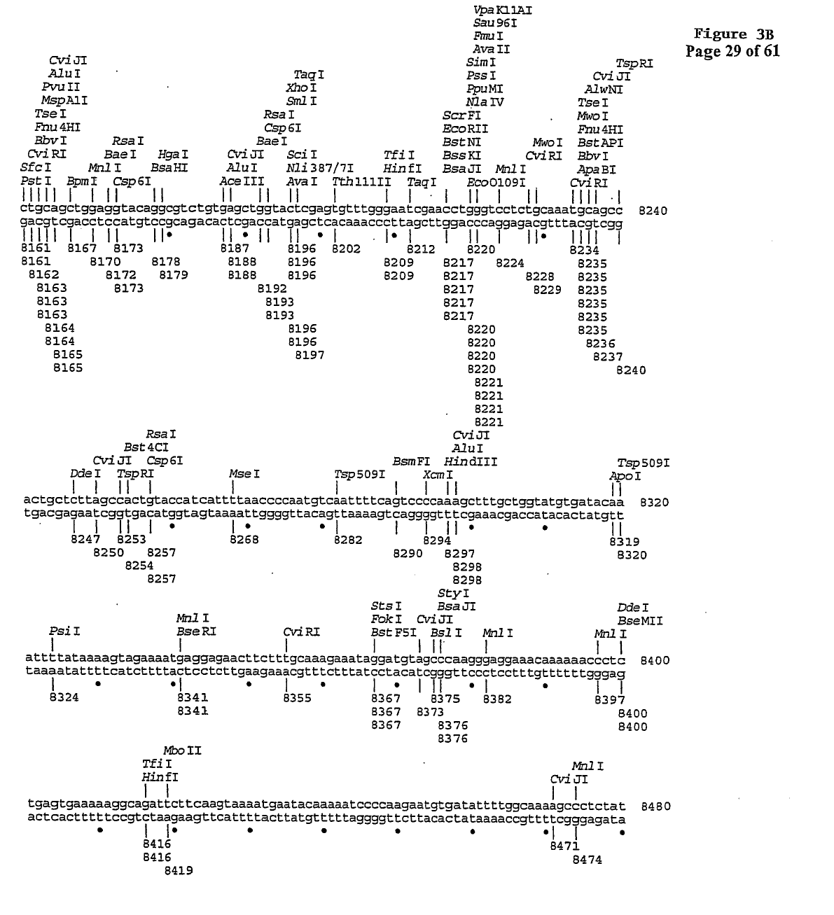

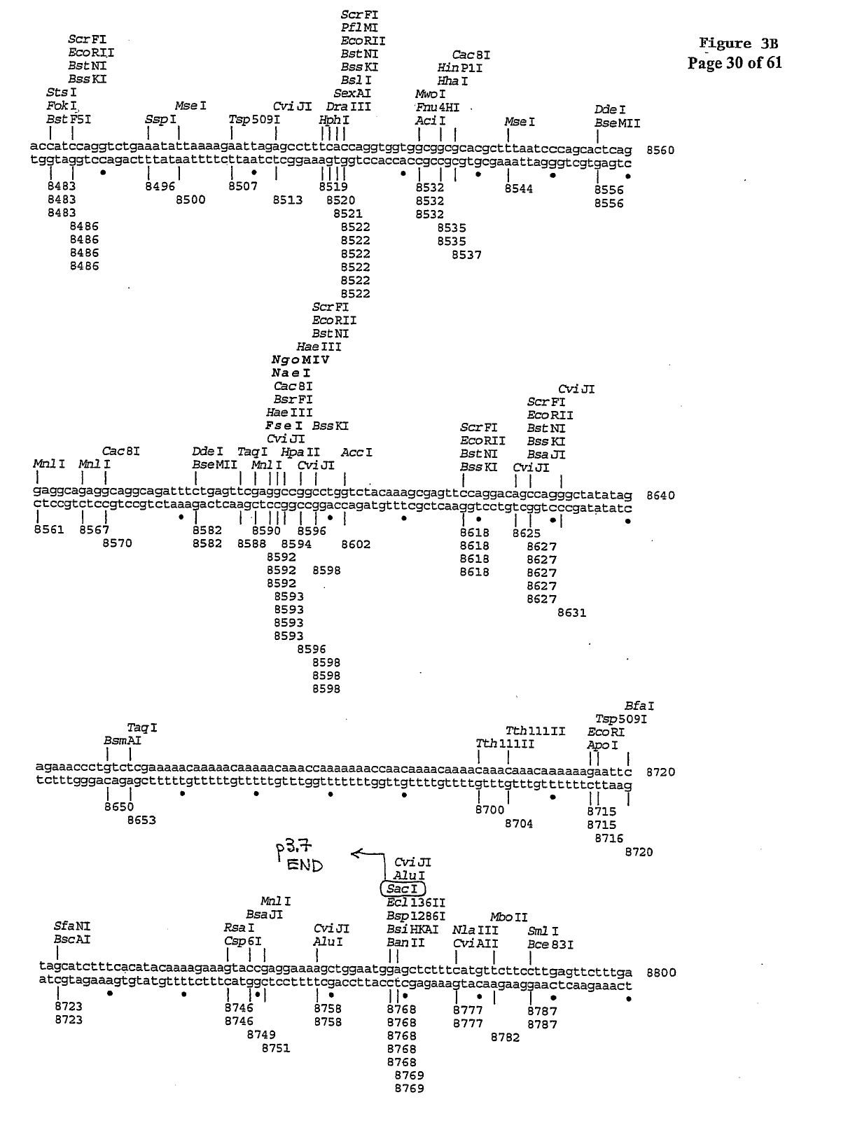

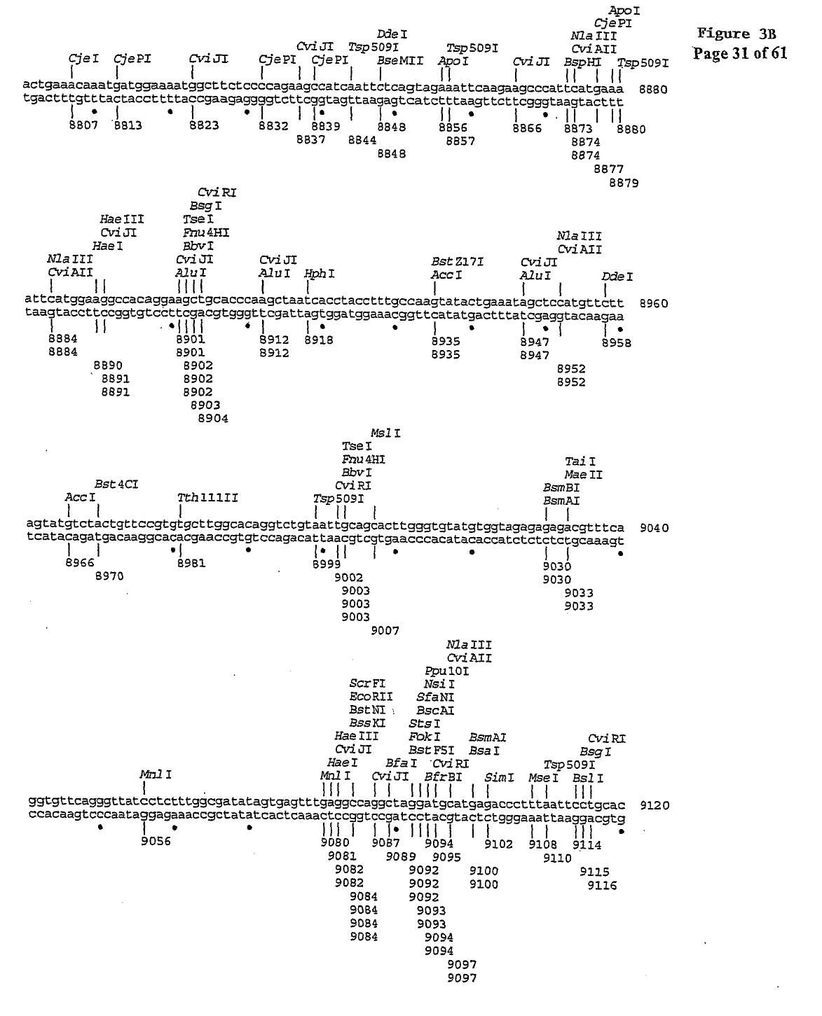

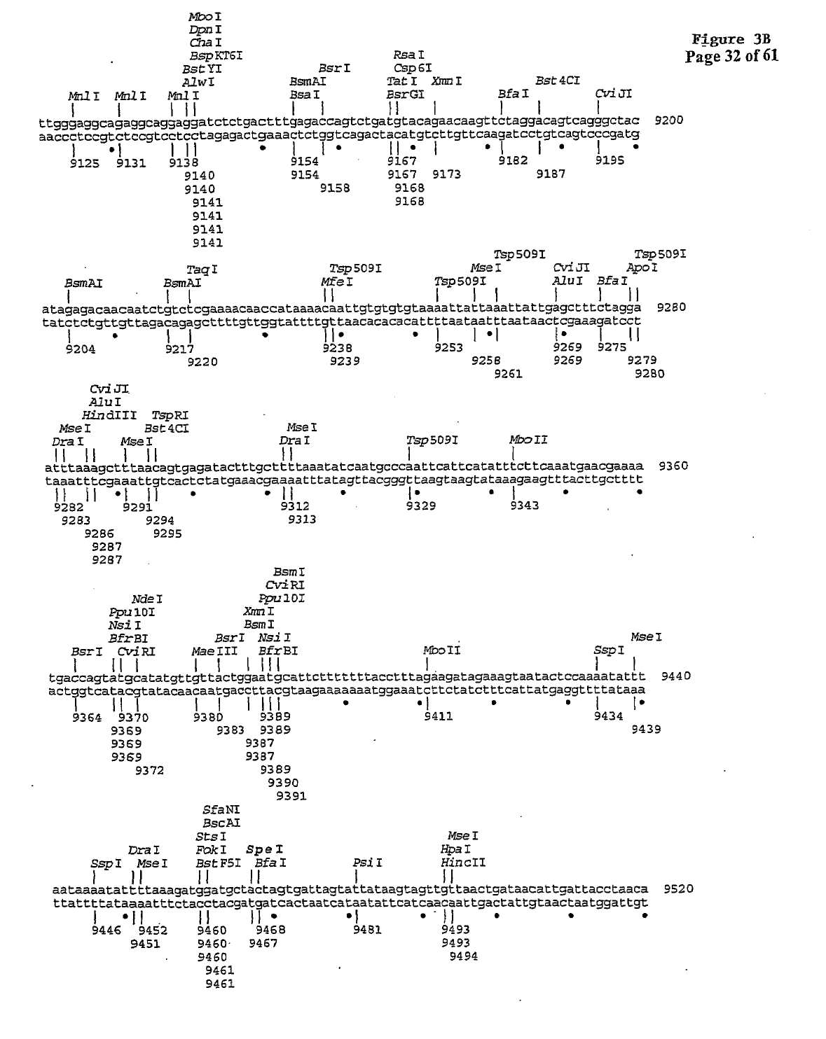

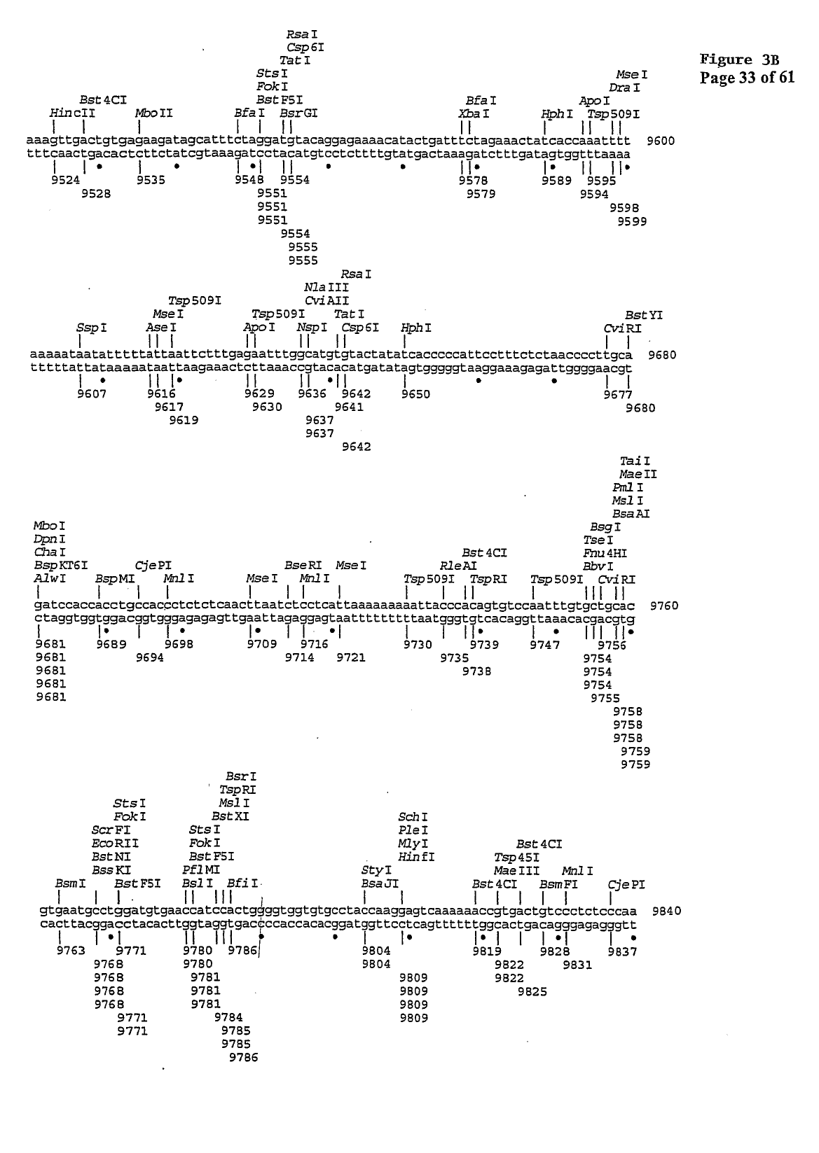

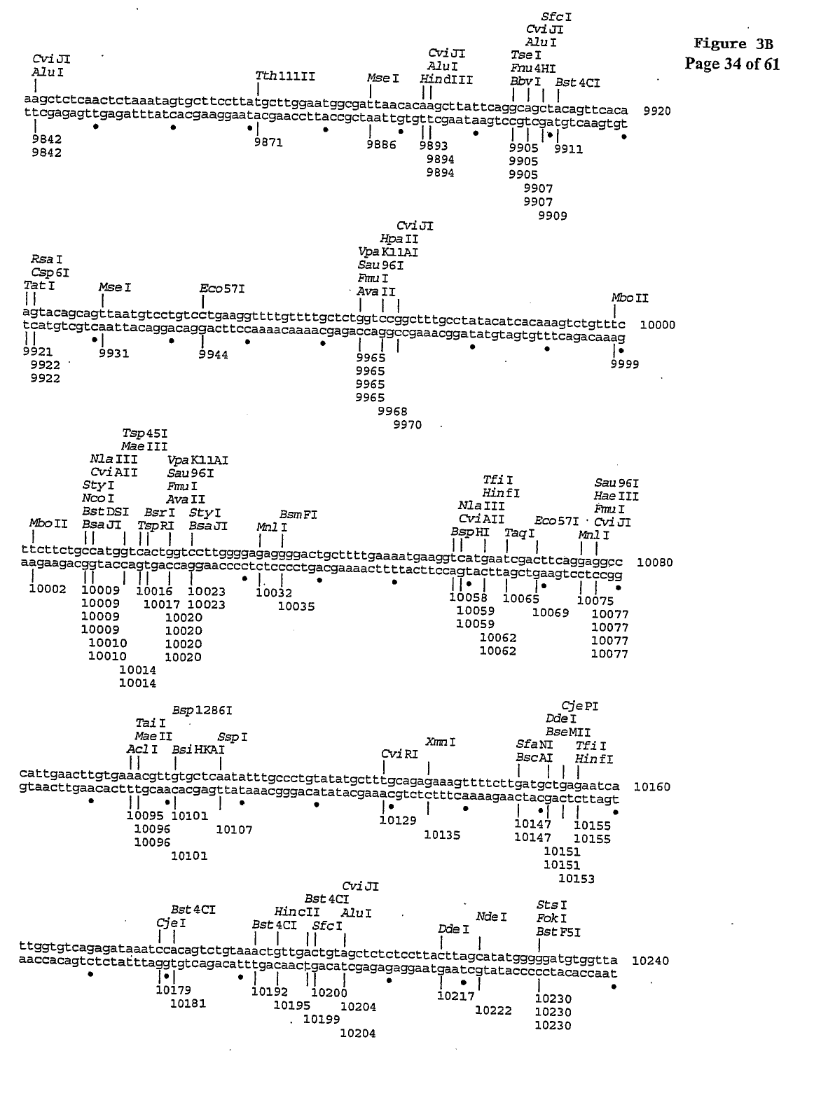

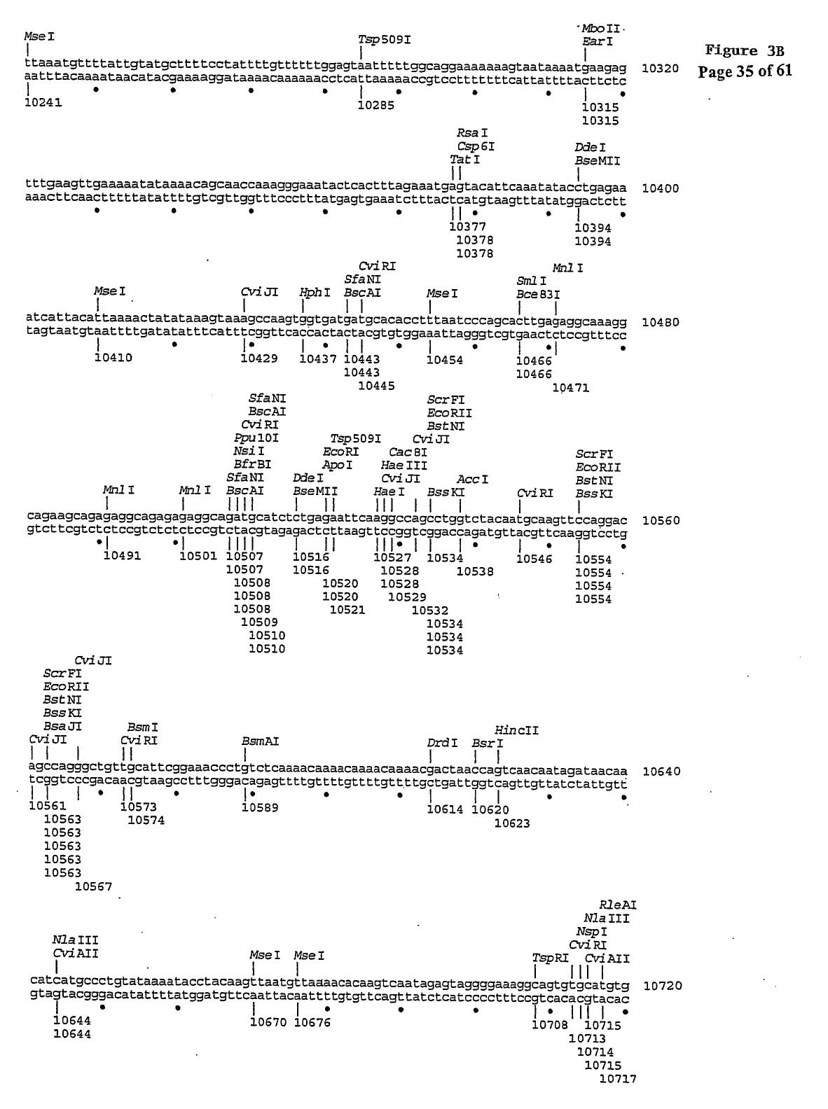

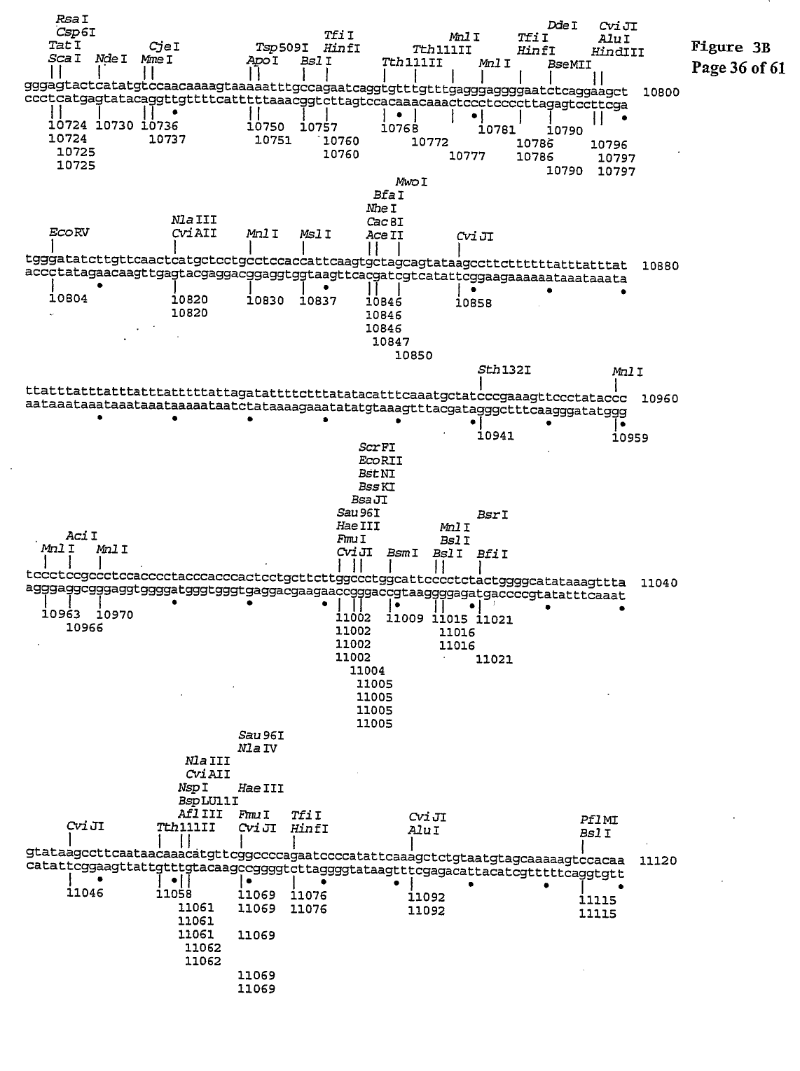

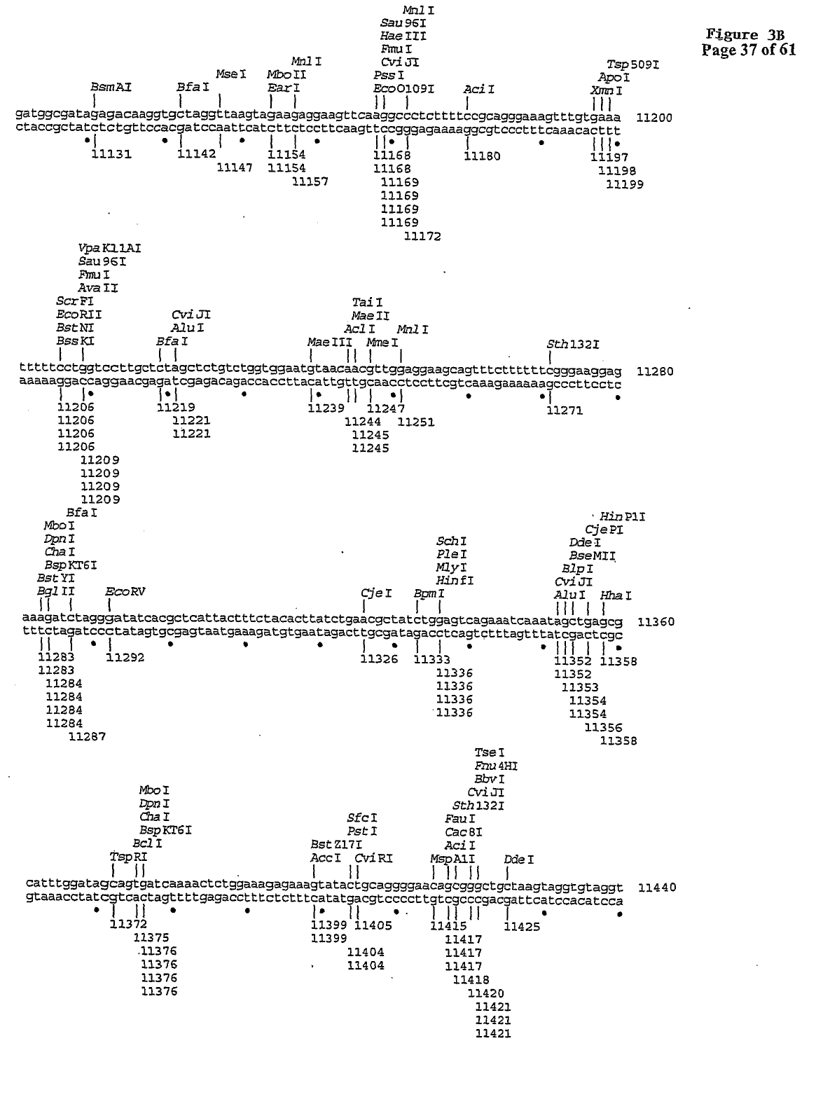

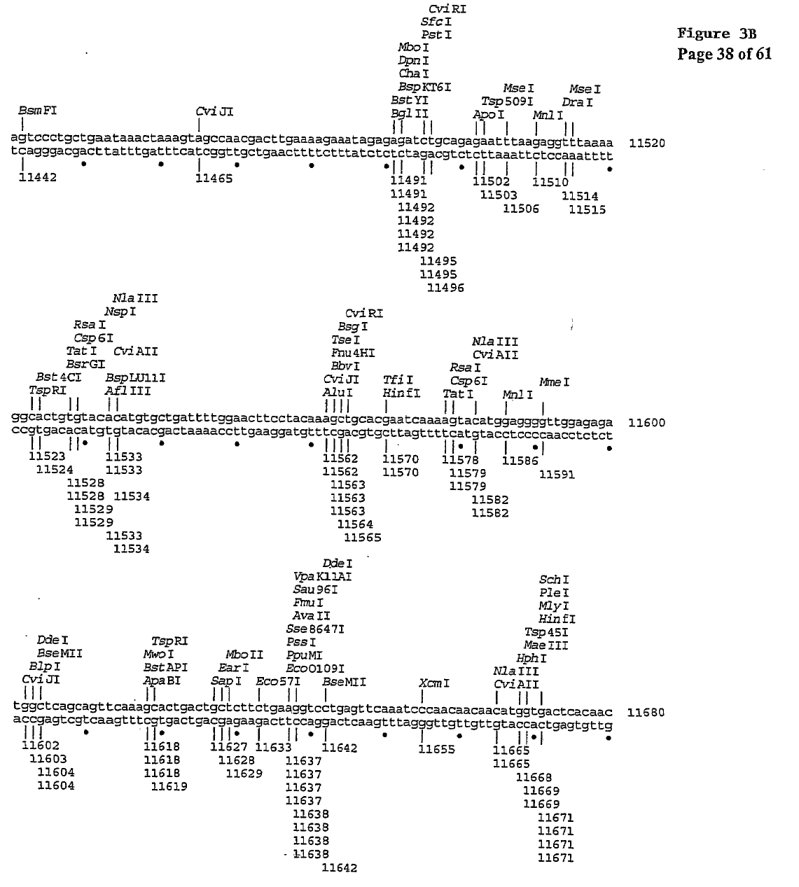

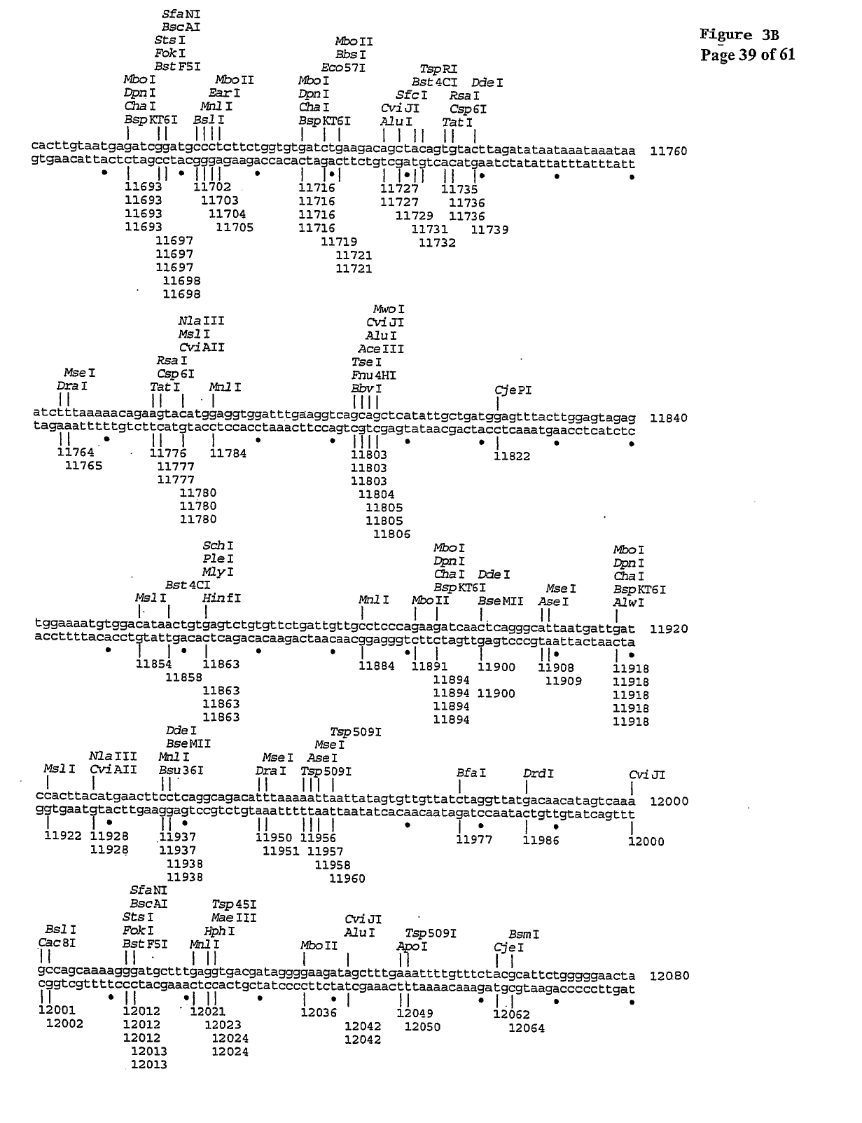

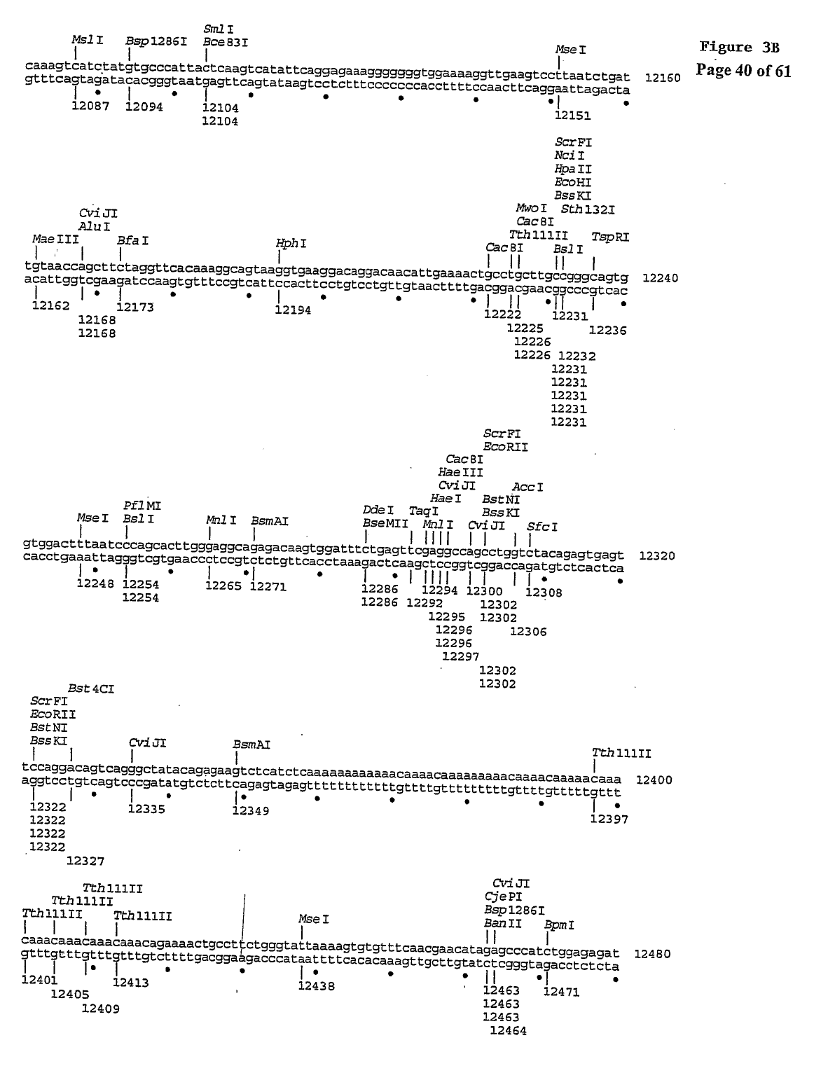

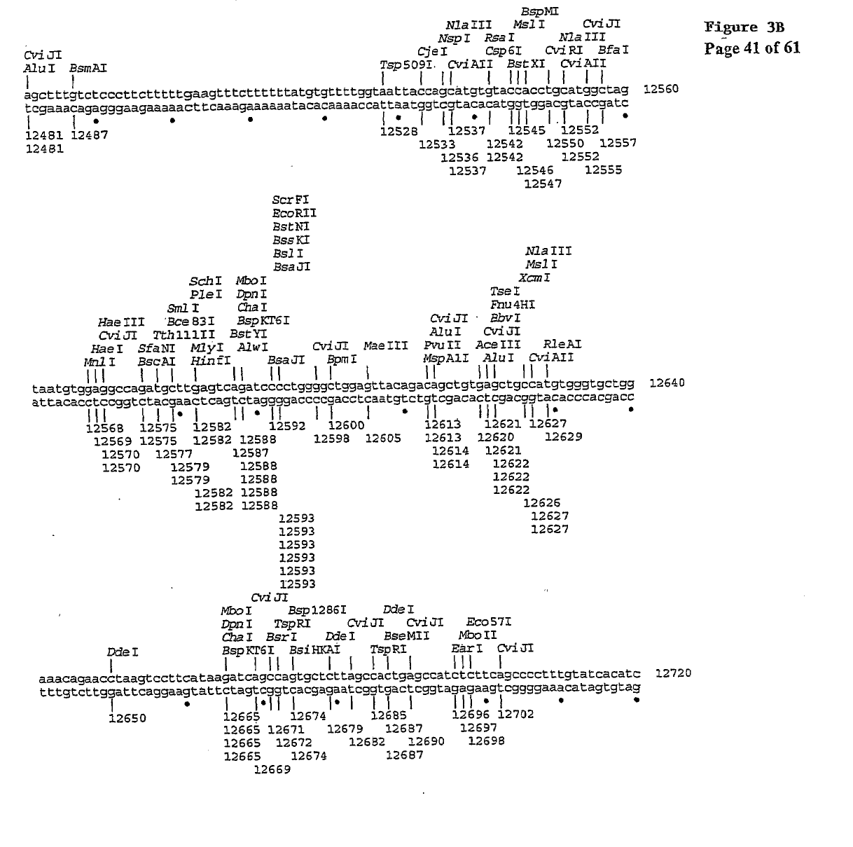

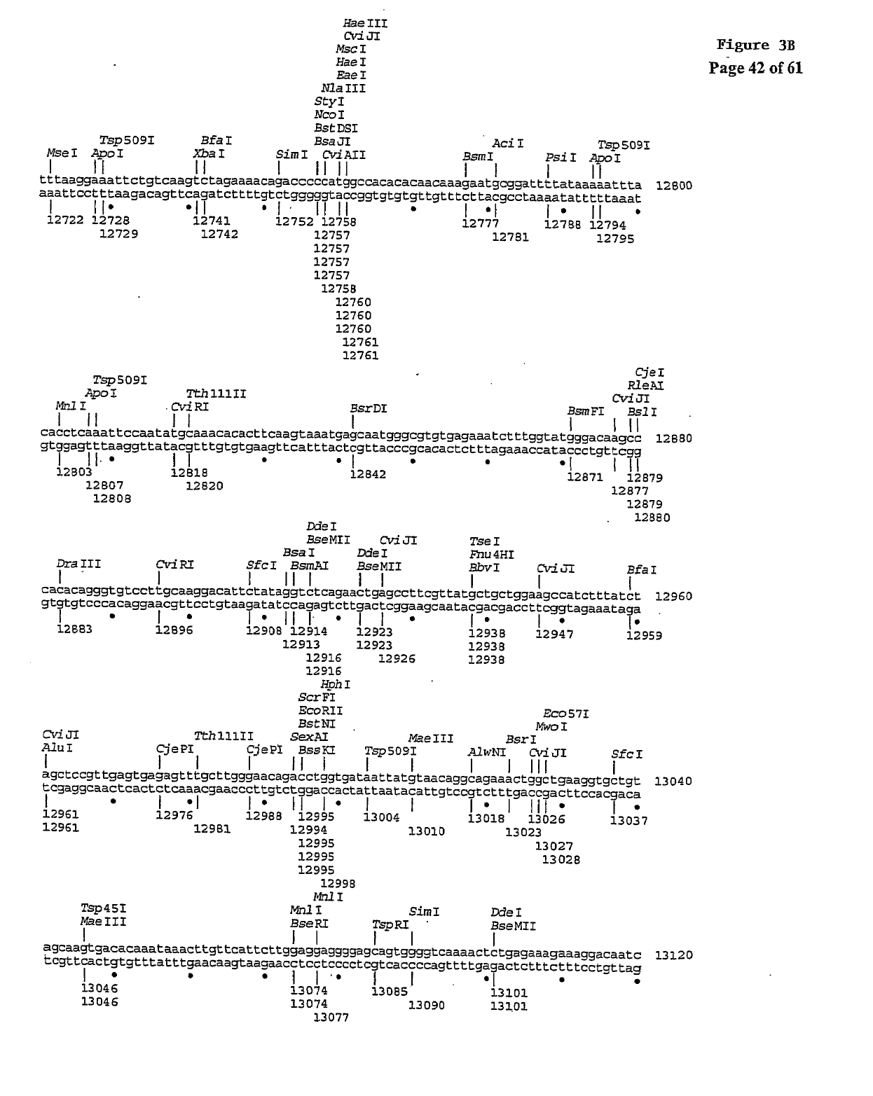

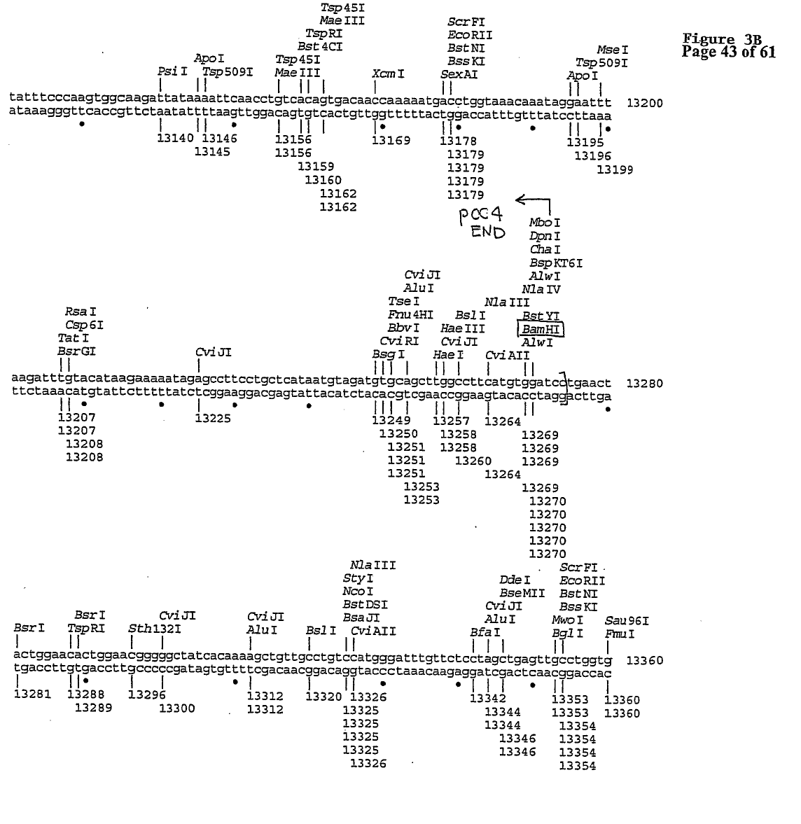

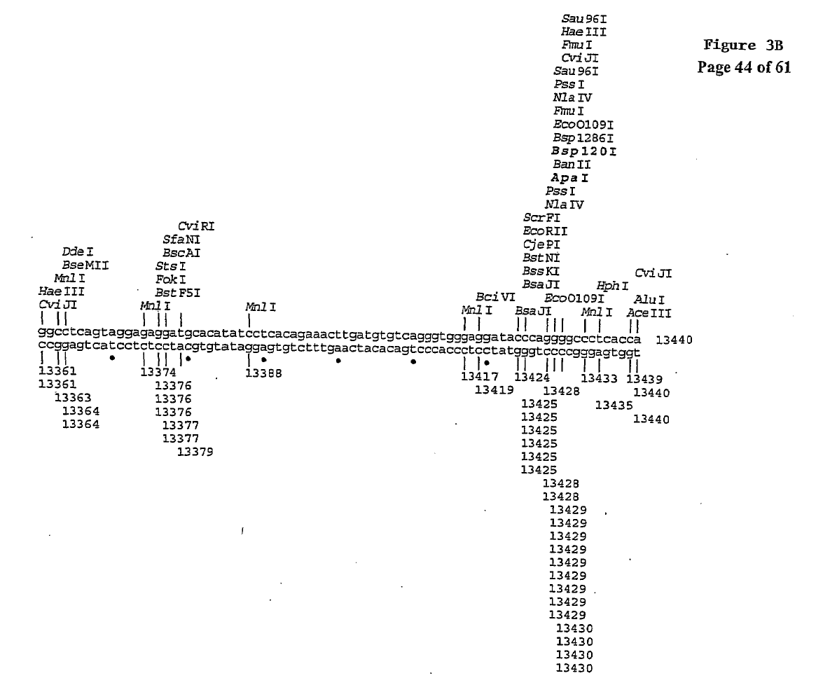

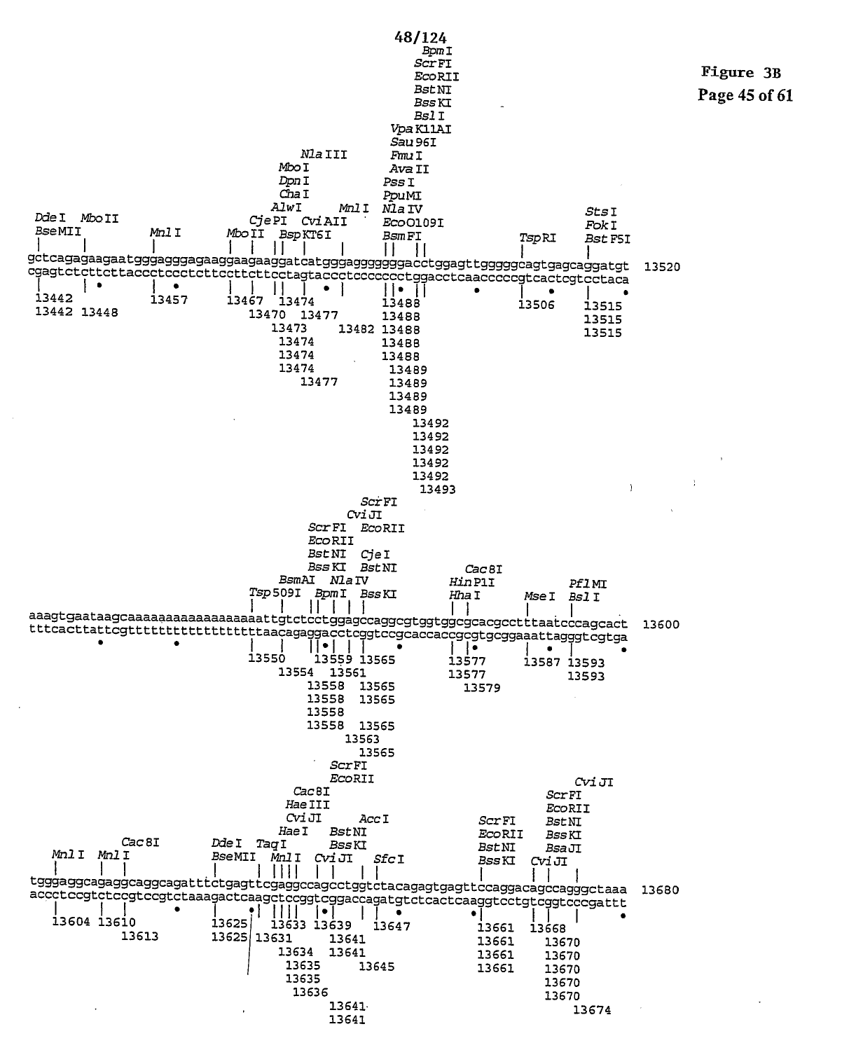

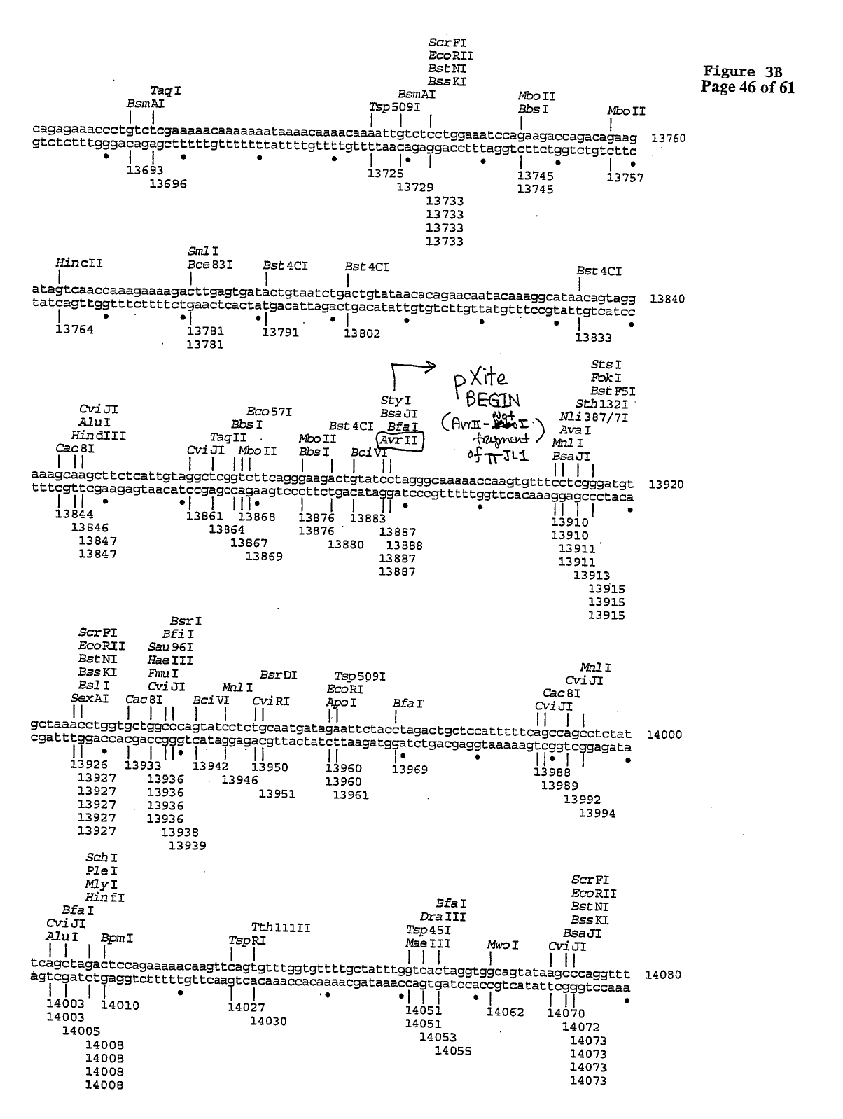

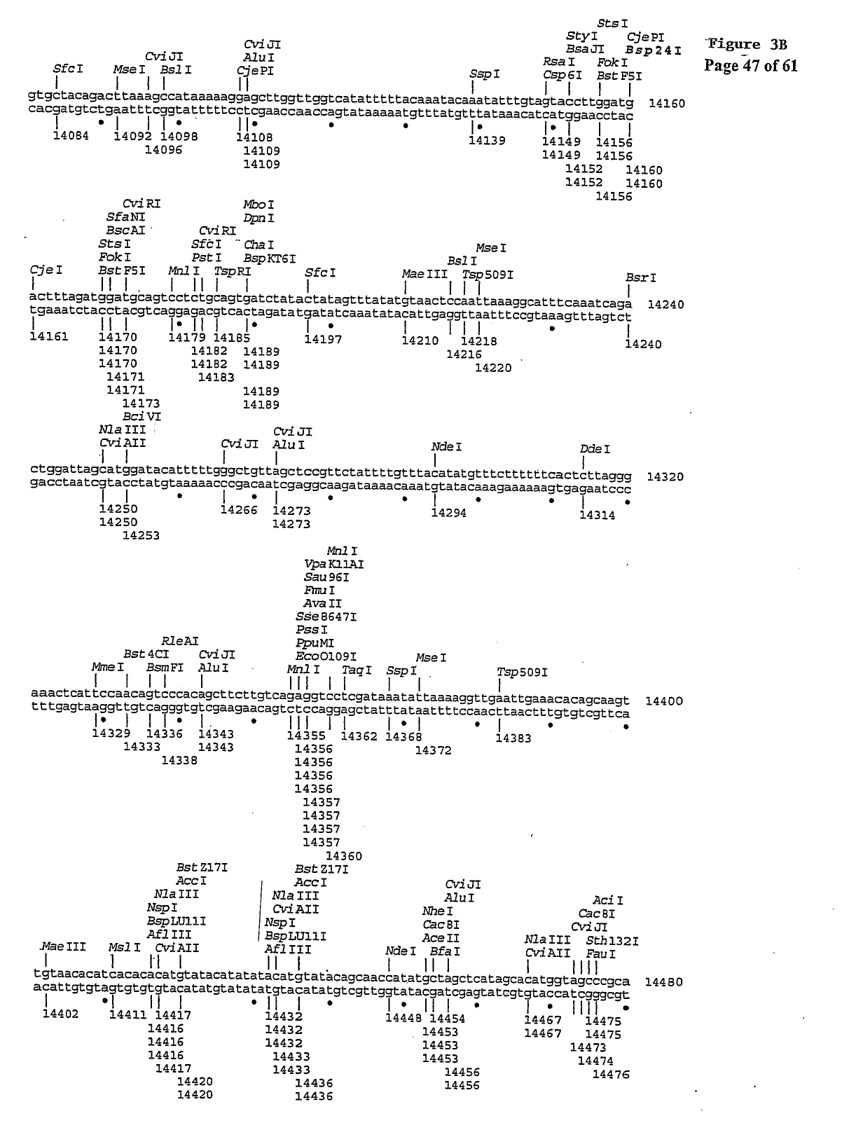

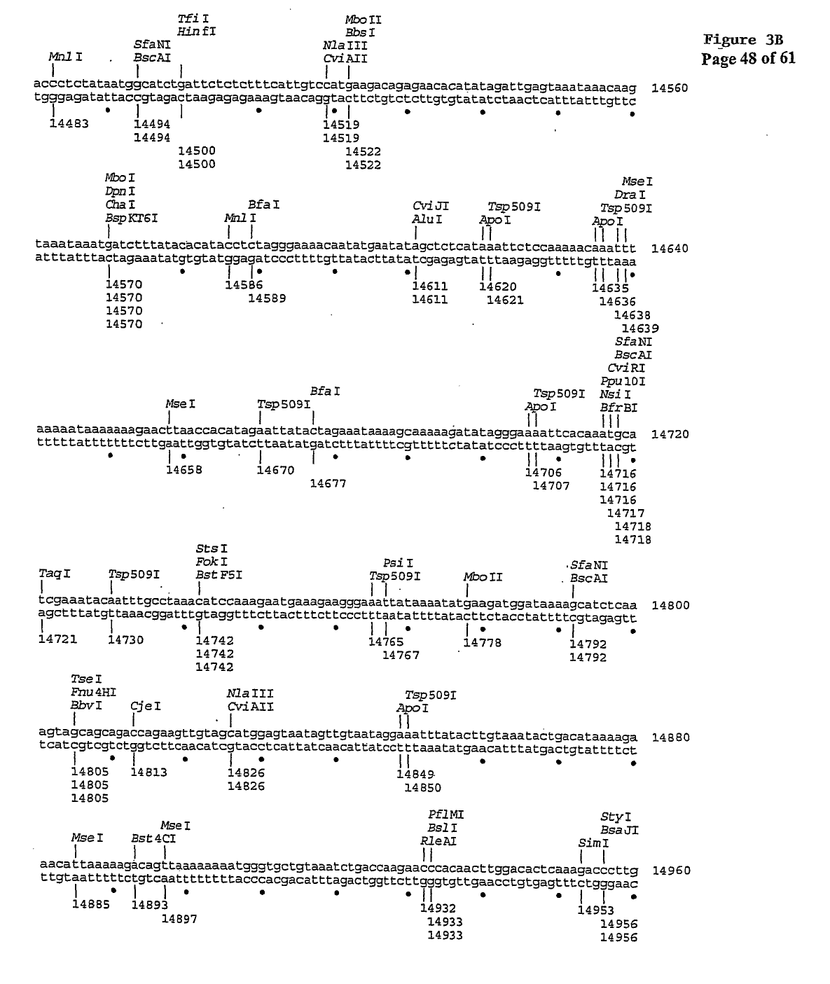

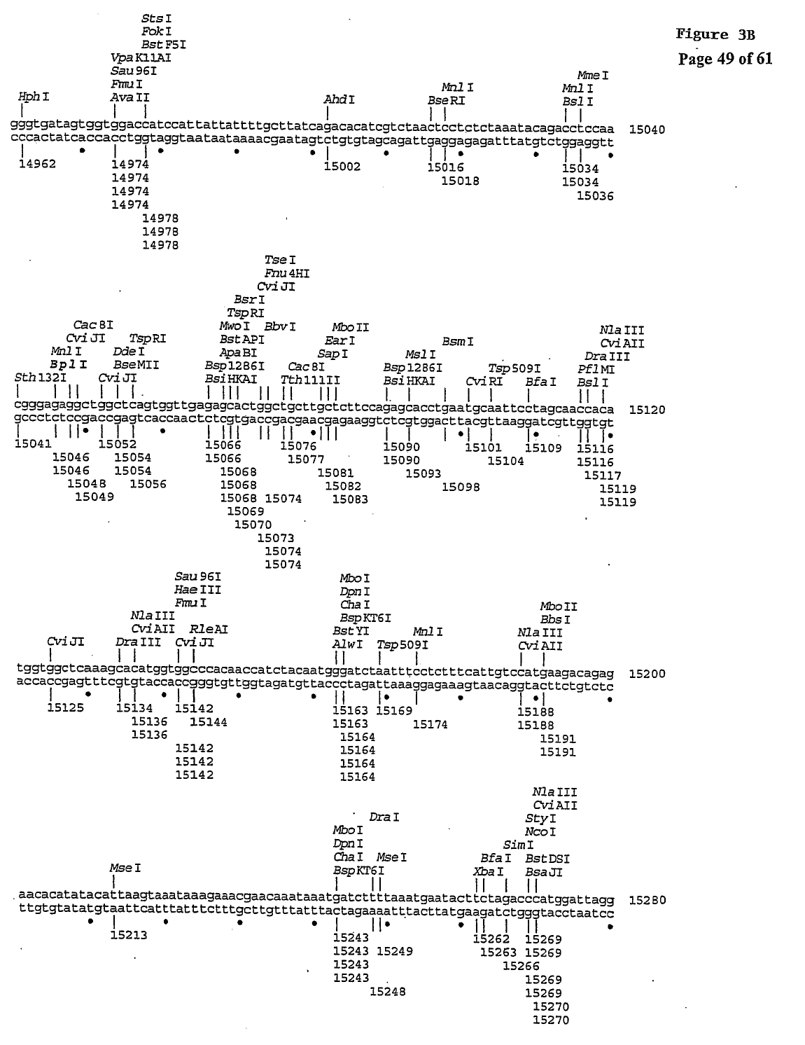

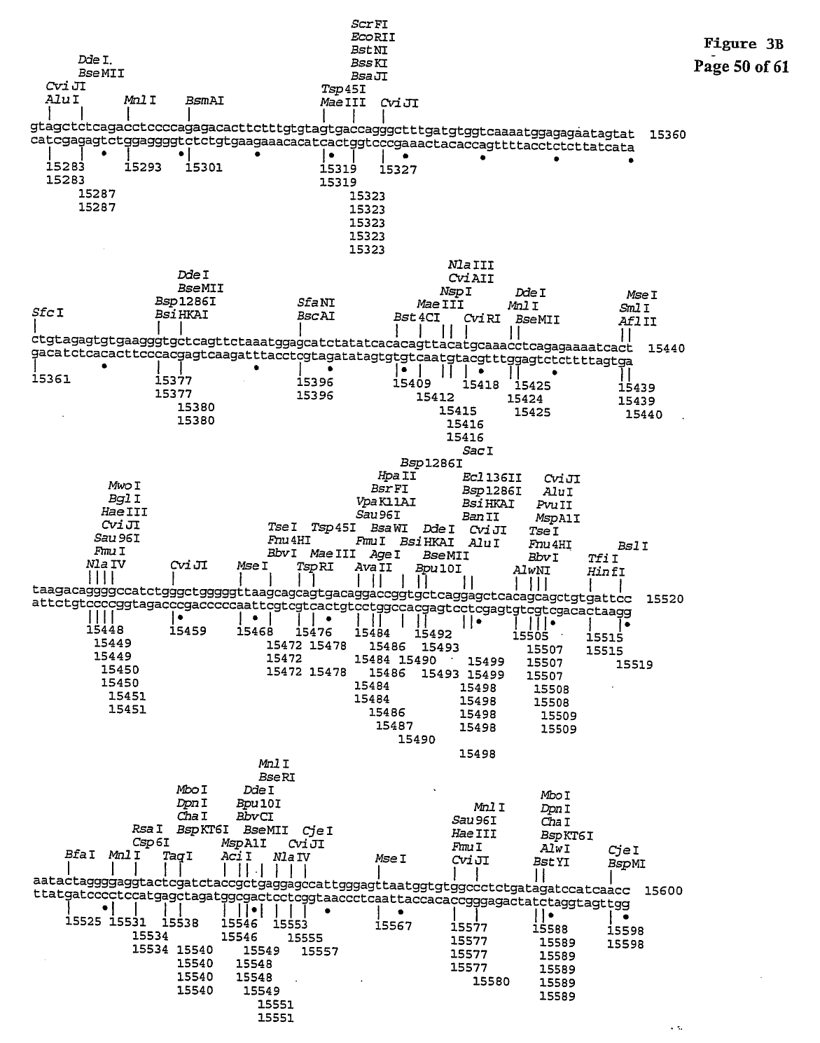

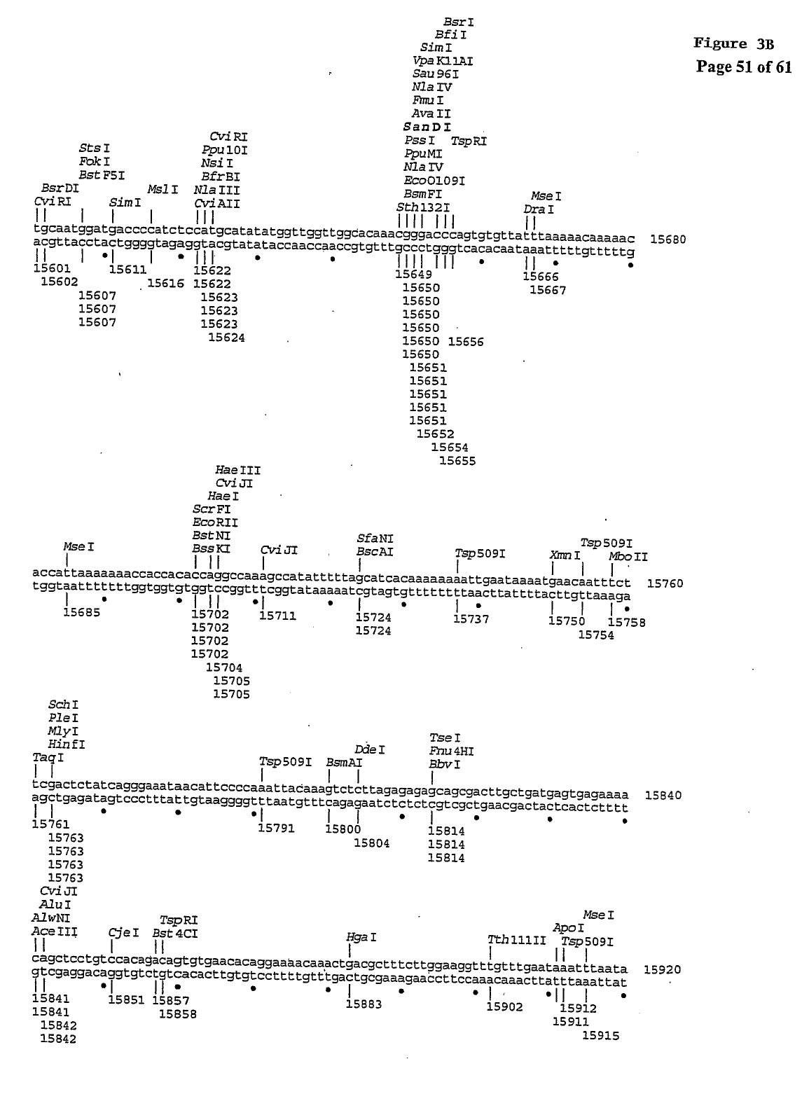

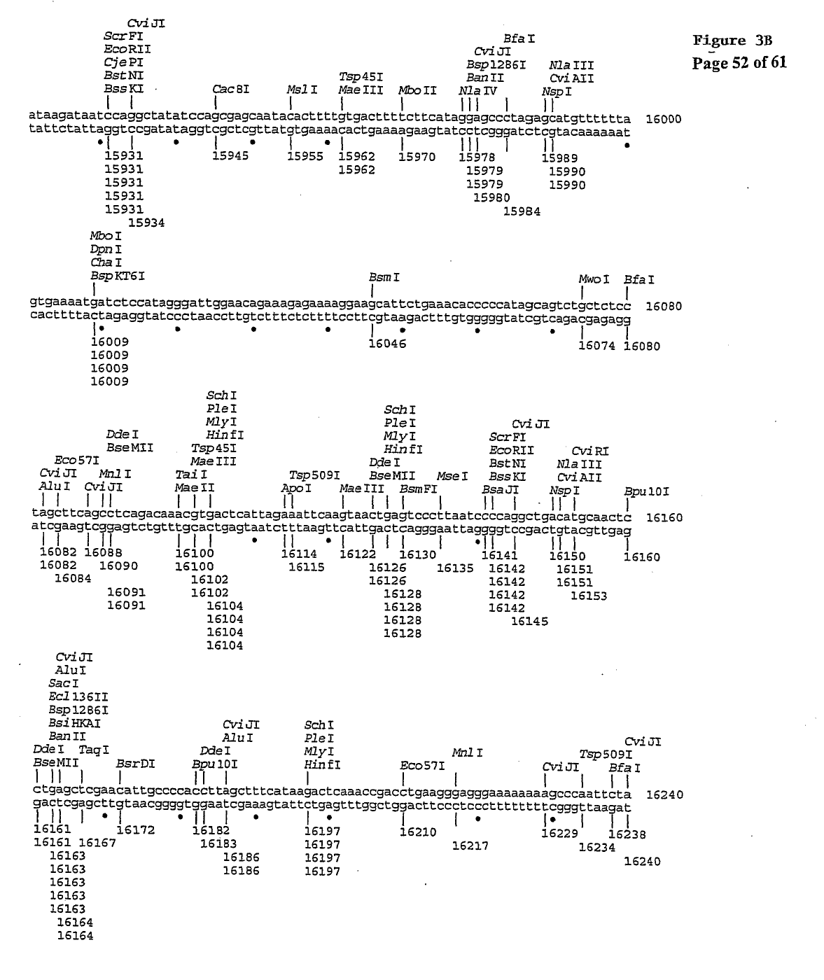

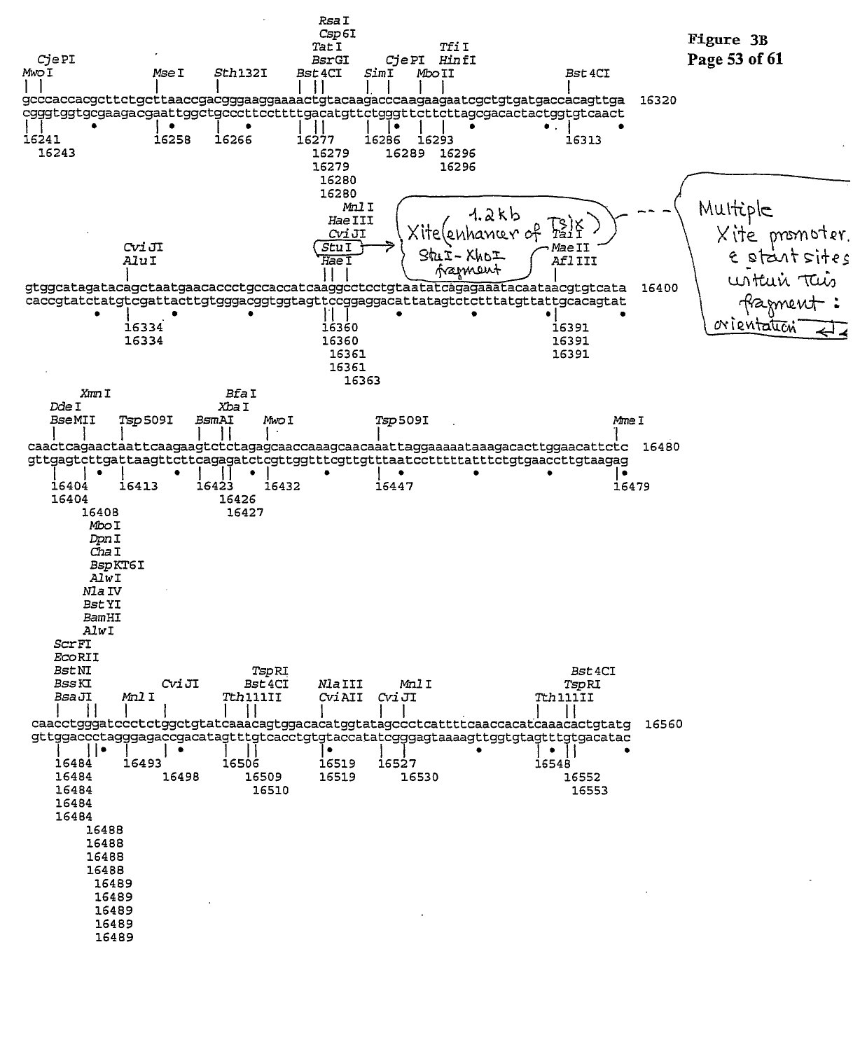

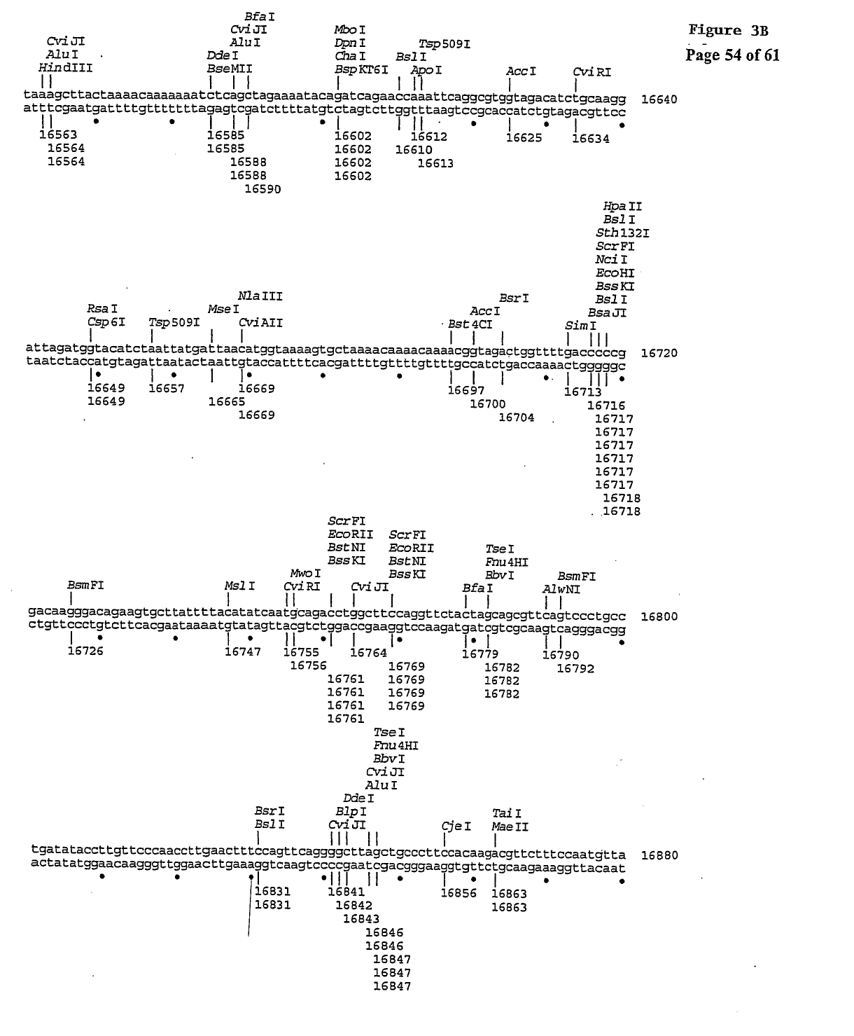

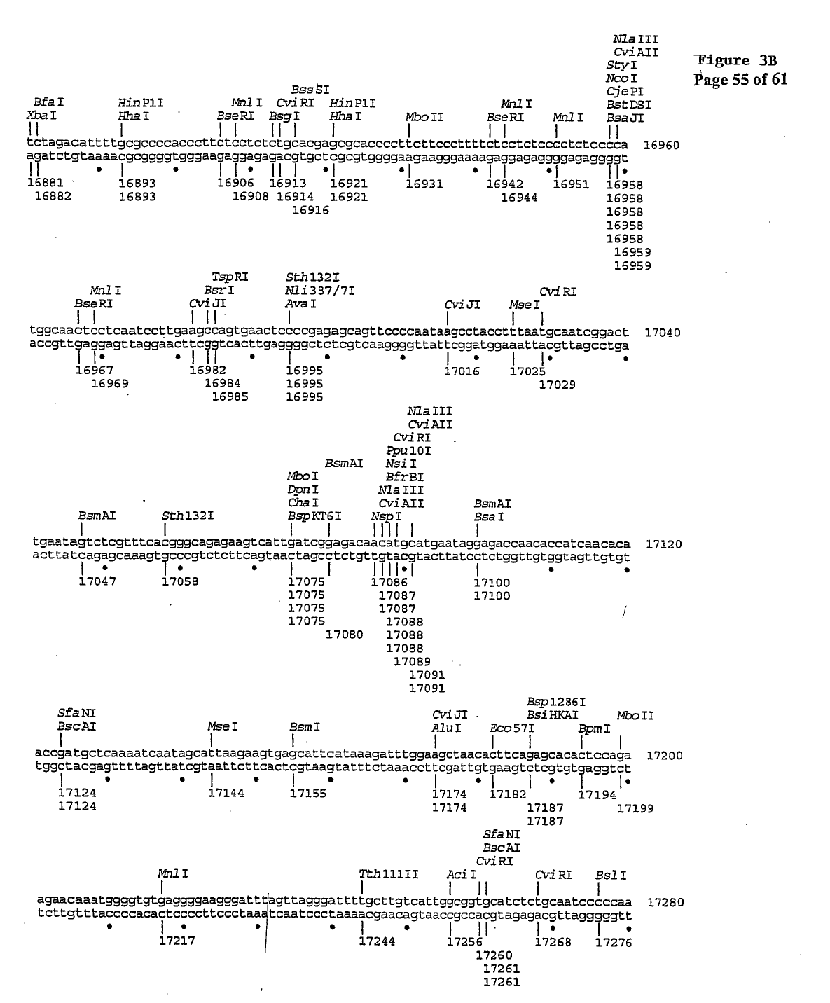

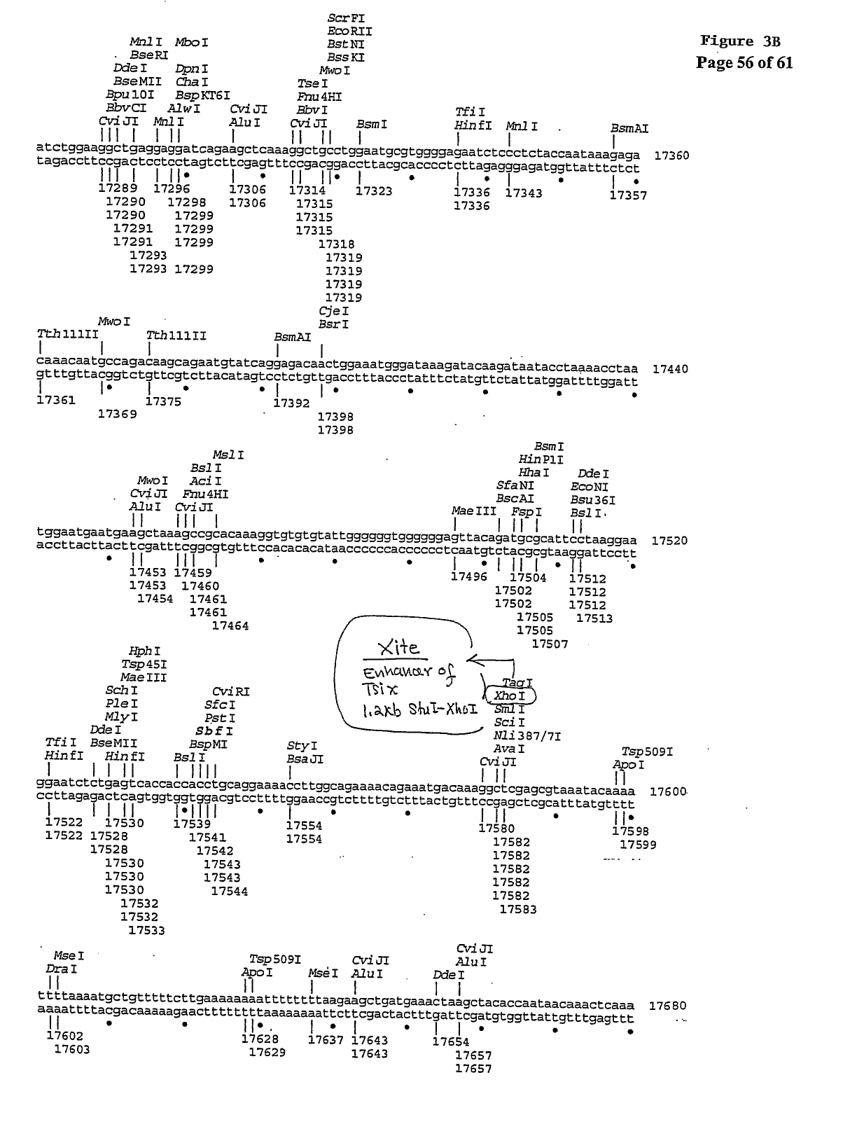

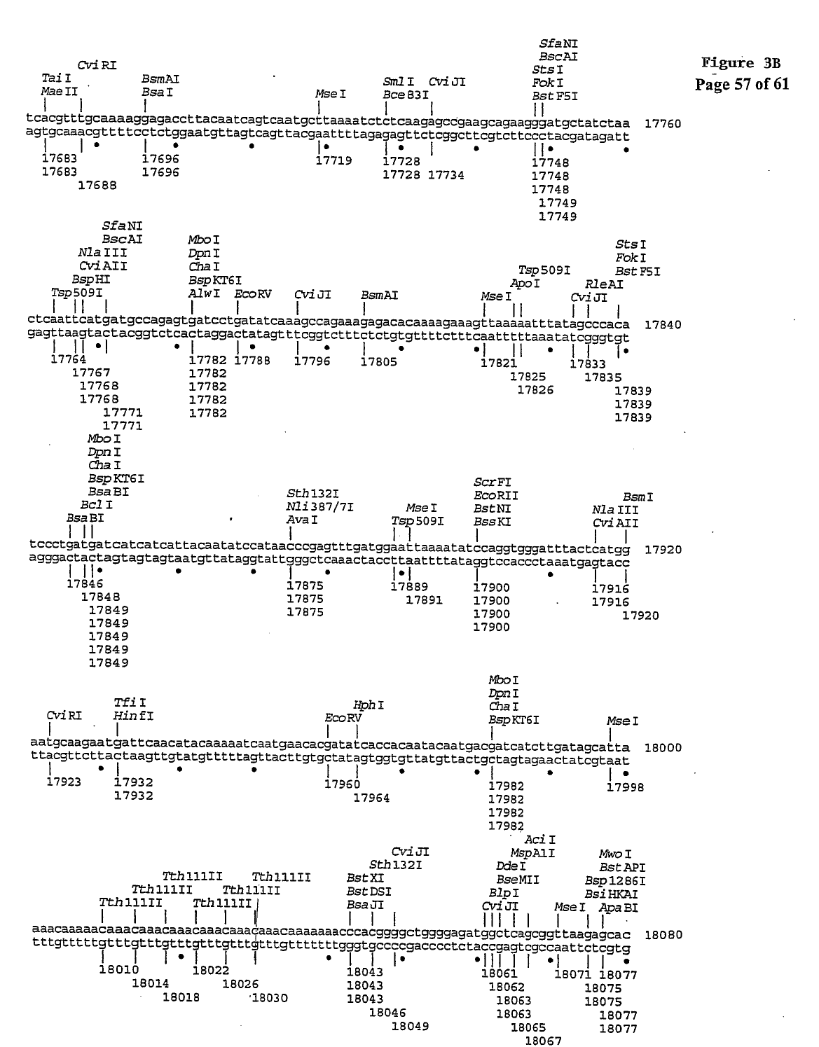

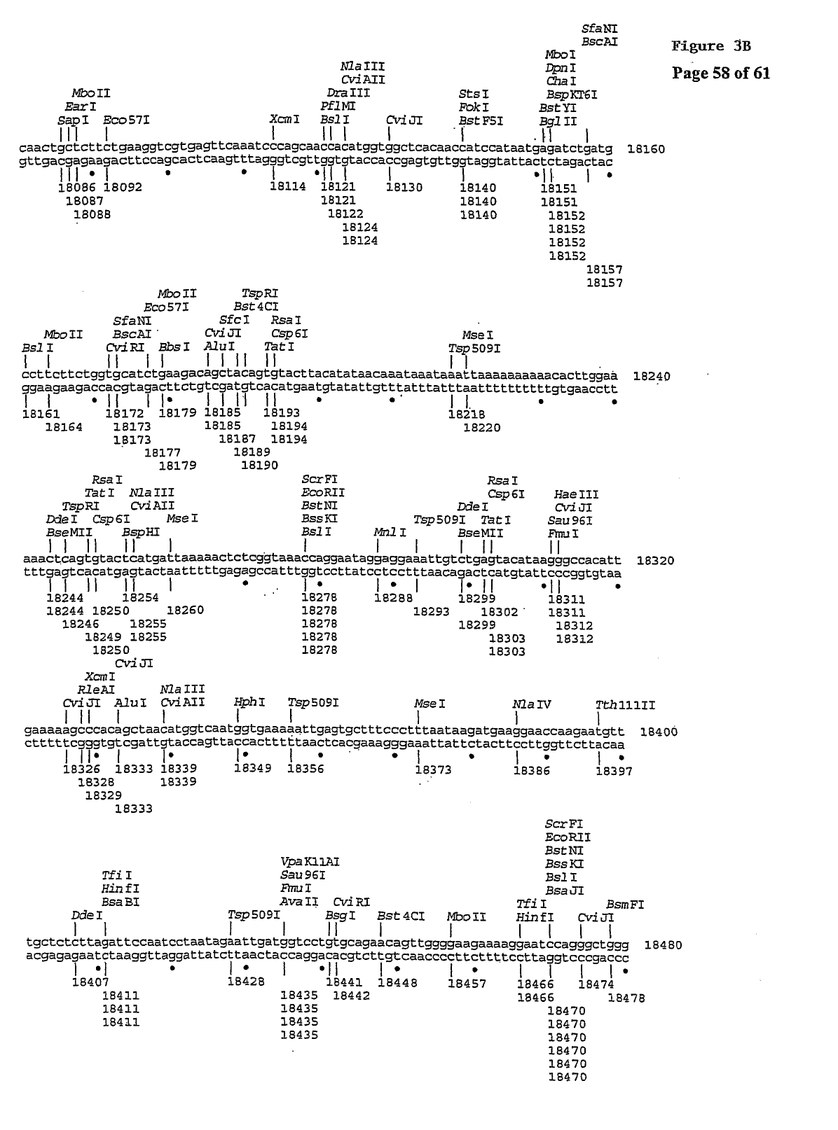

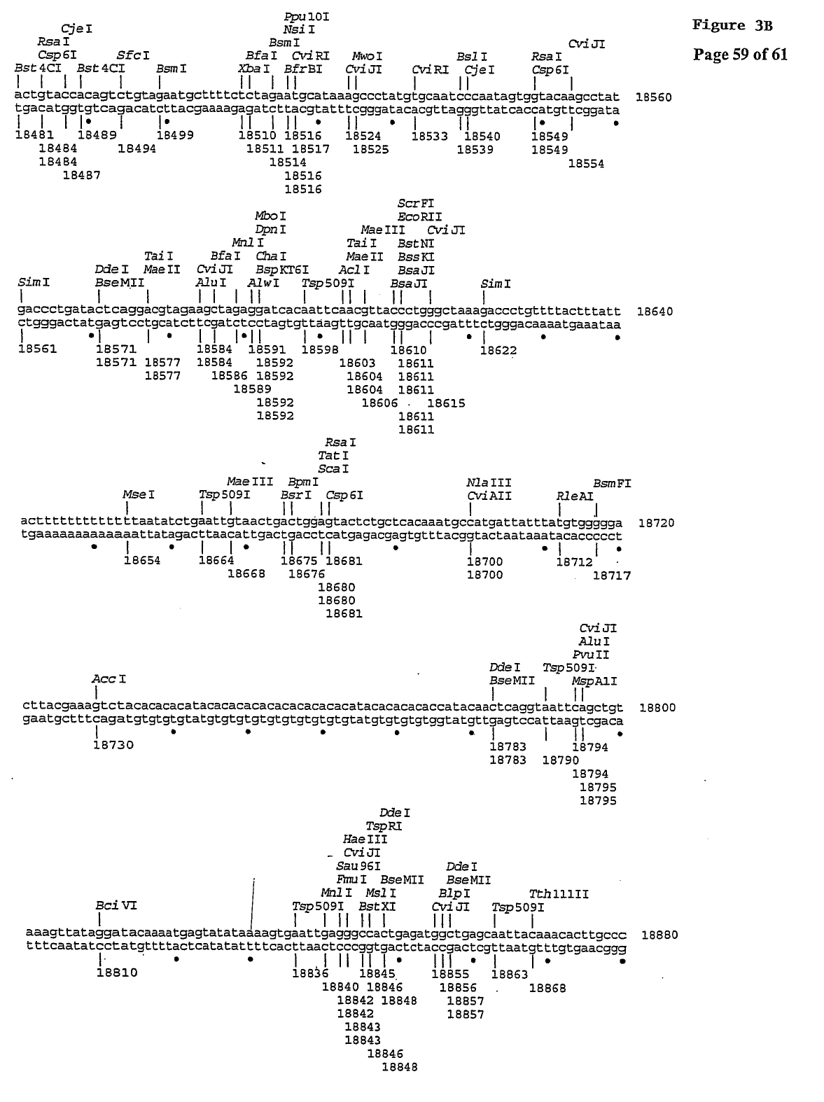

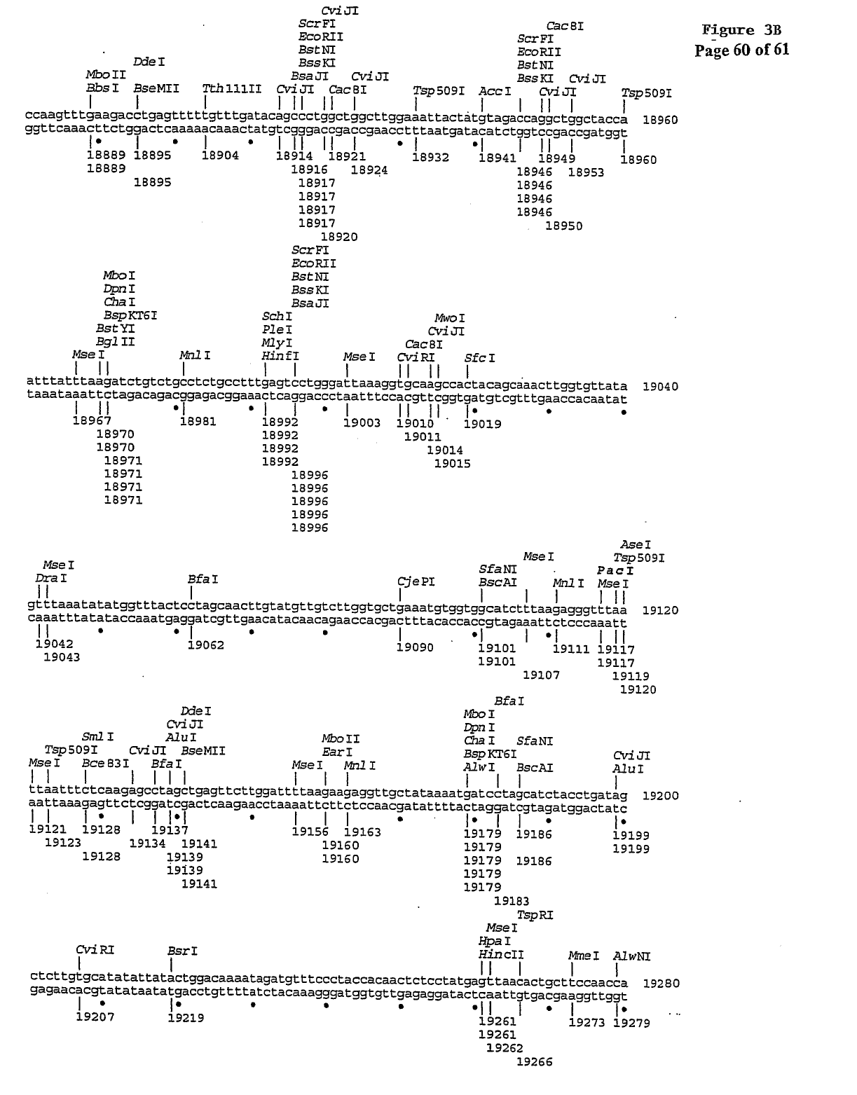

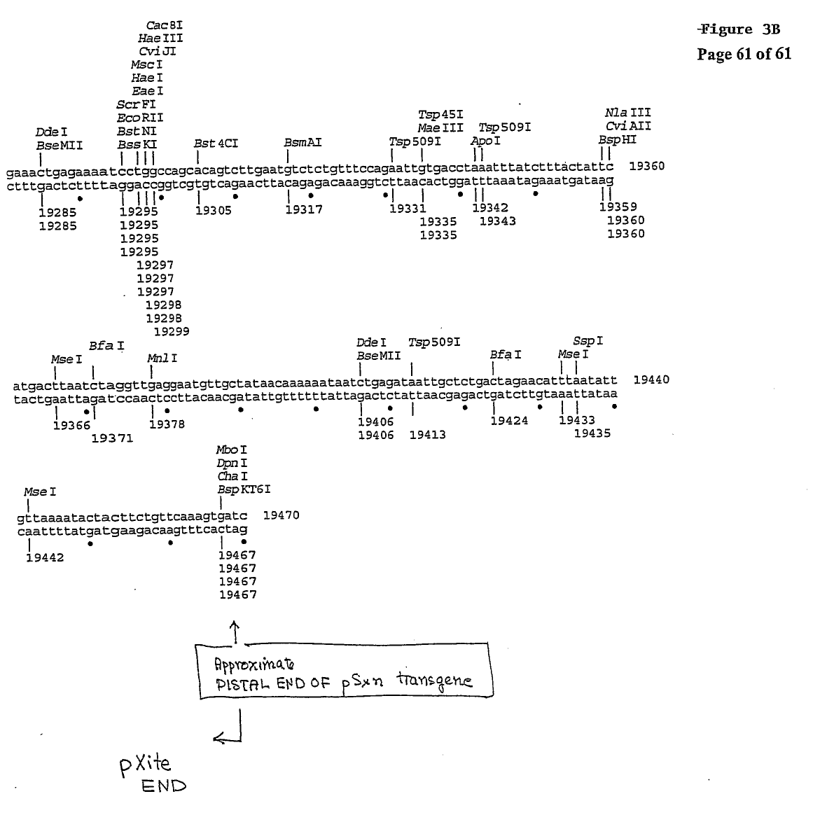

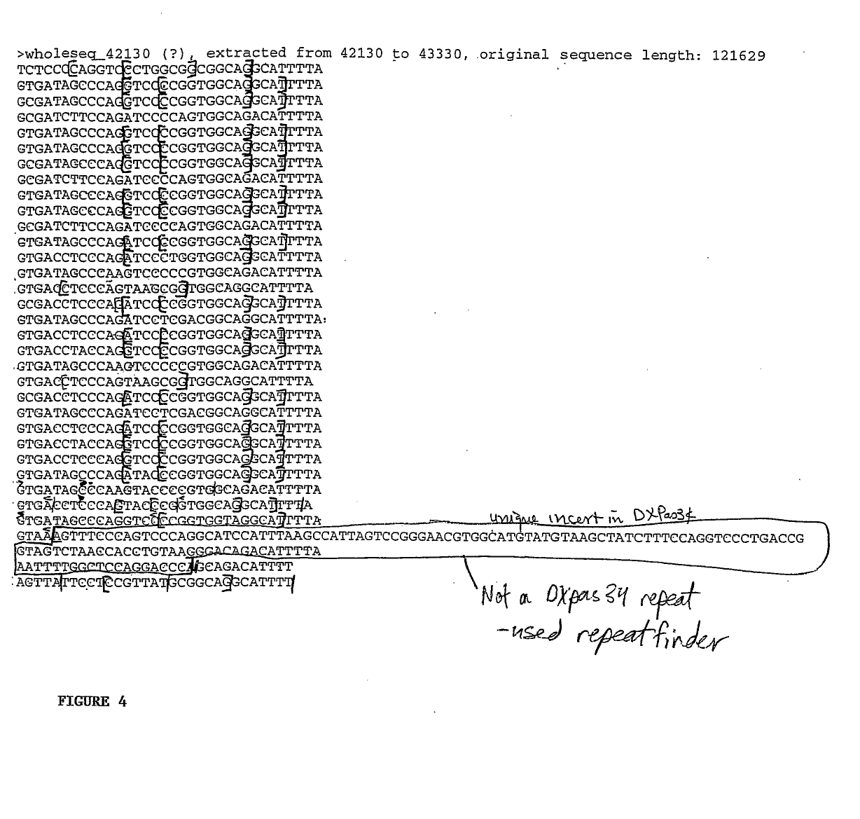

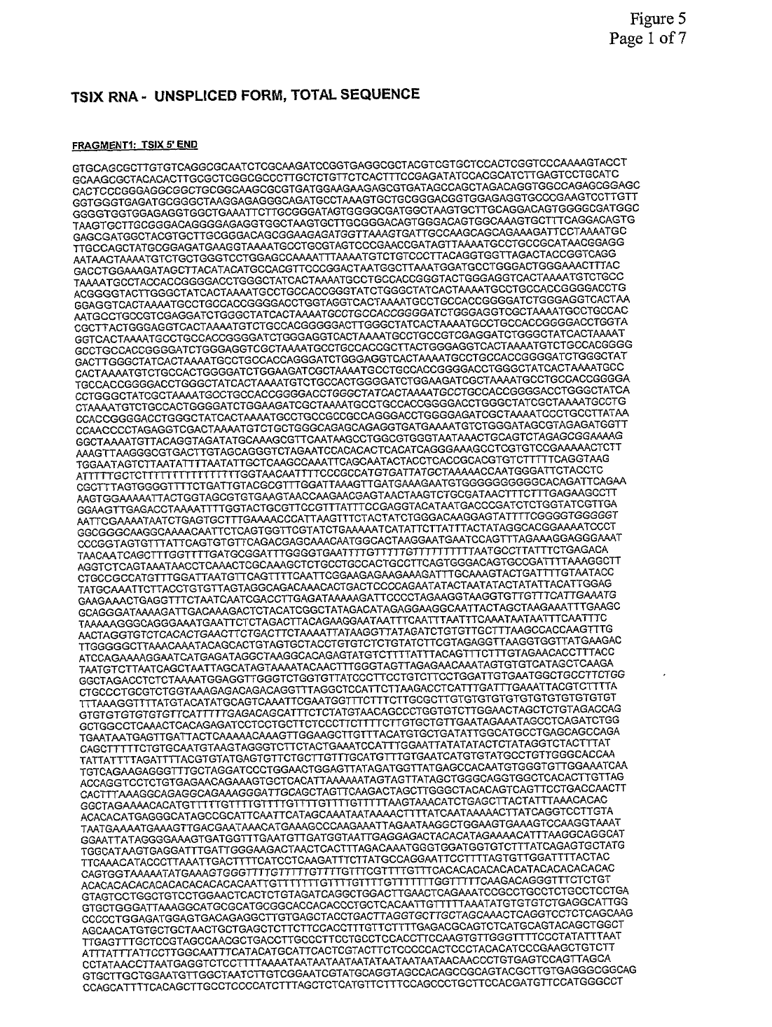

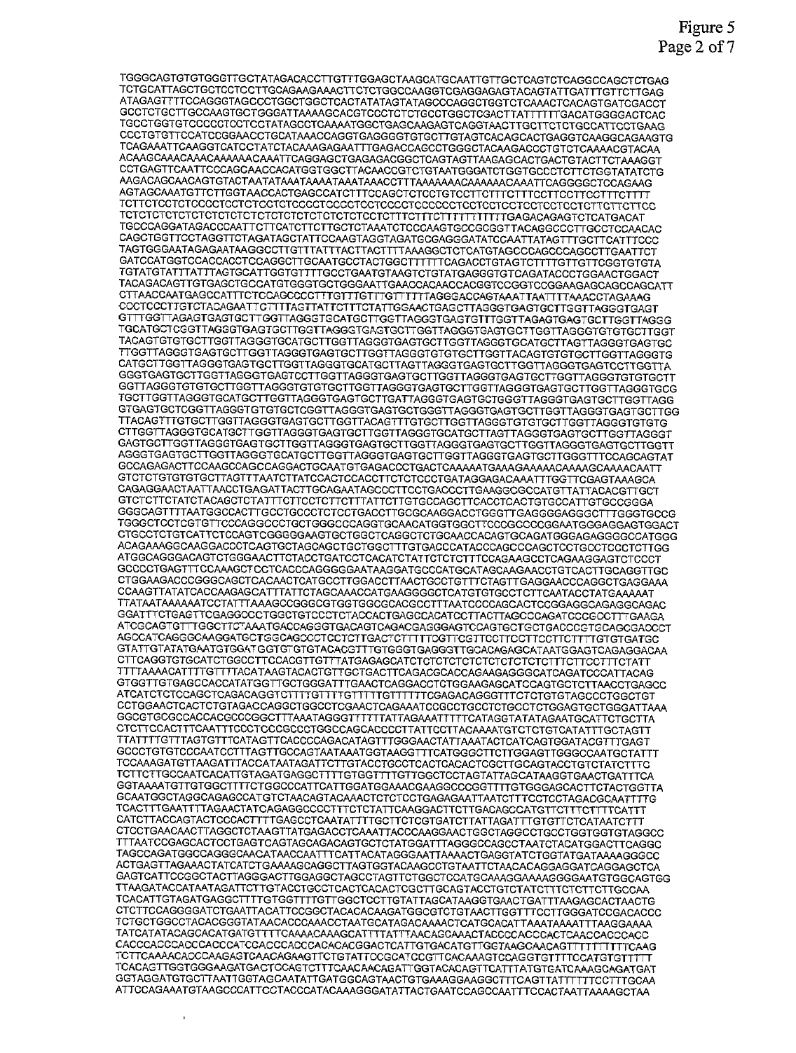

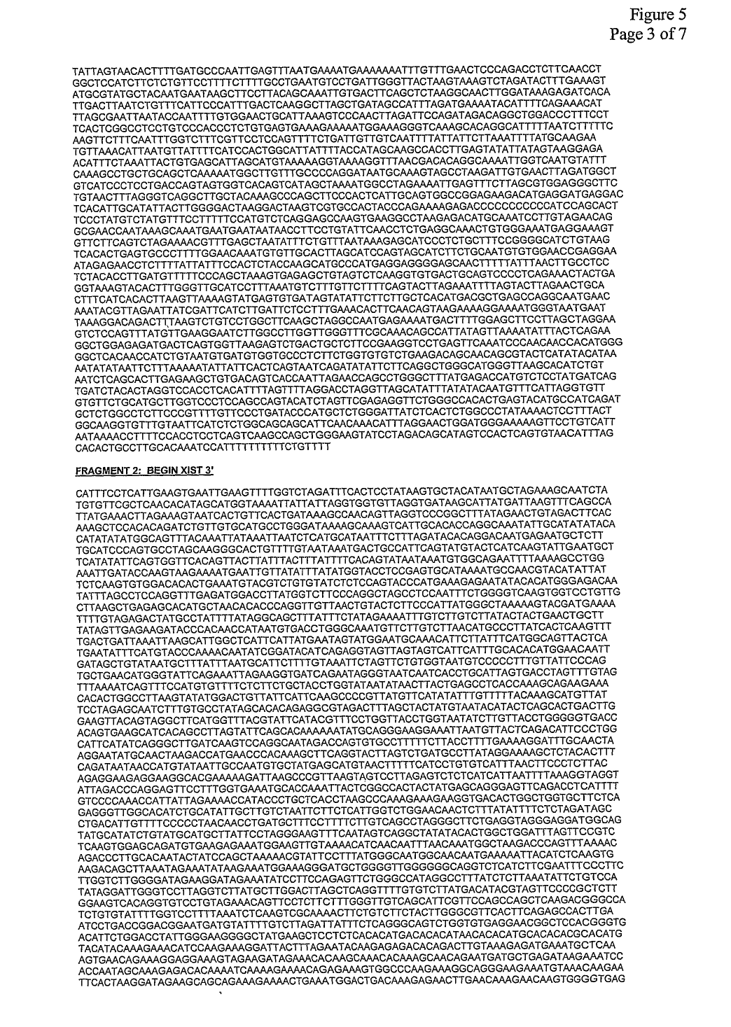

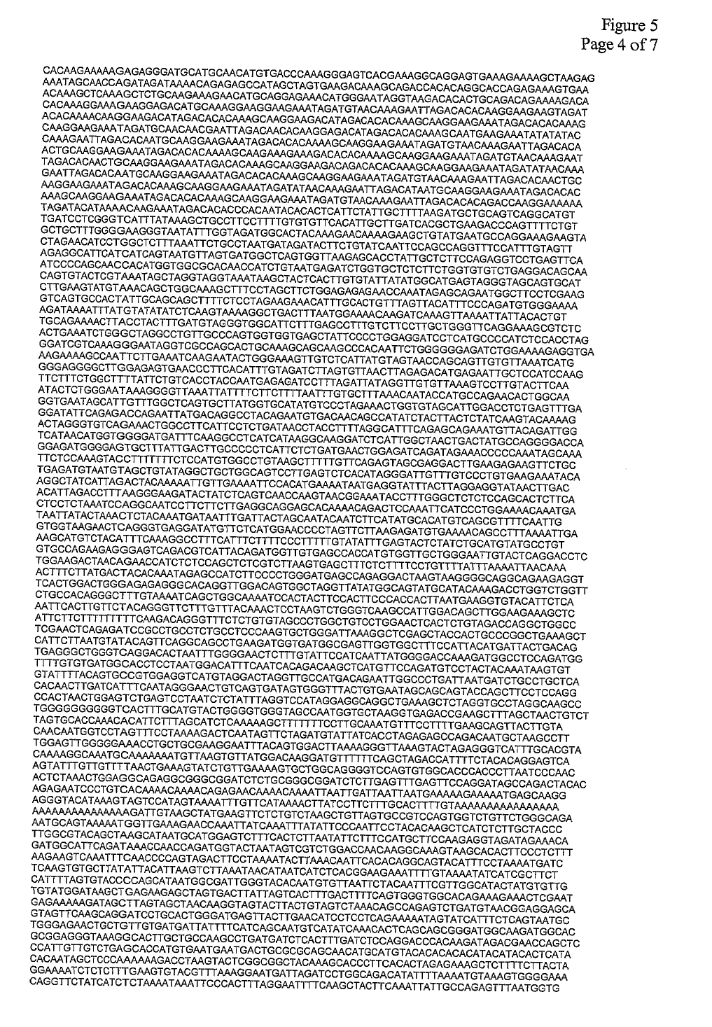

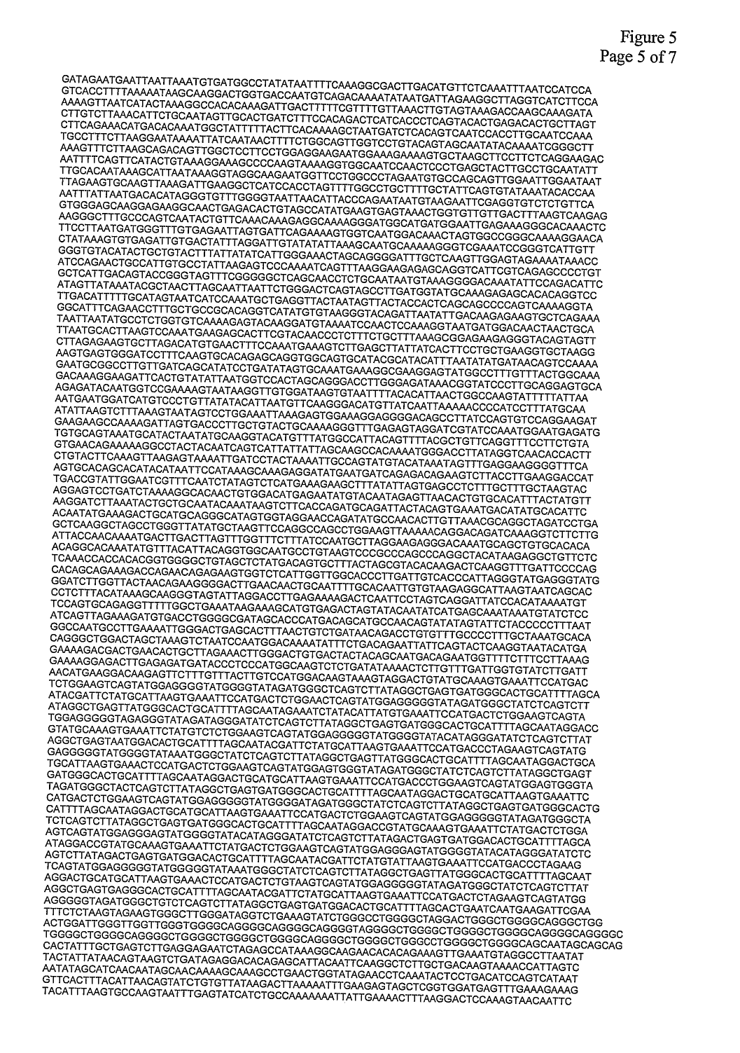

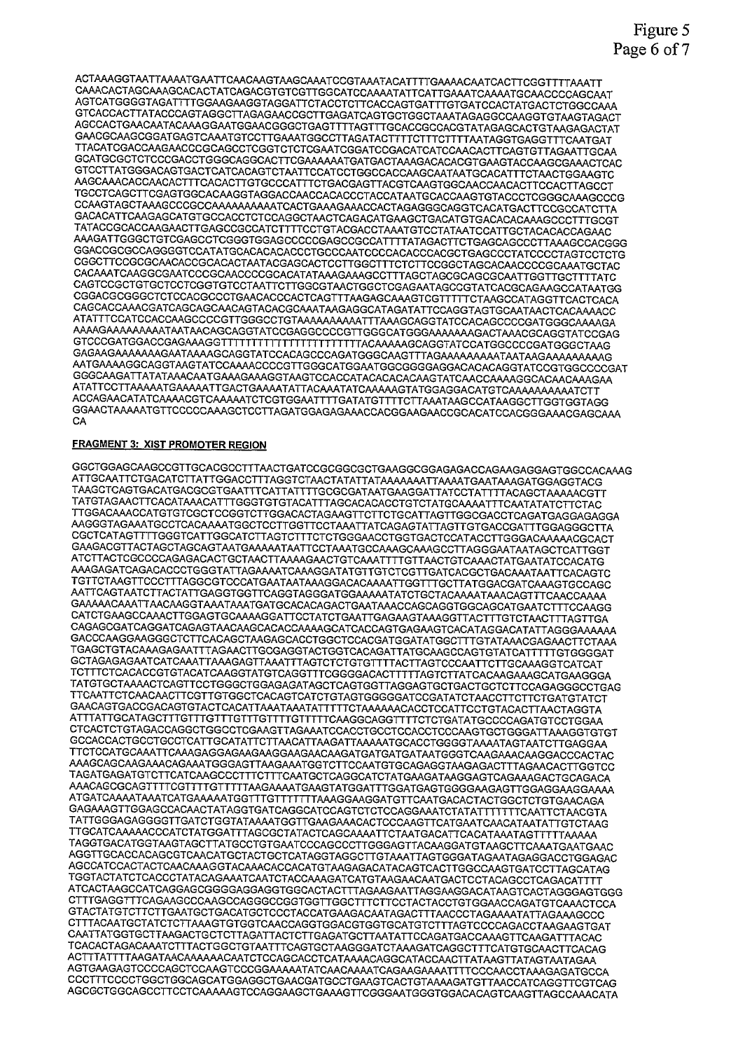

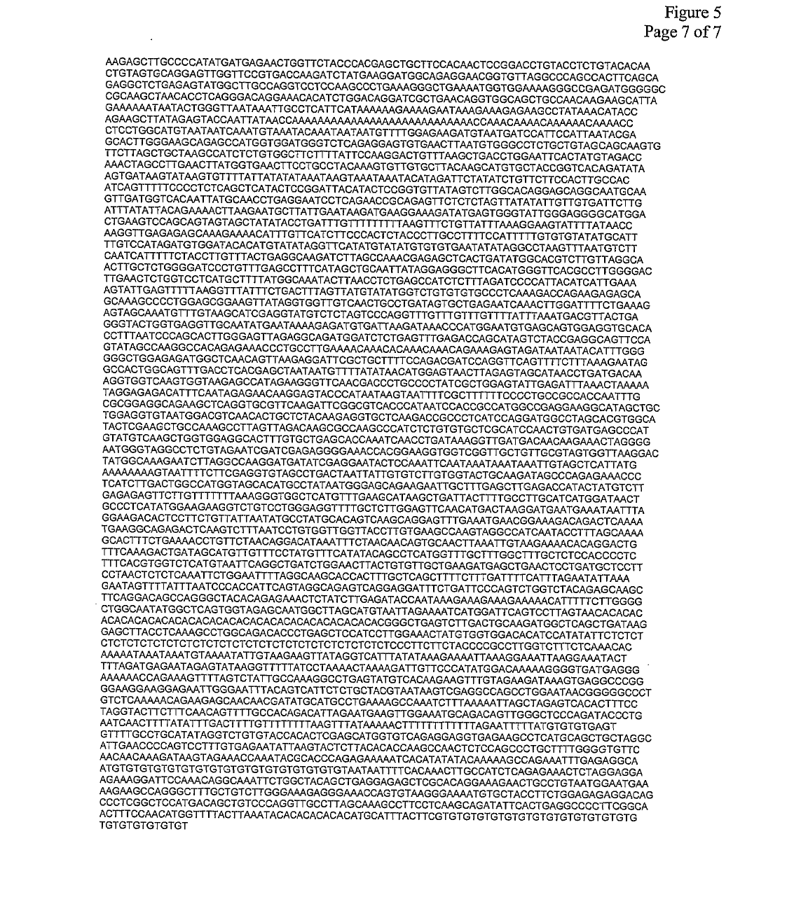

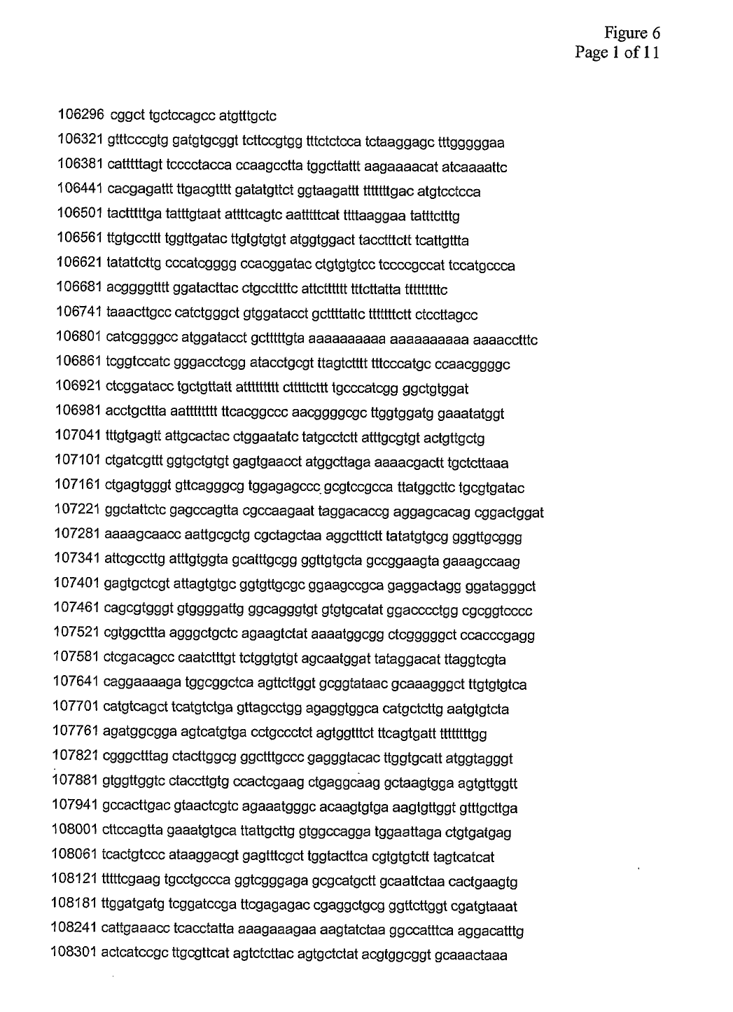

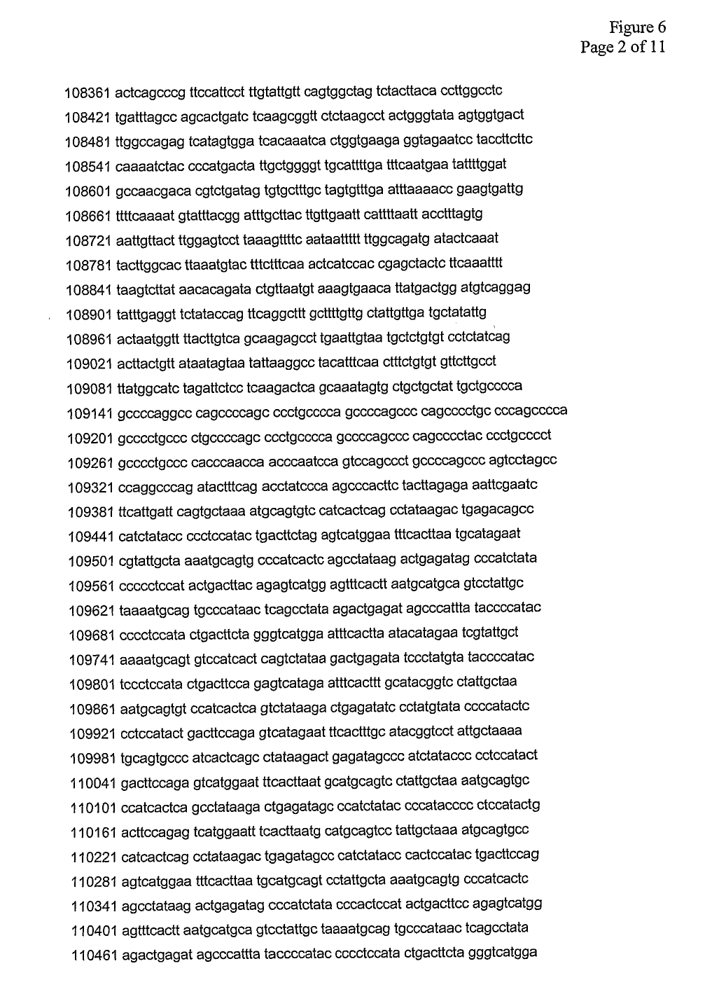

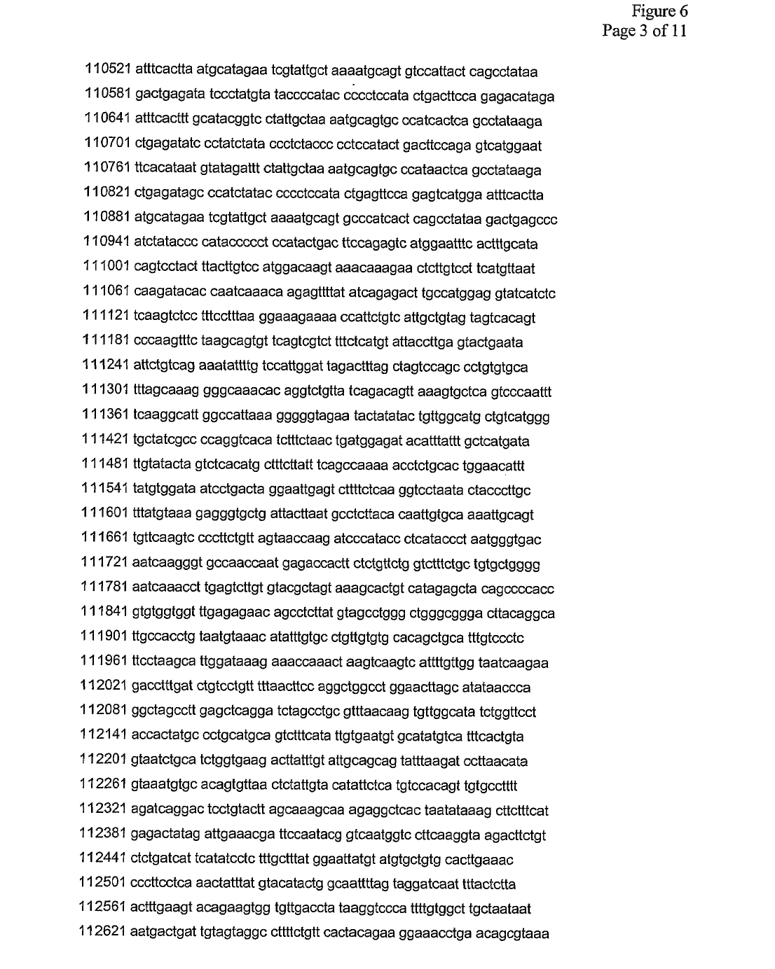

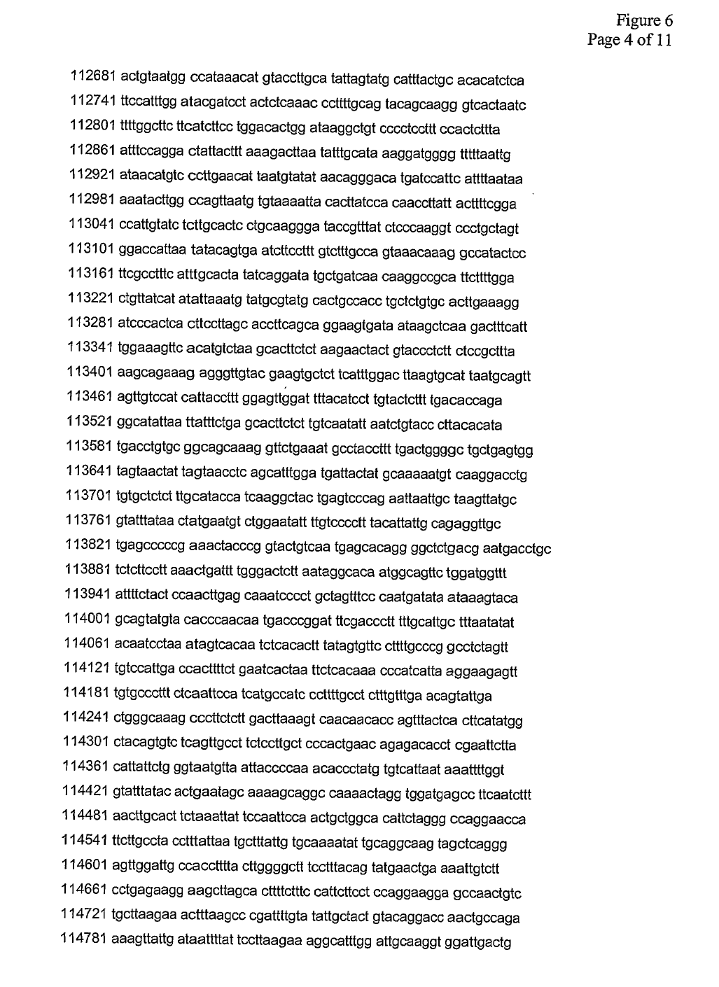

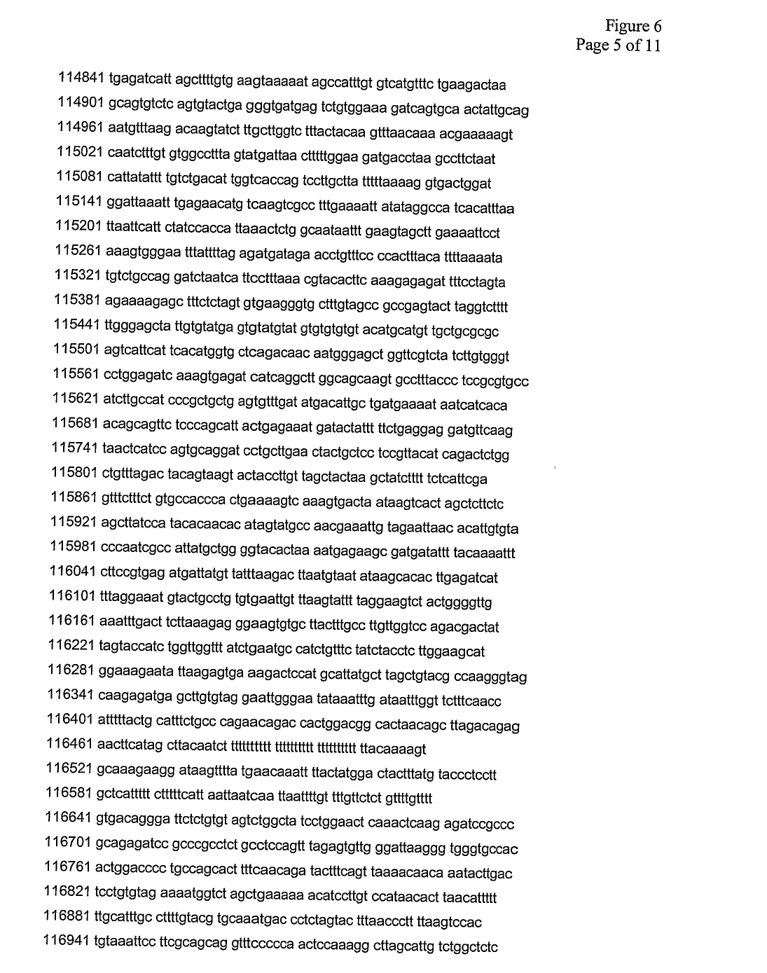

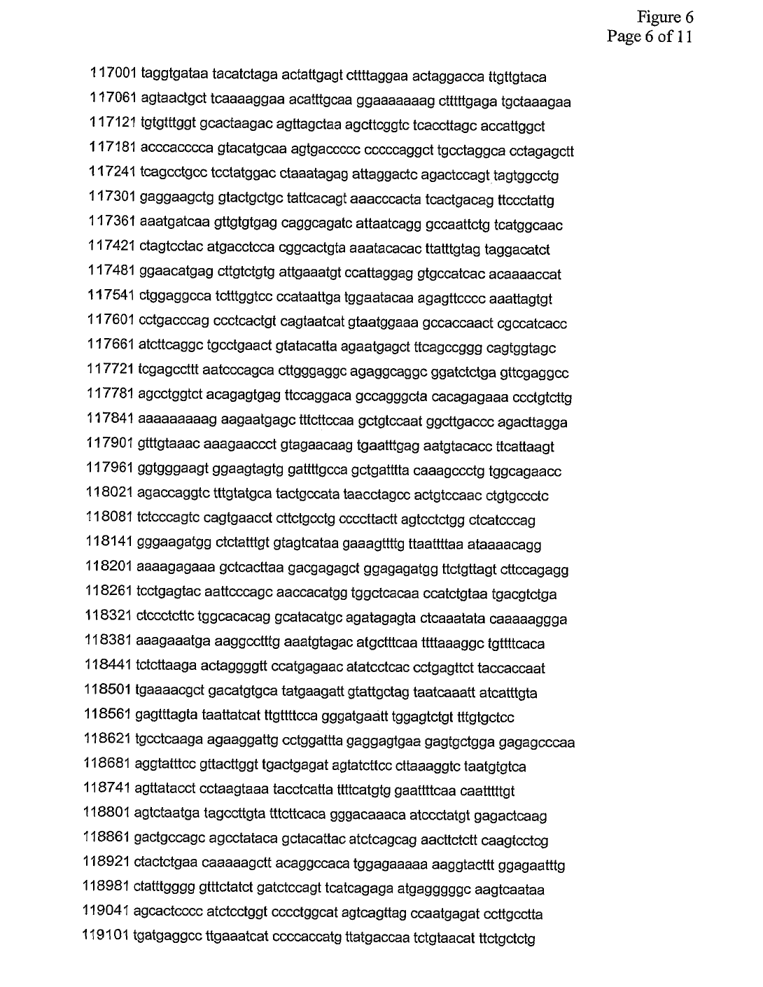

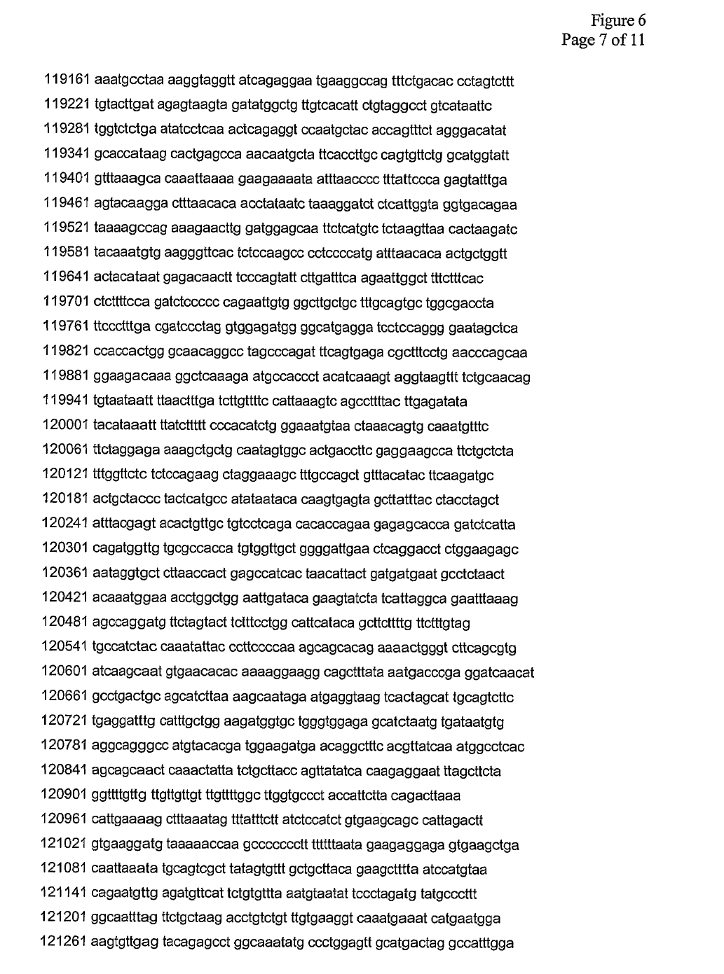

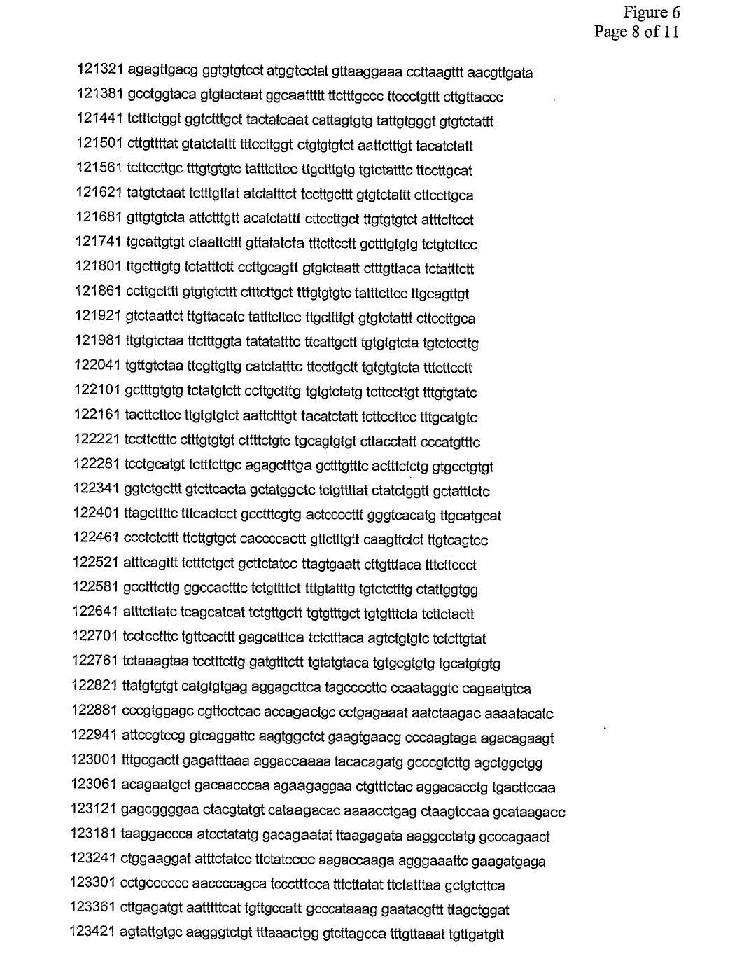

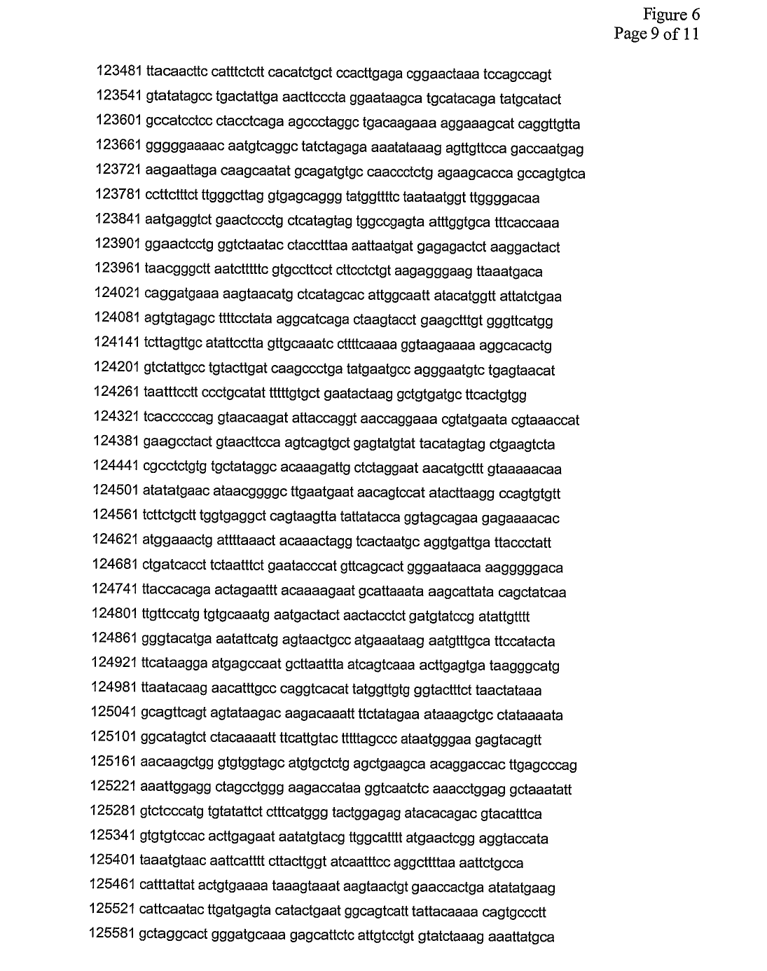

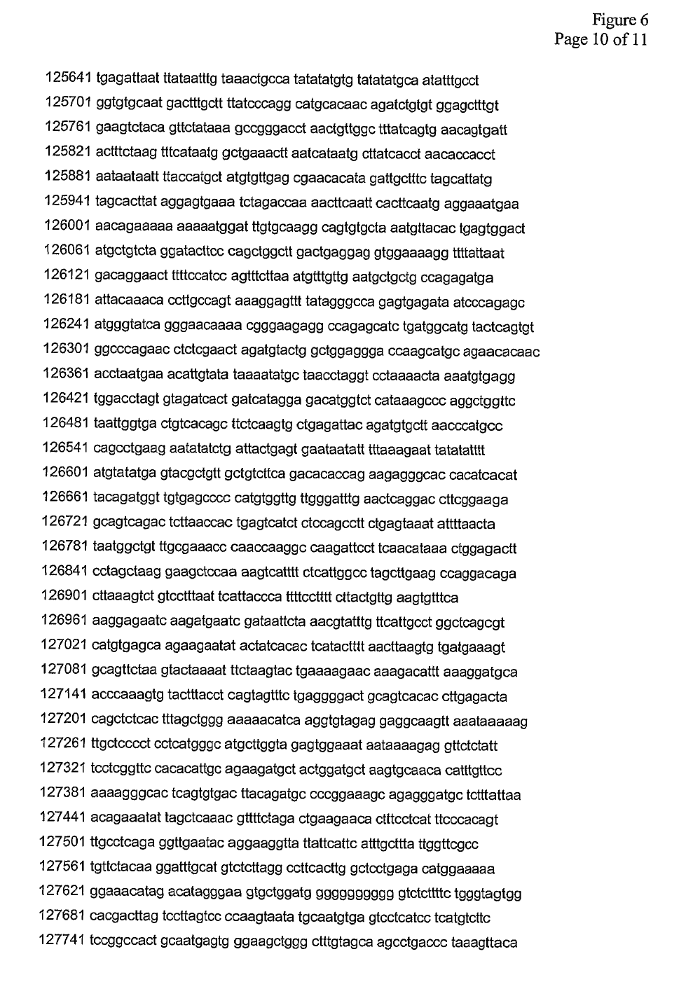

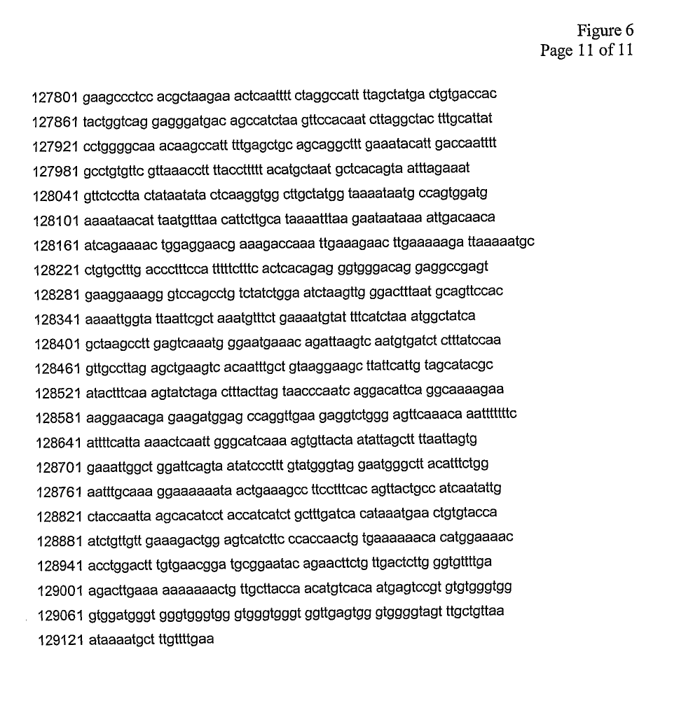

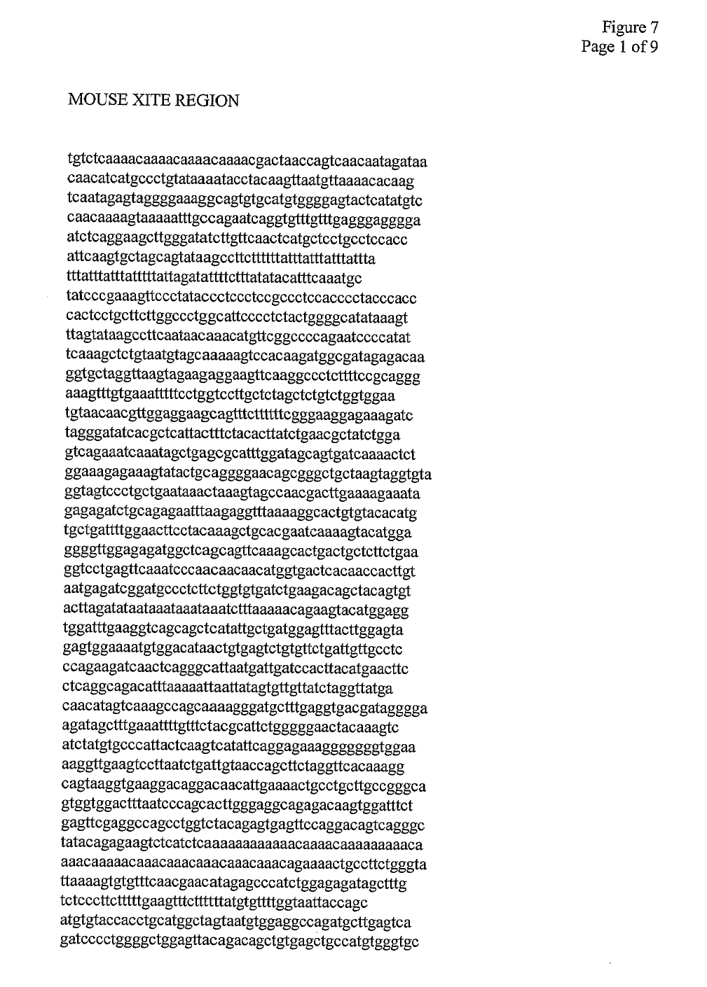

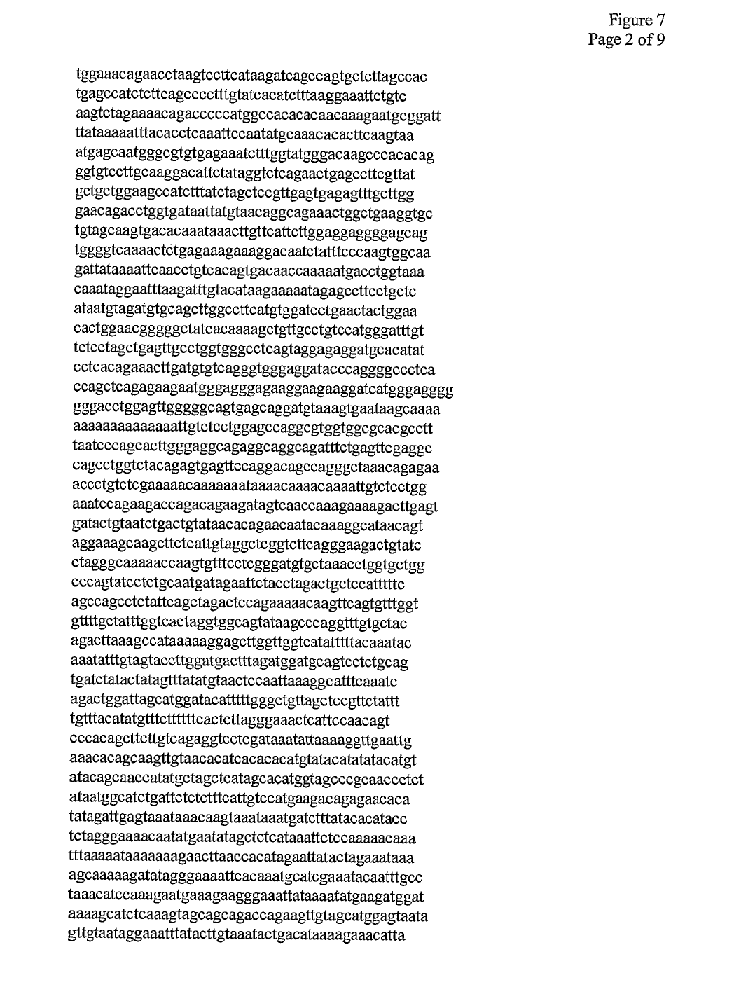

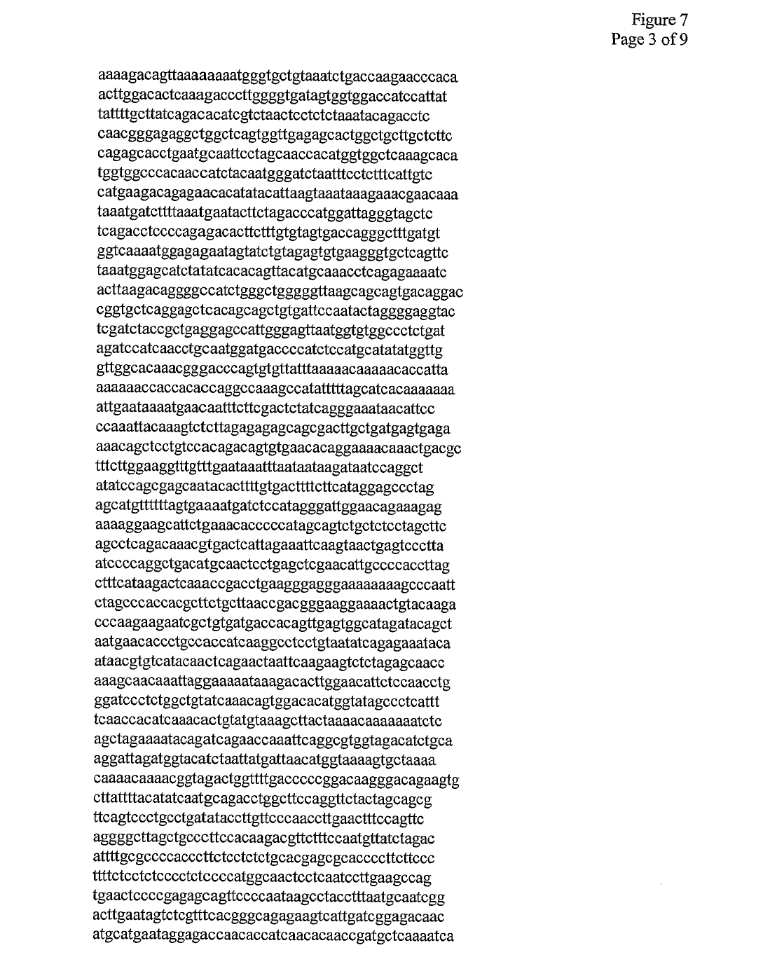

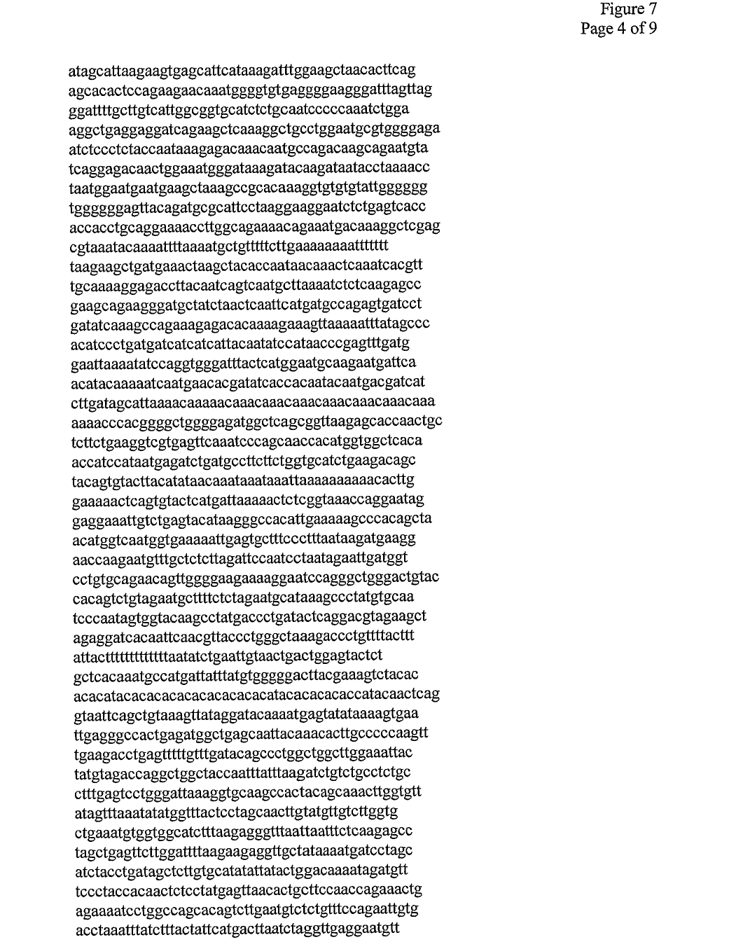

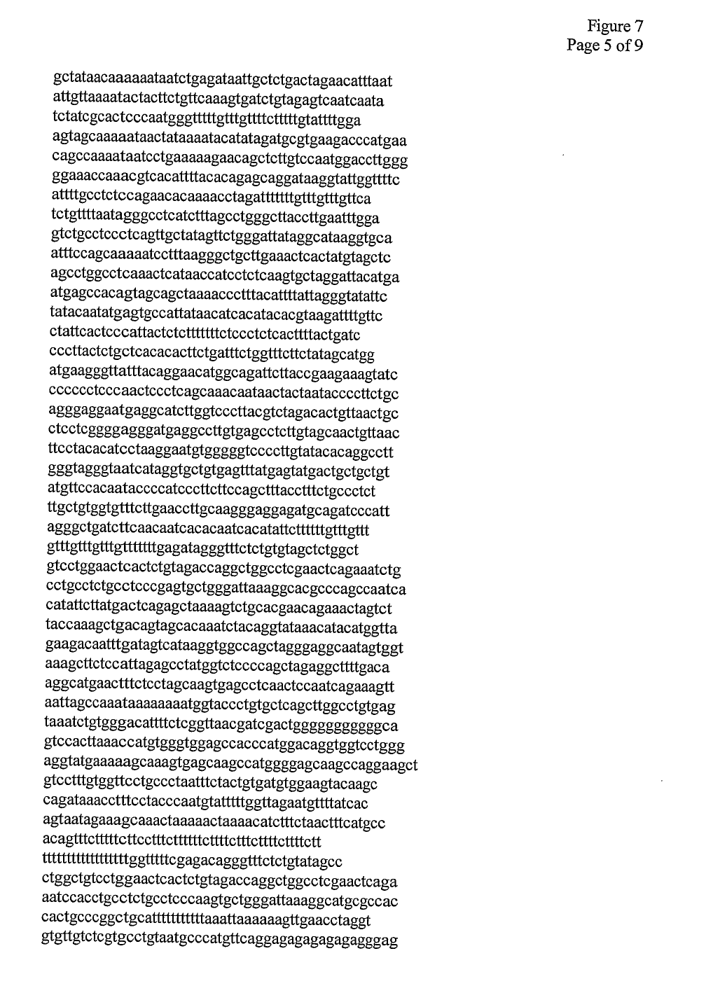

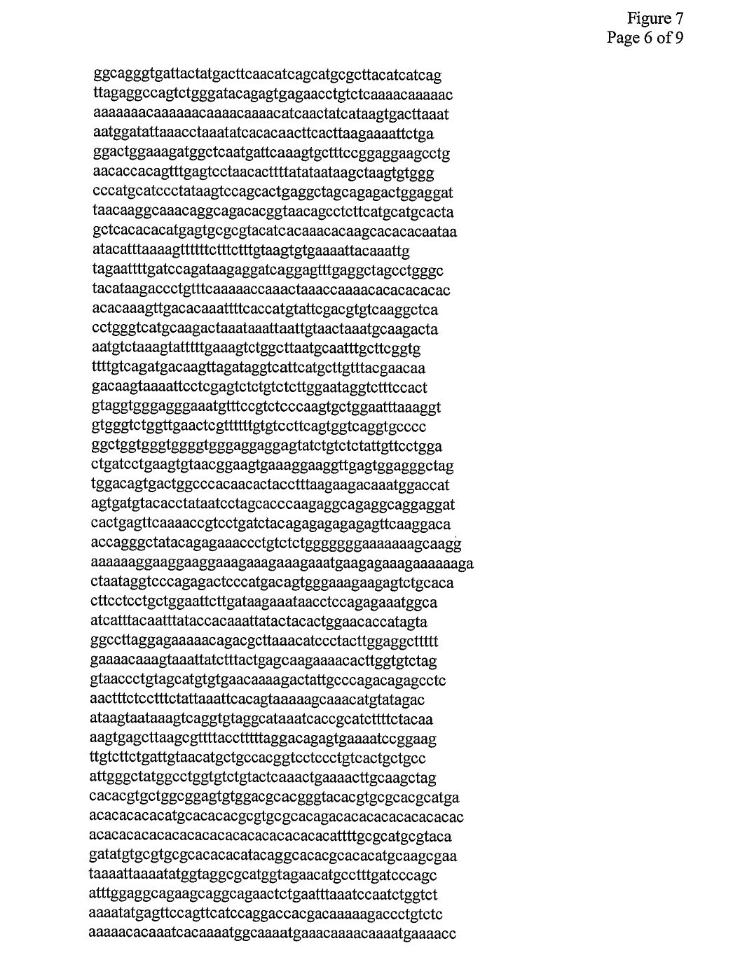

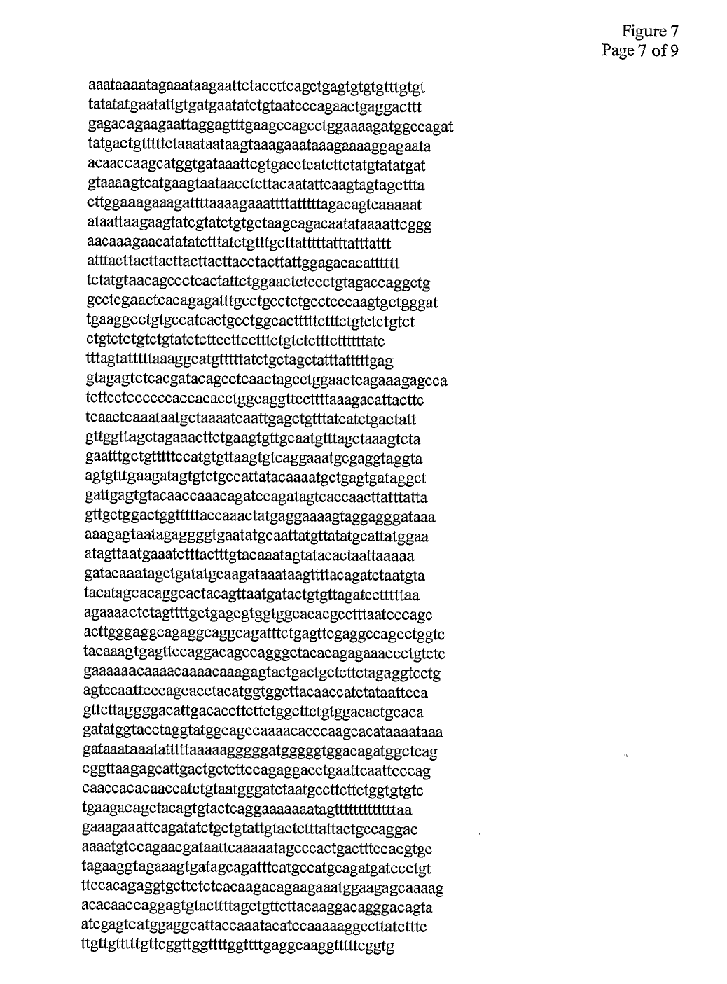

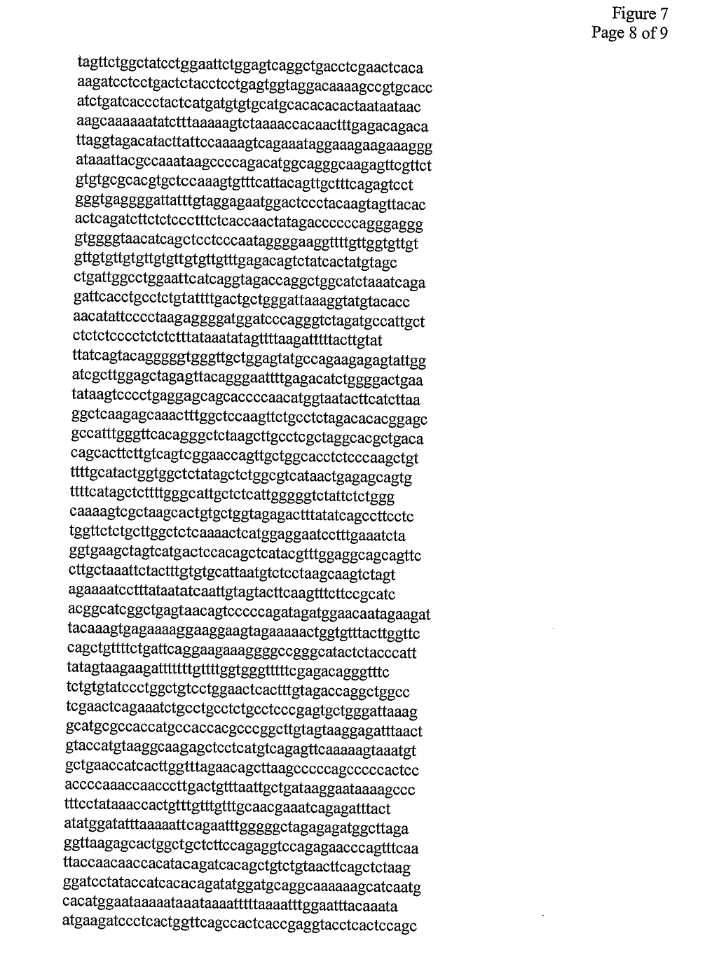

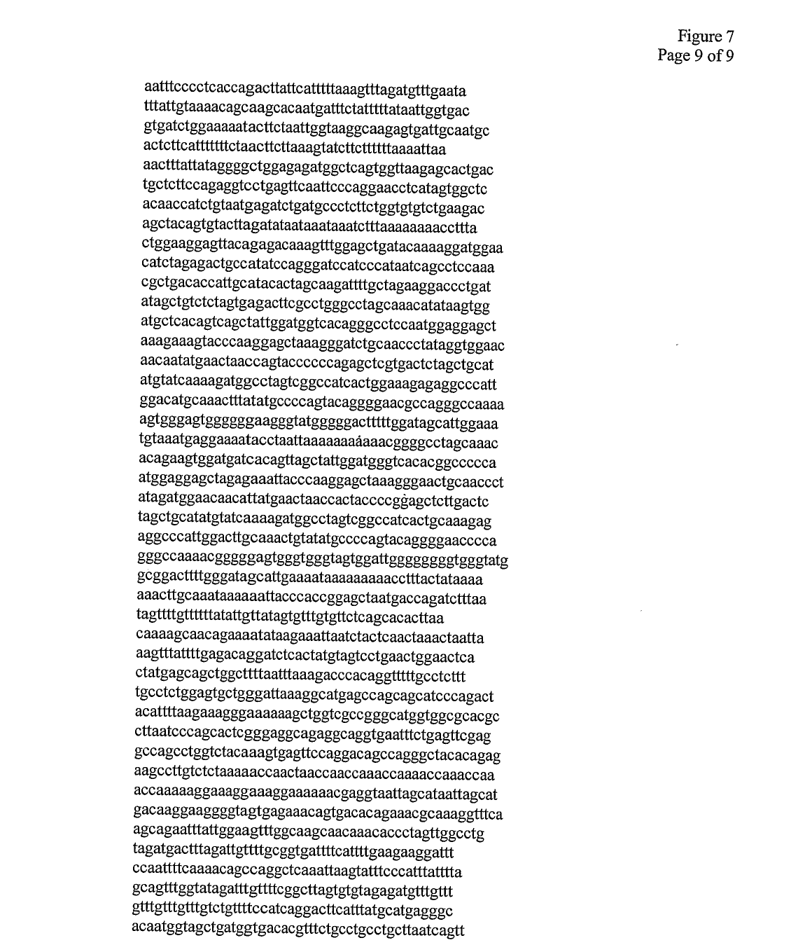

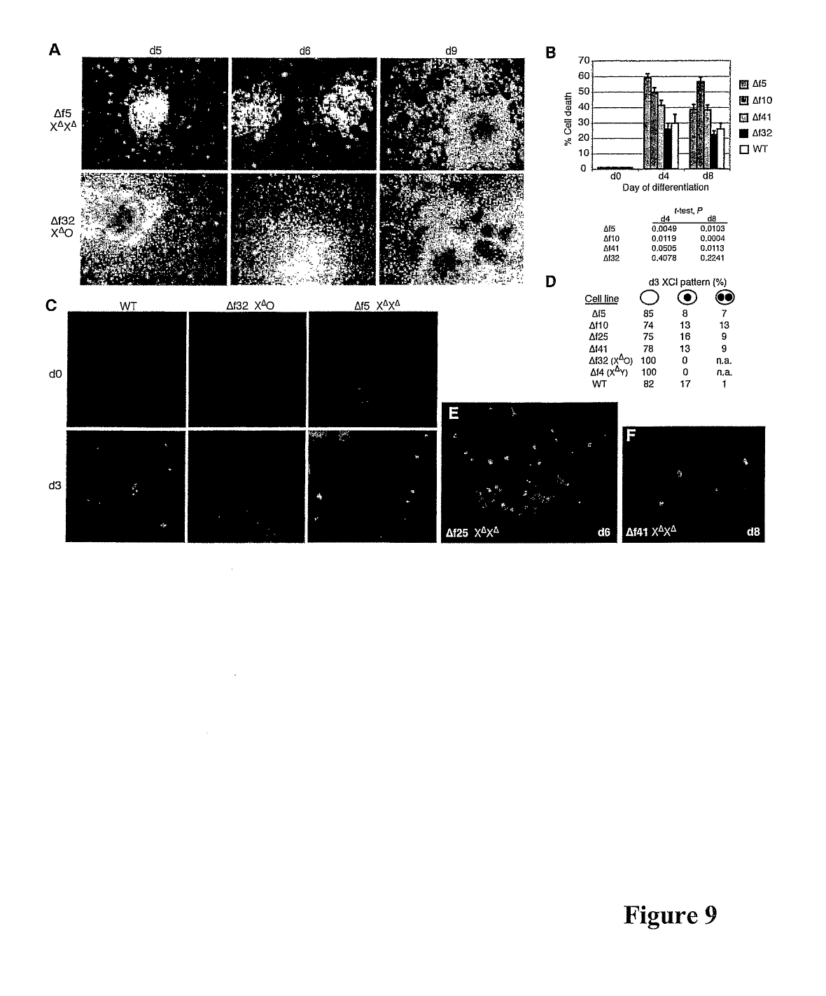

Mouse and Rat Sequences.

| SEQ | | | | |
|---|---|---|---|---|
| ID | | Length | | |
| NO | Name | (approx.) | Nucleotide Sequence | Reference Figure |
| 1 | Xic | 100 kB | nt 80,000 to 180,000 of GenBank AJ421479 | FIG. 4A |
| 2 | πJL2 | 80 kB | Xist + 30 kB up and downstream | FIG. 9A |
| 3 | πJL3 | 80 kB | Xist + 60 kB downstream | FIG. 9A |
| 4 | pSxN | 19.5 kB | see FIGS. 3A and 3B | FIGS. 1, 3A and 3B |
| 5 | Highly conserved | 5 kB | nt 1-5074 of SEQ ID NO: 4 (see FIG. 3B) | FIG. 3A, 3B |
| 6 | Full length Tsix | 40 kB | FIGs 5, nt 157, 186-104,000 of AJ421479 | FIG. 5 |
| 7 | pXist 3′ | 4.9 kB | Not shown | FIG. 1 |
| 8 | pXist 5′ | 4.8 kB | Not shown | FIG. 1 |
| 9 | pCC3 | 4.3 kB | nt 3079-7395 of SEQ ID NO: 4 (see FIG. 3B) | FIGS. 1, 3A and 3B |
| 10 | p3.7 | 3.7 kB | nt 5074-8768 of SEQ ID NO: 4 (see FIG. 3B) | FIGS. 1, 2, 3A and |
| | | | | 3B |
| 11 | pCC4 | 5.9 kB | nt 7395-13274 of SEQ ID NO: 4 (see FIG. 3B) | FIGS. 1, 2, 3A and |
| | | | | 3B |
| 12 | DxPas34 | 1.5 kB | nt 5073-6635 of SEQ ID NO: 4 (see FIG. 3B) | FIGS. 1, 3A and 3B |
| 13 | 34 bp repeat | 34 | Throughout nt 5073-6635 of SEQ ID NO: 4 | FIG. 3B, 4 |
| 14 | 68 bp repeat | 68 | Throughout nt 5073-6635 of SEQ ID NO: 4 | FIGS. 3B, 4 |
| 15 | Full length Xite | 20 kB | FIG. 7, nt 157, 186-104,000 of AJ421479 | FIG. 7 |
| 16 | pXite | 5.6 kB | nt 13887-19467 of SEQ ID NO: 4 (see FIG. 3B) | FIGS. 1, 2, 3A and |
| | | | | 3B |
| 17 | Xite Enhancer | 1.2 kB | nt 16360-17582 of SEQ ID NO: 4 (see FIG. 3B) | FIGS. 1, 3A and 3B |
| 18 | pTsx | 10.8 kB | nt 41, 347-52, 236 of GenBank X99946 | FIG. 1 |
| | | | (SEQ ID NO: 91) | |
| 19 | Bipartite | 10.2 kB | nt 3079-12274 of SEQ ID NO: 4 (see FIG., 3B) | FIGS. 3A, 3B |
| 20 | Full length Xist | 23 kB | FIG. 5, nt 106, 296-129, 140 of AJ421479 | FIG. 6 |
| 21 | ns25 (DXPas34) | 1.6 kB | nt 5485 (SalI) to 7177 (SmaI) of SEQ ID NO: 4 | FIGS. 1, 2, and 3B |
| | | | (see FIG. 3B) | |
| 22 | ns41 | 2.4 kB | nt 3079 (BamHI) to 5486 (SalI) of SEQ ID NO: 4 | FIGS. 1, 2, and 3B |
| | | | (see FIG. 3B) | |
| 23 | ns82 (Tsix | 220 bp | nt 7177 (SmaI) to 7398 (BamHI) of SEQ ID NO: 4 | FIGS. 1, 2 and 3B |
| | promoter) | | (see FIG. 3B) | |
| 24 | ns130 | 1.8 kB | nt 17580 to 19467 of SEQ ID NO: 4 (see FIG.3B) | FIGS. 1, 2 and 3B |
| 25 | ns135 (1.2 kb | 1.2 kB | nt 16360 (StuI) to 17583 (XhoI) of SEQ ID NO: 4 | FIGS. 1, 2, and 3B |
| | Xite enhancer | | (see FIG. 3B) | |
| 26 | ns155 (equivalent | 1.2 kb | nt 16360 (StuI) to 17583 (XhoI) of SEQ ID NO: 4 | FIGS. 1 and 3B |
| | to ns135 | | (see FIG. 3B) | |
| 27 | ns132 | 2.5 kB | nt 13883 (AvrII) to 16363 (StuI) of SEQ ID NO: 4 | FIGS. 1, 2, and 3B |
| | | | (see FIG. 3B) | |
| 28 | Mouse repeat A1 | 34 | GTGAYNNCCCAGRTCCCCGGTGGCAGGCATTTTA | FIG. 30B |
| 29 | Mouse repeat A2 | 32 | NNNNTNNNTNCNNNNNNNNNGCANNCATTTTA | FIG. 30B |
| 30 | Mouse repeat B | 30 | CAAGCACTTAGCCAYCGCYCCACTGTCCCG | FIG. 30B |
| 31 | Rat repeat A | 32 | NNYAYANNYCNNNNNNNYNNNCAGNNATTTTA | FIG. 30B |
| 32 | Rat repeat B | 31 | CARGCACNTYAGCCACCTCNCCACTGWCCCG | FIG. 30B |

“N” refers to any nucleotide
“Y” refers to either pyrimidine
“R” refers to either purine
* Note that the sequences as shown in FIG. 5 and GenBank Accession No. AJ421479 have a 3 kB deletion in the Zeste repeat region. This region cannot be sequenced. These coordinates are based on the sequence provided and do not include the 3 kB gap in the sequence.

TABLE 2
Human Sequences

| SEQ ID NO | Name | Length (approximate) | Nucleotide Sequence (approximate) |
|---|---|---|---|
| 35 | Xist | 32 kB | 11,390,576-11,358,483 of NT_011669 |
| 36 | Tsix | 64 kB | 11,329,000-11,393,000 of NT_011669 |
| 37 | pSxN human | 50-60 kB | 11,358,483-11,300,000 of NT_011669 |
| 38 | Xite | 13 kB | 11,320,000-11,333,000 of NT_011669 |
| 39 | Xic | 80 kB | 11,320,000-11,450,000 of NT_011669 |
| 40 | Repeat A | 16 bp | GCNNCNNGGNGGCAGG, FIG. 30B |

For any of the Xic, Tsix, Xite, or combined Tsix/Xite transgene sequences, it will be understood that mammalian (e.g., human, mouse, primate, bovine, ovine, feline, and canine) homologues, orthologues, paralogues, species variants, or syntenic variants are also included. For example, the human syntenic region includes approximately 15 megabases of contiguous human sequence on the X chromosome (GenBank Accession Number NT_{—}011669, SEQ ID NO: 34). These 15 megabases of sequence include the human Xic region as well as additional sequences on both ends of the Xic region. The syntenic equivalent of Xist is found at approximately nucleotides 11,390,576 to 11,358,483 (SEQ ID NO: 35) of GenBank Accession Number NT_{—}011669. The critical region including Tsix and Xite in the human sequence is predicted to be from approximately nucleotides 11,358,483 to nucleotide 11,300,000 (pSxN, human, SEQ ID NO: 37) of GenBank Accession Number NT_{—}011669. The syntenic equivalent of Tsix (SEQ ID NO: 36) is found at approximately nucleotides 11,329,000-11,393,000 and the syntenic equivalent of Xite (SEQ ID NO: 38) is found at approximately nucleotides 11,320,000 to 11,333,000 of NT_{—}011669. Transgenes that are useful in the methods of the invention can be identified using assays for the ability of the transgene to block X chromosome inactivation or differentiation. Such assays are known in the art and examples are described herein.

RNA interference (RNAi)

The present invention is based on the discovery that disruptions in the XCI process can block differentiation. One method for interfering with XCI involves the use of small RNA molecules, such as siRNA, directed to Xic, Tsix, Xite, or Xist that are introduced into stem cells and prevent the stem cells from undergoing X chromosome inactivation and from differentiating in culture. The use of such small RNA molecules circumvents the need for removal of the transgene because the small RNA molecules have a limited half-life and will naturally degrade.

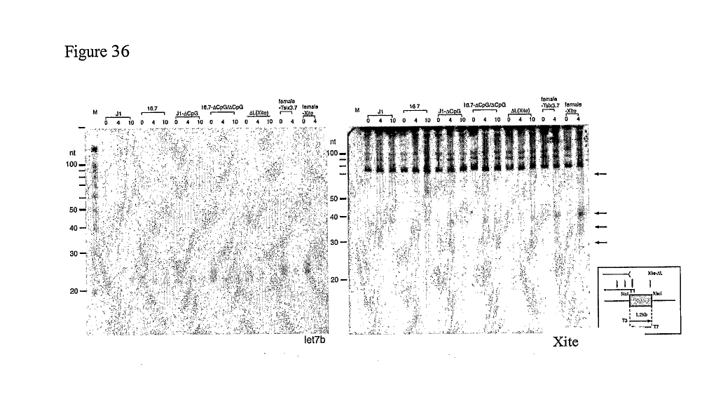

RNAi is a form of post-transcriptional gene silencing initiated by the introduction of double-stranded RNA (dsRNA). Short 15 to 32 nucleotide double-stranded RNAs, known generally as “siRNAs,” “small RNAs,” or “microRNAs” are effective at down-regulating gene expression in nematodes (Zamore et al., Cell 101: 25-33) and in mammalian tissue culture cell lines (Elbashir et al., Nature 411:494-498, 2001, hereby incorporated by reference). The further therapeutic effectiveness of this approach in mammals was demonstrated in vivo by McCaffrey et al. (Nature 418:38-39. 2002). The small RNAs are at least 15 nucleotides, preferably, 17, 18, 19, 20, 21, 22, 23, 24, 25, 26, 27, 28, 29, 30, 31, 32, 33, 34, 35, nucleotides in length and even up to 50 or 100 nucleotides in length (inclusive of all integers in between). Such small RNAs that are substantially identical to or complementary to any region of Xic, Tsix, Xite, or Xist, are included in the invention based on the discovery that Tsix, Xite, and also Xist elements are transcribed and portions of these regions exhibit bidirectional transcription, with the potential therefore for the formation of double-stranded RNAs which may then be subject to the RNAi pathway. In fact, small non-coding RNAs (ncRNAs) ranging from less than 25 nt to approximately 100 nt in size, corresponding to regions of Xite have been identified from both the sense and antisense strands (see FIG. 36). Furthermore, transcription or the ncRNA products of Xic, Tsix Xite, or Tsix/Xite, or both, have been shown to be required for pairing during XCI.

Therefore, the invention includes any small RNA substantially identical to at least 15 nucleotides, preferably, 17, 18, 19, 20, 21, 22, 23, 24, 25, 26, 27, 28, 29, 30, 31, 32, 33, 34, or 35, nucleotides in length and even up to 50 or 100 nucleotides in length (inclusive of all integers in between) of any region of Xic, Tsix, Xite, or Xist, preferably the regions described herein and shown in Tables 1 and 2. The invention also includes the use of such small RNA molecules to block differentiation. It should be noted that, as described below, longer dsRNA fragments can be used that are processed into such small RNAs. Useful small RNAs can be identified by their ability to block differentiation, block pairing, or block XCI using the methods described herein. Small RNAs can also include short hairpin RNAs in which both strands of an siRNA duplex are included within a single RNA molecule.

The specific requirements and modifications of small RNA are known in the art and are described, for example, in PCT Publication No. WO01/75164, and U.S. Application Publication Numbers 20060134787, 20050153918, 20050058982, 20050037988, and 20040203145, the relevant portions of which are herein incorporated by reference. In particular embodiments, siRNAs can be synthesized or generated by processing longer double-stranded RNAs, for example, in the presence of the enzyme dicer under conditions in which the dsRNA is processed to RNA molecules of about 17 to about 26 nucleotides. siRNAs can also be generated by expression of the corresponding DNA fragment (e.g., a hairpin DNA construct). Generally, the siRNA has a characteristic 2- to 3-nucleotide 3′ overhanging ends, preferably these are (2′-deoxy) thymidine or uracil. The siRNAs typically comprise a 3′ hydroxyl group. In some embodiments, single stranded siRNAs or blunt ended dsRNA are used. In order to further enhance the stability of the RNA, the 3′ overhangs are stabilized against degradation. In one embodiment, the RNA is stabilized by including purine nucleotides, such as adenosine or guanosine. Alternatively, substitution of pyrimidine nucleotides by modified analogs e.g. substitution of uridine 2-nucleotide overhangs by (2′-deoxy)thymide is tolerated and does not affect the efficiency of RNAi. The absence of a 2′ hydroxyl group significantly enhances the nuclease resistance of the overhang in tissue culture medium.

siRNA molecules can be obtained through a variety of protocols including chemical synthesis or recombinant production using a Drosophila in vitro system. They can be commercially obtained from companies such as Dharmacon Research Inc. or Xeragon Inc., or they can be synthesized using commercially available kits such as the Silencer™ siRNA Construction Kit from Ambion (catalog number 1620) or HiScribe™ RNAi Transcription Kit from New England BioLabs (catalog number E2000S).

Alternatively siRNA can be prepared using standard procedures for in vitro transcription of RNA and dsRNA annealing procedures such as those described in Elbashir et al. (Genes & Dev., 15:188-200, 2001), Girard et al., (Nature Jun. 4, 2006, e-publication ahead of print), Aravin et al., (Nature Jun. 4, 2006, e-publication ahead of print), Grivna et al., (Genes Dev. Jun. 9, 2006, e-publication ahead of print), and Lau et al., (Science Jun. 15, 2006, e-publication ahead of print). siRNAs are also obtained by incubation of dsRNA that corresponds to a sequence of the target gene in a cell-free Drosophila lysate from syncytial blastoderm Drosophila embryos under conditions in which the dsRNA is processed to generate siRNAs of about 21 to about 23 nucleotides, which are then isolated using techniques known to those of skill in the art. For example, gel electrophoresis can be used to separate the 21-23 nt RNAs and the RNAs can then be eluted from the gel slices. In addition, chromatography (e.g. size exclusion chromatography), glycerol gradient centrifugation, and affinity purification with antibody can be used to isolate the small RNAs.

siRNAs specific to the Tsix, Xite, Xist or Xic regions can also be obtained from natural sources. For example, as shown in FIG. 36, small RNAs are endogenously produced from the various sites within the mouse XIC. Such small RNAs can be purified as described above and used in the methods of the invention.

Short hairpin RNAs (shRNAs), as described in Yu et al. or Paddison et al. (Proc. Natl. Acad. Sci. USA, 99:6047-6052, 2002; Genes & Dev, 16:948-958, 2002; incorporated herein by reference), can also be used in the methods of the invention. shRNAs are designed such that both the sense and antisense strands are included within a single RNA molecule and connected by a loop of nucleotides (3 or more). shRNAs can be synthesized and purified using standard in vitro T7 transcription synthesis as described above and in Yu et al. (supra). shRNAs can also be subcloned into an expression vector that has the mouse U6 promoter sequences which can then be transfected into cells and used for in vivo expression of the shRNA.

A variety of methods are available for transfection, or introduction, of dsRNA into mammalian cells. For example, there are several commercially available transfection reagents useful for lipid-based transfection of siRNAs including but not limited to: TransIT-TKO™ (Mirus, Cat. #MIR 2150), Transmessenger™ (Qiagen, Cat. #301525), Oligofectamine™ and Lipofectamine™ (Invitrogen, Cat. #MIR 12252-011 and Cat. #13778-075), siPORT™ (Ambion, Cat. #1631), DharmaFECT™ (Fisher Scientific, Cat. #T-2001-01). Agents are also commercially available for electroporation-based methods for transfection of siRNA, such as siPORTer™ (Ambion Inc. Cat. #1629). Microinjection techniques can also be used. The small RNA can also be transcribed from an expression construct introduced into the cells, where the expression construct includes a coding sequence for transcribing the small RNA operably linked to one or more transcriptional regulatory sequences. Where desired, plasmids, vectors, or viral vectors can also be used for the delivery of dsRNA or siRNA and such vectors are known in the art. Protocols for each transfection reagent are available from the manufacturer. Additional methods are known in the art and are described, for example in U.S. Patent Application Publication No. 20060058255.

The concentration of dsRNA used for each target and each cell line varies and can be determined by the skilled artisan. If desired, cells can be transfected multiple times, using multiple small RNAs to optimize the gene-silencing effect.

### Cells

Embryonic stem cells (ES), derived from the inner cell mass of preimplantation embryos, have been recognized as the most pluripotent stem cell population and are therefore the preferred cell for the methods of the invention. These cells are capable of unlimited proliferation in vitro, while maintaining the capacity for differentiation into a wide variety of somatic and extra-embryonic tissues. ES cells can be male (XY) or female (XX); female ES cells are preferred.

Multipotent, adult stem cells can also be used in the methods of the invention. Preferred adult stem cells include hematopoietic stem cells (HSC), which can proliferate and differentiate throughout life to produce lymphoid and myeloid cell types; bone marrow-derived stem cells (BMSC), which can differentiate into various cell types including adipocytes, chondrocytes, osteocytes, hepatocytes, cardiomyocytes and neurons; and neural stem cells (NSC), which can differentiate into astrocytes, neurons, and oligodendrocytes. Multipotent stem cells derived from epithelial and adipose tissues and umbilical cord blood cells can also be used in the methods of the invention.

Stem cells can be derived from any mammal including, but not limited to, mouse, human, and primates. Preferred mouse strains for stem cell preparation include 129, C57BL/6, and a hybrid strain (Brook et al., Proc. Natl. Acad. Sci. U.S.A. 94:5709-5712 (1997), Baharvand et al., In Vitro Cell Dev. Biol. Anim. 40:76-81 (2004)). Methods for preparing mouse, human, or primate stem cells are known in the art and are described, for example, in Nagy et al., Manipulating the mouse embryo: A laboratory manual, 3^{rd} ed., Cold Spring Harbor Laboratory Press (2002); Thomson et al., Science 282:1145-1147 (1998), Marshall et al., Methods Mol. Biol. 158:11-18 (2001); Thomson et al., Trends Biotechnol. 18:53-57 (2000); Jones et al., Semin. Reprod. Med. 18:219-223 (2000); Voss et al., Exp. Cell Res. 230:45-49 (1997); and Odorico et al., Stem Cells 19:193-204 (2001).

ES cells can be directly derived from the blastocyst or any other early stage of development, or can be a “cloned” stem cell line derived from somatic nuclear transfer and other similar procedures. General methods for culturing mouse, human, or primate ES cells from a blastocyst can be found in Appendix C of the NIH report on stem cells entitled Stem Cells: Scientific Progress and Future Research Directions (this report can be found online at the NIH Stem Cell Information website, http://stemcells.nih.gov/info/scireport). For example, in the first step, the inner cell mass of a preimplantation blastocyst is removed from the trophectoderm that surrounds it. (For cultures of human ES cells, blastocysts are generated by in vitro fertilization and donated for research.) The small plastic culture dishes used to grow the cells contain growth medium supplemented with fetal calf serum, and are sometimes coated with a “feeder” layer of nondividing cells. The feeder cells are often mouse embryonic fibroblast (MEF) cells that have been chemically inactivated so they will not divide. Additional reagents, such as the cytokine leukemia inhibitory factor (LIF), can also be added to the culture medium for mouse ES cells. Second, after several days to a week, proliferating colonies of cells are removed and dispersed into new culture dishes, each of which may or may not contain an MEF feeder layer. If the cells are to be used to human therapeutic purposes, it is preferable that the MEF feeder layer is not included. Under these in vitro conditions, the ES cells aggregate to form colonies. In the third major step required to generate ES cell lines, the individual, nondifferentiating colonies are dissociated and replated into new dishes, a step called passage. This replating process establishes a “line” of ES cells. The line of cells is termed “clonal” if a single ES cell generates it. Limiting dilution methods can be used to generate a clonal ES cell line. Reagents needed for the culture of stem cells are commercially available, for example, from Invitrogen, Stem Cell Technologies, R&D Systems, and Sigma Aldrich, and are described, for example, in U.S. Patent Application Publication Numbers 20040235159 and 20050037492 and Appendix C of the NIH report, Stem Cells: Scientific Progress and Future Research Directions, supra.

Although the preferred methods of the invention include transfection of the transgene into the stem cell after the stem cell line has been established, it is also possible to generate a chimeric transgenic mouse having the transgene integrated into the mouse chromosome. The transgene would then be present in the germ line and the mouse would be mated to produce embryos with an integrated transgene. The inner cell mass of a preimplantation blastocyst having the integrated transgene is removed from the trophectoderm that surrounds it and used to establish a stem cell line as described above.

### Transfection of Transgenes

After a stem cell line has been established, the cells can be transfected or transduced (for viral vectors), with a transgene of the invention to prevent or control stem cell differentiation. Transgenes may be integrated into the chromosome or may be episomal depending on the methods used for delivery of the transgene. Methods for delivery of a transgene into cells using plasmids or viral vectors are known in the art. Suitable methods for transfecting or infecting host cells can be found in Sambrook et al. (Molecular Cloning: A Laboratory Manual, 2nd Edition, Cold Spring Harbor Laboratory press (1989)); Goeddel et al., (Gene Expression Technology: Methods in Enzymology, Academic Press, San Diego, Calif. (1990); Ausubel et al. (Current Protocols in Molecular Biology John Wiley & Sons, New York, N.Y. (1998); Watson et al., Recombinant DNA, Chapter 12, 2nd edition, Scientific American Books (1992); and other laboratory textbooks. For a review of methods for delivery of a transgene see Stull, The Scientist, 14:30-35 (2000). Recombinant plasmids or vectors can be transferred by methods such as calcium phosphate precipitation, electroporation, liposome-mediated transfection, gene gun, microinjection, viral capsid-mediated transfer, or polybrene-mediated transfer. For a review of the procedures for liposome preparation, targeting and delivery of contents, see Mannino and Gould-Fogerite, (Bio Techniques, 6:682-690, 1988), Felgner and Holm, (Bethesda Res. Lab. Focus, 11:21, 1989) and Maurer (Bethesda Res. Lab. Focus, 11:25, 1989). For viral transduction, viral vectors are generally first transferred to a helper cell culture, using the methods described above, for the production of virus. Viral particles are then isolated and used to infect the intended stem cell line. Techniques for the production and isolation of viral particles and the use of viral particles for infection can also be found in the references cited above and in U.S. Patent Application Publication Number 20040241856.

There are a variety of plasmids and viral vectors useful for delivery of a transgene and these are known in the art. See, for example, Pouwels et al., Cloning Vectors: A Laboratory Manual (1985). Supp. 1987) and the references cited above. Plasmids and viral vectors are also commercially available, for example, from Clontech, Invitrogen, Stratagene, and BD Biosciences.

In general, preferred plasmids or viral vectors include the following components: a multiple cloning site consisting of restriction enzyme recognition sites for cloning of the transgene, and a eukaryotic selectable marker (positive or negative) for selection of transfected or transduced cells in media supplemented with the selection agent. Preferred selectable markers include drug resistance markers, antigenic markers, adherence markers, and the like. Examples of antigenic markers include those useful in fluorescence-activated cell sorting. Examples of adherence markers include receptors for adherence ligands that allow selective adherence. Other selection markers include a variety of gene products that can be detected in experimental assay protocols, such as marker enzymes, amino acid sequence markers, cellular phenotypic markers, nucleic acid sequence markers, and the like. In general, positive selection marker genes are drug resistance genes. Suitable positive selection markers include, for example, nucleic acid sequences encoding neomycin resistance, hygromycin resistance, puromycin resistance, histidinol resistance, xanthine utilization, zeocin resistance, and bleomycin resistance. The positive selection marker can be operably linked to a promoter in the nucleic acid molecule (e.g., a prokaryotic promoter or a phosphoglycerate kinase (“PGK”) promoter).

In general, negative selection marker genes are used in situations whereby the expressed gene product leads to the elimination of the host cell, for example, in the presence of a nucleoside analog, such as gancyclovir. Suitable negative selection markers include, for example, nucleic acid sequences encoding Hprt, gpt, HSV-tk, diphtheria toxin, ricin toxin, and cytosine deaminase.

Plasmids or viral vectors can also contain a polyadenylation site, one or more promoters, and an internal ribosome entry site (IRES), which permits attachment of a downstream coding region or open reading frame with a cytoplasmic polysomal ribosome to initiate translation in the absence of internal promoters. IRES sequences are frequently located on the untranslated leader regions of RNA viruses, such as the Picornaviruses. The viral sequences range from about 450-500 nucleotides in length, although IRES sequences may also be shorter or longer (Adam et al. J Virol 65: 4985-4990 (1991); Borman et al. Nuc. Acids Res. 25: 925-32 (1997); Hellen et al. Curr. Top. Microbiol. Immunol. 203: 31-63 (1995); and Mountford et al. Trends Genet. 11: 179-184 (1995)). The encephalomyocarditis virus IRES is one such IRES which is suitable for use in this invention.

Plasmids or viral vectors can also include a bacterial origin of replication, one or more bacterial promoters, and a prokaryotic selectable marker gene for selection of transformed bacteria and production of the plasmid or vector. Bacterial selectable marker genes can be equivalent to or different from eukaryotic selectable marker genes. Non-limiting examples of preferred bacterial selectable marker genes include nucleic acids encoding ampicillin resistance, kanamycin resistance, hygromycin resistance, and chloramphenicol resistance.

Desirably, plasmids or viral vectors will also include sequences for the excision and removal of the transgene. Recombinase recognition sequences useful for targeted recombination are used for methods of controlling differentiation and are described in detail below. Non-limiting examples of recognition sequences that can be included in the plasmids or vectors used in the invention are loxP sequences or FRT sequences. The loxP site consists of two 13-bp inverted repeats flanking an 8-bp nonpalindromic core region. The loxP sequence is a DNA sequence comprising the following nucleotide sequence (hereinafter this sequence is referred to as the wild type loxP sequence):

(SEQ ID NO: 41)

5′-ATAACTTCGTATA ATGTATGC TATACGAAGTTAT-3′

(SEQ ID NO: 42)

3′-TATTGAAGCATAT TACATACG ATATGCTTCAATA-5′

However, the loxP sequence need not be limited to the above wild type loxP sequence, and part of the wild type loxP sequence may be replaced with other bases as long as the two “recombinase recognition sequences” become substrates for the Cre recombinase. Furthermore, even those loxP sequences (mutant loxP sequences) that normally do not become substrates for recombinase Cre in a combination with the wild type loxP sequence but become substrates for recombinase Cre in a combination with the mutant loxP sequences of the same sequence by base replacement of the wild type loxP sequence (i.e., sequences for which the entire process of cleavage, exchange, and binding of DNA strands takes place) are included in the recognition sequences of recombinase Cre. Examples of such mutant loxP sequences are described in Hoess et al., (Nucleic Acids Res. 14:2287-2300 (1986)), in which one base in a spacer region of the wild type loxP sequence has been replaced and Lee et al., (Gene 14:55-65 (1998)), in which two bases in the spacer region have been replaced.

FLP recognition sequences include any sequence that becomes a substrate for recombinase FLP, wherein FLP causes the entire process of cleavage, exchange, and binding of DNA chains between two recombinase recognition sequences. Examples include the FRT sequence, which is a 34-base DNA sequence (Babineau et al., J. Biol. Chem. 260:12313-12319 (1985)). As described for the Cre recognition sequences above, an FLP recognition sequence is not limited to the above wild type FRT sequence. Part of the wild type FRT sequence may be replaced with other bases as long as two FLP recombinase recognition sequences can become substrates for FLP recombinase. Furthermore, even those FRT sequences (mutant FRT sequences) that normally do not become substrates for recombinase FLP in a combination with the wild type FRT sequence but become substrates for recombinase in a combination with the mutant FRT sequences of the same sequence by base replacement of the wild type FRT sequence (i.e., sequences for which the entire process of cleavage, exchange, and binding of DNA strands takes place), are included in the FLP recognition sequences. For examples of FRT sequences, see McLeod et al., Mol Cell Biol., 6:3357-3367 (1986).

Non-limiting examples of viral vectors useful in the invention include adenoviral vectors, adeno-associated viral vectors, retroviral vectors, Epstein-Barr virus vectors, lentivirus vectors, herpes simplex virus vectors, and vectors derived from murine stem cell virus (MSCV) and hybrid vectors described by Hawley (Curr. Gene Ther. 1:1-17 (2001). Numerous vectors useful for this purpose are generally known and have been described (Miller, Human Gene Therapy 15:14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller and Rosman, Biotechniques 7:980-990, 1989; Rosenberg et al., N. Engl. J. Med 323:370, 1990, Groves et al., Nature, 362:453-457, 1993; Horrelou et al., Neuron, 5:393-402, 1990; Jiao et al., Nature 362:450-453, 1993; Davidson et al., Nature Genetics 3:2219-2223, 1993; Rubinson et al., Nature Genetics 33, 401-406, 2003; Buning et al., (Cells Tissues Organs 177:139-150 (2004)); and Tomanin et al., Curr. Gene Ther. 4:357-372 (2004).

In one preferred example, an Epstein Barr virus (EBV) based vector is used which remains episomal and can propagate indefinitely. In this example, the recombinase sequences are introduced around the EBV replication origin and after treatment with the appropriate recombinase, the origin of replication is lost and the episomal sequences will no longer propagate resulting in loss of the episomal sequences.

Non-limiting examples of plasmids useful in the invention include pSG, pSV2CAT and PXt1 from Stratagene, and pMSG, pSVL, pBPV, and pSVK3 from Pharmacia.

The above-described methods for introducing Tsix or Xite transgenes into stem cells can also be used for delivery of therapeutic genes to the stem cells before or after differentiation has been blocked.

### Assays for Transgene Expression

Once a stem cell culture has been infected, transfected, or microinjected with the transgene or small RNA molecule, cells are cultured in selection media to isolate cells that stably express the plasmid or viral vector that contains the transgene. Selection methods are generally known in the art and include, for example, culturing of cells in media containing a selection agent for selection of cells expressing the appropriate selectable marker gene. The selectable marker gene can encode a negative selection marker, a positive selection marker or a fusion protein with positive and negative selection traits. Negative selection traits can be provided in situations whereby the expressed gene leads to the elimination of the host cell, frequently in the presence of a nucleoside analog, such as gancyclovir. Positive selection traits can be provided by drug resistance genes. Suitable negative selection markers include, for example, nucleic acid sequences encoding Hprt, gpt, HSV-tk, diphtheria toxin, ricin toxin, and cytosine deeaminase. Suitable positive selection markers include, for example, nucleic acid sequences encoding neomycin resistance, hygromycin resistance, puromycin resistance, histidinol resistance, xanthine utilization, Zeocin resistance, and bleomycin resistance. Drug resistant cells can either be pooled for a mixed population or colonies can be individually selected (e.g., small groups of about 25 to 1000 cells, preferably, 25 to 500 cells, and most preferably 25 to 100 cells) and plated to generate clonal cell lines or cell lines in which a high proportion (80%, 85%, 90%, 95% or more) of the cells express the transgene.

Genetic alteration of stem cells is rarely 100%, and the population of cells that have been successfully altered can be enriched, for example, by co-transfection of the transgene with a label such as GFP or an immunostainable surface marker such as NCAM which can be used to identify and isolate transfected cells by fluorescence-activated cell sorting.

Cells expressing the transgene can be assayed for the presence of markers of proliferation, indicators of an undifferentiated cell, or the absence of indicators of differentiation to determine if differentiation has been successfully prevented. Examples of assays for differentiation are described below.

Cell lines that express the transgene and are blocked from differentiating are included in the invention. Such cells can be maintained indefinitely and used for any therapeutic purpose requiring a stem cell, such as those described herein. Such cells can also be genetically modified with a therapeutic transgene. For example, a “master” mammalian (e.g., human) ES cell line or a “master” mammalian (e.g., human) adult stem cell line of the invention can be genetically modified for use in the treatment of neurodegenerative disorders (e.g., Alzheimer's or Parkinson's or traumatic injury to the brain or spinal cord), hematologic disorders (e.g., sickle cell, thalassemias), muscular dystrophies (e.g., Duchenne's muscular dystrophy), endocrine disorders (e.g., diabetes, growth hormone deficiency), Purkinje cell degeneration, heart disease, vision and hearing loss and others.

### Differentiation

Cells in which differentiation is effectively blocked by the introduction of a transgene or small RNA molecule using the methods of the invention can be assayed by detecting phenotypic characteristics of undifferentiated cells or by detecting either the presence of markers specific for undifferentiated cells, or the absence of markers or characteristics of differentiated cells.

The morphology of the undifferentiated stem cell is distinct from that of the differentiated stem cell and morphological characteristics can be used to identify stem cells that are successfully transfected with the transgene and that remain in the undifferentiated state. Generally, ES cells are immortalized and have a rounded morphology, a high radiance level, and very little cellular outgrowth on gelatinized plates. Methods for detecting morphology of the transfected stem cells are also known in the art.

Markers that indicate the undifferentiated state or that indicate the absence of differentiation can also be used. In the first instance, markers such as stage-specific embryonic antigen (SSEA) 1, 3, and 4, surface antigens TRA-1-60 and TRA-1-81, alkaline phosphatase, Nanog, Oct-4, and telomerase reverse transcriptase are all indicators of the undifferentiated state of the stem cell for mouse, primate, or human cells. A molecular profile of additional genes expressed by undifferentiated ES cells that can be used to monitor ES cell differentiation are described in Bradenberger et al., (BMC Dev. Bio. 4:10 (2004)).

In the second instance, undifferentiated cells can be identified by the absence of markers of differentiation. Exemplary markers of differentiation include any protein or mRNA that is characteristic of a particular differentiated cell and will be known to the skilled artisan. For example, cells that have differentiated into neurons will express tyrosine hydroxylase, cells that have differentiated into oligodendrocytes will express NG2 proteoglycan, A2B5, and PDGFR-α, and will be negative for NeuN, cells that have differentiated into T lymphocytes will express CD4 and CD8, and cells that have differentiated into a mature granulocyte will express Mac-1.

Additional examples of markers of differentiated and undifferentiated cell types can be found at the in Appendix E of the NIH report stem cells entitled Stem Cells: Scientific Progress and Future Research Directions, supra. Methods for detecting the expression of protein markers, transcription factors, or surface antigens or the mRNA or genes encoding these (e.g., the Pou5f1 gene that encodes the Oct-3/Oct-4 transcription factor) are known in the art and include, for example, immunstaining, immunoblotting, immunohistochemistry, PCR, southern blotting, northern blotting, RNase protection assays, and in situ hybridization.

### Inactivation of Transgenes

For applications (e.g., therapeutic applications) that require control of the switch from the undifferentiated state to the differentiated state, the transgene is inactivated to reduce or eliminate the block to differentiation. In preferred embodiments, the transgene is inactivated by removal of the transgene using, for example, site specific recombination methods. For such applications, the genetically modified stem cell is maintained for a suitable time period sufficient for manipulation or handling (e.g., 1 to 90 days, preferably 1 to 45 days, more preferably 1 to 30 days or 1 to 10 days) prior to removal of the transgene.

Any site specific recombinase/DNA recognition sequence known in the art can be used to remove the transgene from the stem cells of the invention. One example of a site-specific recombinase is Cre recombinase. Cre is a 38-kDa product of the cre (cyclization recombination) gene of bacteriophage P1 and is a site-specific DNA recombinase of the Int family (Sternberg et al., J Mol. Biol. 187: 197-212 (1986). Cre recognizes a 34-bp site on the P1 genome called loxP (locus of X-over of P1) and efficiently catalyzes reciprocal conservative DNA recombination between pairs of loxP sites. The loxP site consists of two 13-bp inverted repeats flanking an 8-bp nonpalindromic core region. Cre-mediated recombination between two directly repeated loxP sites results in excision of DNA between them as a covalently closed circle. Cre-mediated recombination between pairs of loxP sites in inverted orientation will result in inversion of the intervening DNA rather than excision. Breaking and joining of DNA is confined to discrete positions within the core region and proceeds one strand at a time by way of transient phophotyrosine DNA-protein linkage with the enzyme. Additional examples of site-specific recombination systems include the integrase/att system form bacteriophage lambda and the FLP (flippase)/FRT system from the Saccharomyces cerevisiae 2pi circle plasmid. Additional details on these and additional or modified recombinase/DNA recognition sequences and methods for using them can be found, for example, in U.S. Pat. Nos. 4,959,317; 5,527,695; 6,632,672; and 6,734,295; Kilby et al. Trends Genet. 9:413-421 (1993); Gu et al. Cell 73:1155-1164. (1993); Branda et al., Dev. Cell. 6:7-28 (2004); Sauer Endocrine 19:221-228 (2002; Pfeifer et al., Proc. Natl. Acad. Sci. 98:11450-11455 (2001), and Ghosh et al., Methods 28:374-83 (2002).

### Assays for Transgene Inactivation

After the genetically altered stem cells have been maintained for the desired period of time, successful inactivation of the transgene or small RNA molecule (for example, by natural degradation) can be assayed using a variety of techniques that will be known to the skilled artisan. For example, the ability of the cells to grow in selection media can be used as an assay for the successful removal of the transgene. In this example, the use of the recombinase eliminates all transgene sequences (except for one remaining recognition site) including the selectable marker gene. As a result, the cells lose the ability to grow in positive selection media. Cells can be seeded and grown into clonal cell lines using standard limiting dilution methods. Clonal cell lines can be replica plated and one set can be cultured in the presence of the selection agent while the second is cultured in the absence of selection agent. Cells that have lost their ability to grow in the selection media are identified as cells that have lost the transgene. The matched set of these cells can then be grown in the absence of the selection media, expanded, and used as desired.

While removal of the trans gene should be sufficient to induce X chromosome inactivation and potentiate differentiation of the cells, in some cases additional factors may be required to fully induce differentiation or to induce differentiation into a desired cell type. Such factors are described, for example, in U.S. Patent Application Publication Number 20050037492 and in Appendix D of the NIH report stem cells entitled Stem Cells: Scientific Progress and Future Research Directions, supra.

Identification of phenotypic characteristics of differentiation or markers of differentiation, as described above, can also be used to identify cells in which the transgene is inactivated and the cells have successfully undergone differentiation.

As described above, the transgenes are known to block X chromosome inactivation. Accordingly, assays for X chromosome inactivation, include nucleation of chromosome pairing, can also be used to identify cells in which the transgene is inactivated and/or that no longer harbor the transgene. Examples of such assays are described herein (e.g., fluorescent in situ hybridization (Ogawa et al., supra) or in Lee et al., Cell (1999), supra, Stavropoulos et al., Proc. Natl. Acad. Sci. 98:10232-10237 (2001), Lee, Nature Genetics (2002), supra, and Ogawa et al., supra.

### Combination Methods

Any of the transgenes described herein can be used in combination with additional transgenes described herein to enhance the desired effects. In addition, a combination of the use of siRNA with one or more transgenes of the invention can also be used to achieve the desired effects. If desired, the methods described herein may be combined with additional methods known in the art to reduce differentiation in stem cells. Such methods include growth on a feeder layer of mouse embryonic fibroblast cells, growth in Matrigel™, the addition of leukemia inhibitory factor to the culture medium, and the addition of map kinase kinase inhibitors such as PD98059 (Sigma, catalog number P215-5MGA), LIF, Oct-4, Gab1, STAT3, or FGF, (or factors that activate the activity or expression of these proteins) to the culture media (see, for example, the methods described in Xu et al., Nature Biotech. 19:971 (2001), Amit et al., Biol. Reprod. 70:837-45 (2004), PCT Publication Number WO 01/51616, and U.S Patent Application Publication Numbers 20040235159 and 20050037492).

### Therapeutic Applications

The methods for regulating differentiation of stem cells described herein have numerous clinical, agricultural, and research uses that will be appreciated by the skilled artisan. Stem cells have enormous clinical potential because of their ability to differentiate into any cell type of the body. The cells can be used as the starting point for the generation of replacement tissue or cells, such as cartilage, bone or bone cells, muscle or muscle cells, neuronal cells, pancreatic tissue or cells, liver or liver cells, fibroblasts, and hematopoetic cells. Using the methods described herein, the clinician or researcher can introduce the appropriate transgene into the stem cells to prevent differentiation and then remove the transgene just prior to administering the cell product to the patient. If small RNA is used, such small RNA will generally degrade naturally and does not need to be removed.

The methods for regulating differentiation of mammalian stem cells described herein, for example, can be used for the treatment of diseases treatable through transplantation of differentiated cells derived from ES cells. The ES cells are maintained in the undifferentiated state for a period of time sufficient to genetically manipulate the cells prior to differentiation either to reduce immunogenicity or to give new properties to the cells to combat specific diseases. Furthermore, the use of the methods for regulating differentiation described herein not only allow the practitioner sufficient time to genetically modify the stem cells but, because of the ability of the stem cell to self-renew, allow for the gene to be maintained throughout successive cell divisions, thereby circumventing the need for repeated transgene introduction.

Stem cells of the invention or produced using the methods of the invention can be used to treat, for example, neurodegenerative disorders (e.g., Alzheimer's or Parkinson's or traumatic injury to the brain or spinal cord), hematologic disorders (e.g., sickle cell, thalassemias), muscular dystrophies (e.g., Duchenne's muscular dystrophy), endocrine disorders (e.g., diabetes, growth hormone deficiency), Purkinje cell degeneration, heart disease, vision and hearing loss and others in any mammal, preferably a human. Additional examples of the use of genetically modified stem cells in experimental gene therapies are described in Chapter 11 of NIH report stem cells entitled Stem Cells: Scientific Progress and Future Research Directions, supra and also in Shufaro et al., Best Pract. Res. Clin. Obstet. Gynaecol. 18:909-927 (2004).

The cells and methods of the invention can also be used for agricultural purposes to clone desirable livestock (e.g., cows, pigs, sheep) and game. For such purposes, the appropriate species of stem cell line and transgene are used.

### Research Applications

The invention can also be used for research purposes for the study of differentiation or development, and for the generation of transgenic animals useful for research purposes. The stem cells and the methods for regulating the differentiation of the stem cells described herein can be used, for example, to identify signaling pathways or proteins involved in differentiation processes, which can lead to the identification of future therapeutic targets for the treatment of a variety of diseases. The stem cells and methods of the invention can also be used to study the effects of a particular gene or compound on stem cell differentiation, development, and tissue generation or regeneration.

### EXAMPLES

The following examples are provided for the purposes of illustrating the invention, and should not be construed as limiting.

### Example 1

### Models for XCI and the Counting Elements Involved

X-chromosome inactivation achieves dosage compensation in mammals by establishing equal X-chromosome expression in XX (female) and XY (male) individuals (Lyon, Nature 190: 372-373 (1961)). The XCI pathway involves a series of molecular switches that include X-chromosome counting, epigenetic choice, and chromosome-wide silencing (Avner et al., Nature Rev. Genet. 2:59-67 (2001)). ‘Counting’ ensures that XCI occurs only in nuclei with more than one X (n>1). When n>1, a ‘choice’ mechanism epigenetically selects one X as the active X (Xₐ) and the second X as the inactive X (Xᵢ). During choice, the parallel action of three noncoding, cis-acting genes—Xist (Brown et al., Cell 71:527-542 (1992); N. Brockdorff et al., Cell 71:515-526 (1992)), Tsix (Lee et al., Nature Genet. 21:400-404 (1999)), and Xite (Ogawa et al., supra)—establishes the respective fates of each chromosome. On the designated Xᵢ, Xist RNA (produced in cis) envelopes the X-chromosome (Clemson et al., J. Cell Biol. 132:259-275 (1996)) and initiates chromosome-wide silencing on the X in cis (Penny et al., Nature 379:131-137 (1996); Marahrens et al., Genes & Dev. 11:156-166 (1997)). On the designated Xₐ, the antisense action of Tsix together with the enhancing action of Xite blocks the promulgation of Xist RNA to maintain chromosome activity (Ogawa et al., supra; Lee et al., Cell (1999), supra; Lee et al., Cell (2000), supra; Sado et al., supra). In short, the choice and silencing steps of XCI are controlled by the opposing and dynamic action of RNA-producing genes.

Although the counting mechanism makes up the apical switch, little is known about how it functions. General rules have been inferred from studies of sex chromosome aneuploids (Lyon, supra; Rastan, J. Embryol. Exp. Morph. 78:1-22 (1983); Rastan et al., J. Embryol. Exp. Morph. 90:379-388 (1985)). For example, the number of Xs subject to inactivation follows the ‘n−1’ rule, whereby all but one X is inactivated in diploids. Therefore, XX cells silence one X, while XXX cells silence two. Counting is also influenced by ploidy, as shown by the fact that, while diploids maintain only one Xₐ, tetraploids can maintain two Xₐ and octaploids can maintain four Xₐ (Lyon, supra; Webb et al., Genet. Res. 59:205-214 (1992); Jacobs et al., Am. J. Hum. Genet. 31:446-457 (1979)). Therefore, the mammalian counting mechanism is not determined by the absolute number of X-chromosomes, but rather by the X-to-autosome (X:A) ratio. This implies that specific X-linked and autosomal factors (X-factor and A-factors, respectively) are measured during early development.

Two types of models for counting have been advanced in recent years. The most widely accepted model (Avner et al., supra; Lyon, supra; Rastan, (1983), supra)—herein named the ‘singularity model’—posits that counting is achieved by a single ‘blocking factor’ which binds and protects a single X from inactivation in diploids. All other X's are silenced by default. An alternative ‘duality’ model (et al., Lee et al., Cell (1999), supra) postulates regulation by two factors: a blocking factor that protects the future Xₐ and a ‘competence factor’ that induces XCI on the future Xᵢ. A key difference between the models is that, while the singularity model stipulates that Xᵢ's are formed by default, the duality model requires purposeful action to achieve both the Xₐ and Xᵢ. The current evidence does not conclusively favor either.

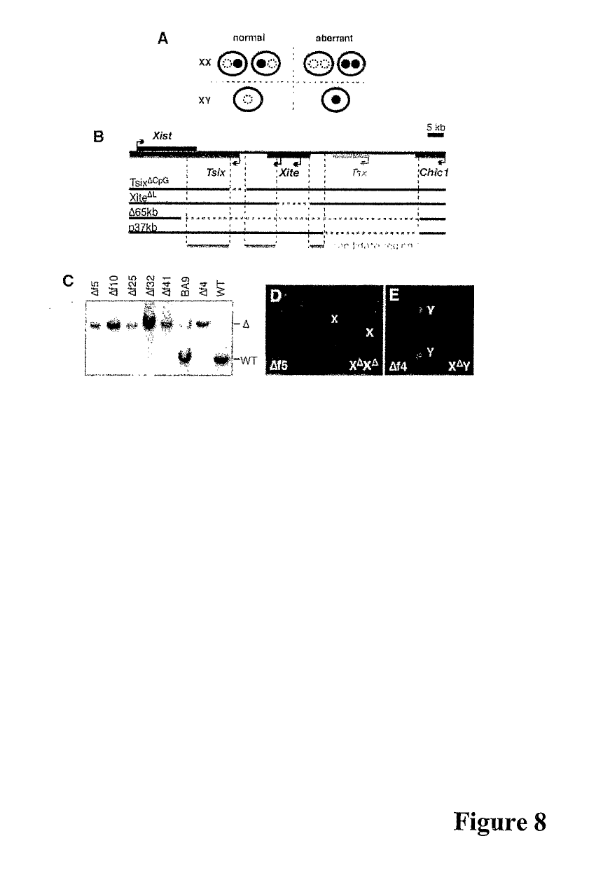

To date, specific X-linked and autosomal factors have not been identified, despite a growing catalogue of XCI mutations. A priori, mutations in the counting pathway could be recognized by any deviation from the expected number of Xᵢ. These include the appearance of an Xᵢ in XY or XO cells, absence of any Xᵢ in XX cells, or the appearance of a second Xᵢ in XX cells (FIG. 8A). Using mouse embryonic stem (ES) cells as an ex vivo system to study XCI, transgenic analyses have shown that elements within or near the X-inactivation center (Xic) are involved in counting (Lee et al., Cell (1996), supra; Heard et al., Molec. Cell. Biol. 19:3156-3166 (1999); Lee et al., Proc. Natl. Acad. Sci. U.S.A. (1999), supra; Migeon et al., Genomics 59:113-121 (1999)). XY cells display ectopic XCI when additional copies of Xic sequence are introduced. In ES cells, knockout analyses have also suggested the presence of counting factors at the Xic in a region that spans Xist, Tsix, Xite, and Tsx (a testis-specific gene). A 65 kb deletion (Δ65 kb; FIG. 8B) of this region leads to ectopic Xᵢ in a subset of XO and XY cells (Clerc et al., Nature Genet. 19:249-253 (1998)). Adding back 37 kb of sequence up to but not including Tsx eliminates this population of abnormal cells (FIG. 8B, p37 kb)(Morey et al., Embo J 23:594-604 (2004)).

Based on available genetic experiments, a candidate counting element is thought to lie downstream of Xist, exclusive of the 5′ ends of Tsix and Xite (FIG. 8B)(Morey et al., Embo J23:594-604 (2004)). Evidence for this idea includes that knocking out the CpG island of Tsix (Lee et al., Cell (1999) supra; Sado et al., supra; Luikenhuis et al., Mol. Cell. Biol. 21:8512-8520 (2001)) and the major hypersensitive sites of Xite (Ogawa et al., supra; Sado et al., supra) does not produce an aberrant number of Xᵢ (FIG. 8B, TSix^{ΔCpG} and Xite^{ΔL}). Δ/Y males appropriately block XCI and Δ/+females exhibit a single Xᵢ. However, although Tsix and Xite heterozygous females seem to count appropriately, they are defective in choice, as there is a primary effect on selecting the mutated X as Xᵢ (Ogawa et al., supra; Lee et al., Cell (1999) supra; Lee et al., Cell (2000), supra; Sado et al., supra; Luikenhuis et al., Mol. Cell. Biol. 21:8512-8520 (2001); Stavropoulos et al., Proc. Natl. Acad. Sci. U.S.A. 98:10232-10237 (2001); Morey et al., Hum. Mol. Genet. 10:1403-1411 (2001)). These experiments demonstrate that counting and choice are, in fact, genetically separable.

Yet, while the knockout studies are clear with respect to Tsix's role in choice and not in counting, a recent observation has raised new possibilities (Lee, Nature Genet. (2002), supra). Specifically, although TsixΔ/+(henceforth X^{Δ}X) mice invariably inactivate the mutated X, TsixΔ/Δ (henceforth X^{Δ}X^{Δ}), homozygotes apparently revert to random XCI. Paradoxically though, X^{Δ}X^{Δ} embryos show greater in utero loss than their X^{Δ}Y and X^{Δ}X counterparts. These observations suggest that Tsix may play additional roles which are evident only when both alleles are mutated. Indeed, it has been proposed that Tsix not only selects the future Xₐ in cis but also ensures mutually exclusive choice by allowing cross-talk between the two antisense alleles (Lee, Nature Genet. (2002) supra). Loss of both alleles may therefore result in a state of ‘chaotic choice,’ whereby the choice decision occurs without coordination between the X's and leads to aberrant patterns of XCI. By chance alone, some X^{Δ}X^{Δ} cells may arrive at a normal pattern of XₐXᵢ, while others perish as a result of abnormal dosage compensation.

The model makes clear and testable predictions. If X^{Δ}X^{Δ} cells undergo chaotic choice, multiple aberrant patterns of XCI might be detectable in differentiating XX cells, as manifested by occurrence of some nuclei with two Xᵢ, some with one Xᵢ, and others with no Xᵢ (total chaos). A chaotic choice pattern bears striking resemblance to aberrant counting (FIG. 8A). Thus, the chaotic choice model further predicts that Tsix itself might be involved in counting. The study below tests this hypothesis and finds that specific noncoding genes play a role in counting. The unusual manifestations in XX and XY cells favors a duality model, which now provides a new conceptual framework for understanding the details of the counting mechanism.

### Chaotic XCI in a Homozygous Tsix ES Model

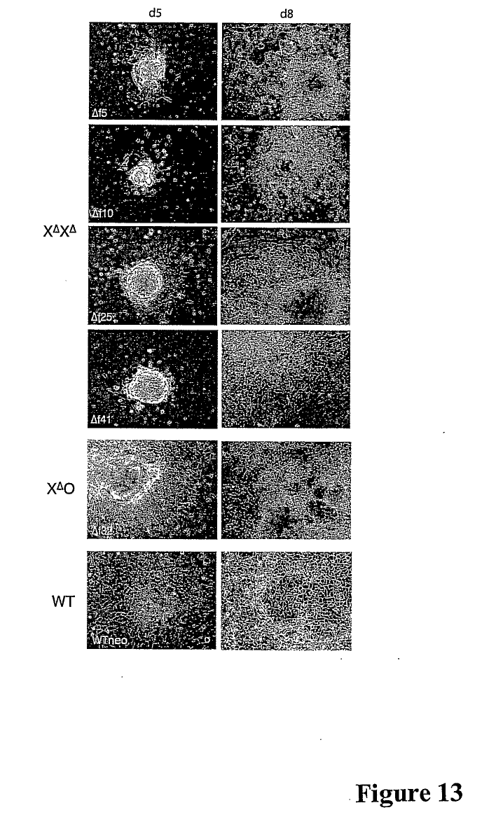

Because random XCI takes place in the epiblast (E4.5-5.5), the initiation of XCI is difficult to characterize in X^{Δ}X^{Δ} embryos due to limited cell numbers and potential contamination by abundant embryonic cells. To circumvent this problem, I set up X^{Δ}X^{Δ}×X^{Δ}Y crosses and cultured resulting blastocysts to generate X^{Δ}X^{Δ} ES cells. In mice, ES cells have provided a powerful ex vivo system to study XCI because they recapitulate XCI during cell differentiation. Ninety-three blastocysts were isolated from 11 crosses. Consistent with previous observation (Lee, Nature Genet. (2002) supra), a significant fraction of the blastocysts yielded poor quality ICM outgrowths. In total, five X^{Δ}X^{Δ} ES lines were established and identified by Southern blotting (FIG. 8C). Genomic DNAs were digested with BamHI, subjected to gel electrophoresis, blotted onto membrane, and hybridized to a 1.4 kb NdeI-MluI fragment downstream of the Tsix start site (Lee et al., Cell (1999), supra). WT, wild type female (16.7); BA9, X^{Δ}X control. These results indicated that it is possible to isolate ES cells with a homozygous Tsix deficiency despite the poor overall fitness of X^{Δ}X^{Δ} embryos (Lee, Nature Genet. (2002) supra). Clones Δf1, Δf5, Δf10, Δf25, and Δf41 were confirmed as female by lack of a Y-chromosome as determined by Y-chromosome painting, absent bands in Zfy PCR experiments, and occurrence of two Xs in a diploid background as determined by fluorescence in situ hybridization (FISH) (FIG. 8D). FISH carried out as described previously (Ogawa et al., Mol Cell 11:731-743 (2003); Lee et al., supra). Because all five X^{Δ}X^{Δ} clones behaved similarly (Table 3, FIG. 13), results below will be shown only for representative clones. X^{Δ}Y male (Δf4) and X^{Δ}O female (Δf21, Δf32) lines were also isolated as controls. Clones Δf4, Δf21, and Δf32 all carry a single X-chromosome, and Δf4 also carries a Y-chromosome (FIG. 8E). Once established in culture, X^{Δ}X^{Δ} ES lines grew no differently from WT (wildtype) female ES cells in the undifferentiated state. However, upon differentiation into embryoid bodies (EB), several differences were immediately evident. To generate EB, adherent ES cells were lightly trypsinized to generate detached clusters on d0, grown in suspension culture as clusters (EB) for 4 days in DME+15% fetal calf serum without LIF, and plated on gelatin on d4 for another 4-6 days to generate EB outgrowths (Lee et al., Proc. Natl. Acad. Sci. U.S.A. (1999), supra). First, all ^{XΔXΔ} EB grew poorly as compared to WT, X^{Δ}X, and X^{Δ}O controls. X^{Δ}X^{Δ} EB tend to remain small, break up during culture, and generate an unusual amount of cellular debris (FIG. 9A). To quantitate cell death, EB were grown as usual and all cells (both loose in suspension or adherent on plates) were harvested on d0, d4, and d8 for staining with trypan blue. To calculate the percent dead, the number of cells staining blue (dead) was divided by the total number of cells staining blue (dead) and excluding dye (alive). Cell death analysis revealed that a large fraction of X^{Δ}X^{Δ} cells lose viability as compared to WT (P<0.01), beginning immediately after formation of EB and culminating on day 8 (FIG. 9B). This contrasted with a rate for the X^{Δ}O control (Δf32) that is comparable to WT (P>0.2). Interestingly, despite massive cell death, a fraction of X^{Δ}X^{Δ} EB could attach on day 4 and give rise to EB outgrowths, albeit not so robustly as WT, X^{Δ}X, or X^{Δ}O EB (FIGS. 9A, d9, FIG. 13). This result agreed with the in vivo finding that X^{Δ}X^{Δ} embryos are frequently lost in utero but can occasionally survive to birth (Lee, Nature Genet. (2002) supra).

To assess the XCI status of mutant clones, I next performed Xist RNA FISH to determine whether an Xᵢ (Xist RNA nuclear domain) formed at the single-cell level. Like WT female lines, X^{Δ}X^{Δ} ES lines maintained two Xₐ in the undifferentiated state, as deduced by the absence of Xist accumulation on day 0 of differentiation (FIG. 9C). FISH carried out as described previously (Ogawa et al., Mol Cell 11:731-743 (2003)). Upon differentiation, the pattern of XCI in X^{Δ}X^{Δ} cells became significantly different from that of controls, including WT, X^{Δ}X, X^{Δ}Y, and X^{Δ}O cells (FIGS. 9C,D; Table 3). Between days 2 and 4, when XCI normally initiates, mutant EB were characterized by three types of XCI patterns: those with two Xₐ, those with one Xᵢ, and those with two Xᵢ (FIGS. 9C,D). In contrast, WT and X^{Δ}X cells yielded only those with one Xᵢ and those with two Xₐ (reflecting a subpopulation that had not yet differentiated) (FIGS. 9C,D; Table 3). The controls, X^{Δ}O (Δf32) and X^{Δ}Y (Δf4), yielded no Xist expression on any day (FIG. 9C, Table 3).

Table 3 shows a summary of mutant ES cell lines and their characteristics. Multiple clones of each knockout and transgenic series were analyzed in this study, with three to four of each series shown. Only one male clone of pCC3, pCC4, p3.7, pXite, and pSxn series was examined, because the larger pSx7 transgenic lines indicated no phenotype in an XY background. All cell lines were generated in this study, except J1 (Li et al., Cell 69:915-926 (1992)), 16.6 (Lee et al., Cell (1999), supra), and BA9 (a 1-lox neo-minus derivative of 3F1)(Lee et al., Cell (1999), supra). Transgene copy numbers determined by Southern blot analysis, phosphorimaging, and FISH signal density and size. Low, 1-4 copies. High >5 copies. Tg, transgenic. n.a., not applicable.

TABLE 3
Summary of mutant ES cell lines and phenotypes

| | | | Tg | | | | | Xi number per 2n | |
|---|---|---|---|---|---|---|---|---|---|
| Category | Cell line | Genotype | Copies* | Growth/differentiation** | Cell Death | | | cell (days 2-4) | Counting |
| — | — | — | — | — | d 0 | d 4 | d 8 | — | — |
| WT controls | 16.6 (WT) | Female WT ES | n.a. | robust | 0.5% | 29.6% | 26.7% | 0, 1 | normal |
| | | (ref. 2) | | | | | | | |
| | J1 | Male WT ES | n.a. | robust | 0.3% | 25.5% | 31.4% | 0 | normal |
| | | (ref. 15) | | | | | | | |
| Tsix hetero- | BA9 | Female XΔX | n.a. | robust (same as WT) | 0.7% | 33.8% | 34.5% | 0, 1 | normal |
| zygote | | (ref. 2) | | | | | | | |
| Tsix hemi- and | Δf4 | Male XΔY | n.a. | robust (same as WT) | 0.2% | 30.9% | 31.4% | 0 | normal |
| homo- zygotes | Δf21 | Female XΔ◯ | n.a. | robust (same as WT) | 0.4% | 25.6% | 31.7% | 0 | normal |
| | Δf32 | Female XΔ◯ | n.a. | robust (same as WT) | 0.4% | 26.9% | 22.7% | 0 | normal |
| | Δf1 | Tsix homozygous | n.a. | small EB, slow | 0.9% | 42.0% | 56.3% | 0, 1, 2 | aberrant |
| | | XΔXΔ | | outgrowth | | | | | |
| | Δf5 | Tsix homozygous | n.a. | small EB, slow | 0.5% | 59.5% | 38.9% | 0, 1, 2 | aberrant |
| | | XΔXΔ | | outgrowth | | | | | |
| | Δf10 | Tsix homozygous | n.a. | small EB, slow | 0.7% | 50.4% | 57.0% | 0, 1, 2 | aberrant |
| | | XΔXΔ | | outgrowth | | | | | |
| | Δf25 | Tsix homozygous | n.a. | small EB, slow | 1.2% | 42.6% | 58.6% | 0, 1, 2 | aberrant |
| | | XΔXΔ | | outgrowth | | | | | |
| | Δf41 | Tsix homozygous | n.a. | small EB, slow | 0.6% | 41.4% | 38.2% | 0, 1, 2 | aberrant |
| | | XΔXΔ | | outgrowth | | | | | |
| Female Tg - | π2.1B | Female πJL2 Tg | low | small EB, minimal | 0.8% | 54.4% | 51.9% | 0, 1 (Xist | aberrant |
| (large Tg) | | | | outgrowth, d 8 dead | | | | predominantly | |
| | | | | | | | | on Tg) | |
| | π2.18 | Female πJL2 Tg | high | small EB, minimal | 0.8% | 56.7% | 67.1% | 0, 1 (Xist | aberrant |
| | | | | outgrowth, d 8 dead | | | | predominantly | |
| | | | | | | | | on Tg) | |
| | π2.22 | Female πJL2 Tg | low | small EB, minimal | 0.5% | 61.7% | 59.2% | 0, 1 | aberrant |
| | | | | outgrowth, d 8 dead | | | | (Xist on X or Tg) | |
| | π3.2B | Female πJL3 Tg | high | small EB, minimal | 0.9% | 62.1% | 62.7% | 0, 1, 2 | aberrant |
| | | | | outgrowth, d 8 dead | | | | (Xist on X) | |
| | π3.10 | Female πJL3 Tg | low | small EB, minimal | 0.5% | 58.1% | 54.1% | 0, 1 | aberrant |
| | | | | outgrowth, d 8 dead | | | | (Xist on X) | |
| | π3.15 | Female πJL3 Tg | high | small EB, minimal | 0.8% | 54.9% | 69.1% | 0, 1 | aberrant |
| | | | | outgrowth, d 8 dead | | | | (Xist on X) | |
| | Sx7.1B | Female pSx7 Tg | low | small EB, minimal | 0.5% | 55.9% | 62.5% | 0, 1 | aberrant |
| | | | | outgrowth, d 8 dead | | | | (Xist on X) | |
| | Sx7.4 | Female pSx7 Tg | high | small EB, minimal | 0.7% | 52.3% | 60.3% | 0, 1, 2 | aberrant |
| | | | | outgrowth, d 8 dead | | | | (Xist on X) | |
| | Sx7.6 | Female pSx7 Tg | low | small EB, minimal | 0.1% | 51.1% | 57.6% | 0, 1, 2 | aberrant |
| | | | | outgrowth, d 8 dead | | | | (Xist on X) | |
| Female Tg - | Sxn-4 | Female pSxn Tg | high | small EB, minimal | 0.0% | 63.7% | 62.0% | 0 | aberrant |
| (small Tg) | | | | outgrowth, d 8 dead | | | | | |
| | Sxn-6 | Female pSxn Tg | high | small EB, minimal | 0.3% | 70.3% | 70.5% | 0 | aberrant |
| | | | | outgrowth, d 8 dead | | | | | |
| | Sxn-7 | Female pSxn Tg | high | small EB, minimal | 0.1% | 69.0% | 66.3% | 0 | aberrant |
| | | | | outgrowth, d 8 dead | | | | | |
| | Sxn-12 | Female pSxn Tg | high | small EB, minimal | 0.3% | 68.5% | 57.8% | 0 | aberrant |
| | | | | outgrowth, d 8 dead | | | | | |
| | 3.7-5 | Female p3.7 Tg | high | d 5, large stunted EB; | 0.5% | 71.0% | 72.4% | 0 | aberrant |
| | | | | d 8, dead | | | | | |
| | 3.7-8 | Female p3.7 Tg | high | d 5, large stunted EB; | 0.7% | 72.8% | 67.9% | 0 | aberrant |
| | | | | d 8, dead | | | | | |
| | 3.7-10 | Female p3.7 Tg | high | d 5, large stunted EB; | 0.8% | 71.7% | 73.5% | 0 | aberrant |
| | | | | d 8, dead | | | | | |
| | 3.7-11 | Female p3.7 Tg | high | d 5, large stunted EB; | 0.9% | 60.1% | 70.1% | 0 | aberrant |
| | | | | d 8, dead | | | | | |
| | Xite-8 | Female pXite Tg | high | d 5, large stunted EB; | 0.2% | 68.5% | 76.1% | 0 | aberrant |
| | | | | d 8, dead | | | | | |
| | Xite-10 | Female pXite Tg | high | d 5, large stunted EB; | 0.3% | 75.4% | 76.9% | 0 | aberrant |
| | | | | d 8, dead | | | | | |
| | Xite-11 | Female pXite Tg | high | d 5, large stunted EB; | 1.0% | 70.0% | 72.4% | 0 | aberrant |
| | | | | d 8, dead | | | | | |
| | Xite-14 | Female pXite Tg | high | d 5, large stunted EB; | 0.9% | 72.2% | 74.9% | 0 | aberrant |
| | | | | d 8, dead | | | | | |
| | CC3-9 | Female pCC3 Tg | high | d 5, medium stunted EB; | 0.3% | 54.9% | 55.4% | 0 | aberrant |
| | | | | d 8, dead | | | | | |
| | CC3-11 | Female pCC3 Tg | high | d 5, medium stunted EB; | 1.1% | 57.4% | 55.8% | 0 | aberrant |
| | | | | d 8, dead | | | | | |
| | CC3-13 | Female pCC3 Tg | high | d 5, medium stunted EB; | 0.6% | 60.4% | 58.9% | 0 | aberrant |
| | | | | d 8, dead | | | | | |
| | CC3-15 | Female pCC3 Tg | high | d 5, medium stunted EB; | 0.4% | 59.4% | 52.0% | 0 | aberrant |
| | | | | d 8, dead | | | | | |
| | CC4-2 | Female pCC4 Tg | high | d 5, medium stunted EB; | 0.9% | 60.3% | 58.9% | 0 | aberrant |
| | | | | d 8, dead | | | | | |
| | CC4-8 | Female pCC4 Tg | high | d 5, medium stunted EB; | 0.9% | 56.1% | 52.7% | 0 | aberrant |
| | | | | d 8, dead | | | | | |
| | CC4-11 | Female pCC4 Tg | high | d 5, medium stunted EB; | 1.0% | 58.4% | 55.0% | 0 | aberrant |
| | | | | d 8, dead | | | | | |
| | CC4-17 | Female pCC4 Tg | high | d 5, medium stunted EB; | 0.3% | 54.5% | 50.1% | 0 | aberrant |
| | | | | d 8, dead | | | | | |
| Control - | WTneo1 | Female WT neo | n.a. | robust (same as WT XX) | 0.2% | 29.3% | 37.9% | 0, 1 | normal |
| (female Tg) | | Tg | | | | | | | |
| | Xist5′-5 | Female pXist5′ Tg | low | similar to WT XX | 0.5% | 36.6% | 39.4% | 0, 1 | normal |
| | Xist5′-6 | Female pXist5′ Tg | high | similar to WT XX | 2.6% | 37.1% | 37.3% | 0, 1 | normal |
| | Xist5′-7 | Female pXist5′ Tg | low | similar to WT XX | 0.1% | 28.7% | 32.4% | 0, 1 | normal |
| | Xist5′-8 | Female pXist5′ Tg | high | similar to WT XX | 0.1% | 21.6% | 28.6% | 0, 1 | normal |
| | Xist3′-1 | Female pXist3′ Tg | low | similar to WT XX | 0.4% | 32.7% | 33.5% | 0, 1 | normal |
| | Xist3′-2 | Female pXist3′ Tg | low | similar to WT XX | 0.8% | 35.4% | 33.8% | 0, 1 | normal |
| | Xist3′-3 | Female pXist3′ Tg | high | similar to WT XX | 0.2% | 32.1% | 34.0% | 0, 1 | normal |
| | Xist3′-4 | Female pXist3′ Tg | high | similar to WT XX | 0.7% | 37.1% | 31.3% | 0, 1 | normal |
| | Tsx-1 | Female pTsx Tg | low | similar to WT XX | 1.0% | 42.6% | 33.3% | 0, 1 | normal |
| | Tsx-2 | Female pTsx Tg | low | similar to WT XX | 0.8% | 35.6% | 33.2% | 0, 1 | normal |
| | Tsx-3 | Female pTsx Tg | high | similar to WT XX | 0.7% | 31.3% | 30.4% | 0, 1 | normal |
| | Tsx-4 | Female pTsx Tg | high | similar to WT XX | 1.4% | 43.8% | 42.1% | 0, 1 | normal |
| Control - | Sx7.6 | Male pSx7 Tg | low | similar to WT XY | 0.7% | 29.7% | 23.7% | 0 | normal |
| (male Tg) | Sx7.7 | Male pSx7 Tg | low | similar to WT XY | 0.9% | 27.8% | 28.1% | 0 | normal |
| | Sx7.8 | Male pSx7 Tg | high | similar to WT XY | 0.8% | 26.3% | 27.9% | 0 | normal |
| | 3.7-1 | Male p3.7 Tg | low | similar to WT XY | 0.8% | 30.4% | 25.2% | 0 | normal |
| | CC3-1 | Male pCC3 Tg | high | similar to WT XY | 0.4% | 23.0% | 28.9% | 0 | normal |
| | CC4-1 | Male pCC4 Tg | low | similar to WT XY | 0.8% | 26.4% | 27.1% | 0 | normal |
| | Xite-1 | Male pXite Tg | high | similar to WT XY | 0.8% | 33.5% | 30.3% | 0 | normal |
| | Sxn-1 | Male pSxn Tg | high | similar to WT XY | 0.8% | 28.1% | 24.1% | 0 | normal |

In X^{Δ}X^{Δ} cultures, there was a close correlation between success of EB outgrowth and achievement of proper dosage compensation. For example, EB that showed poor outgrowth often displayed clusters of cells with two prominent Xᵢ (FIG. 8E). On later days of differentiation, the increasing presence of EB outgrowths correlated with a rise in number of cells with the proper Xᵢ number (FIG. 8F). In general, there was significant stochastic variation among any X^{Δ}X^{Δ} EB culture with respect to EB outgrowth and achievement of dosage compensation.

From the experiments thus far, several observations suggest that XCI indeed proceeds in a chaotic fashion in X^{Δ}X^{Δ}—first, the occurrence of nuclei with two, one, and no Xᵢ within the same differentiating population; second, the massive cell death associated with aberrant Xᵢ number; and third, the gradual dominance of cells which showed a single Xᵢ, presumably as a result of selection against those that incorrectly chose none or multiple X's. Of particular interest is the fact that the abnormal characteristics were specific to the X^{Δ}X^{Δ} genotype (FIGS. 9A-9F). X^{Δ}Y and X^{Δ}O ES lines were not affected even though they also lack any Tsix function. Moreover, X^{Δ}X lines were spared. This argued that the defect is not related to sex per se, nor is the effect simply the result of absent Tsix function. Instead, the phenotype requires two co-existing conditions: the loss of both Tsix alleles and an XX background.

### Counting Defects Revealed Through Transgenesis

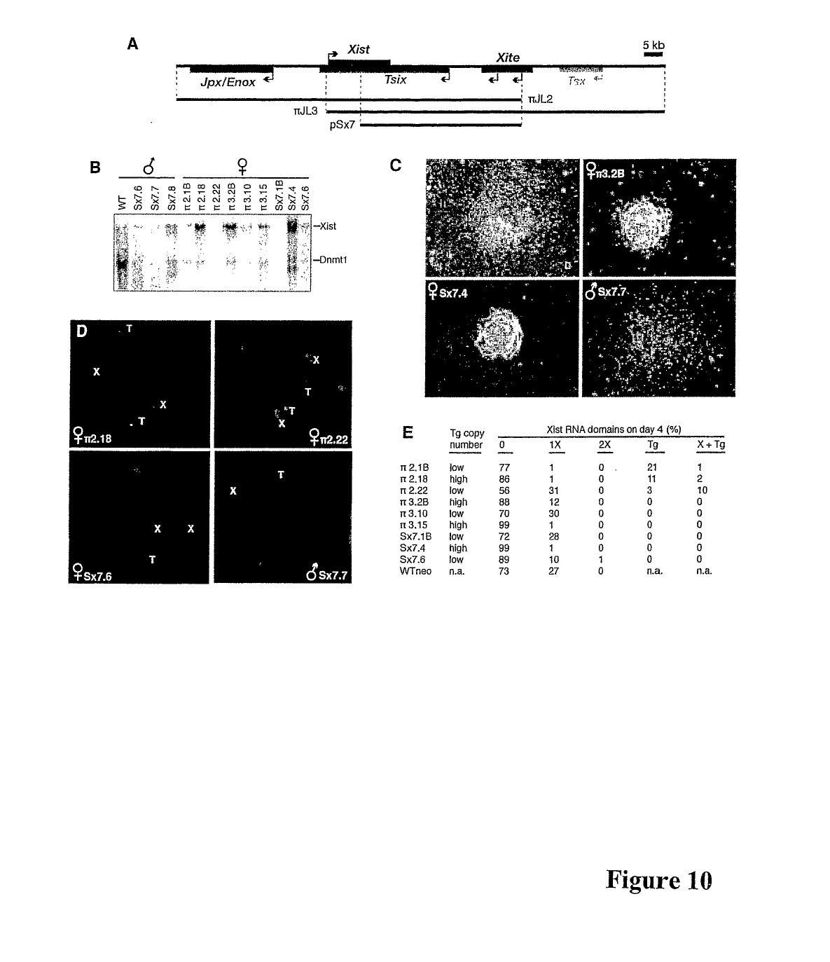

The massive cell death and the accompanying appearance of aberrant Xᵢ numbers suggested a counting defect (FIG. 8A) was responsible for the unusual X^{Δ}X^{Δ} phenotype. In principle, any counting element has to be precisely titrated in the cell. Therefore, if Tsix affects counting, supernumerary copies of Tsix would also disrupt XCI. To test this idea, I used ES transgenesis to introduce extra copies of Tsix. Because the Tsix deletion phenotype was dramatically different in XX and XY cells, I first determined whether Tsix transgenesis would also affect XX and XY cells differently. In XY cells, it was previously shown that introducing 80-460 kb of Xic sequence into XY ES cells led to ectopic inactivation of the X or the autosome in cis to the transgene (Lee et al., Cell (1996), supra; Heard et al., Molec. Cell. Biol. 19:3156-3166 (1999); Lee et al., Proc. Natl. Acad. Sci., (1999) supra; Migeon et al., supra), leading to the idea that a counting element resides within the Xic. Here, I re-created the transgenics in an XX background using the 80 kb plasmids, πJL2 and πJL3 (FIG. 10A)(Lee et al., Proc. Natl. Acad. Sci. U.S.A. (1999) supra). Multiple female clones for each transgene were isolated and three representative clones with low and high copy numbers were characterized in detail (FIG. 10B).

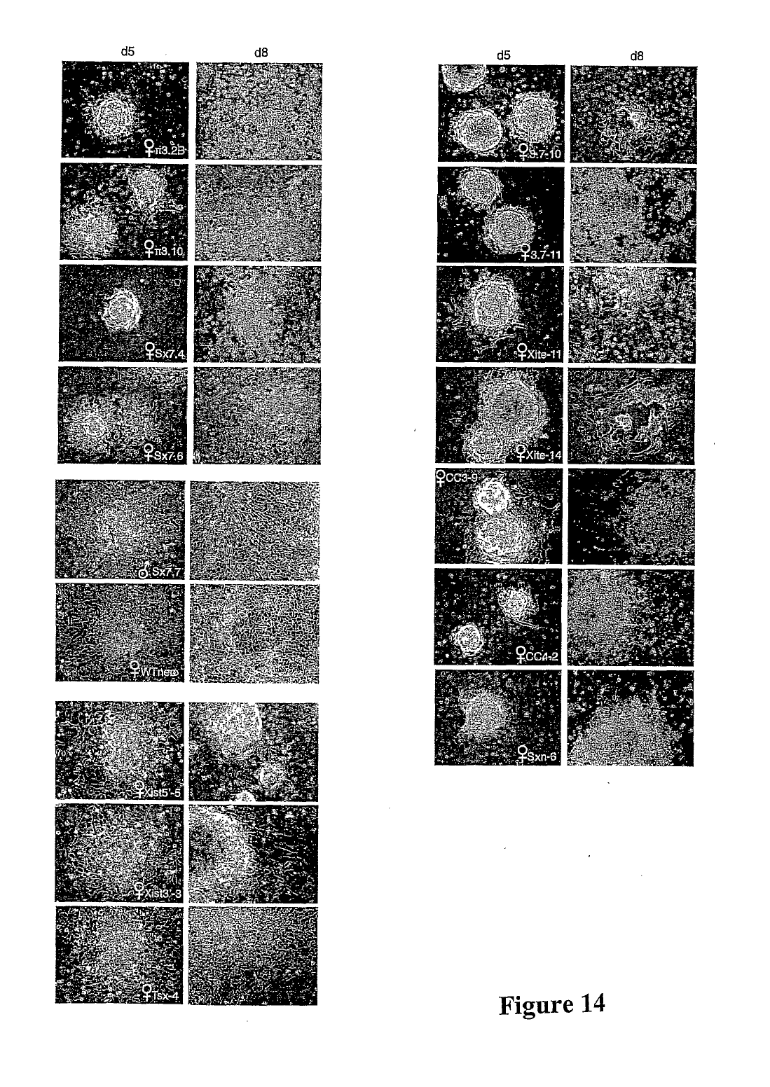

All female transgenics showed poor differentiation, with very few EB demonstrating outgrowth on day 5 of culture (FIG. 10C). In contrast, the previously generated male πJL2 and πJL3 transgenics (Lee et al., Proc. Natl. Acad. Sci. U.S.A. (1999) supra) and female controls carrying only the neo selectable marker (WTneo) yielded moderate to abundant outgrowth (FIG. 10C; Table 3, FIG. 14). Because XY transgenics apparently could differentiate into EB (Lee et al., Proc. Natl. Acad. Sci. U.S.A. (1999), supra), these results suggested XX and XY differences in a transgenic assay as well. FISH analysis of πJL2 and πJL3 transgenic females revealed Xist RNA accumulation on either the X's or transgene-bearing autosome (FIG. 10D), as have been reported for XY transgenics previously (Lee et al., Proc. Natl. Acad. Sci. U.S.A. (1999) supra).

A potential complication of these transgenes was the presence of Xist, which could stunt growth by ectopic autosomal inactivation (Lee et al., Proc. Natl. Acad. Sci. U.S.A. (1999), supra). To separate the effects of autosomal inactivation from that of a counting defect, I eliminated Xist expression by using pSx7 (FIG. 10A). pSx7 manifested profoundly different phenotypes in XX cells as compared to XY. While XY transgenics showed robust differentiation, XX cells yielded little to no EB outgrowth (FIG. 10C, FIG. 14). These results argued that the stunted EB growth occurred independently of Xist-induced autosomal inactivation. Examination of Xᵢ formation by Xist RNA FISH led to a further disparity between XX and XY cells: while pSx7 females underwent Xᵢ formation, pSx7 males never showed XCI (FIG. 10D, FIG. 14). This disparity provided additional insight into the counting process and is discussed in the next section.

Intriguingly, the frequency of Xist expression inversely correlated with transgene copy number (FIGS. 10D,E). In the πJL2 series, the low-copy clones (π2.1B, π2.22) showed an Xist domain in 23-44% of cells on day 4, but the high-copy clone (π2.18) possessed an Xist domain in only 14% of cells. In the latter, Xist RNA often appeared sparse (FIG. 10D, arrows) rather than the robust clusters seen in WT and low-copy clones. In πJL2 clones, Xist RNA accumulated on either the X or autosome, consistent with results found in XY cells (Lee et al., Proc. Natl. Acad. Sci. U.S.A. (1999) supra). Copy number likewise influenced Xist expression frequency in the πJL3 and pSx7 series. In these clones, however, Xist RNA accumulated only on the X and never on the autosome in these cells, consistent with πJL3's breakpoint in the Xist promoter reported previously in XY cells (Lee et al., Proc. Natl. Acad. Sci. U.S.A. (1999) supra) and with pSx7's deletion of the 5′ end of Xist. This dose-response relationship suggested the property of titrability and argues for counting element(s) in the pSx7 region.

### Tsix and Xite are Counting Elements

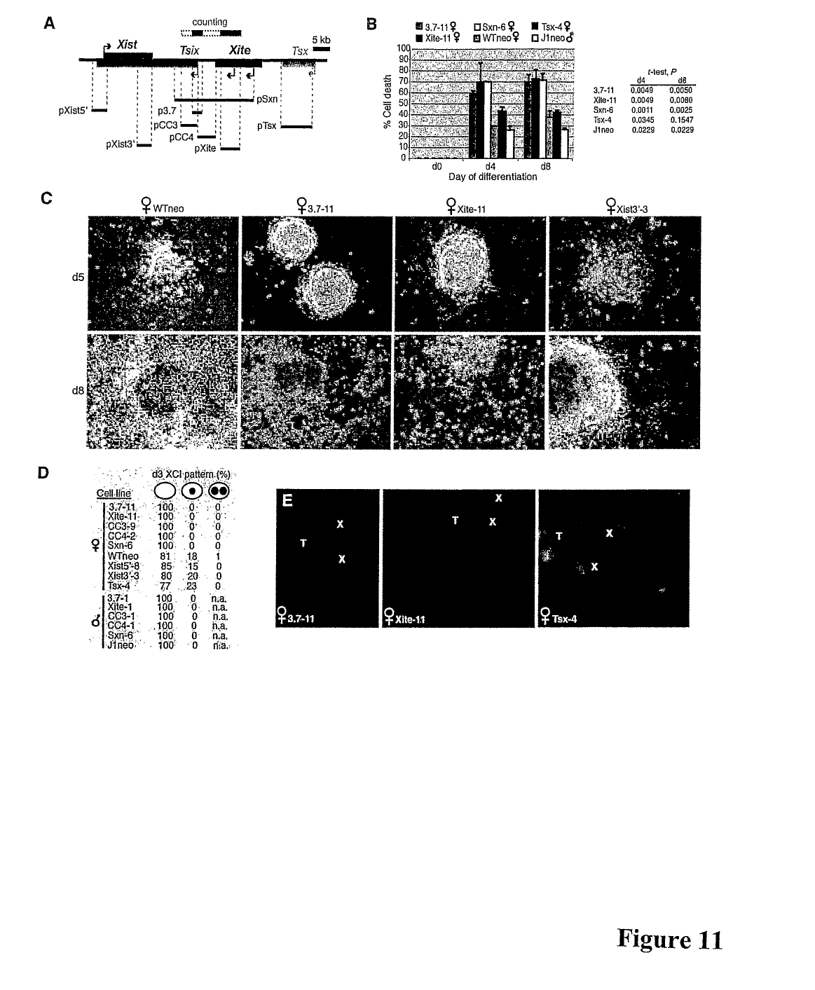

To pinpoint potential counting element(s), I fragmented the pSx7 transgene to segregate known landmarks (FIG. 11A). pSxn (19.5 kb) deletes all of Xist and includes the 5′ half of Tsix and Xite; p3.7 (3.7 kb) contains the full sequence deleted in Tsix^{ΔCpG} (Lee et al., Cell (1999) supra), including the Tsix promoter, DXPas34 repeat (Courtier et al., Proc. Natl. Acad. Sci. U.S.A. 92:3531-3535 (1995)), and part of the Tsix bipartite enhancer (Stavropoulos et al., Mol Cell Biol 25:2757-2769 (2005)); pCC3 (4.3 kb) contains several CTCF binding sites (Chao et al., Science 295:345-347 (2002)) and DXPas34; pCC4 (5.9 kb) contains the proximal half of bipartite enhancer (Stavropoulos et al., Mol Cell Biol (2005) supra); and pXite (5.6 kb) contains the major Xite intergenic transcripts, DNaseI hypersensitive sites, and a second Tsix enhancer (Ogawa et al., supra; Stavropoulos et al., Mol Cell Biol (2005) supra).

All five transgene series exhibited a dramatic disparity between XX and XY cells. The effects were most profound in the p3.7 and pXite series, the two containing promoters for Tsix and Xite. In general, XX transgenic EB colonies showed markedly fast radial growth between days 0 and 5, reaching a much larger size than the wildtype XX and XY controls or even πJL2 and πJL3 transgenics by day 5 (FIG. 11C). Intriguingly, despite 5-6 days of differentiation, the XX transgenic EB seemed to remain undifferentiated, as their colonies resembled day 0 ES cells in having a rounded morphology, high radiance level, and very little cellular outgrowth on gelatinized plates (FIG. 11C).

However, while the XX transgenics grew rapidly from days 2-5, their growth became severely impaired after day 6. While p3.7, pCC3, pCC4, pXite, and pSxn transgenics all shared these characteristics, the effects were again most pronounced in p3.7 and pXite transgenics. Day 6-8 cultures were characterized by accumulation of dead, detached cells (FIG. 11B; FIG. 11C: note disorganized masses in 3.7-11 and Xite-11 on d8). The extent of cell death reached as much as 70-75% of the total population between days 4 and 8 (FIG. 11B). To understand the cellular basis for these abnormalities, I examined the pattern of XCI in single cells using FISH. Remarkably, there was a complete absence of Xist accumulation at any time during cell differentiation in p3.7, pXite, pCC3, pCC4, pSxn transgenics (FIGS. 11D,E). This result contrasted with those for the larger πJL2, πJL3, and pSx7 and implied that critical elements for Xist induction have been deleted in the smaller transgenes. The absence of Xist RNA was not the result of an XO aneuploidy, as DNA FISH clearly demonstrated an XX constitution. Thus, supernumerary copies of 5′ Tsix and Xite sequences arrested XCI. This arrest apparently retards or blocks ES cell differentiation and eventually decimates the culture. Notably, the results for the small transgenics are opposite of those for Tsix X^{Δ}X^{Δ} (FIGS. 9A-9F). Between days 2-5, X^{Δ}X^{Δ} EB showed slow, fragmented growth, while XX transgenic EB showed high radial growth. Between days 6-9, X^{Δ}X^{Δ} EB showed overall improvement in differentiation (due to selection of appropriately dosage compensated cells), while XX transgenic EB degenerated. Additionally, while X^{Δ}X^{Δ} EB showed all possible numbers of Xᵢ, the XX transgenics failed to form an Xᵢ. These contrasting findings suggested that they represent opposite extremes of a counting defect: a deficiency of the critical element causes inappropriate XCI, while an excess suppresses XCI. The dose-dependent effects seen in πJL2, πJL3, and pSx7 transgenics further argued for a numerator that requires precise titration. Importantly, none of the XY transgenics manifested the defects. In multiple clones examined for p3.7, pDNT, pCC3, and pCC4, the XY EB showed normal growth throughout differentiation, normal cell death rates, and absence of an Xᵢ domain indicative of proper dosage compensation (FIGS. 11B,D and Table 3; one representative XY clone is shown). Thus, the peculiar phenotype can only be observed in transgenic cells with an XX constitution.

Furthermore, none of the transgenics carrying other Xic sequences manifested these defects. I tested multiple clones carrying the Tsx coding region and various fragments from Xist. In cell differentiation assays, the Tsx and Xist XX transgenics showed slightly slower growth rates and minimally elevated cell death rates (FIGS. 11B,C). However, by RNA FISH analysis, none of these controls lines displayed abnormal XCI patterns, as a single Xi domain appeared with normal kinetics during differentiation (FIGS. 11D,E). Thus, insofar as Xist and Tsx sequences had a mildly toxic effect on XX EB, this effect was not related to aberrant counting. These results demonstrated that the counting elements lie specifically within a 14 kb sequence (FIG. 11A), with the 5′ ends of Tsix and Xite displaying the strongest counting effects (solid purple bars) and adjacent regions also exerting effects, though less strongly (dashed purple bars). Thus, just as with the steps of choice and silencing, the initial step of counting is also controlled by noncoding genes.

### Noncoding Genes and the Duality Model

The mechanism of X-chromosome counting has remained a most elusive aspect of XCI. Models for counting must now reconcile several paradoxes uncovered by this study. Most intriguingly, why is the X^{Δ} active in Tsix X^{Δ}Y males (Lee et al., Cell (1999) supra), while it is silenced in X^{Δ}X (Lee et al., Cell (1999), supra; Sado et al., supra; Luikenhuis et al., Mol. Cell. Biol. 21:8512-8520 (2001); Lee, Nature Genet. (2002), supra) and silenceable in X^{Δ}X^{Δ} A critical factor must be missing in X^{Δ}Y but present in the female mutants. Furthermore, why do cells carrying large Xic transgenes permit X-inactivation (Lee et al., Cell (1996), supra; Heard et al., Molec. Cell. Biol. 19:3156-3166 (1999); Lee et al., Proc. Natl. Acad. Sci. (1999) supra; Migeon et al., supra), while those carrying small Tsix and Xite transgenes fail at XCI? Finally, why do the small transgenes affect XX cells but spare XY cells? The current work enables us to draw several novel conclusions.

First, Tsix and Xite regulate the counting process through elements at their 5′ ends. This conclusion seems at odds with the previous idea that a bi- or tri-partite structure contains the counting element (FIG. 8B)(Ogawa et al., supra; Lee et al., Cell (1999) supra; Sado et al., supra; Clerc et al., Nature Genet. 19:249-253 (1998); Morey et al., Embo J 23:594-604 (2004); Luikenhuis et al., Mol. Cell. Biol. 21:8512-8520 (2001)). However, past conclusions have been based largely on heterozygous mutants. The current study underscores the importance of extending analysis to homozygotes, as the observed peculiar effects were only evident once the second Tsix allele was eliminated. Homozygosing the Xite^{ΔL}, Δ65 kb, p37 kb, and ΔXist mutations will now be of interest (Ogawa et al., supra; Penny et al., Nature 379:131-137 (1996); Marahrens et al., Genes & Dev. 11:156-166 (1997); Clerc et al., Nature Genet. 19:249-253 (1998); Morey et al., Embo J 23:594-604 (2004)), as the phenotype relating to counting may be unexpected in the other homozygotes as well.

Importantly, this work does not distinguish between whether the critical element is a titratable DNA sequence or an RNA product of the two noncoding genes. A titratable DNA sequence seems more consistent, given the transgene data which suggest that promoterless sequences within the 14 kb domain (e.g., pCC3, pCC4) can exert a counting phenotype. I suggest that specific DNA elements near or in the promoters of Tsix and Xite act as binding sites for trans-acting factors, such as the putative blocking or competence factors.

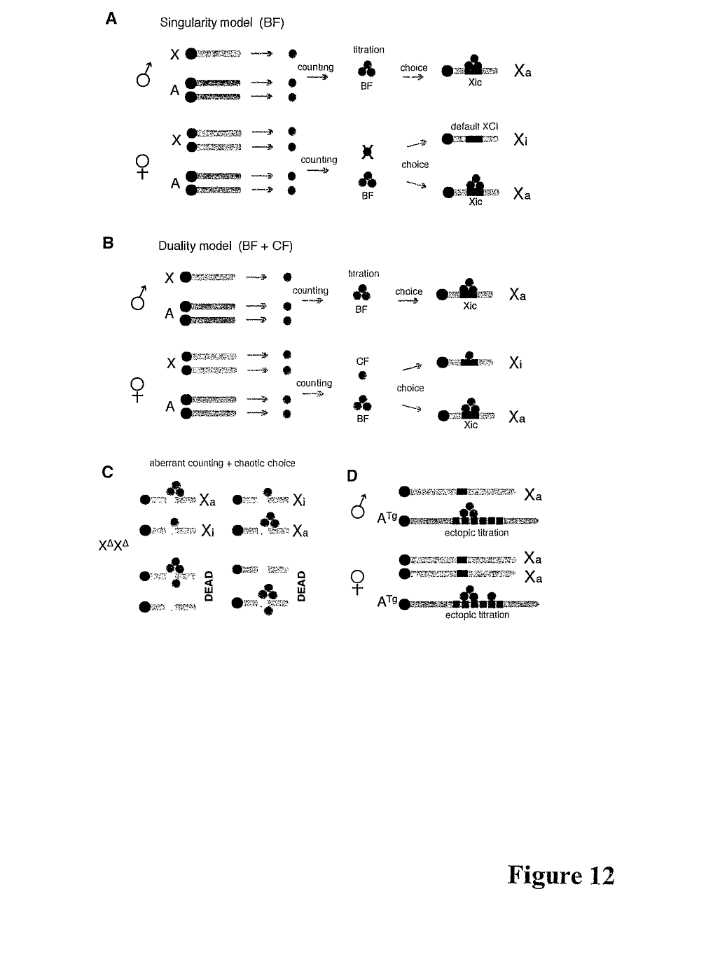

A second major conclusion of this study relates to the merits of the singularity vs. the duality models for counting. In the singularity model (FIG. 12A)(Avner et al., supra; Lyon, supra; Rastan, J Embryol. Exp. Morph. 78:1-22 (1983)), the X-chromosome and various autosomes produce unique X- and A-factors in limited quantities commensurate with their copy number per cell. The complexing of X- and A-factors results in the formation of the putative blocking factor (BF), which binds to and represses the firing of one Xic per cell. In this model, the remaining X's do not bind the singularity and become Xᵢ's by default. The model is elegantly simple—the binding of a single factor achieves both counting and choosing of a single Xₐ. No purposeful selection is required for the Xᵢ. However, while the singularity model neatly explains the ‘n−1’ rule and presence of additional Xₐ's in polyploids, it cannot easily reconcile the latest observations. First, the absence of XCI in XX cells with small Tsix and Xite transgenes is inexplicable. In the singularity model, BF would bind one X or the transgenic autosome, leading to the default inactivation of one or both X's. This was not observed. Moreover, if XCI indeed occurs by default, then X^{Δ}X^{Δ} cells should always form one Xᵢ (not two or none) because the single BF would in theory bind one X, leaving the remaining X for inactivation by default. From a different perspective, given the observation that X^{Δ}X^{Δ} cells can inactivate one or both X^{Δ}'s, the singularity model would also predict that X^{Δ}Y cells inactivate X^{Δ}. This, however, was also not observed.

These discrepancies instead appeal to a ‘duality model’ (Lee et al., Cell (1999) supra). The mutant phenotypes of X^{Δ}X and X^{Δ}X^{Δ} in the absence of any phenotype in X^{Δ}Y argues one clear point: while Tsix is required to repress XCI, an additional factor is necessary to induce XCI. As proposed, in addition to BF that represses XCI on the future Xₐ, a competence factor (CF) is required to induce XCI on the future Xᵢ. A priori, CF must comprise factors present in XX but not XY cells. Prima facie, an additional female X-chromosome is the single entity which satisfies this criterion, implying that CF is X-linked.

In the duality model (FIG. 12B), the counting mechanism measures the X:A ratio through specific X- and A-factors, each produced in limited quantities proportional to the chromosome copy number. The act of counting represents a ‘titration’ of X- and A-factors. The A-factors complex with one another and together titrate away one X-factor, the sum of which becomes one BF. Left without A-factor partners, the remaining X-factor(s) becomes CF. The ensuing act of choice reflects the stochastic binding of BF and CF to the two X's, with the BF repressing the Xic on the future Xₐ and the CF inducing the Xic on the future Xᵢ. BF and CF must bind in a mutually exclusive fashion. The duality model differs from the singularity model only in the stipulation that Xᵢ formation requires the purposeful action of CF, rather than being a default process.

The current data substantiates the existence of a CF. In the duality model, the different outcomes of X^{Δ}Y vs. X^{Δ}X and X^{Δ}X^{Δ} mutants result from the absence of CF in X^{Δ}Y cells and presence in XΔX and X^{Δ}X^{Δ} cells. In X^{Δ}X^{Δ} mutants, chaotic choice is presumed to occur because the Tsix deletions result in loss of mutual exclusion between the binding of BF and CF to the X's (FIG. 12C)(Lee, Nature Genet. (2002), supra). In this model, the binding of both BF and CF to one X and the absence of BF and CF on the other X lead to a confused state in which the X's can either remain active or be inactivated, thereby producing the observed two Xₐ and two Xᵢ phenotypes. The duality model also explains the various outcomes in transgenic cells. In XX cells carrying small Tsix and Xite transgenes, supernumerary copies of the noncoding sequences act as a sink and titrate away BF and CF, resulting in two Xₐ's (FIG. 12D). The high transgene copy numbers of these cell lines make the autosomes more competitive for the factors than the endogenous Xic, explaining why an Xᵢ is rarely, if ever, seen. In XY cells, BF is titrated away by the transgenes without consequence, because ectopic XCI cannot occur without CF.

Offering further insight into the counting process is the fact that πJL2, πJL3, and pSx7 gave rise to cells competent for XCI while the smaller transgenes failed to induce XCI. This suggests that the larger transgenes harbor another critical element that is missing in the smaller transgenes, possibly CF itself or something that can substitute for it. Further study is necessary to identify and characterize that element within πJL2. How can one reconcile the existence of CF with the Δ65 kb knockout (Clerc et al., Nature Genet. 19:249-253 (1998); Morey et al., Embo J 23:594-604 (2004))? In this knockout, XO and XY cells underwent XCI in the absence of the putative CF. One possible explanation may be that Δ65 kb is a neomorph. Because of the deletion size, regulators of the apposed Chic1 gene might exert ectopic influences on Xist. More likely, the 65 kb region spanning Xist, Tsix, Xite, Tsx, and Chic1 may actually contain additional regulators whose deletion leads to XCI in cis. This idea is consistent with above conclusions of transgene analysis, which also imply additional regulators at the Xic. Thus, Δ65 kb cannot be equated with Xite^{ΔL} nor Tsix^{ΔCpG}.

A third conclusion of this work addresses the question of where BF and CF might bind. The phenotypes of the knockouts and transgenics argue that BF and CF must interact with elements in or around the promoters of Tsix and Xite (purple bar, FIG. 11A), either directly or through other factors. In the transgene analysis, the promoter regions of the two genes elicited the strongest phenotype (filled purple bars), but the fragments immediately adjacent to them also elicited a counting phenotype (dotted purple bars). Thus, multiple cis-elements within the ˜14 kb region may act cooperatively in counting. Significantly, two enhancers have recently been mapped to this region, including a 1.2 kb element that coincides with the Xite promoter and a bipartite element that flanks the Tsix promoter (Stavropoulos et al., Mol Cell Biol (2005), supra). The idea that BF and CF might bind Tsix enhancers is inherently satisfying, as the fate of each X is indeed determined by whether Tsix expression persists (Xₐ) or is switched off (Xᵢ) in the differentiating cell.

Finally, the current work demonstrates that counting and choice are molecularly coupled. Although they are genetically separable by virtue of differential effects in Tsix X^{Δ}Y, X^{Δ}X, and X^{Δ}X^{Δ} cells, the observations here indicate shared control elements. Specifically, Tsix and Xite mutations that were known to affect choice (Ogawa et al., supra; Lee et al., Cell (1999), supra; Sado et al., supra; Luikenhuis et al., Mol. Cell. Biol. 21:8512-8520 (2001); Stavropoulos et al., Proc. Natl. Acad. Sci. U.S.A. 98:10232-10237 (2001); Morey et al., Hum. Mol. Genet. 10:1403-1411 (2001); Lee, Nature Genet. (2002), supra) are now also shown to affect counting. In the duality model, counting and choice occur sequentially, with counting representing the titration of X- and A-factors and choice representing the binding of BF to Xₐ and CF to Xᵢ. Thus, counting and choice involve the same set of X- and A-factors and the same noncoding genes. What might these X- and A-factors be? Interestingly, CTCF, Xiaf1, and Xiaf2 have been identified as candidate trans-factors for the choice step (Chao et al., Science 295:345-347 (2002); Percec et al., Science 296:1136-1139 (2002)). In light of the current work, it will be interesting to ask if they have potential roles in counting as well. Future efforts in identifying transacting factors for counting will focus on DNA-binding proteins of the Xite and Tsix enhancers, cis-elements characterized here as having numerator properties.

### Example 2

### Smaller Transgenes of p3.7 and pXite can Arrest Cell Differentiation

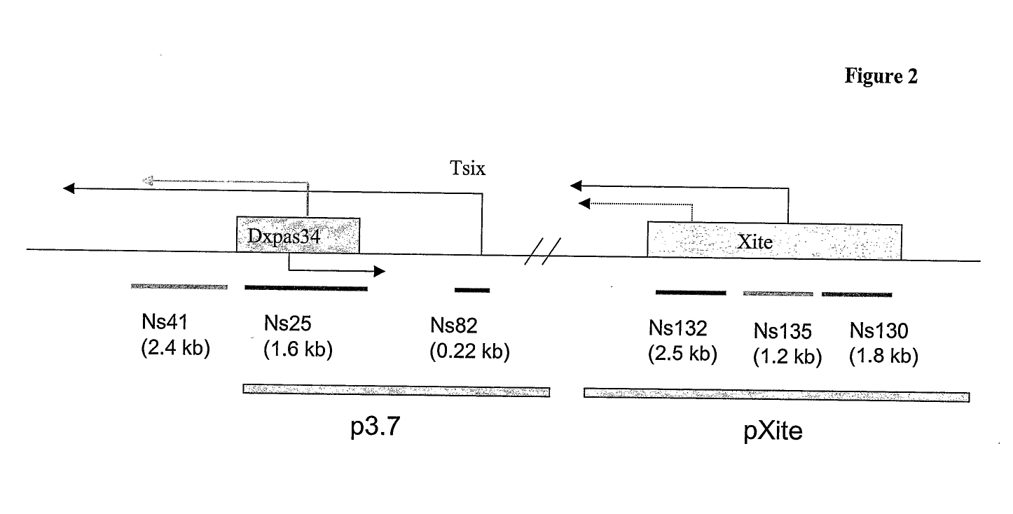

Based on the original discovery, described above, that regions of the Xic can be used to block cell differentiation, I generated smaller fragments of Tsix and Xite to identify the minimal critical region required for counting, pairing, and arrest of cell differentiation. These smaller transgenes are shown in FIGS. 1 and 2, described in Table 1, and summarized below.

ns25 (SEQ ID NO: 21): 1.6 kb DXPas34 fragment within Tsix that contains Repeats A1, A2, and B (see FIG. 30B).
ns41 (SEQ ID NO: 22): 2.4 kb fragment of Tsix that is located immediately downstream of DXPas34 (downstream with respect to Tsix transcription). ns41 is the SalI-BamHI fragment of pCC3.
ns130 (SEQ ID NO: 24): 1.8 kb of Xite as defined in Table I of Stavropoulos et al., (2005) supra. It includes sequences from bp-12,045 to -10,229 with respect to the Tsix major start site.
ns135 (SEQ ID NO: 25) and ns155 (SEQ ID NO: 26): the relevant fragments of each are the 1.2 kb Xite enhancer as defined in Stavropoulos et al., (2005) supra. They include bp-10,234 to -9,010 with respect to the Tsix major start site.
ns132 (SEQ ID NO: 27): 2.5 kb fragment of Xite also as defined in Stavropoulos et al., supra. It includes bp-9,009 to -6,535 with respect to the Tsix major start site.
ns82 (SEQ ID NO: 23): 220 base pair fragment of Tsix promoter.

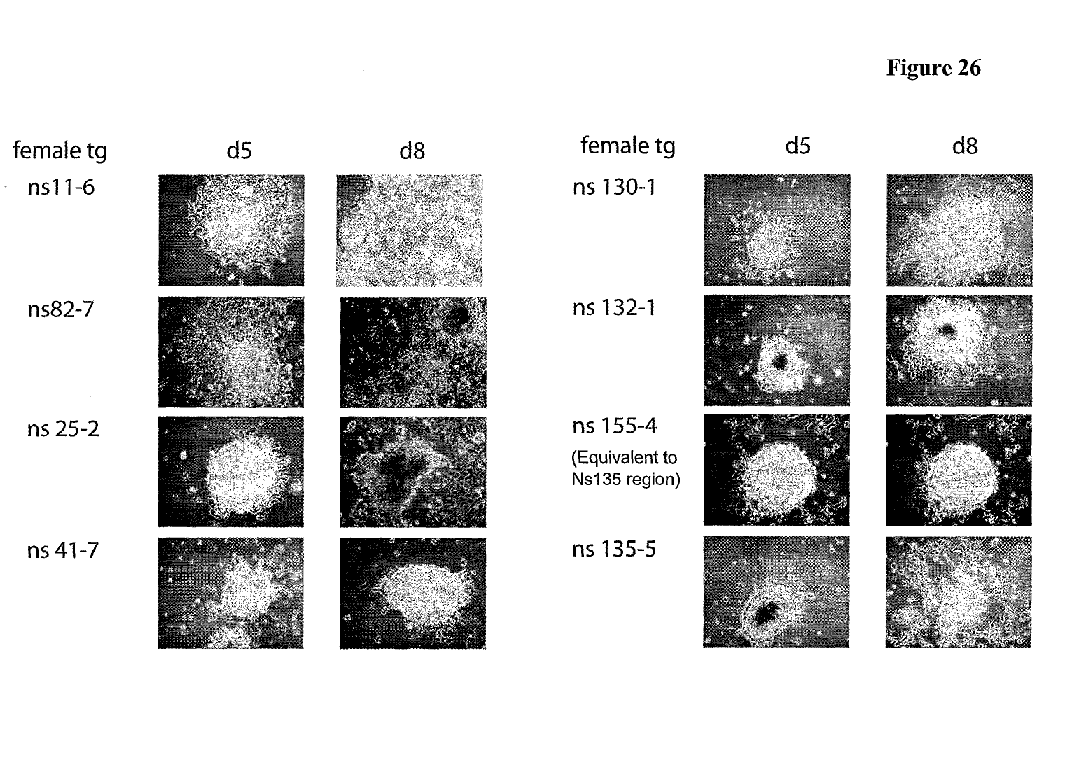

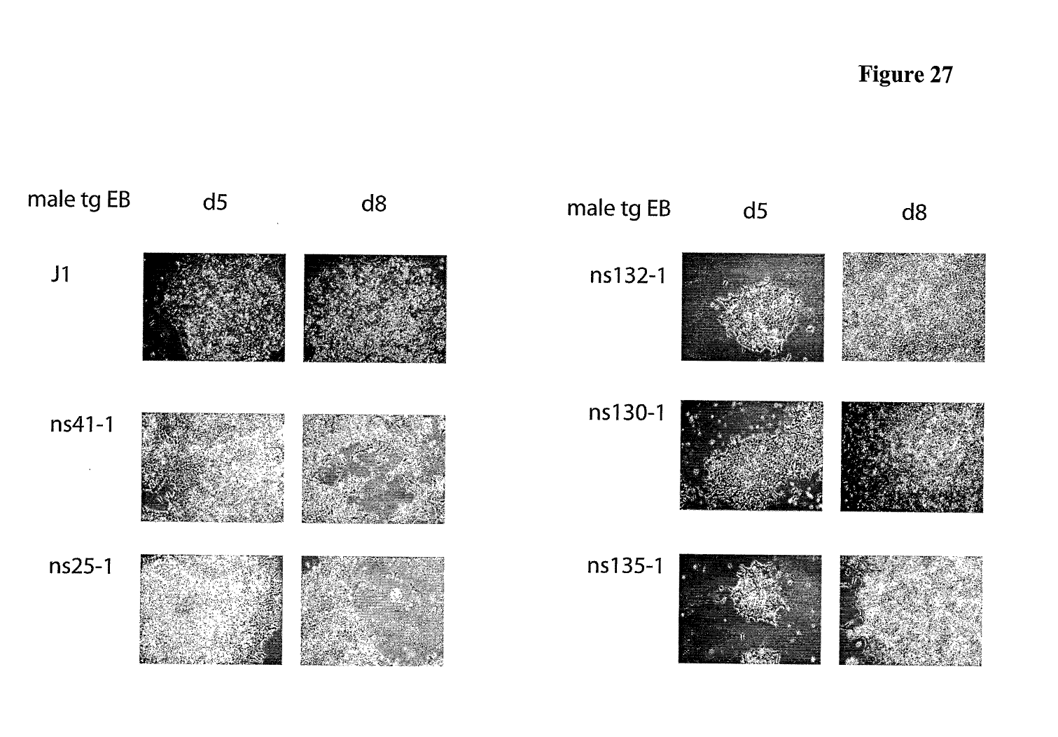

As shown in FIG. 26, subfragments ns41, ns25, ns132, ns135, and ns130, which range in size from 1.2 to 2.5 kb, all cause female ES cells to look “undifferentiated” even under differentiation conditions for 5 days. This effect is seen in the absence of feeder cells. This effect is not seen in male ES cells indicating that the effect is sex-specific (FIG. 27). Of these smaller transgenes, ns25 and ns135 are the smallest in size and both contain promoter and enhancer activity for the two noncoding RNAs. Ns25 contains repeats A1, A2, and B of DXPas34, which are described in more detail below in Example 4. For these experiments, transgenic ES cell lines were differentiated into embryoid bodies as described in Lee, Science 309:768 (2005) and EB were photographed, harvested for expression analysis and cell death analysis on the days of differentiation as indicated.

One noted exception was ns82, which contains only the Tsix major promoter (Table I of Stavropoulos et al., (2005) supra). This fragment does not affect counting or choice, cannot nucleate pairing by itself (as described in detail in Example 3, below), nor can it arrest ES differentiation in females. However, this fragment can enhance the block to differentiation seen with the other fragments. Therefore, ns82 may be used in combination with any of the other fragments described herein to block cell differentiation or to affect counting, choice, or pairing.

### Example 3

### Transient Homologous Chromosome Pairing Marks the Onset of XCI

The random form of X-chromosome inactivation (XCI) [reviewed in Avner and Heard, Nat. Rev. Genet. 2:59 (2001)] is regulated by a “counting” mechanism that enables XCI only when more than one X is present in a diploid nucleus. A “choice” mechanism then stochastically designates one Xₐ (active X), on which the X-inactivation center (Xic) is blocked from initiating silencing, and one Xᵢ (inactive X), on which the Xic is induced to initiate chromosome-wide silencing. Regulatory elements have been mapped to three noncoding Xic genes, including Xist (Brown et al., Cell 71:527 (1992); Brockdorff et al., Cell 71:515 (1992); Penny et al., Nature 379:131 (1996)), its antisense partner Tsix (Lee et al., Cell 21:400 (1999); Lee and Lu, Cell 99:47 (1999); Sado et al., Development 128:1275 (2001)), and Xite (Ogawa and Lee, Mol. Cell. 11:731 (2003)). Whereas Xite and Tsix together regulate counting and choice (Lee and Lu, (1999) supra; Sado et al., (2001) supra; Lee, Nat. Genet. 32:195 (2002); Morey et al., EMBO J. 23:594 (2004); Lee, Science 309:768 (2005)), Xist predominantly regulates chromosome-wide silencing (Penny et al., (1996) supra; Clemson et al., J. Cell Biol. 132:259 (1996); Marahrens et al., Genes Dev. 11: 156 (1997); Wutz and Jaenisch, Mol. Cell 5:695 (2000)). Interestingly, each gene acts in cis, with Xite activating the linked Tsix allele, Tsix repressing the linked Xist allele, and Xist repressing other genes on the same X.

Although cis-acting genes dominate the Xic, Xic function must extend in trans. Notably, the choice of Xₐ and Xᵢ always occurs in a mutually exclusive manner, so when one X is designated Xₐ, the other is accordingly designated Xᵢ. The idea of crosstalking is supported by a Tsix^{-/-} knockout, in which choice becomes “chaotic” with the occurrence of 2 Xᵢ, 1 Xᵢ, or 0 Xᵢ per cell (Lee, (2002) supra; Lee et al., Science (2005) supra). Though trans-interaction seems necessary (Lee et al., Nature Genetics (2002) supra, Marahrens, Genes Dev. 13:2624 (1999)), direct evidence has been lacking. In principle, trans-sensing could be accomplished by feedback signaling cascades, diffusible X-linked factors, or direct interchromosomal pairing such as that proposed for T cell differentiation (Spilianakis et al., Nature 435:637 (2005)).

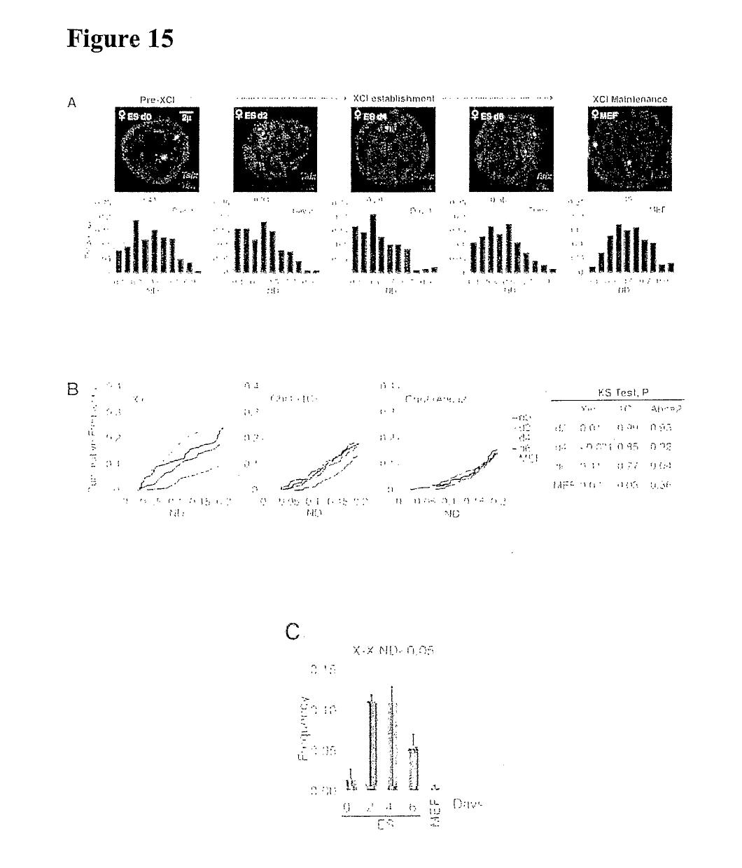

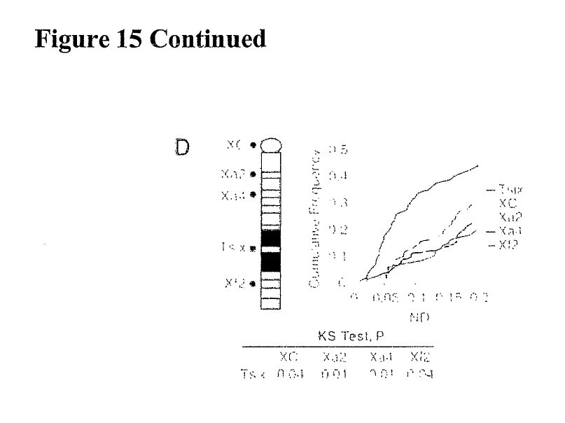

Because somatic homolog pairing does not generally occur in mammals, I surmised that pairing—should it occur on the X—must take place transiently. Here, I followed the movement of the chromosomes over time using fluorescence in situ hybridization (FISH) in differentiating mouse embryonic stem (ES) cells, a model that recapitulates XCI in culture. I measured the X-X interchromosomal distances for day 0 (pre-XCI), day 2 and day 4 (XCI onset), day 6 ES cells, and mouse embryonic fibroblasts (MEFs) (FIGS. 15A-D). By combining two non-overlapping probes, I obtained 99% X detection rates (single probes gave 85 to 90% rates). Only nuclei with two resolvable signals were scored. For each experiment, 150 to 250 nuclei were scored, and similar results were obtained in three independent tests.

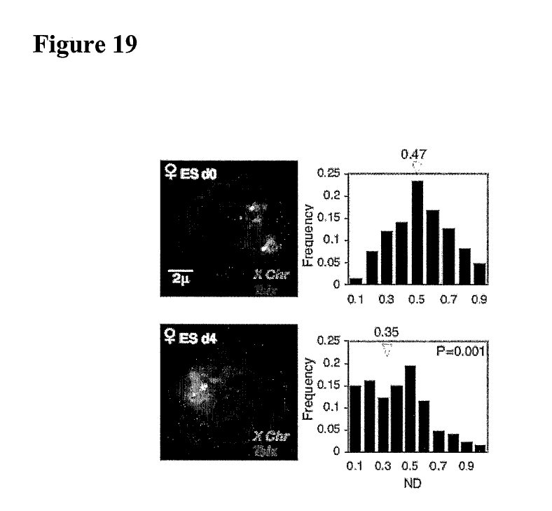

In wild-type XX cells, the X-X distance was highly dynamic during cell differentiation (FIG. 15 A). On day 0, the interchromosomal distances approximated a normal distribution, suggesting near-randomness. Interestingly, on day 2, a high proportion of cells began to display close X-X distances, as shown by a left shift in the distribution (FIG. 15A) [Kolmogorov-Smirnov (KS) test, P=0.01]. This trend continued into day 4 (P<0.001) and partially returned to baseline on day 6 (P=0.41). The MEF distribution was completely random, somewhat more so than for day 0 ES cells, perhaps reflecting spontaneous differentiation of some ES cells. Cumulative frequency curves (FIG. 15B) showed that day 2 and day 4 displayed the highest frequency of “proximity pairs,” or pairs with normalized X-X distances (ND)<0.2 (<2.0 μl). Among proximity pairs, one-third displayed 0.2- to 0.5-μ separation (FIG. 15C), a fraction greater by factors of 6 and 16 than in day 0 ES and MEFs, respectively. X painting confirmed the presence of two Xs (FIG. 19), thus excluding the possibility of visualizing sister chromatids within XO cells.

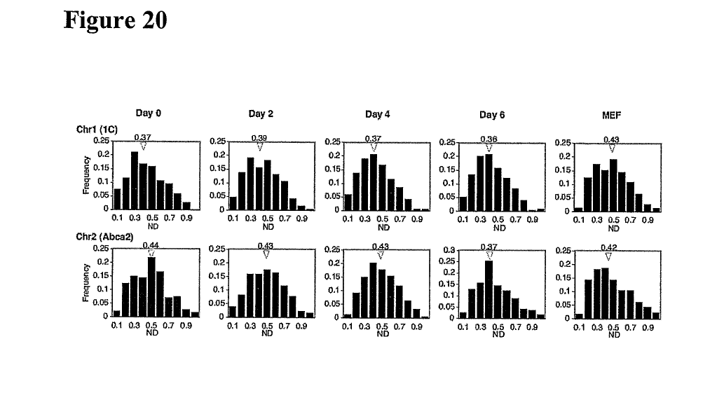

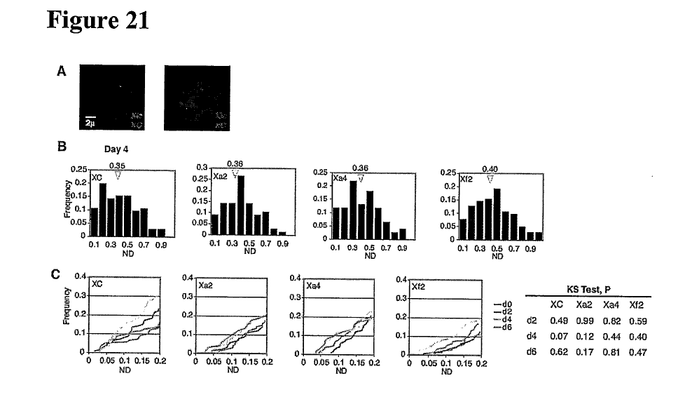

Measurement of interautosomal (A-A) distances at 1C [chromosome 1 (Chr1) centromere], Abca2 (Chr2), and chromosome 3 centromere showed normal distributions at all time points (FIG. 15B and FIG. 20), demonstrating that proximity pairing was not generally observed. To determine the extent of pairing on the X, I tested four bacterial artificial chromosome (BAC) probes in combination with an Xic probe (FIG. 15D and FIG. 21A-C) and found that, whereas Xic movement was constrained by homologous interaction, the flanking regions adopted relatively free positions, with each locus showing near-random distributions across time (FIG. 21A-C). Thus, X-X interactions were restricted to the Xic.

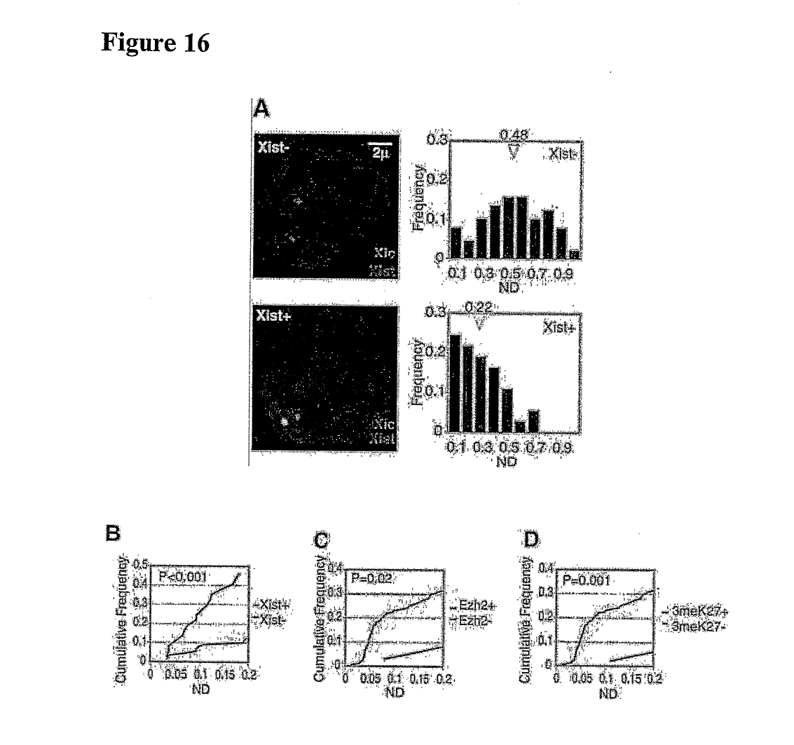

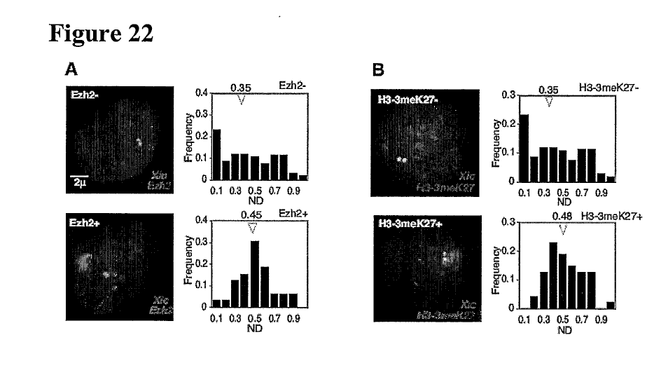

The pairing kinetics suggested linkage to XCI, which coincidentally initiates between day 2 and day 4 of differentiation. Because Xist RNA up-regulation is the earliest known cytologic feature of XCI (Avner and Heard, (2001) supra), I asked whether pairing could be observed more frequently in Xist⁺ cells. Indeed, Xist⁺ cells showed 46% with X-X association (FIGS. 16 A-B), indicating that pairing occurs just before or during Xist up-regulation. To pinpoint the time frame, the additional temporal markers, Ezh2 and H3-3meK27, were used. These markers accumulate on the Xᵢ shortly after Xist up-regulation during the “early Xᵢ maintenance” phase [reviewed in (Heard, Curr. Opin. Genet. Dev. 15:482 (2005))]. On day 2, trans-associations were significantly enriched in Ezh2⁻ cells and in H3-3meK2⁻ cells relative to Ezh2⁺ and H3-3meK27⁺ cells (FIGS. 16 C-D and FIGS. 22A-B). These results restricted trans-interactions to Xist-expressing cells that have not yet recruited Ezh2 and H3-3meK27, thus demonstrating a very early time frame, well before the XCI maintenance phase.

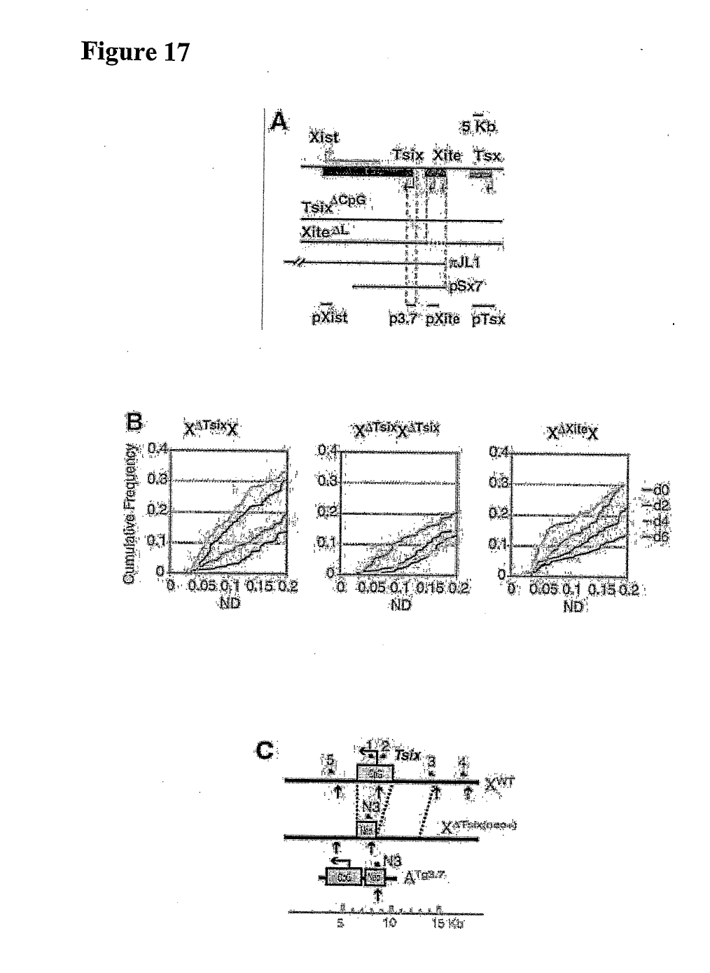

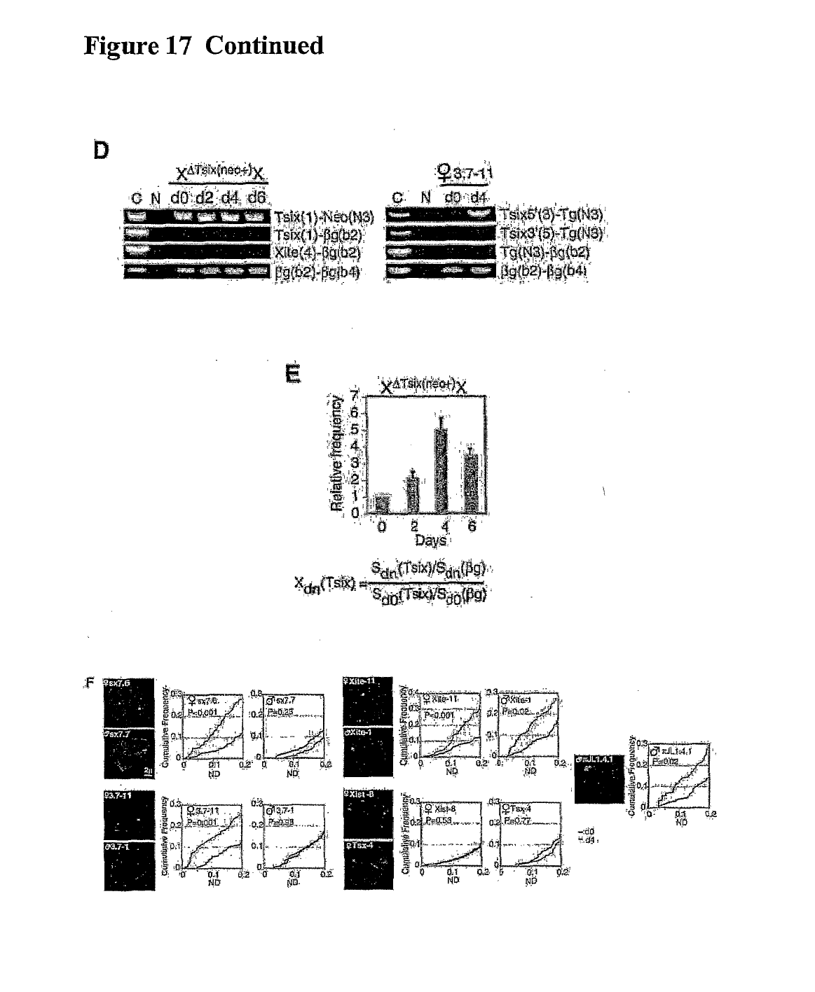

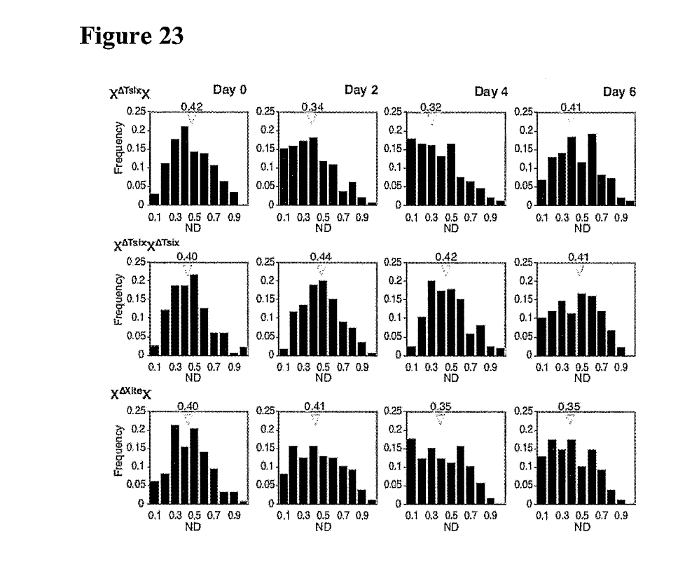

We therefore tested the relation of transinteractions to counting and choice, the two earliest steps of XCI, both of which are regulated by Tsix and Xite. It was previously shown that Tsix^{+/-} mice (X^{ΔTsix}X) are disrupted for choice and silence only X^{Δ} (Lee and Lu, (1999) supra; Sado et al., (2001) supra; Morey et al., (2004) supra; Lee, Cell 103:17 (2000)), whereas X^{ΔTsixX}X^{ΔTsix} mice are disrupted for both counting and choice (Lee et al., Nature Genetics (2002) supra; Lee et al., Science (2005), supra). Xite mutations have similarly affected counting/choice (Ogawa and Lee, (2003) supra; Lee et al., Science (2005), supra). X^{ΔXite}X cells showed a marked delay in X-X association (FIGS. 17A-B, and FIG. 23), implying that losing one Xite allele is sufficient to partially disrupt pairing. This partial effect correlated with aberrant choice in X^{ΔXite}X. However, X^{ΔTsix}X cells showed the expected frequency of homologous association, indicating that losing one Tsix allele does not affect pairing. In contrast, X^{Tsix}X^{ΔTsix} cells showed near-random distributions across all time points (FIG. 17B and FIG. 23), which supports the argument that deleting both Tsix alleles is required to abolish pairing. Although not statistically significant, day 6 populations showed a slight left shift suggestive of a delayed or weakened attempt to associate. These data demonstrated that Tsix and Xite are required for pairing and implied a tight link between pairing and counting/choice.

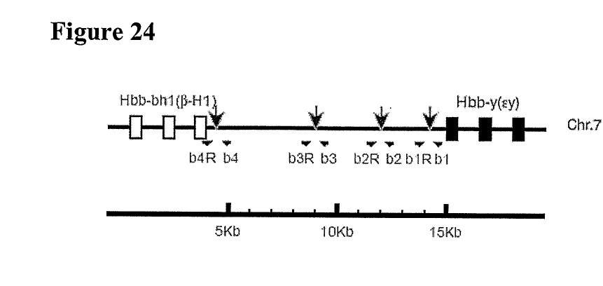

“Chromosome conformation capture” (3C) was used to learn whether the homologous association represented true physical pairing, (Dekker et al., Science 295:1306 (2002)), whereby two interacting loci can be detected by crosslinking, intermolecular ligation, and polymerase chain reaction. To obtain necessary polymorphisms for 3C, the pairing competent X^{ΔTsix(neo+)}X line was used, in which one Xic is distinguished by Neo (FIG. 17C) (wild-type could not be used because they lack informative polymorphisms within required restriction fragments). Using three distinct primer pairs [Tsix1-N3 (shown), and TSEN2-N1 and Tsix1-N2], I consistently detected physical contact between the two Tsix loci, whereas no contacts were observed between various Tsix and autosomal controls or the incorrectly oriented Tsix2 primer and N3 (FIG. 17D and FIG. 24). The inter-Tsix interaction was strongest on day 4 (FIG. 17E), consistent with FISH analysis. Therefore, inter-Xic pairing indeed underlies homologous association.

To identify sequences that direct pairing, I introduced Xic fragments into ES cells (FIG. 17A and Table 4) (Lee, Science (2005), supra) and asked whether autosomal insertions could induce de novo X-autosome (X-A) pairing and affect counting/choice. Intriguingly, autosomal pSx7 led to ectopic X-A pairing in females (FIG. 17F), correlating with aberrant counting and XCI initiation in pSx7 females (Lee et al., Science (2005), supra). By contrast, female Xist and Tsx transgenics showed no X-A pairing above background (FIG. 17F), consistent with their normal XCI (Lee et-al., Science (2005), supra). Furthermore, male pSx7 transgenics did not exhibit X-A pairing (FIG. 17F), consistent with their normal counting and XCI suppression (Lee et al., Science (2005) supra).

TABLE 4
Summary of transgenic cell lines and their pairing characteristics.

| | | | X-A | Tg |
|---|---|---|---|---|
| Tg | ES line | XCI | Pairing | Copy |
| pSx7 | ♀sx7.6 | aberrant | + | low |
| | ♂sx7.7 | normal | − | low |
| p3.7 | ♀3.7-11 | aberrant | + | high |
| | ♂3.7-1 | normal | − | low |
| pXite | ♀Xite-11 | aberrant | + | high |
| | ♂Xite-1 | normal | + | high |
| pXist | ♀Xist-8 | normal | − | high |
| pTsx | ♀Tsx-4 | normal | − | high |
| πJL1 | ♂1.4.1 | aberrant | + | high |

To dissect specific requirements within pSx7, I tested p3.7, the 3.7 kb Tsix fragment deleted in the pairing-incompetent X^{ΔTsix}X^{ΔTsix}. p3.7 was remarkably efficient at inducing de novo X-A pairing in XX cells (FIG. 17F), with 3C analysis confirming direct physical interaction between p3.7 and the X (FIG. 17D). The ectopic pairing paralleled the failure of counting/choice and XCI initiation in p3.7 females (Lee et al., Science (2005), supra). In contrast, p3.7 males did not induce X-A pairing and accordingly did not manifest a counting defect (Lee et al., Science (2005), supra). pXite (a 5.6-kb fragment deleted in the pairing-compromised X^{ΔXite}X) were also tested and showed efficient X-A pairing (FIG. 17F), consistent with pXite's profound effect on counting/choice (Lee et al., Science (2005), supra). Interestingly, pXite males could also initiate pairing, although they did not exhibit ectopic XCI. Because pXite males are thought to lack an X-linked “competence factor” for initiating XCI, I next tested males carrying full-length Xic transgenes (Lee et al., Science (2005), supra) to determine whether pairing and XCI could be achieved together. Indeed, πJL1.4.1 males displayed ectopic X-A pairing (FIG. 17F) and, accordingly, initiated counting/choice and silencing (Lee et al., Proc. Natl. Acad. Sci. U.S.A. 96:3836 (1999)), further supporting the tight linkage between pairing and XCI initiation. These experiments demonstrated that Tsix and Xite, with sequences as small as 3.7 and 5.6 kb, are sufficient to recapitulate pairing and that, in turn, pairing is required for the earliest steps of XCI.

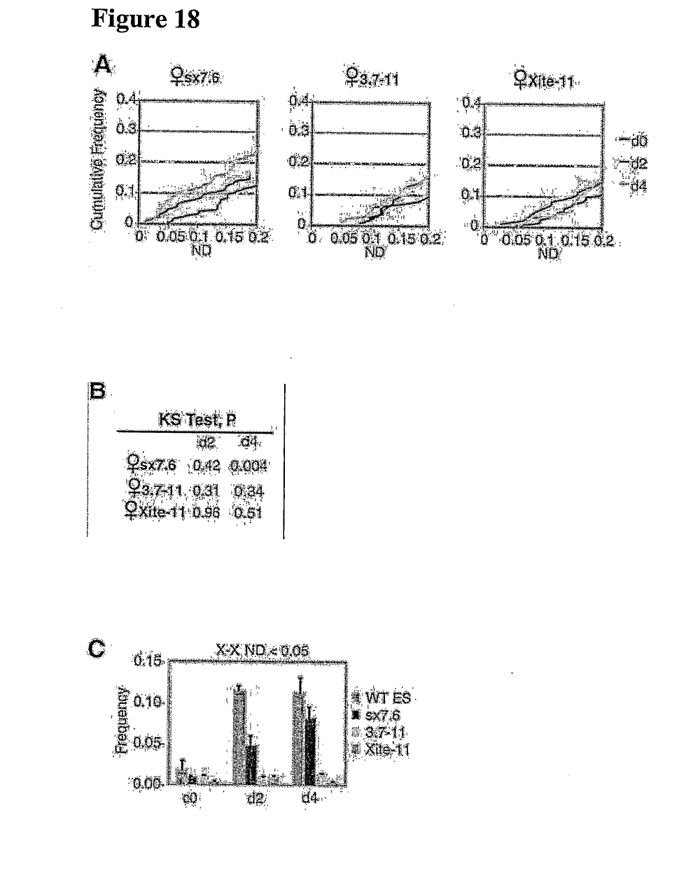

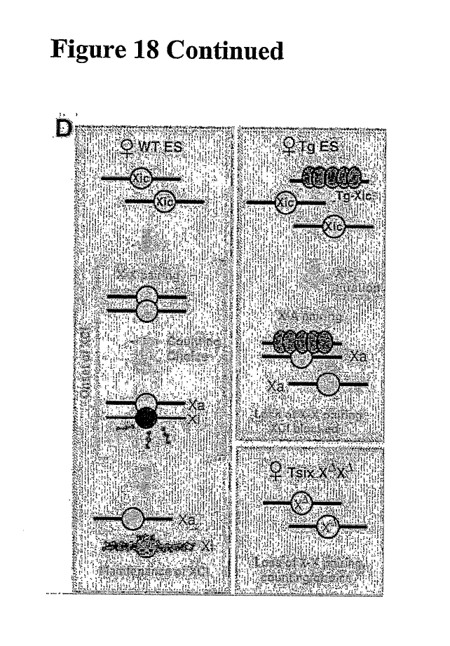

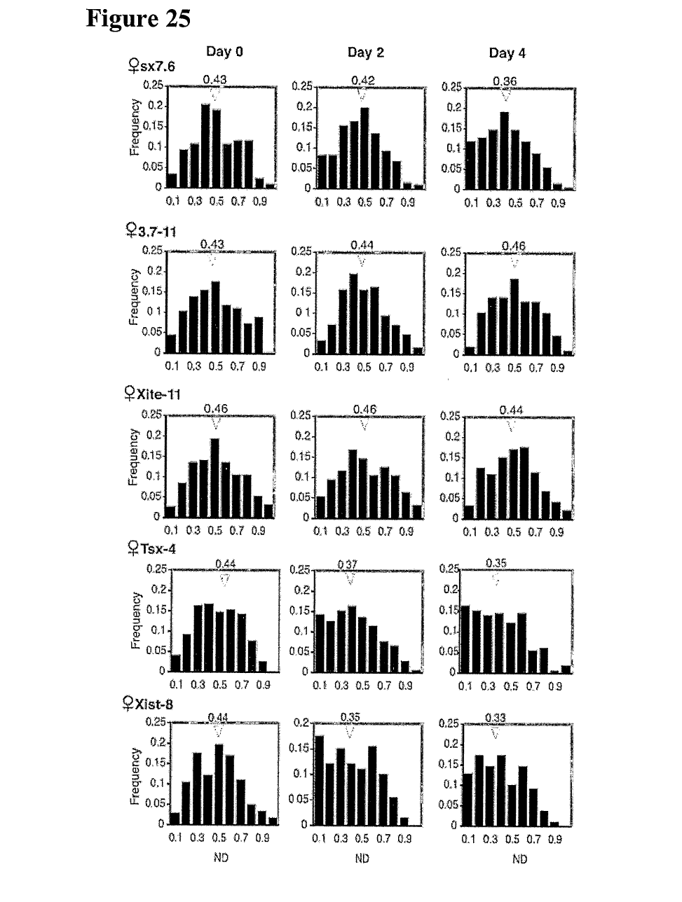

In transgenic females, I hypothesize that the failure to initiate XCI may be due to a competitive inhibition of X-X interactions by de novo X-A interactions. Indeed, the frequency of X-X interactions was significantly diminished for pSx7, p3.7, and pXite females as compared with wild-type (FIG. 18A versus FIG. 15B). In pSx7 females, X-X pairing rates were less than X-A pairing rates. In p3.7 and pXite females, X-X pairing appeared to be abolished completely (FIG. 18A and FIG. 25), with day 2 and day 4 distribution profiles being indistinguishable from day 0 (FIG. 188B) and <2% of nuclei (background) with ND<0.05 (FIG. 18C). In contrast, X-X pairing remained robust in pTsx and pXist controls (FIG. 25). Therefore, ectopic X-A interactions measurably detracted from endogenous X-X interactions. The frequency of X-X pairing directly predicts the frequency of XCI. I propose that the titration of X-X interactions by ectopic Tsix/Xite accounts for the pervasive failure of counting/choice and XCI in transgenic females.

On the basis of this work, I postulate that X-X pairing acts upstream of Xist by mediating counting/choice and providing the necessary crosstalk for mutually exclusive XCI. Pairing interactions clearly do not require Xist expression. In our model (FIG. 18D), two Xs assume random independent positions in pre-XCI cells and then pair homologously at the onset of XCI, with Tsix and Xite acting as nucleation centers. The ensuing crosstalking achieves asymmetric marking of one X to become Xₐ and the other to become Xᵢ. With counting/choice reflecting the binding of a “blocking factor” to the Xₐ and the competence factor to the Xᵢ (Lee and Lu, (1999) Supra; Lee et al., Science (2005) supra), pairing ensures that the two factors bind mutually exclusively.

Remarkably, 3.7 kb of Tsix or 5.6 kb of Xite is sufficient to initiate de novo pairing. Thus, these genes play dual cis-trans roles in XCI by functioning in trans to coordinate pairing/counting/choice and in cis to antagonize Xist. These events may take place simultaneously in time and space. Subtle pairing differences between Tsix and Xite mutants likely reflect length requirements, as indeed X^{ΔXite}X shows weaker pairing than X^{ΔTsix}X, and Xite transgenic males pair better than Tsix counterparts. Consistent with this, full-length transgenic πJL1.4.1 males not only pair well but also initiate XCI. Why do X-A interactions generally outnumber X-X interactions? The multicopy transgene nature might increase the avidity of the autosome relative to the X. The ability of X-A pairing to inhibit X-X pairing now provides a mechanism for failed XCI in Tsix/Xite transgenic females: If pairing were required for proper counting/choice, the failure to pair would pose a specific block to XCI. The proposed regulation by interchromosomal pairing creates a new dimension to the problem of gene regulation and is likely to become a recurrent theme in epigenetic phenomena (Spilianakis et al., (2005) supra; LaSalle and Lalande, Science 272:725 (1996)).

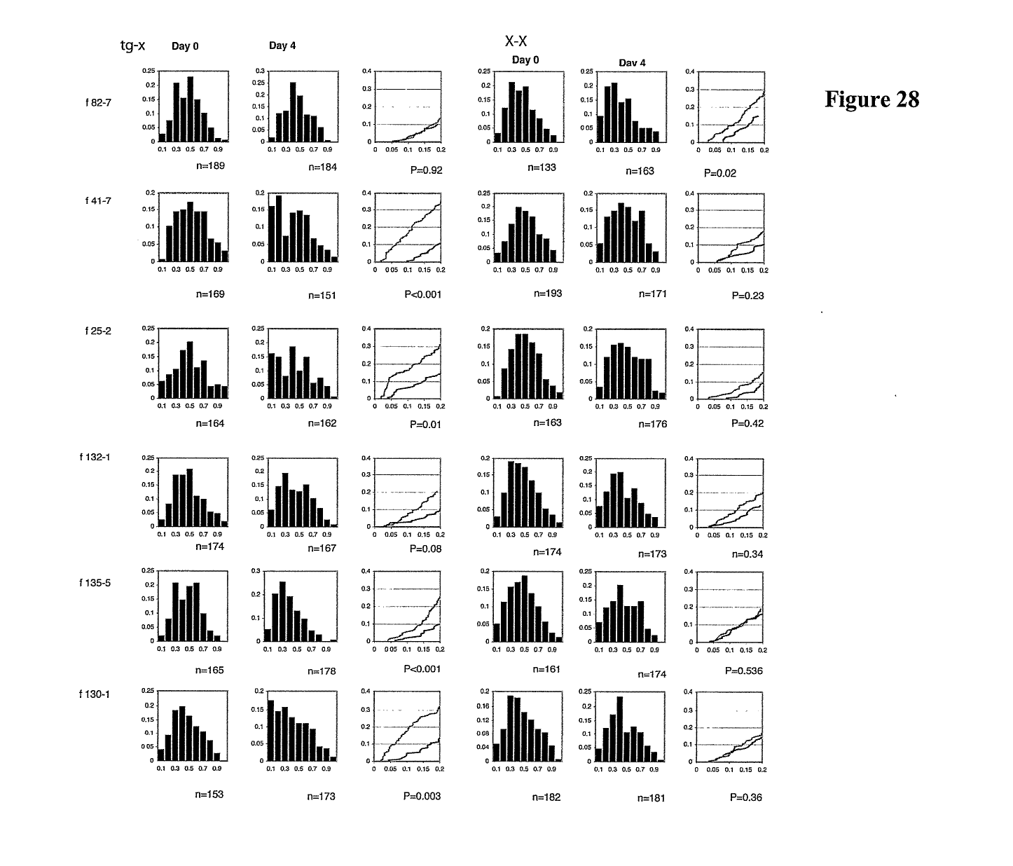

In order to determine if the smaller transgenes described in Example 2 could also nucleate pairing between chromosomes, I inserted subfragments into autosomes and tested the ability of the autosome to pair with the X. For these experiments, ES cells were harvested on day 0 (undifferentiated) or day 4, dispersed, fixed onto glass slides, and examined by FISH. They were then imaged and analyzed by Improvision software as described below and in Xu et al., Science 311:1149-1152 (2006). FISH was carried out using probes from the XIC (using fragments equivalent to each transgene sequence). As shown in FIG. 28, the smaller transgenes also nucleate de novo “pairing” between the X and the autosome into which the transgenes had inserted. The only exception to this was the ns82 fragment, which only includes the Tsix promoter. (Note that the nomenclature refers to the transgene-particular clone carrying the transgene. For example, ns82-7 refers to clone 7 carrying the ns82 transgene.) These results show that the ectopic X-A pairing inhibits endogenous X-X pairing. I propose that this is why X-inactivation is inhibited and why cell differentiation cannot occur in the female ES cells. Thus, any fragment which causes ectopic pairing between the Xic's could be used to block cell differentiation.

The following materials and methods were used in the experiments described above.

### ES Cell Culture

Wildtype male J1 (40XY), wildtype female 16.7 (40XX), and all mutant mouse ES cell lines and their culture conditions have been described previously (Lee and Lu, Cell 99:47 (1999); Lee et al., Nat. Genet. 21:400 (1999); Lee, Science 309:768 (2005)). Transgenic ES lines were maintained under 300 μg/ml G418 selection. ES differentiation was induced by suspension culture for 4 days and withdrawing leukemia inhibitory factor (LIF). On day 4, embryoid bodies were attached to gelatinized plates to promote outgrowth of differentiated cells. Fibroblasts were derived from d13.5 mouse embryos using standard protocols.

### FISH Analysis

ES clusters were trypsinized into single cells and cytospun on glass slides prior to

paraformaldehyde fixation. DNA and RNA FISH were carried out as described (Lee et al., (1999) supra). Probes were labeled with fluorescein-12-dUTP or cy3-dUTP by nick-translation. pSxn, pSx9, or pTsx sequences were used as probe for the Xic region (Lee, (2005) supra). BAC probes 1C, XC, Xa2, Xa4, and Xf2 were obtained from Open Biosystems. Abca2 BAC was a gift of Drs. Brian Seed and Ramnik Xavier. For specific detection of transgenes, a promoterless Neo fragment was used. For detection of Xist RNA, a single-stranded riboprobe cocktail was used (Ogawa and Lee, Mol Cell 11:731 (2003)). Immuno-DNA FISH was carried out using anti-H3-3meK27 or anti-Ezh2 rabbit polyclonal antibody (Upstate), followed by secondary goat-anti-rabbit antibody conjugated with cy3. Images were taken with the Zeiss axioscope and processed using OpenLab software (Improvision). 2D representation of 3D images were created by merging z-sections of 0.2μ intervals taken across whole nuclei depth. The X-X distances (x) and nuclear areas (A) were calculated using the measurement module in OpenLab.

Only nuclei with two resolvable X-signals were scored—single-dots were excluded to avoid counting XO cells, which accounted for <<5% of total culture (FIG. 20). Nuclear diameter (d)=2*(nuclear area/π)^{0.5}. Normalized distance (ND)=X-X distance/d. Days 2 through 6 ES nuclei generally had a similar size and shape as compared to day 0 nuclei. There are intrinsic limitations to this methodology. A typical ES nucleus is nearly perfectly round, measuring 10μ in diameter. However, MEFs tend to be ovoid in shape, a point that may give rise to slightly different distribution profiles for MEFs in FIGS. 19A and 20.

### Chromosome Conformation Capture (3C) Assay

The 3C assay was adapted for mammalian cells (Tolhuis et al., Mol. Cell. 10:1453 (2002)). For the necessary polymorphisms to detect interactions between homologous chromosomes, utilized the pairing-competent X^{ΔTsix(neo+)}X line, in which one Xic is distinguished by Neo (FIG. 21C) was utilized. (Note: WT lines could not be used because there were insufficient naturally occurring informative polymorphisms within the required restriction fragments). To distinguish Tsix^{ΔCpG(neo+)} from X^{WT}, a BamHI digest and primers were used as indicated in FIG. 21C. In brief, single cell suspension of 10⁷ cells was diluted in ES medium, crosslinked with 4% formaldehyde for 10 minutes at room temperature, quenched with 0.125M glycine, pelleted, and washed with PBS. 10⁶ cells were lysed in 10 ml of ice-cold lysis buffer (100 mM Tris-HCL pH8.0, mM NaCl, 0.2% NP-40, protease inhibitor) and the nuclei were pelleted, resuspended in NEB buffer 2 with 0.3% SDS, and incubated for 1 hour at 37° C. with shaking. To sequester SDS, Triton X-100 was added to 1.8% for 1 hour at 37° C. with shaking. The sample was incubated with 400 Units of BamHI overnight at 37° C. with shaking, the enzyme inactivated at 65° C. with 1.6% SDS, and then incubated with 1× ligation buffer (10 ml) and Triton X-100 (1%) at 25° C. with shaking. Ligation was carried out at low DNA concentration (<2.5 ng/μl) with 200 units of T4 DNA ligase for 4 hours at 16° C. Proteinase K was added (100 μg/ml) to reverse crosslinking at 65° C. overnight. The sample was then treated with RNase A (0.5 μg/ml) for 30 minutes at 37° C., the DNA extracted with phenol/chloroform and isopropanol-precipitated. Control samples without crosslinking or without T4 ligase were treated in parallel. <20 ng of template was used in each PCR reaction and each reaction occurred within the exponential phase of amplification to achieve accurate product quantitation.

For α-globin control templates, PCR products spanning BamHI sites of interest (primer pairs b1/b1R, b2/b2R, b3/b3R, b4/b4R) were digested, mixed at equal molar ratio, and ligated to each other (for βg-βg tests) or ligated to digested pSxn (for βg-Tsix and βg Xite) to create all possible pairwise ligations. Primer pair b2/b4 consistently showed cisinteractions (FIG. 21D), as did b1/b4 and b3/b4 (data not shown). To normalize the Tsix results, the use of any pair gave similar results. For control templates for X-X interactions, pSxn and the Tsix^{ΔCpG} knockout vector were digested, mixed at equal molar ratio, and ligated to create a pool of all possible pairwise ligations. For control templates for X-A interactions, p3.7 was digested and ligated with digested πJL2 (a full-length Xic P1 plasmid (Lee et al., Proc. Acad. Sci. USA 96:3836 (1999))) or ligated to digested β3g PCR fragments (for transgene p3.7-βg interactions). All primers were designed to have annealing temperature of 62-64° C., and all yielded products of the predicted size. All test PCR products were sequenced to confirm specificity and identity. None of the Tsix-βg primer pair combinations gave a specific PCR product. All minus-crosslinking and minus-ligation controls also gave no product. Two to three independent experiments were carried out for each interaction and PCR reactions were repeated at least twice for each experiment. The primers used were as follows:

| | |
|---|---|
| Tsix1(Bam12): | |
| 5′-CTCTGGCCACCTGTCTAGCTG | (SEQ ID NO: 47) |
| Tsix2(DSN35): | |
| 5′-TAGGTACCTAGGCAGATTGC | (SEQ ID NO: 48) |
| Tsix3(Bam13a): | |
| 5′-GGCTGAAGGTGCTGTAGCAAG | (SEQ ID NO: 49) |
| Tsix4(Bam14): | |
| 5′-CTGAGCTCGAACATTGCCCCAC | (SEQ ID NO: 50) |
| Tsix5(Bam11): | |
| 5′-CTAACAAGTGTGAGCCACCTGCC | (SEQ ID NO: 51) |
| Tsix TSEN2: | |
| 5′-CCACCTGTCTAGCTGGCTATCA | (SEQ ID NO: 52) |
| N1(NeoF): | |
| 5′-TTAGCCACCTCTCCCCTGTC | (SEQ ID NO: 53) |
| N2(NeoF2): | |
| 5′-TGTCCGGTGCCCTGAATGAACTGC | (SEQ ID NO: 54) |
| N3(NeoF3): | |
| 5′-ACGTTGTCACTGAAGCGGGAAGGG | (SEQ ID NO: 55) |
| b2(beta2): | |
| 5′-GTTTCCAGGAGGGGTTCAGGTTTA | (SEQ ID NO: 56) |
| b2R(beta2R): | |
| 5′-CACAAACCCAAACACAGATAAATG | (SEQ ID NO: 57) |
| b3(beta3): | |
| 5′-TTCATACACAGGACATCTACACAA | (SEQ ID NO: 58) |
| b3R(beta3R): | |
| 5′-TAAAATACAATCCACCAGTCATAC | (SEQ ID NO: 59) |
| b4(beta4): | |
| 5′-GCAAGGTCCAGGGTGAAGAATAAA | (SEQ ID NO: 60) |
| b4R(beta4R): | |
| 5′-ATTTTGATTTCCTCCTTGGGTCTT | (SEQ ID NO: 61) |

### Statistical Analysis

The significance of the difference in inter-chromosomal distance distributions were tested using the Kolmogorov-Smirnov (KS) test, a non-parametric test to examine the null hypothesis that two data-sets exhibit the same underlying distribution. The P value was calculated by the statistics software, SPSS 12.0. A P<0.05 was considered statistically significant.

### Example 4

### Role of Transcription in the Regulation of XCI

Despite their potentially disruptive effects, transposable elements (TEs) have been widely disseminated and now account for nearly 50% of the mammalian genome. Their ubiquity suggests that host genomes may benefit from TEs, although evidence for this has been scant. The X-inactivation center is known for its abundance of TEs. Here, I provide evidence that the 34mer DXPas34 repeat within Tsix is a retrotransposon remnant and establish that this repetitive element functions during X-chromosome inactivation (XCI). DXPas34 contains bidirectional promoter activity, producing overlapping forward and reverse transcripts. Three new Tsix alleles were generated and used to demonstrate that, while the Tsix promoter is unexpectedly dispensable, DXPas34 plays dual positive-negative functions. At the onset of XCI, DXPas34 stimulates Tsix expression as a component of its bipartite enhancer. Once XCI is established, however, DXPas34 becomes repressive and is required for stable silencing of Tsix. These data ascribe a new function to repetitive DNA elements. I propose a scheme by which TEs could be co-opted by nearby genes for epigenetic regulation.

Since their discovery by Barbara McClintock over 50 years ago, transposable elements (TEs) have been identified in nearly all organisms and account for a large fraction of eukaryotic genomes. Remarkably, transposons and their recognizable remnants now comprise at least 50% of some mammalian genomes (Lander et al., Nature 409:860-921 (2001)) and as much as 90% of plant genomes (SanMiguel et al., Science 274:765-768 (1996)). Because these elements were viewed as genetic parasites, they and their recognizable remnants have frequently been characterized as “junk DNA.” Particularly common in mammals are retrotransposons, a class of TEs that mobilize through an RNA intermediate and require reverse transcription for integration into the genome. Retrotransposons include both short and long interspersed nucleotide elements (SINEs and LINEs, respectively), as well as the endogenous retroviruses (ERV) and LTR families of repeat sequences. Though ancient in origin, TEs still actively transpose in mice and humans (Kazazian, Science 303:1626-1632 (2004)) despite their potentially disruptive and mutagenic effects.

Why have eukaryotes been so tolerant of transposons, perhaps even promoting their expansion over time? Their propagation in spite of inherent risks suggests that the host genome may actually benefit from mobile TE. In mammals, transposed elements can confer novel regulatory activities to existing genes. For example, they may have introduced novel promoter activities to mouse Lama3 (Ferrigno et al., Nat. Genet. 28:77-81 (2001)) and Agouti (Morgan et al., Nat. Genet. 23:314-318 (1999)). During the oocyte-to-embryo transition, TEs may have been co-opted by the mouse as alternative promoters and first exons for a significant fraction of expressed transcripts, thereby coordinating the synchronous, developmentally regulated expression of a diverse array of genes (Peaston et al., Dev. Cell 7:597-606 (2004)). Further evidence for TE subversion comes from the occurrence of SINEs within >1000 human gene promoters (Oei et al., 2004), their potential for creating novel splice sites (Kreahling and Graveley, Trends Genet. 20:1-4 (2004)), and their contribution to enhancer regulation (Bejerano et al., Nature 441:87-90 (2006)). In these ways, TEs may drive genome evolution and provide a means for rapid adaptation to everchanging environmental demands.

Because transposition disrupts local chromatin and gene function, evolutionarily stable integration events are known to concentrate in non-coding regions (Lippman et al., Nature 430:471-476 (2004)). The mammalian X-inactivation center (Xic), which contains multiple non-coding genes, has been noted for its abundance of TEs (Chureau et al., Genome Res. 12:894-908 (2002); Migeon et al., Am. J. Hum. Genet. 69:951-960 (2001); Nesterova et al., Dev. Biol. 235:343-350 (2001); Simmler et al., Hum. Mol. Genet. 5:1713-1726 (1996)) and therefore serves as a model to investigate functional interactions between TEs and epigenetic processes. X-chromosome inactivation (XCI) equalizes X-linked gene expression between mammalian males and females (Lyon, Nature 190: 372-373 (1961)) and progresses through a series of steps that include X-chromosome counting, the purposeful choice of one active X (Xₐ) and one inactive X (Xᵢ), and the initiation and establishment of silencing on the designated Xi. These steps are controlled by the noncoding genes, Xite (Ogawa and Lee, Mol. Cell. 11:731-743 (2003)), Tsix (Lee et al., Nat. Genet. 21:400-404 (1999)), and Xist (Borsani et al., Nature 351:325-329 (1991); Brockdorff et al., Nature 351:329-331 (1991); Brown et al., Nature 349:38-44 (1991)), each of which is characterized by TE infiltration.

Xist produces a large nuclear RNA that is expressed exclusively from the Xi, coats that chromosome in cis (Clemson et al., J. Cell Biol. 142:13-23 (1998)), and directs silencing of the linked chromosome by recruiting heterochromatin (Borsani et al., (1991) supra; Brockdorff et al., (1991) supra; Brown et al., (1991) supra). The antisense gene, Tsix, acts as a binary switch for Xist expression: On the future Xi, the loss of Tsix expression permits the upregulation of Xist and chromosome silencing in cis; on the future Xa, the persistent expression of Tsix during female cell differentiation protects that X from the silencing effects of Xist (Lee and Lu, Cell 99:47-57 (1999); Luikenhuis et al., Mol. Cell Biol. 21:8512-8520 (2001); Stavropoulos et al., Mol. Cell Biol. 25:2757-2769 (2001)). Tsix persistence on the Xa depends on Xite (Ogawa and Lee, (2003) supra; Stavropoulos et al., Mol Cell Biol. 25:2757-2769 (2005)), which cooperates with Tsix to regulate both counting and choice (Lee, Science 309:768-771 (2005)).

The repressive properties of Tsix on Xist requires the 5′ end of the antisense gene, with evidence implicating either specific DNA elements (Chao et al., Science 295:345-347 (2002); Lee and Lu, (1999) supra; Morey et al., Hum. Mol. Genet. 10:1403-1411 (2001)) or transcription via the major Tsix promoter (Luikenhuis et al., (2001) supra; Sado et al., Development 128:1275-1286 (2001); Shibata and Lee, Curr. Biol. 14:1747-1754 (2004); Stavropoulos et al., (2001) supra). The Tsix^{ΔCpG} knockout (Lee and Lu, (1999) supra) has defined a 3.7 kb critical domain that includes the major Tsix promoter and bipartite enhancer (Stavropoulos et al., (2005) supra), the repeat element DXPas34 (Courtier et al., Proc. Natl. Acad. Sci. 92:3531-3535 (1995); Debrand et al., Mol Cell Biol. 19:8513-8525 (1999)), and CTCF-binding sites with potential function in allelic choice (Chao et al., (2002) supra). Clearly, however, some aspect of antisense transcription is also important, as forced expression (Luikenhuis et al., (2001) supra; Stavropoulos et al., (2001) supra) and premature transcript termination (Luikenhuis et al., (2001) Supra; Sado et al., (2001) supra; Shibata and Lee, (2004) supra) both lead to skewed X inactivation choice. While these analyses have uncovered many potential elements, whether and to what extent each contributes to control of Xist is currently not clear.

The experiments described below seek to define the specific required elements by generating new knockout alleles within the 3.7 kb Tsix^{ΔCpG} domain. First, to determine whether antisense transcription is actually required, I deleted various promoter fragments of Tsix and found, much to our surprise, that the mutations produced no XCI phenotype. Computational analysis of the remaining sequence were used and identified DXPas34 as a remnant of an ancient retrotransposon with two distinct functions. The molecular characteristics and genetic significance of DXPas34 is reported below herein.

### Results

### Targeted Deletions of the Tsix Promoter do not Impair Tsix Function

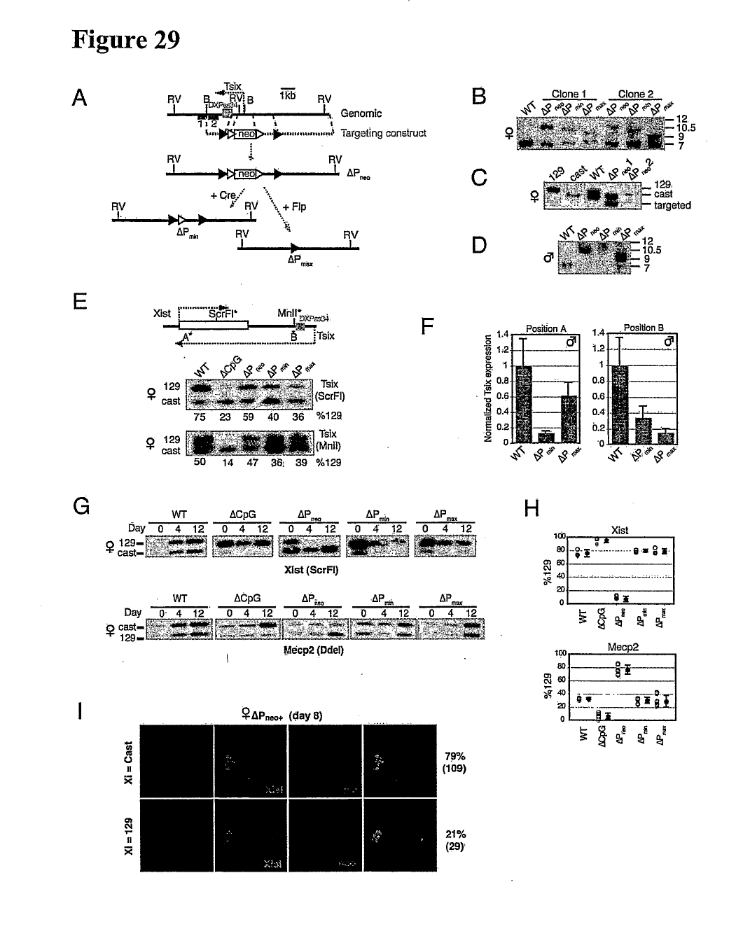

To determine whether transcriptional activity is required for Tsix function, I created deletions around the major Tsix promoter. The major promoter has been mapped to a 276 bp fragment spanning −160 to +116 bp of the Tsix start site (Stavropoulos et al., (2005) supra) [Note: a minor 6 promoter has been described upstream, but its deletion has no consequence for XCI (Ogawa and Lee, (2003) supra; Sado et al., (2001) supra; Stavropoulos et al., (2001) supra)]. Because the immediate flanking regions may also contain crucial elements, two types of promoter deletions were generated, one which removes ˜700 bp of sequence around the start site (ΔPₘᵢₙ) and the other which removes ˜2100 bp that extends up to but does not include DXPas34 (ΔPₘₐₓ). To simplify the targeting effort, both the Cre-Lox and Flp-Frt site-specific recombinase systems (Meyers et al., Nat. Genet. 18:136-141 (1998)) were used to create a pair of nested deletions (FIG. 29A).

I transfected the promoter targeting construct into mouse embryonic stem (ES) cells, a system routinely used to model XCI in culture. Both XX and XY ES lines were tested in order to detect any potential effects of the mutations on counting and choice. Targeting into the female 16.7 line resulted in two homologous recombinants out of 3000 screened (FIG. 29B). Because 16.7 contains one X-chromosome of Mus castaneus origin (“cast”) and a second X of Mus musculus origin (“129”), we could use restriction fragment length polymorphisms (RFLP) arising from strain-specific differences in DXPas34 repeat number (Avner et al., Genet. Res. 72:217-224 (1998)) to determine which X was targeted in the female cells. In both cases, the 129 allele was targeted (FIG. 29C). Targeting into the male J1 line yielded two homologous recombinants out of 500 colonies screened (FIG. 29D). Transient transfection of targeted cell lines with Cre and Flp recombinases, respectively, yielded ΔPₘᵢₙ and ΔPₘₐₓ (FIGS. 29B,D). The independently isolated clones behaved similarly, so a single representative clone of each deletion type and sex is discussed below.

To determine their effects on Tsix expression, allele specific RT-PCR analysis based on polymorphic MnlI and ScrFI sites was performed (FIG. 29E). As predicted, Tsix expression from the mutant allele (129) in females was significantly reduced as compared to the wild-type. Tsix expression from the mutant allele in hemizygous male cells was also significantly reduced as determined by real-time RT-PCR analysis (FIG. 29F). Both ΔPₘₐₓ and ΔPₘᵢₙ had a much milder impact on Tsix transcription than Tsix^{ΔCpG}, suggesting that significant promoter activity could be found outside of the ΔPₘₐₓ region (see below).

To determine whether the promoter mutations affected XCI, male and female cells were differentiated into embryoid bodies (EB) in culture to initiate the XCI pathway and looked for effects on counting and choice. Cells with abnormal counting have been shown to either differentiate poorly or die during differentiation (Clerc and Avner, Nat. Genet. 19:249-253 (1998); Lee, (2005) supra). However, ΔPₘᵢₙ and ΔPₘₐₓ EB in both XX and XY backgrounds differentiated and grew normally, with no quantitative increase in cell death. These observations argued against a defect in counting, a result that was predictable based on the absence of a counting phenotype for Tsix^{ΔCpG} in the hetero- and hemi-zygous states (Lee and Lu, (1999) supra).

To query effects on choice, the relative allelic contribution to the expression of Xist and the X-linked gene, Mecp2 were examined. Surprisingly, neither ΔPₘᵢₙ nor ΔPₘₐₓ had any effect on allelic choice in the XX cells (FIGS. 29G,H). This result contrasted with that of Tsix^{ΔCpG}, which exhibited completely skewed XCI patterns in the heterozygous female (Lee and Lu, (1999) supra). Thus, although forced Tsix transcription (Luikenhuis et al., (2001) supra; Stavropoulos et al., (2001) supra) or premature Tsix termination (Luikenhuis et al., (2001) supra; Sado et al., (2001) supra; Shibata and Lee, (2004) supra) skews the pattern of XCI choice, transcription initiating from the major Tsix promoter is surprisingly not required for Tsix's regulation of random choice.

Oddly, however, while the ΔPₘᵢₙ and ΔPₘₐₓ alleles caused no XCI phenotype, their precursor allele, ΔPₙₑₒ, resulted in nonrandom XCI, with inactivation occurring predominantly on the wildtype X, as observed by both allele-specific RT-PCR and FISH (FIG. 29G-I). Therefore, despite the elimination of the 700 bp promoter region, ΔPₙₑₒ behaved oppositely of all Tsix knockout alleles generated to date (Lee and Lu, (1999) supra; Luikenhuis et al., (2001) supra; Morey et al., (2001) supra; Sado et al., (2001) supra; Shibata and Lee, (2004) supra) and more closely resembled Tsix overexpression alleles (Luikenhuis et al., (2001) supra; Stavropoulos et al., (2001) supra), where the mutated X remains preferentially active as cells differentiate. But unlike the Tsix^{EFIα} overexpression allele in which nonrandom XCI was secondary to cell selection (Stavropoulos et al., (2001) supra), ΔPₙₑₒ exhibited a primary defect in choice. No excessive cell death was observed over the course of differentiation (data not shown), indicating that the mutant cells each chose the mutant X as the Xa. These results indicated that deleting the Tsix promoter in combination with insertion of a Pgk-Neo marker created a neomorphic allele. Because Pgk-Neo was inserted in the opposite orientation to Tsix, the phenotype could not have resulted from Neo read-through transcription into Tsix. Rather, a Pgk enhancer linked to the Pgk-Neo construct (McBurney et al., Nucleic Acids Res. 19:5755-5761 (1991); Sutherland et al., Gene Expr. 4:265-279 (1995)) could have created ectopic interactions that bypassed established the normal mechanism of choice (see Discussion).

### DXPas34 is a Conserved Element

Given that the major Tsix promoter is dispensable for regulation, I focused on DXPas34—a prominent motif comprising the 3.7 kb Tsix^{ΔCpG} sequence not deleted in ΔPₘₐₓ. Consisting of tandem repeats of ˜34 bp in the mouse (Avner et al., (1998) supra; Courtier et al., (1995) supra), DXPas34 harbors binding sites for the chromatin insulator, CTCF (Chao et al., (2002) supra), and contributes to the activity of a bipartite Tsix enhancer (Stavropoulos et al., (2005) supra). However, because early studies indicated that DXPas34 is not conserved outside of the mouse (Avner et al., (1998) supra; Chureau et al., Genome Res. 12:894-908 (2002); Courtier et al., (1995) supra; Debrand et al., Mol. Cell Biol. 19:8513-8525 (1999); Migeon et al., (2001) supra; Nesterova et al., (2001) supra), its functional significance has been unclear.

To test whether DXPas34 is conserved after all, bioinformatic analysis using mouse, rat, and human Xic sequences were carried out. Interestingly, dot-plot analysis identified a set of heretofore unrecognized repeats in the rat Xic at a location syntenic to mouse DxPas34 (FIG. 30A). The dot-plot also revealed that two distinct repeat clusters are present in this domain: ‘Repeat A’, which corresponds to DXPas34 (Avner et al., (1998) supra; Courtier et al., (1995) supra), and the previously undescribed ‘Repeat B’. Repeat A, the major repeat in mouse, is greatly expanded in mouse and could be further subclassified as A1 and A2. A1 is 34 bp in length corresponds to the repeat unit identified previously (Courtier et al., (1995) supra), and is present in 29 tandem copies in the 129 strain (AgeI to MluI fragment). The A2 repeat unit, which is only 32 bp in length, is found in five tandem copies located between the A1 array and the Tsix promoter. Only one type of Repeat A is found in rat, and it is present in 13 highly degenerate copies. The Mouse A2 consensus and Rat A consensus share the distinctive 6-bp ATTTTA motif with the major A1 repeat of DXPas34. The mouse A2 and the rat A consensus do not contain the GGTGGC motif present in A1. This motif coincides with the core of the CTCF binding sites previously mapped within DXPas34 (Chao et al., (2002) supra). Repeat B, which lies immediately upstream of A2, is 30 bp in length and occurs in only six tandem copies in the 129 strain. In contrast to Repeat A, Repeat B has a 31 bp consensus sequence in rat, where it is expanded to 35 tandem copies. Interestingly, both mouse and rat B consensus sequences contain an inversion of the CTCF-motif (5′-GCCACC-3′) as well as a nearby partial inversion (CCACT) (FIG. 30B).

We next compared mouse and human sequences. Wile previous analysis had detected three regions of homology between mouse and human (R1, R2, R3 (Lee et al., (1999) supra)), no homology was obvious around DXPas34. In fact, human TSIX possesses a 14-kb insertion between R2 and R3 that does not occur in the mouse, where R2 and R3 are contiguous (FIG. 30C). Because the mouse is phylogenetically more distant to human than to rat, it was expected that any potential human DXPas34 element and the adjacent Repeat B might easily escape detection. Therefore, degenerate Repeat A and B search strings for closer inspection were developed and showed that, although the B-motif showed no obvious orthologue in human TSIX, a closely related A-cluster consisting of 16 base-pairs (bp) that span the CTCF recognition site was evident downstream of the human TSIX start site (FIG. 30C-D). This element is repeated seven times, oriented in the same direction, and dispersed across a 3 kb domain between R2 and R3. Repeat B of mouse and rats also contains the same 16-bp CTCF-containing domain. A general search for this type of co-oriented, highly clustered repeat array uncovered no other at the Xic/XIC.

On the basis of this analysis, I conclude that DXPas34 is indeed conserved among mammals in the following manner: (i) the DXPas34 region is actually composed of two distinct but related repeat clusters, Repeat A (within the originally described DXPas34) and Repeat B (adjacent to the original DXPas34). These repeats are found in at least two species of mammals. (ii) Rodent Repeat A is a composite of three regions, including a central 16-bp CTCF-containing domain, an upstream 11-bp domain, and a downstream 6-bp ATTTTA domain. (iii) Human Repeat A appears more compact, consisting only of the 16-bp CTCF-containing domain without obvious 11- or 6-bp flanking domains. (iv) Repeat B, present in mouse and rats, contains an inverted, partial CTCF motif (GGNGG).

### Origins of DXPas34 in an ERV Retrotransposon

In mammals, at least 575 families of repetitive DNA elements are known to exist (Jurka et al., Genome Res. 110:462-467 (2005)) (http://www.girinst.org/repbase/update/index.html). While testing DXPas34's conservation among mammals, I made the unexpected discovery that the human A-repeats are part of larger retrotransposon units occurring in tandem within the 14 kb gap between R2 and R3 (FIG. 30C-D). Intriguingly, five of the seven Repeat A motifs resided within LTR/ERV or SINE/Alu-subclasses of repeat elements, suggesting that DXPas34 may have descended from ancient retrotransposons. To investigate this further, I asked whether the motifs in FIG. 30B were generally present in rodent and human repetitive elements or whether they were found only in specific families (the rodrep.ref and humrep.ref files from RepBase10.11, obtained from http://www.girinst.org/server/RepBase/index.php). The 16-bp CTCF core domain of human Repeat A matched three repetitive elements in the human database, including a hAT-type DNA repeat element, MER45R, and two related endogenous retroviral elements (ERV), ERVL and HERVL (FIG. 30E). Likewise, mouse Repeat A contained the same 16-bp CTCF domain (FIG. 30B) and matched the corresponding MER45R, MERVL, and RatERVL sequences in the rodent database. [Notes regarding terminology: (i) Although LTRs represent the ends of endogenous retroviruses (ERV), LTR and ERV elements are subclassified separately in the RepBase; (ii) The ‘H’ in HERVL refers to ‘human’, while the ‘M’ in MERVL refers to mouse]. Elements shared by the human and mouse databases represent ancient repeats that predated the divergence of primates and rodents.

Scrutiny of the context of these alignments led to another surprising finding: The 16-bp CTCF domain was not the only region of homology. In fact, the upstream 11-bp domain upstream of the mouse A1 core (FIG. 30B) also matched the upstream sequences in HERVL and MERVL. These matches occurred in the Integrase-coding region of endogenous retroviruses (Benit et al., J. Virol. 73:3301-3308 (1997)). Thus, DXPas34 and the ERVs aligned at two contiguous domains, the 16-bp core domain and the 11-bp upstream domain. While the probability of carrying the 16-bp motif within one element is ˜2.1×10−9 (considering all nucleotide permutations, 4⁻¹⁵×2⁻¹), the probability of carrying both the 11-bp and 16-bp motifs is 2.8×10⁻¹⁴ ([4⁻¹⁵×2⁻¹]×[4⁻⁸×2⁻¹×1⁻¹×1⁻¹]). This is a very improbable phenomenon, as the chance of the coincidence is once in 2.8×10¹⁴ nucleotides−five orders of magnitude greater than the size of the mammalian genome. These arguments therefore supported kinship between DXPas34 and HERVL/MERVL retrotransposons.

To test this hypothesis, two analyses were carried out. First, I reasoned that if DXPas34 were derived from MERVL, then hits in the mouse genome should be MERVL-related. Indeed, a sampling of mouse Chromosome 3 (160 Mb) uncovered 48 hits at a level of 5 mismatches or fewer. Significantly, 43 out of 48 were recognized by Repeatmasker as MERVL-related, based on the Mouse Genome Table Browser (http://genome.ucsc.edu/cgi-bin/hgTables). For the remaining five, a closer inspection revealed that at least one also bore resemblance to MERVL but was not recognized by Repeatmasker as such. This conclusion was based on alignment of a 227-bp context enclosing the pattern match with MERVL using the BAST-2-sequence alignment method (http://ncbi.nlm.nih.gov/blast/b12seq/wblast2.cgi). A sampling of the X-chromosome revealed 50 hits at 5 or fewer mismatches in the 165 Mb region (excluding hits within DXPas34 itself). Of the 50 hits, 48 were similarly shown to be MERVL-related. A query of the entire mouse genome yielded a total of 846 pattern matches to DXPas34. Based on extrapolation of these results, of these matches, approximately 795 would be in MERVL sequences. Thus, of all hits identified by DXPas34 in the mouse genome, ˜95% occur within sequences annotated as MERVL.

Second, if DXPas34 were derived from MERVL, the 16 bp+11 bp string should identify no other repeat elements in RepBase. Searches of the RepBase using the fused 11+16 bp motif, 5′-GTGAYNNCCCAGRTCCCCGGTGGCAGG-3′ (SEQ ID NO: 90) were performed. In the human database, this 27-bp sequence matched only HERVL and no other types of retrotransposons at a stringency of four or fewer mismatches (FIG. 30E; Note that the 11-bp motif is present in the HERVL sequence). In the rodent database, it similarly matched only the corresponding RatERVL and MERVL and no other retrotransposons at a stringency of five or fewer mismatches (FIG. 30E). [Note: This search did not identify MER45R because MER45R matched only the 16-bp motif, not the 11-bp motif. Thus, the 27-bp search yielded a more stringent and specific result]. To determine the probability with which these matches could have occurred by chance alone, Monte Carlo analysis was performed using a statistical method independent of the way the matches were found. I shuffled bases in the 27-bp motif and tested whether the randomized 27-bp string (with an otherwise identical base-composition) would find matches in RepBase at the same stringency and frequency. In a test of 100 randomized permutations of the 27-bp pattern, no matches were observed in the rodent repeat database at a stringency of four or fewer mismatches. Importantly, this was qualitatively similar to the match between DXPas34 and HERVL. Only 3 out of 100 permutations gave hits at five mismatches, which is qualitatively similar to the overall fit with rodent ERVL. Taking these data together, I conclude that the HERVL, RatERVL, and MERVL hits identified by DXPas34 were not the result of chance.

In sum, among 575 families of repetitive DNA, DXPas34 specifically resembles ERV retrotransposons (HERVL, RatERVL, and MERVL). This sequence similarity may result from DXPas34's origin in one or a small number of elements in the ERV family of retrotransposons. Given the detectable level of conservation between rodents and humans, it is very likely that DXPas34 emerged by the time of the primates-rodents divergence some 60-80 million years ago.

### Novel Activities Within DXPas34

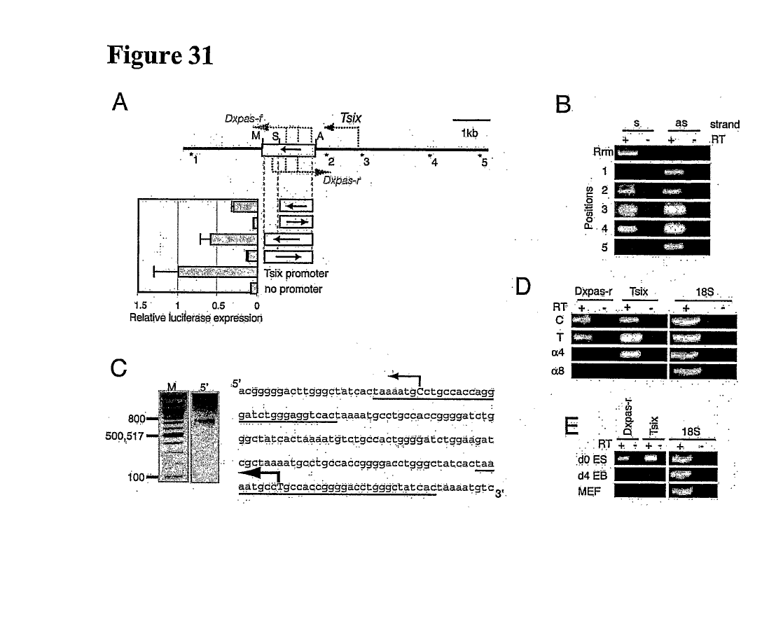

LTR/ERV, like other mammalian retrotransposons, often possess promoter activity and may be transcribed at low levels from both strands (reviewed in (Kazazian, (2004) supra)). A promoter activity within DXPas34 could potentially substitute for the loss of the major Tsix promoter in ΔPₘᵢₙ and ΔPₘₐₓ and explain their minimal phenotype. Indeed, I found that DXPas34 could serve as promoter when placed in its native orientation in a luciferase reporter assay (FIG. 31A). Consistent with this, Tsix cDNAs have been found to initiate within DXPas34 (Shibata and Lee, Hum. Mol. Genet. 12:135-136 (2003)). To look for associated transcripts, I carried out strand-specific RT-PCR in ES cells and observed the expected antisense transcripts downstream of DXPas34 (FIG. 31B, position 1). Intriguingly, between DXPas34 and the Tsix promoter (position 2), transcription in the reverse (sense) as well as forward (antisense) orientations was observed. This novel reverse transcript was less abundant than Tsix RNA, proceeded through a ˜3 kb region (positions 3 and 4), and terminated near position 5. Using a primer at position 2, 5′ RACE products revealed multiple transcription initiation sites within DXPas34, each coincident with a discrete Repeat A1 unit (FIG. 31C). Therefore, each A1 Repeat unit may serve as an origin of transcriptional activity. The forward and reverse transcriptional units are referred to as Dxpas-f and Dxpas-r, respectively.

LTR/ERVs are known to be transcribed by RNA Pol II (Havecker et al., Genome Biol. 5:225 (2004)). Because the Repeat A1 units do not bear obvious resemblance to Pol II promoters, I asked which RNA polymerase is actually responsible for transcription of Dxpas-r by treating undifferentiated ES cells for 4 hours with either α-amanitin or tagetin, which specifically inhibit Pol II or Pol III, respectively. Strand-specific RT-PCR showed that the Dxpas-r RNA was severely diminished in α-amanitin-treated cells, while it is unaffected in tagetin-treated cells (FIG. 31D), arguing that Dxpas-r is transcribed by Pol II. Treatment with α-amanitin for an additional 4 hours (8 hours total) also abolished Tsix expression (FIG. 31D, α4 vs. α8), indicating that Pol II also transcribes Tsix and that this transcript has a longer half-life. These results support the conclusion that DXPas34 possesses bidirectional Pol II activity, providing further evidence that Repeat A resembles an LTR/ERV retrotransposon.

I then examined the developmental profile of Dxpas-r by analyzing undifferentiated (day 0) ES cells, differentiating EB (day 4), and fully differentiated mouse embryonic fibroblasts (MEFs). Interestingly, Dxpas-r's expression pattern was similar to that of Tsix (FIG. 31E): Expression was most robust on day 0, diminished by day 4, and absent in MEFs. This expression pattern correlated precisely with the reported methylation profile of DXPas34, which is unmethylated in ES cells and hypermethylated on the Xₐ of differentiated cells (Avner et al., (1998) supra; Courtier et al., (1995) supra), and the status of a recently described ES-cell-specific DNase I hypersensitive sites in DXPas34 (Luikenhuis et al., (2001) supra; Stavropoulos et al., (2001) supra; Stavropoulos et al., (2005) supra).

### Dual Positive-Negative Regulation of Tsix by DXPas34

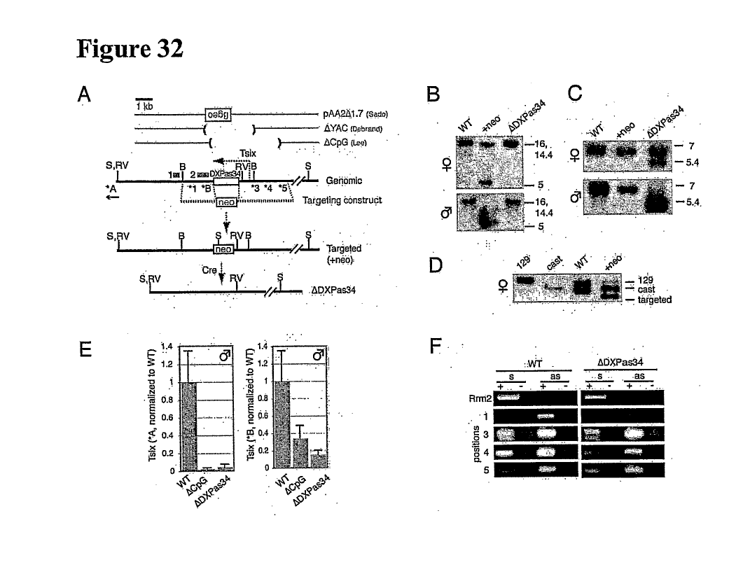

In light of these discoveries, I asked whether DXPas34 plays a role in XCI. Although several other Tsix knockout alleles have included DXPas34 (FIG. 32A), a deletion strictly of this 1.6 kb element had not been created previously (Debrand et al., (1999) supra; Lee and Lu, (1999) supra; Luikenhuis et al., (2001) supra; Sado et al., (2000) supra). Therefore, the necessity of DXPas34 itself for XCI regulation has remained unclear. Aa targeted deletion of Repeat A1 in XX and XY cells was created, obtaining two correctly targeted male clones out of 300 colonies screened and one correctly targeted female clone out of 3000 (FIG. 32B). The Neo marker was then removed by transient transfection with Cre (FIG. 32B-C, right most lanes) and RFLP analysis confirmed the 129 allele was targeted in the XX line (FIG. 32D). For both male and female cells, clones with and without the neomycin marker behaved similarly, so a representative clone without the neomycin marker is discussed. Deleting DXPas34 resulted in a significant reduction of Tsix expression, similar to that in the Tsix^{ΔCpG} allele (FIG. 32E). These results demonstrated that DXPas34 is a positive transcriptional regulator of Tsix, in accordance with the fact that DXPas34 comprises part of the Tsix bipartite enhancer (Stavropoulos et al., (2005) supra). It was noted that deleting DXPas34 diminished but did not completely eliminate expression of Dxpas-r (FIG. 32F), possibly because of minor Dxpas-r start sites mapped by RACE to positions just outside the deleted region.

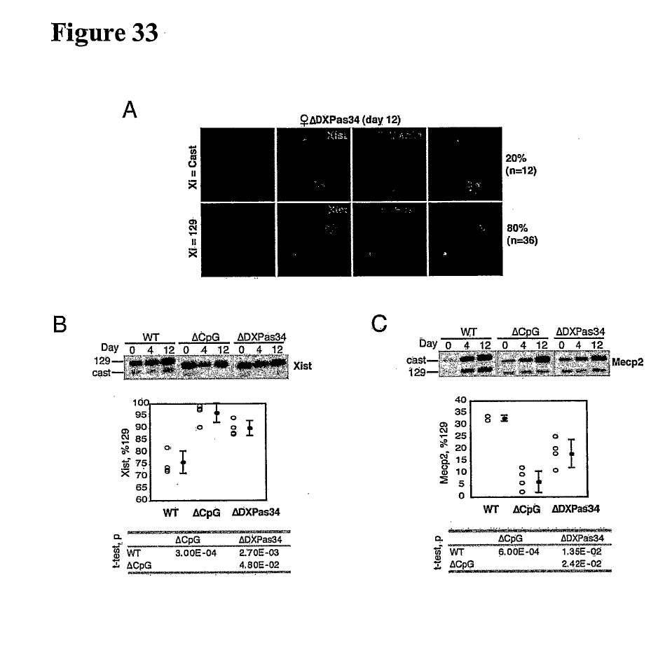

ΔDXPas34 exerted no obvious effects on XCI counting in the hetero- and hemi-zygous states, as all XX and XY clones grew and differentiated normally without elevated cell death (data not shown). By contrast, ΔDXPas34 produced clear effects on XCI choice and recapitulated the nonrandom XCI phenotype associated with Tsix^{ΔCpG}. In the heterozygous ΔDXPas34/+ cell line, allele-specific analysis of Xist and Mecp2 showed biased expression of the M. castaneus alleles (FIG. 33A-C). The bias may be somewhat milder for ΔDXPas34 than ΔCpG, perhaps more reminiscent of the inabsolute skewing seen for Xite+/−heterozygotes (Ogawa and Lee, (2003) supra). Like the Tsix^{ΔCpG} and Xite^{ΔL} heterozygotes, the ΔDXPas34 EB also did not exhibit elevated cell death when compared to wildtype XX cells (data not shown), suggesting that the nonrandom XCI patterns was due to a primary effect on the choice function of Tsix rather than a secondary effect of cell loss. Thus, the nonrandom XCI caused by Tsix^{ΔCpG} could mainly be attributed to the loss of DXPas34, rather than to promoter loss.

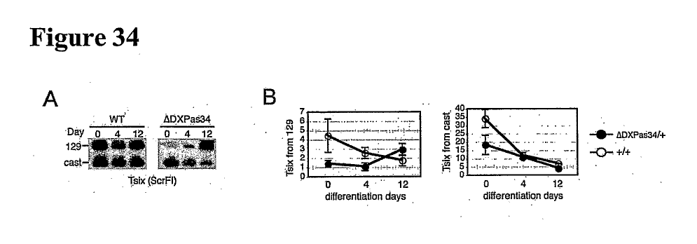

A distinct paradoxical effect of deleting DXPas34 during late days of differentiation was uncovered in these experiments. In heterozygous females, deleting DXPas34 led to an apparent derepression of Tsix as measured at the ScrFI polymorphic site by allele-specific RT-PCR (FIG. 34A). This de-repression was first evident on day 4 of differentiation and became sufficiently robust on day 12 that the 129 (mutated) Tsix transcripts greatly exceeded the contribution from the wild-type castaneus allele. This flip in the relative ratio could be due to either a true increase in 129 transcripts or rather to a precipitous drop in the castaneus transcripts (which would therefore give the appearance that the 129 transcripts increased over time). To distinguish between the possibilities, quantitative, allele-specific RT-PCR was carried out using the housekeeping gene, Rpo2, as an internal calibrator. In wildtype XX cells, both the 129 and castaneus Tsix transcript levels decreased significantly from days 0 to 12 as expected (FIG. 34B). However, in ΔDXPas34 cells, Tsix from the mutant (129) chromosome actually increased over time. In the same cells, however, the wild-type castaneus chromosome behaved similarly to the castaneus chromosome in wildtype cells, showing the expected down-regulation of Tsix once XCI was complete. These observations demonstrated that, during the establishment and maintenance phases of XCI, DXPas34 is required to stably repress Tsix transcription. Thus, DXPas34 serves two sequential functions with respect to Tsix: Stimulation of antisense transcription at the onset of XCI, followed by stable silencing of Tsix after XCI is established.

Note, however, that the de-repression of Tsix during late-stages did not reverse XCI. I believe that this is due to the ES cells' having passed the “reversible phase” of XCI (Wutz and Jaenisch, Mol. Cell. 5:695-705 (2000))—that is, loss of Tsix expression may be necessary but not sufficient to reactivate Xist. These results suggest that the ancient MERVL retrotransposon may have been usurped to play both activating and repressive roles on Tsix regulation. I propose that DXPas34 is a dual regulator of Tsix expression, with its activating role occurring first and its repressive role occurring after the establishment of XCI.

### Discussion

### DXPas34 Originates in an LTR/ERV Retrotransposon

We have provided evidence that DXPas34 originated from an LTR/ERVL retrotransposons. DXPas34 itself consists of two recognizable clusters of related repeats, Repeats A and B. Of 575 repeat families represented in RepBase, these repeats specifically matched the HERVL, RatERVL, and MERVL families. Sequence matches to these related endogenous retroviruses occur in the 11-bp upstream domain of Repeat A, the 16-bp central domain of Repeat A, and the GGNGG core of Repeat B. Interestingly, the 16-bp domain contains the consensus for the chromatin insulator, CTCF, previously shown to bind mouse DXPas34 (Chao et al., (2002) supra). The sequence similarity between DXPas34 and the ERV repeats is apparently not a random occurrence, as the probability of this coincidence is once in 2.8×10¹⁴ nucleotides−five orders of magnitude greater than the size of the mammalian genome. Thus, I propose that DXPas34 originated from one or a small number of ERVs.

ERVs arose in mammals more than 70 million years ago, prior to the divergence of simians and rodents (Benit et al., (1999) supra). Today, some 5,000-20,000 copies of HERVL and MERVL are present in the human and mouse genomes. Previous work had shown that retrotransposons are subject to extensive internal expansion of GC-rich repeat units in mouse (Bois et al., Genomics 49:122-128 (1998); Bois et al., Mamm. Genome 12:104-111 (2001)). The GC-richness of present-day mouse DXPas34 may indicate that a similar infiltrative process occurred at this locus. It seems likely that the retroviral elements which ultimately gave rise to DXPas34 underwent extensive degradative mutagenesis and repeat expansion over the past 80-100 million years, after the point of primate-rodent divergence. This degenerative process perhaps preserved only those sequences which fortuitously serve some function at the primordial Tsix, rendering DXPas34 minimally recognizable today as a former member of the LTR/ERV retrotransposon family.

### A Novel Function for Junk ‘DNA’ in Epigenetic Regulation

Genetic analysis of DXPas34 performed herein now ascribes novel function to such repetitive elements historically regarded as ‘junk DNA’. DXPas34 displays bidirectional transcription and plays two roles in the epigenetic regulation of Tsix. Using a combination of bioinformatic, molecular, and genetic techniques, I have placed its role in the context of other 5′ Tsix regulators. In light of previous work showing that forced Tsix expression results in a gainof-function allele (Luikenhuis et al., (2001) supra; Stavropoulos et al., (2001) supra), I had expected a Tsix null allele and skewed XCI choice upon deleting the promoter. However, neither ΔPₘᵢₙ nor ΔPₘₐₓ had any obvious effect on Tsix's regulation of Xist despite a significant decrease in antisense expression. Thus, although transcription through Tsix is sufficient to block Xist function, transcription from the major promoter is not absolutely required for random choice. Through Dxpas-f transcription, DXPas34 rescues the loss of transcription initiation from the major Tsix promoter, with contribution from upstream initiation sites possibly also playing a role (Ogawa and Lee, (2003) supra; Sado et al., (2001) supra). In the reverse direction, Dxpas-r transcription is readily detected and has developmental dynamics similar to that of Tsix, perhaps thereby also playing a role in Tsix regulation.

The knockout analysis clearly shows that DXPas34 has both positive and negative effects on Tsix. Previous work has revealed that Tsix is regulated by two enhancers, a bipartite enhancer that contains DXPas34 and an upstream enhancer embedded within Xite (Ogawa and Lee, (2003) supra; Stavropoulos et al., (2005) supra). Consistent with the fact that DXPas34 is critical for bipartite enhancer action (Stavropoulos et al., (2005) supra), I have now shown that its deletion results in a dramatic loss of Tsix transcription from the major promoter, indicating that DXPas34's positive regulatory influence is achieved through its action as enhancer. Unexpectedly, its deletion also results in a late-stage re-activation of Tsix in cis, indicating that DXPas34 must also act in the stable repression of the antisense gene. Thus, ironically, DXPas34 has also become a first candidate repressor of Tsix. These roles may be conserved in humans as well, as bidirectional transcription has also been detected from the syntenic region of TSIX(Chow et al., Genomics 82:309-322 (2003)).

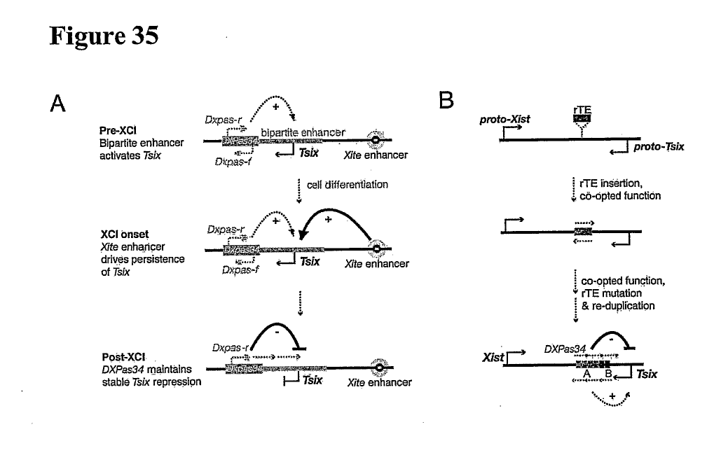

In the context of available data, our current work leads to a three-step model in which two enhancers and two functions of DXPas34 act in sequence to control distinct aspects of Tsix dynamics (FIG. 35A). With ΔDXPas34's effects are already evident in undifferentiated ES cells, I propose that the bipartite enhancer acts in pre-XCI cells to achieve biallelic Tsix expression. By contrast, the Xite enhancer works primarily at the onset of XCI, as its deletion has little effect on Tsix before XCI but results in a premature loss of Tsix expression during XCI (Ogawa and Lee, (2003) supra). (Note: The Xite enhancer may also facilitate Tsix expression in pre-XCI cells, but this effect has not been uncovered so far by genetic analysis.) That is, while the bipartite enhancer is required for de novo expression of Tsix, the Xite enhancer is necessary for persistent Tsix expression on the future Xₐ. Therefore, the future Xₐ and Xᵢ are distinguished by the action of the Xite enhancer, with the enhancer acting asymmetrically on the Xₐ and not on the Xᵢ. Following the establishment of XCI, Tsix expression is itself extinguished. I propose that this repression requires the late-stage second function of DXPas34. Given the existence of Dxpas-r transcripts, this antiparallel transcription may suppress Tsix in a manner similar to Tsix-mediated antisense repression of Xist expression. In the context of this model, the gain-offunction ΔPₙₑₒ phenotype may be a direct consequence of an ectopic Pgk-Neo enhancer that bypasses a requirement for Xite. By being upstream of Dxpas-f transcription, the ectopic enhancer could short-circuit endogenous regulatory networks and create a constitutively persistent Tsix allele.

While the effects of the heterozygous deletions on random choice are clear, the current work does not address the effects of ΔPₘᵢₙ, ΔPₘₐₓ, ΔPₙₑₒ, and ΔDXPas34 on other aspects of XCI regulation, such as X-chromosome counting, the mutual exclusiveness of choice, and XCI imprinting. In the case of Tsix^{ΔCpG}, an aberration in counting became evident only in homozygous knockout cells (Lee, (2002) supra; Lee, (2005) supra), so homozygosing the promoter and DXPas34 deletions may uncover a role in other (and no less important) aspects of XCI regulation.

Finally, the possible evolutionary origin of DXPas34 in endogenous retroviruses (ERVs) remains intriguing. With each retrotransposon being a self-sufficient gene expression module containing promoters, enhancers, and insulators (Gerasimova and Corces, Curr. Opin. Genet. Dev. 6:185-192 (1996); Willoughby et al., J. Biol. Chem. 275:759-768 (2000)), each insertion introduces a new repertoire of regulatory elements that could be utilized by genes at the site of integration. We suggest that a fortuitous ERV insertion into the primordial Tsix gene led to a co-opting of the element by the Xic to regulate Tsix (FIG. 35B). Indeed, the DXPas34 element contains promoter, enhancer, and insulator activities that have each been proposed as components of the regulatory machinery (Chao et al., (2002) supra; Stavropoulos et al., (2005) supra). Over time, the ERV might have lost nearly all of its original sequences, excepting those with beneficial effects on Tsix. Such beneficial elements might then be re-duplicated to yield the repetitive structure seen today at DXPas34. DXPas34 therefore adds to a growing list of possible functions carried out by TEs formerly considered junk DNA (Ferrigno et al., Nat. Genet. 28:77-81 (2001); Morgan et al., (1999) supra; Oei et al., Genomics 83:873-882 (2004); Peaston et al., (2004) supra). Others have noted that TEs exhibit a commanding presence at other epigenetically regulated loci such as autosomally imprinted domains of plants and mammals and centromeres of fission yeast and plants (Cain et al., Nat. Genet. 37:809-819 (2005); Lippman et al., (2004) supra; Noma et al., Nat. Genet. 36:1174-1180 (2004); Seitz et al., Nat. Genet. 34:261-262 (2003); Sleutels and Barlow, Academic Press, San Diego, Calif. pp. 119-154 (2002); Volpe et al., Science 297:1833-1837 (2002)). Thus, TE-associated elements may comprise a general mechanism of epigenetic gene control in fungi, plants, and mammals. Accordingly, any of the minimal DXPas34 consensus motifs shown in FIG. 30B (SEQ ID NOs: 28-32 and 40), or multimers thereof, or any ERV derived multimer of the canonical sequence can be used to inhibit cell differentiation.

The following materials and methods were used for the experiments described above.

### Bioinformatic Analysis

Interspecies sequence comparisons were performed using dot-plot methods from the GCG Software Package (http://www.accelrys.com/products/gcg/). Window size and stringency parameters were adjusted to generate the most visible signal above the background. When repeat regions were suggested by horizontal or vertical rectangular areas, dot-plots of the probable repeat region against itself were performed. This generated plots with lines parallel to the diagonal from which it was estimated the total number of direct repeat copies as well as the length of the repeat unit (bp). In order to find the best repeats at the base pair level within a cluster of tandem repeats the following programs were used: Repeat (GCG package), Equicktandem (EMBOSS package http://emboss.sourceforge.net/ and Etandem (EMBOSS). A cluster was then divided up into individual repeats based on this information. Web implementations of ClustalW (http://www.ch.embnet.org/software/ClustalW.html and http://www.ebi.ac.uk/clustalw/) were used to align the individual repeats. The alignments were used to determine a consensus sequence. TEs present in the human, rat and mouse sequences were identified using the Repeatmasker web program (http://www.repeatmasker.org/cgi-bin/WEBRepeatMasker). Searches for additional DXPas34-like sites in the mouse genome by BLAST search were performed using either the 34 bp mouse A1 consensus sequence (FIG. 30B) or a 1056 bp fragment (bp 139145-140200) that includes the DXPas34 region against the mouse genome (http://www.ncbi.nlm.nih.gov/genome/seq/MmBlast.html) or the human genome (http://www.ncbi.nlm.nih.gov/genome/seq/HsBlast.html). The parameters were set to use blastn, the current reference contigs and an Expect value of 10 so that shorter hits with mismatches would be recovered as well as perfect hits. The human Repeat A pattern was based on previously described CTCF binding motifs (Bell and Felsenfeld, Nature 405:482-485 (2000); Chao et al., (2002) supra; Hark et al., Nature 405:486-489 (2000)). I searched for the degenerate repeat consensus using the pattern matching program Fuzznuc (EMBOSS package), which allows the specification of the number of mismatches allowed as well as consideration of the forward and/or complementary strand. I analyzed the pattern matches list from the Fuzznuc program applied to human genomic sequence (build 35 from http://www.ncbi.nlm.nih.gov/Ftp/) to determine the frequency of finding at least 7 pattern matches in the same orientation within a 3 kb region without any matches in the opposite orientation.

### Cells Lines and Targeted Mutagenesis

Male J1 (40XY) and female 16.7 (40XX) ES cell lines and culture techniques have been described previously ((Lee and Lu, (1999) Supra) and references therein). 16.7 carries X chromosomes of 129 and Mus castaneus origins. To target the Tsix promoter, an EcoRV-BamHI fragment (bp 77,816-81,950 of Genbank X99946 (Simmler et al., Mamm. Genome 4:523-530 (1993))) and a NheI-KpnI fragment (bp 70,569-77,118) were each cloned into pGEM-7Zfy(+), forming pSA and pLA. These plasmids were digested with AgeI and SacI respectively, and synthetic FRT sites comprised of two annealed oligos (DEC1 and DEC2 for pSA and DEC3 and DEC4 for pLA) were inserted, forming pSA-FRT and pLA-FRT. Each synthetic FRT site contains a BamHI restriction site. Oligo sequences were:

(SEQ ID NO: 62)

| | |
|---|---|
| DEC1: | GAAGTTCCTATTCTCTAGAAAGTATAGGAACTTCGGATCCAGCT |

(SEQ ID NO: 63)

| | |
|---|---|
| DEC2: | GGATCCGAAGTTCCTATACTTTCTAGAGAATAGGAACTTCAGCT |

(SEQ ID NO: 64)

| | |
|---|---|
| DEC3: | CCGGGAGTTCCTATTCTCTAGAAGTATAGGAACTTCGGATCC |

(SEQ ID NO: 65)

| | |
|---|---|
| DEC4: | CCGGGGATCCGAAGTTCCTATACTTTCTAGAGAATAGGAACTTC |

The assembled homology arms with FRT sites were sequenced to determine FRT site orientation. Homology arms were liberated from pSA-FRT and pLA-FRT and ligated into the NotI and NheI sites in pPGK-neo-BpA-lox2-dTA, a generous gift of Phil Soriano. The resulting targeting construct, pDECKO, was linearized with PvuI, and 40 μg of linearized DNA were transfected into ˜10⁷ ES cells using Lipofectamine 2000 (Invitrogen). Lipofection complexes were left in contact with ES cells for 8 hours. Transfected cells were selected with 300 μg/mL G418 and resistant colonies were picked on days 7-9 of selection. Targeted clones were then transfected with either Cre or FLP encoding plasmids and neomycin sensitive clones were examined by Southern blot to identify correct excision events. Probe 1 is a PCR fragment (bp 82,247-82,615) amplified with primers:

| | | |
|---|---|---|
| Ext1: | CACATGAGGGCATAGCCGCATTC | (SEQ ID NO: 66) |
| Ext2: | CCTGGCATAAGAAATCTTGAGGAT | (SEQ ID NO: 67) |

Probe 2 is a 1.4 kb MluI-NdeI restriction fragment (bp 79,949-81,461)

The targeting construct for ΔDXPas34 (pKO3) consists of a 2 kb MluI-BamHI fragment (79949-81950 of Genbank X99946) cloned into the SalI site of pLNTK (Gorman et al., Immunity 5:241-252 (1996)). The resulting plasmid was linearized with XhoI and a 6.6 kb BamHI-AgeI fragment (71,753-78,396) was added, creating pKO3. Approximately 10⁷ ES cells were electroporated with 40 μg of pKO3 linearized with PvuI. Colonies were picked after 7-9 days selection with 300 μg/mL G418 and 2 μM gancyclovir. Correctly targeted clones were then transfected with pMC-CreN and neomycin-sensitive clones were analyzed by Southern blot.

### RNA/DNA FISH

RNA/DNA FISH was performed as described previously (Lee and Lu, (1999) supra). For detection of the 129 X in the female ΔPₙₑₒ line, a 2 kb Neo fragment (SalI-XhoI from pGKRN) was labelled with Cy3-dUTP (Amersham) by nick-translation (Roche). For detection of the castaneus X in the female ΔDXPas34 line, a 1.2 kb DXPas34 fragment (AgeI-SalI from pCC3) was used. Xist RNA was detected with P1 plasmid, pSx9, labelled with FITC-dUTP (Roche).

### RT-PCR

Strand-specific RT-PCR was performed using primers as shown in Table for each position. All RT reactions were performed using M-MLV reverse transcriptase and 3 μg of total RNA isolated from undifferentiated male ES cells using Trizol (Invitrogen). Reactions were carried out at a temperature of 50° C. to avoid non-specific priming. PCR was performed using the following conditions: 95° C., 3 minutes; (95° C., 45 seconds; 55° C., 45 seconds; 72° C., 1 minute) for 38 cycles, followed by a 10 minute extension at 72° C. Allele specific RT-PCR for Xist, Tsix, and Mecp2 was performed as described previously (Stavropoulos et al., (2001) Supra). For quantitative, allele-specific RT-PCR, 3 μg of RNA were reverse transcribed at 50° C. using primers ns66 and Rpo2B. Rpo2 was amplified with primers Rpo2-1A (Stavropoulos et al., (2001) supra) and Rpo2B, and detected with Rpo2A. Tsix was amplified with ns66 and ns67 and detected with ns60. Pilot experiments determined that the linear range for these PCRs was 23-27 cycles, and samples were analyzed after 25 cycles using methods described previously (Stavropoulos et al., (2001) supra).

TABLE 5

Primers used for strand specific RT-PCR

| | | |
|---|---|---|
| Position | Sense | Anti-sense |

| | | |
|---|---|---|
| 18S-RNA | 18S-FOR: | 18S-REV: |
| | TCAAGAACGAAAGTCGGAGGTT | GGACATCTAAGGGCATCACAG |
| | (SEQ ID NO: 70) | (SEQ ID NO: 71) |
| Rrm2 | RRM-2A: | RRM- 2C: |
| | AAGCGACTCACCCTGGCTGAC | GACTATGCCATCACTCGCTGC |
| | (SEQ ID NO: 72) | (SEQ ID NO: 73) |
| Rpo2 | RPO2B: | RPO2A: |
| | CTTCACCAGGAAGCCCACAT | GCCAAACATGTGCAGGAAA |
| | (SEQ ID NO: 74) | (SEQ ID NO: 75) |
| 1 | CC3-3C: | CC3-3D: |
| | GCTACCTGTGTGTCTGTATC | ACACACACAAGGGCAAGAAAG |
| | (SEQ ID NO: 76) | (SEQ ID NO: 77) |
| 2 | CC3-1C: | CC3-1B: |
| | AATGCCTGCGTAGTCCCGAA | CGGGAACGTGGCATGTATGT |
| | (SEQ ID NO: 78) | (SEQ ID NO: 79) |
| 3 | CC3-3R: | CC3-4F: |
| | GATCCCGCGCCTCAAGAG | TGGGACCGAGTGGAGCACG |
| | (SEQ ID NO: 80) | (SEQ ID NO: 81) |
| 4 | NGP-41: | NGP-42: |
| | ATGAGAGCATCAGATCTCCC | TCACATACCAGCAAAGCTTTG |
| | (SEQ ID NO: 82) | (SEQ ID NO: 83) |
| 5 | CC4-1A: | CC4-1B: |
| | ATCGCCATTCCAAGCATAAG | CCACAGTGTCCAATTTGTGC |
| | (SEQ ID NO: 84) | (SEQ ID NO: 85) |
| A* | Position-7.1 | Position-7.2 |
| | AGGTGGCAGTGCATACGCATACAT | GGAGAGCGCATGCTTGCAATTCTA |
| | (SEQ ID NO: 86) | (SEQ ID NO: 87) |
| B | DEC105: | DEC106: |
| | CAGTGGCAGGCAGAGCTTTG | GAGCAAACAATGGCACTAAGG |
| | (SEQ ID NO: 88) | (SEQ ID NO: 89) |

*from Shibata and Lee, 2003

### 5′RACE

RACE was performed using the GeneRacer kit (Invitrogen) and 5 μg of total RNA isolated from undifferentiated male ES cells using Trizol (Invitrogen) according to the manufacturer's instructions. Reverse transcription was performed using Thermoscript reverse transcriptase at 65° C. (Invitrogen) and the primer

| | |
|---|---|
| CC3-1DL: | |
| GATAGCTTACATACATGCCACGTTCCCGG | (SEQ ID NO: 68) |

RT products were amplified using CC3-1DL and the provided 5′ Generacer primer using touchdown PCR protocol recommended by the manufacturer, using a one minute extension time for all steps and 25 cycles with an annealing temperature of 65° C. and extension at 68° C. Nested PCR was performed on 1 μL of the primary PCR using primer:

| | |
|---|---|
| CC3-1DN: | |
| GGATGCCTGGGACTGGGAAACTTTACT | (SEQ ID NO: 69) |

and the provided 5′ nested primer for 25 cycles using the recommended cycling conditions. Nested PCR products were gel purified using Qiaquick columns (Qiagen), and cloned using the Topo-TA cloning system (Invitrogen). Cloning products were transformed into the provided chemically competent TOP10 cells and plated on LB-amp-IPTG-X-gal plates. White colonies were picked for further analysis.

### Transcription Inhibitor Experiments

α-amanitin (Sigma) or tagetin (Epicentre) were diluted in ES+LIF medium to a final concentration of 75 μg/mL or 45 μM, respectively. 85% confluent undifferentiated male ES cells were grown under media containing either of the above drugs for 4 or 8 hours. RNA was isolated from each well with Trizol (Invitrogen) and analyzed by RT-PCR.

### Example 5

### Detection of Small RNA Molecules at the X Inactivation Center

Given the results described above indicating that bidirectional transcription and dsRNA occur at Tsix and at Xite and that transcription through Tsix/Xite or the RNA products of Tsix/Xite, or both are required for pairing, it is possible that RNAi is occurring naturally to regulate XCI and differentiation.

Northern blot analyses were performed to determine if small RNAs were present within Xite. For these experiments, 20 μg of total cellular RNA is loaded onto each lane, electrophoresed into an agarose gel, and then hybridized to T3- or T7-generated riboprobes as shown in each diagram. As shown in FIG. 36 small RNAs of 25-30, 35-40, and 50+ nucleotides from both strands (sense and antisense) are detected. The let7b blot is a positive control that shows that the known miRNA (let7b) can be detected by our technique. Bands of interest are depicted by arrows. The same bands are detected regardless of the strand-specificity of the probe. That is, both sense and antisense-strand probes can pick up the small RNAs, suggesting that the small RNAs are double-stranded. Cell lines shown are those from Lee, Science (2005) supra, and Xu et al. Science (2006) supra, and Ogawa and Lee Mol. Cell. (2003) supra. Briefly, J1, wildtype male ES; 16.7, wildtype female ES; J1-ΔCpG is Tsix-deleted male ES; 16.7 Δ/Δ is Tsix−/− female ES; ΔL(Xite) is a 12.5 kb deletion of Xite; female-Tsix3.7 is transgenic female ES with 3.7 kb Tsix sequence deleted in the Tsix-allele; Female-Xite is transgenic female ES with 5.6 Xite transgene. Lanes 0, 4, 10 refer to days of cell differentiation for each cell line.

These methods can be used to identify small RNAs from any region of Xic, Xite, Tsix, Tsix/Xite, or Xist, ranging in size from at least 15 nucleotides, preferably, 17, 18, 19, 20, 21, 22, 23, 24, 25, 26, 27, 28, 29, 30, 31, 32, 33, 34, 35, nucleotides in length and even up to 50 or 100 nucleotides in length (inclusive of all integers in between) that are substantially identical to or complementary to Xic, Xite, Tsix, or Xist and that can be used to interfere with the normal counting and pairing process and to arrest ES cell differentiation.

### Other Embodiments

All publications, patent applications, and patents, mentioned in this specification, and including U.S. Provisional Application Ser. No. 60/697,301 filed on Jul. 7, 2005, are incorporated herein by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

What is claimed is:

1. A method for delaying differentiation of a stem cell, said method comprising introducing into said stem cell a transgene selected from the group consisting of an Xic transgene, a Tsix transgene, an Xite transgene, a Tsix/Xite transgene, and fragments thereof.

2. The method of claim 1, wherein said Xic transgene comprises a nucleic acid sequence substantially identical to a sequence selected from the group consisting of SEQ ID NOs: 1, 2, and 3.

3. The method of claim 1, wherein said Xic transgene comprises a nucleic acid sequence substantially identical to the human Xic sequence set forth in SEQ ID NO: 39.

4. The method of claim 1, wherein said Tsix transgene comprises a nucleic acid sequence substantially identical to a sequence selected from the group consisting of SEQ ID NOs: 5, 6, 9, 10, 12, 13, 14, 21, 22, 23, and 28-32.

5. The method of claim 4, wherein said Tsix transgene comprises the nucleic acid sequence of SEQ ID NOs: 9, 10, 12, 21, 22, or 28-32.

6. The method of claim 4, wherein said Tsix transgene comprises at least one copy of the sequence set forth in SEQ ID NOs: 13, 14, or 28-32.

7. The method of claim 6, wherein said Tsix transgene comprises at least two copies of the sequence set forth in SEQ ID NOs: 13, 14, or 28-32.

8. The method of claim 1, wherein said Tsix transgene comprises a nucleic acid sequence substantially identical to the human Tsix sequences set forth in SEQ ID NO: 36 or 40.

9. The method of claim 8, wherein said Tsix transgene comprises at least two copies of the sequence set forth in SEQ ID NO: 40.

10. The method of claim 1, wherein said Xite transgene comprises a nucleic acid sequence substantially identical to a sequence selected from the group consisting of SEQ ID NOs: 15, 16, 17, 24, 25, 26, and 27.

11. The method of claim 1, wherein said Xite transgene comprises a nucleic acid sequence substantially identical to the human Xite sequence set forth in SEQ ID NO: 38.

12. The method of claim 1, wherein said Tsix/Xite transgene comprises a nucleic acid sequence substantially identical to a sequence selected from the group consisting of SEQ ID NOs: 4, 11, and 19.

13. The method of claim 1, wherein said Tsix/Xite transgene comprises a nucleic acid sequence substantially identical to the human Tsix/Xite sequence set forth in SEQ ID NO: 37.

14. The method of claim 1, wherein said transgene can block endogenous X-X pairing.

15. The method of claim 1, wherein said stem cell is an embryonic stem cell.

16. The method of claim 15, wherein said embryonic stem cell is female or male.

17. The method of claim 15, wherein said embryonic stem cell is mammalian.

18. The method of claim 17, wherein said embryonic stem cell is human or mouse.

19. (canceled)

20. The method of claim 15, wherein said embryonic stem cell is a blastocyst stage stem cell, an embryonic germ cell, or a cloned stem cell from a somatic nuclei.

21. The method of claim 15, wherein said embryonic stem cell is from an agricultural animal.

22. A method for delaying differentiation of a stem cell, said method comprising introducing into said stem cell a small RNA substantially identical to or complementary to at least 15 nucleotides of a transgene selected from the group consisting of an Xic transgene, a Tsix transgene, an Xite transgene, a Tsix/Xite transgene, an Xist transgene, and fragments thereof.

23-31. (canceled)

32. A method of controlling differentiation of a stem cell, said method comprising
(a) introducing into said stem cell at least one transgene selected from the group consisting of an Xic transgene, a Tsix transgene, an Xite transgene, a Tsix/Xite transgene, and fragments thereof, thereby delaying differentiation of said stem cell; and
(b) when desired, inactivating the transgene, thereby allowing differentiation of said stem cell.

33-44. (canceled)

45. The method of claim 32, wherein the transgene further comprises a selectable marker.

46. The method of claim 32, wherein the transgene is flanked by recombinase recognition sequences.

47. (canceled)

48. The method of claim 32, wherein said inactivating comprises removing said transgene from said stem cell.

49. The method of claim 48, wherein said transgene is removed from said stem cell by expression in said stem cell of a recombinase.

50-52. (canceled)

53. The method of claim 32, wherein said stem cell is an embryonic stem cell.

54-59. (canceled)

60. A stem cell comprising one or more of the following: an Xic transgene substantially identical to a sequence selected from the group consisting of SEQ ID NOs: 1, 2, 3, 39, and fragments thereof; a Tsix transgene substantially identical to a sequence selected from the group consisting of SEQ ID NOs: 5, 6, 9, 10, 12, 13, 14, 21, 22, 23, 28-32, 36, 40, and fragments thereof; an Xite transgene substantially identical to a sequence selected from the group consisting of SEQ ID NOs: 15, 16, 17, 24, 25, 26, 27, 38, and fragments thereof; and Tsix/Xite transgene substantially identical to a sequence selected from the group consisting of SEQ ID NOs: 4, 11, 19, 37, and fragments thereof.

61-75. (canceled)

76. A isolated small RNA molecule comprising a nucleic acid sequence substantially identical to or complementary to at least 15 nucleotides of a transgene selected from the group consisting of an Xic transgene, a Tsix transgene, an Xite transgene, a Tsix/Xite transgene, an Xist transgene, and fragments thereof.

77-85. (canceled)

86. A composition comprising the isolated small RNA molecule of claim 76, formulated to facilitate entry of the small RNA molecule into a cell.

87. A pharmaceutical composition comprising the isolated small RNA molecule of claim 76.

88. A vector comprising the isolated small RNA of claim 76, wherein said small RNA is operably linked to one or more transcriptional regulatory sequences.
